(19) United States
(12) Patent Application Publication
(10) Pub. No.: US 2024/0002936 A1
(43) Pub. Date: Jan. 4, 2024

(54) USE OF BRAIN ORGANOIDS AND SINGLE CELL GENOMICS TO UNDERSTAND AND TREAT NEURODEVELOPMENTAL AND NEUROPSYCHIATRIC DISORDERS

(71) Applicants: Paola ARLOTTA, Cambridge, MA (US); Aviv REGEV, Hillsborough, CA (US); President and Fellows of Harvard College, Cambridge, MA (US); The Broad Institute, Inc., Cambridge, MA (US); Massachusetts Institute of Technology, Cambridge, MA (US)

(72) Inventors: Paola Arlotta, Cambridge, MA (US); Joshua Z. Levin, Sharon, MA (US); Amanda J. Kedaigle, Arlington, MA (US); Aviv Regev, Hillsborough, CA (US); Bruna Da Silveira Paulsen, Cambridge, MA (US); Silvia Velasco, Carlton (AU); Martina Pigoni, Cambridge, MA (US)

(21) Appl. No.: 18/034,876

(22) PCT Filed: Nov. 1, 2021
(86) PCT No.: PCT/US2021/057632

§ 371 Date: May 1, 2023

Related U.S. Application Data
(60) Provisional application No. 63/108,246, filed on Oct. 30, 2020.

(51) Int. Cl. C12Q 1/6883; G01N 33/50

(52) U.S. Cl. CPC C12Q 1/6883; G01N 33/5088; C12Q 2600/156

(57) ABSTRACT

The present disclosure is directed to methods of screening for pathological mechanisms in neurodevelopmental and neuropsychological disorders using brain organoids. The present disclosure is also directed to methods of screening agents using the brain organoids.

### RELATED APPLICATION(S)

This application claims the benefit of U.S. Provisional Application No. 63/108,246, filed on Oct. 30, 2020, the contents of which are hereby incorporated by reference in their entirety.

### BACKGROUND OF THE INVENTION

Autism spectrum disorder (ASD) is a childhood-onset neurodevelopmental disorder characterized by cognitive, motor, and sensory deficits. ASD has a strong genetic component, with risk contribution from hundreds of genes. Individuals bearing mutations in the same gene can also present differing severity of clinical manifestations, likely reflecting a modulatory effect of the overall genomic context in which risk genes act. The shared developmental effects that cause this large and heterogeneous collection of genes to converge on the phenotypic features of ASD, whether they act through shared or distinct pathways, and how they may be influenced by genetic and epigenetic context, remain poorly understood.

These questions have been difficult to address in part due to the lack of experimental models of human brain development, a process that largely occurs in utero and is therefore poorly accessible for study. The emergence of human brain organoids represents a major advance in modeling the cellular complexity of the developing human brain in vitro. In combination with single-cell profiling methods, these models have the potential to enable the unbiased identification of the cell types, gene programs, developmental events, and molecular mechanisms that are affected by ASD risk-associated genes, across a diversity of human genomic contexts.

### SUMMARY OF THE INVENTION

Genetic risk for autism spectrum disorder (ASD) has been associated with hundreds of genes spanning a wide range of biological functions. The phenotypic alterations in the human brain resulting from mutations in ASD risk genes remain unclear, and the level at which these alterations converge on shared disease pathology is poorly understood. Furthermore, the phenotypic manifestation of mutations in disease-associated genes varies across individuals. As described herein, reproducible organoid models of the human cerebral cortex were leveraged to identify cell type-specific developmental abnormalities associated with haploinsufficiency in ASD risk genes, SUV420H1 (KMT5B), ARID1B, PTEN, and CHD8, in multiple cell lines from different donors. Comprehensive single-cell RNA-seq (scRNA-seq) was performed, and proteomic analysis was performed on individual organoids sampled at different developmental stages to identify phenotypic convergence in the effects of these genes.

Within a defined period of early cortical development, the mutations demonstrate asynchronous development of two of the main cortical neuronal lineages, GABAergic neurons and deep layer excitatory projection neurons, but these defects occurred through largely distinct molecular pathways. Although this effect is consistent across cell lines, these phenotypes are influenced to different degrees by the broader genomic context. Calcium imaging in intact organoids shows that these early-stage developmental changes may be related to later abnormal circuit activity, consistent with differential accessibility by single-cell ATAC-seq (scATAC-seq) of regions near genes associated with synaptic function. This work uncovers cell type-specific neurodevelopmental abnormalities shared across ASD risk genes that are finely modulated by human genomic context, revealing convergence in the neurobiological basis of how different risk genes contribute to ASD pathology.

Disclosed herein are methods of screening for pathological mechanisms in neurodevelopmental disorders. The methods may include introducing one or more genetic variations to an organoid; and profiling the mutant organoids using single-cell RNA sequencing, thereby identifying phenotypic alterations as a result of the genetic variation.

In some embodiments, the genetic variation causes haploinsufficiency in a gene. In some embodiments, the gene is selected from the group consisting of SUV420H1, PTEN, ARID1B and CHD8, and in certain embodiments is selected from the group consisting of SUV420H1, ARID1B and CHD8.

In some embodiments, the neurodevelopmental disorder is autism spectrum disorder (ASD).

In some embodiments, the mutant organoids are screened one month, three months, and/or six months after introduction of the genetic variation. In some embodiments, the mutant organoids are further profiled using immunohistochemistry, whole-proteome analysis, single cell Assay for Transposase-Accessible Chromatin sequencing (ATAC-seq), and/or electrophysiological analysis.

In some embodiments, the phenotypic alternation comprises asynchronous development of a cortical neuronal lineage, e.g., asynchronous development of deep layer projection neuron lineage. In some embodiments the phenotypic alteration comprises premature expansion of GABAergic neuron lineage.

Also disclosed herein are methods of screening a therapeutic agent. The methods of screening the therapeutic agent may include contacting an organoid having one or more genetic variations with one or more candidate agents; and testing effects of the one or more candidate agents on asynchronous development of a cortical neuronal lineage.

In some embodiments, the asynchronous development of a cortical neuronal lineage comprises asynchronous development of deep layer projection neuron lineage, e.g., the asynchronous development of a cortical neuronal lineage comprises premature expansion of GABAergic neuron lineage. In some embodiments, the genetic variation causes haploinsufficiency in a gene. In some embodiments, the gene is selected from the group consisting of SUV420H1, ARID1B and CHD8.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1F demonstrate a developmental atlas of cells in human brain organoids shows expected enrichment of ASD risk genes. FIGS. 1A-1C show organoids cultured for one, three and six months in vitro. SOX2, a marker of neural progenitors and TBR1, a neuronal marker, are shown at one month (FIG. 1A). SATB2, a marker of callosal projection neurons, and CTIP2, a marker of corticofugal projection neurons, are shown at three months (FIG. 1B). The astroglia markers S100B and GFAP are shown at six months (FIG. 1C). Scale bars are 100 μm. FIG. 1D provides t-distributed stochastic neighbor embedding (t-SNE) plots of 58,568 cells from 9 organoids from the GM08330 cell line, at one, three and six months, after Harmony batch correction. Cells are colored according to cell type (left) and timepoint (right). FIG. 1E shows gene set expression scores for a set of 102 genes associated with ASD risk⁸ across cell types, in cells from FIG. 1D. Scores above 0 indicate enriched expression over similar sets of randomly chosen genes. FIG. 1F provides t-SNE plots showing normalized expression of SUV420H1, PTEN, and CHD8, the three genes analyzed in this article, across the cells in FIG. 1D. PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells; IN interneurons.

FIGS. 2A-2F demonstrate SUV420H1^{+/-} mutant organoids show an increased number of deep layer projection neurons in one-month organoids. FIG. 2A provides optical clearing and light-sheet microscopy of a control organoid and a SUV420H1^{+/-} organoid cultured for 35 d.i.v. Scale bars are 500 um. SOX2, a marker of neuronal progenitors, is shown in red, and nuclei are shown in blue. FIG. 2B shows quantification of the size of control and SUV420H1^{+/-} organoids at 18 and 27 d.i.v. The ratio of organoid area compared to the average of control organoids in each batch is plotted. n=42 18 d.i.v. control organoids, 42 18 d.i.v. mutant organoids, 47 27-d.i.v. control organoids, and 46 27-d.i.v. mutant organoids, from 4 differentiation batches. P-values from a two-sided t test. FIGS. 2C, 2E provide scRNA-seq data from 28 d.i.v. (FIG. 2C) and 35 d.i.v. (FIG. 2E) control and SUV420H1^{+/-} mutant organoids. Upper left shows combined t-SNE plots of control and mutant organoids (n=3 single organoids per genotype). Cells are colored by cell type, and the number of cells per plot is indicated. Right, t-SNE plots for control and mutant individual organoids. Deep layer neuron populations are highlighted in color. Lower left, barcharts showing the percentage of cells for the highlighted cell populations in each control and mutant organoid. Adjusted p values for a difference in cell type proportions between control and mutant, based on logistic mixed models (see Methods) are shown. FIG. 2D shows volcano plot showing fold change versus p-value of measured proteins in MS experiments on SUV420H1^{+/-} versus control organoids cultured for 35 days (n=4 single organoids per genotype). Significant DEPs are shown in red (adjusted p<0.1). Proteins mentioned in the text are highlighted. FIG. 2F show scRNA-seq data from three month control and SUV420H1^{+/-} mutant organoids, as in FIG. 2C. d.i.v, days in vitro; PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells. DEPs: differentially expressed proteins; MS: mass spectrometry.

FIGS. 3A-3I demonstrate PTEN^{+/-} and CHD8^{+/-} show an increased number of callosal projection neurons and interneurons, respectively. FIG. 3A provides quantification of the size of control and PTEN^{+/-} organoids at 18 and 35 d.i.v. The ratio of organoid area compared to the average of control organoids in each batch is plotted. n=114 18-d.i.v. control organoids, 77 18-d.i.v. mutant organoids, 39 35-d.i.v. control organoids, and 34 35-d.i.v. mutant organoids, from 3 differentiation batches. P-values from a two-sided t test. FIG. 3B provides volcano plot showing fold change versus p-value of measured proteins in MS experiments on PTEN^{+/-} versus control organoids cultured for 35 days (n=4 single organoids per genotype). Significant DEPs are shown in red (adjusted p<0.1). Proteins mentioned in the text are highlighted. FIG. 3C shows a comparison of the level of protein expression change between 35 d.i.v. and 70 d.i.v. for control vs. mutant organoids. Gray lines connect values for the same protein in the two genotypes. P value from a paired signed Wilcoxon rank test. Only significant DEPs are shown; for the same analysis done with all detected proteins see FIG. 8H. FIG. 3D shows a comparison of protein expression changes in PTEN^{+/-} vs. control organoids at 35 d.i.v. (bottom) to changes in 35 d.i.v. vs. 70 d.i.v. control organoids (top). Color and y-axis indicate log 2 fold change. Only proteins with significantly differential expression between control and mutant at 35 d.i.v. are shown. (n=3 single organoids per genotype have been used at 70 d.i.v). FIG. 3E shows scRNA-seq data from PTEN^{+/-} and control organoids cultured for three months. Upper left shows combined t-SNE plots of control and mutant organoids (n=3 single organoids per genotype). Cells are colored by cell type and the number of cells per plot is indicated. Right, t-SNE plots for control and mutant individual organoids. Callosal projection neuron populations are highlighted in color. Lower left, barcharts showing the percentage of callosal projection neurons in each control and mutant organoid. Adjusted p values for a difference in cell type proportions between control and mutant, based on logistic mixed models (see Methods) are shown. FIG. 3F provides quantification of the size of control and CHD8^{+/-} organoids at 16 and 25 d.i.v. The ratio of organoid area compared to the average of control organoids in each batch is plotted. n=19 16 d.i.v. control organoids, 19 16 d.i.v. mutant organoids, 16 25 d.i.v. control organoids, and 17 25 d.i.v. mutant organoids, from 2 differentiation batches. P-values from two-sided t test. FIG. 3G provides volcano plot showing fold change versus p-value of measured proteins in MS experiments on CHD8^{+/-} versus control organoids cultured for 35 days (n=3 single organoids per genotype). Significantly DEPs are shown in red (adjusted p<0.1). FIGS. 3H-3I provide scRNA-seq data from CHD8^{+/-} and control organoids cultures for 3.5 months, from differentiation batch I (FIG. 3H) and II (FIG. 3I). Upper left shows combined t-SNE plots of control and mutant organoids (n=3 single organoids per genotype, except batch II mutant for which n=2, see Methods). Cells are colored by cell type and the number of cells per plot is indicated. Right, t-SNE plots for control and mutant individual organoids. Interneurons and their progenitor populations are highlighted in color. Lower left, barcharts showing the percentage of interneurons and their progenitors in each control and mutant organoid. Adjusted p values for a difference in cell type proportions between control and mutant, based on logistic mixed models (see Methods) are shown. d.i.v, days in vitro; PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells, IN: interneurons. DEPs: differentially expressed proteins; MS: mass spectrometry.

FIGS. 4A-4K demonstrate mutant organoids show accelerated differentiation of specific neuronal cell types. FIGS. 4A-4F provide pseudotime UMAPs for the 35 d.i.v. SUV420H1^{+/-} (FIGS. 4A-4B) and 3 month PTEN^{+/-} (FIGS. 4C-4D) and 3.5 month CHD8^{+/-} (FIGS. 4E-4F) organoids, as well as their matching controls. Cells are colored according to cell type (FIGS. 4A, 4C, 4E), pseudotime blue (early) to yellow (late) (FIGS. 4B, 4D, 4F left), and genotype (control, blue; mutant, red) (FIGS. 4B, 4D, 4F right). n=3 single organoids per batch per condition. Cells were downsampled to an equal number between control and mutant. For SUV420H1 and CHD8, one partition is shown, the part of the trajectories leading to the cell types previously shown to be increased in each mutant. For full trajectories, see FIG. 10A and FIGS. 11A-11B. all cells are shown for PTEN. FIGS. 4G-4I show modules of highly correlated genes identified through co-expression network analysis on cells within the partitions of interest. Examples of modules that are differentially expressed between control and mutant organoids are shown; names were assigned to each module based on the genes included. Left, module scores per cell are shown on associated UMAPs. Right, violin plots show the distribution of module scores between controls and mutants. Horizontal bars show median scores, and dots show average score per organoid. Cells were downsampled to give an equal number in each organoid. P-values show differences between control and mutant based on linear mixed models (see Methods). Additional modules are shown in FIGS. 10-11. FIG. 4J shows enriched GO terms for genes upregulated in mutant vs. control. Genes were calculated using cells from the partitions of interest (i.e., the cells shown in FIGS. 4G-4I). Size of dot indicates the proportion of genes belonging to each term found in the list of upregulated genes. Color indicates enrichment adjusted p-value. FIG. 4K shows gene expression changes of all genes significantly changing (adjusted p<0.05) in the same direction in SUV420H1^{+/+} 35 d.i.v. and in CHD8^{+/-} 3.5 month cells shown in FIGS. 4G, 4I. Genes marked in bold with an asterisk were also trending towards upregulation in PTEN^{+/-} three month callosal projection neuron lineage cells (adjusted p<0.2). Color and bar size indicates log 2 fold change. PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells, IN: interneurons.; padj: adjusted p-value.

FIGS. 5A-5C demonstrate cortical organoids cultured for one, three and six months generate the cellular diversity of the human cerebral cortex with high organoid-to-organoid reproducibility. FIG. 5A shows immunohistochemistry for neuronal (MAP2), dorsal forebrain neural progenitors (EMX1, SOX2), CFuPN (CTIP2), and CPN (SATB2) markers in GM08330 organoids at one, three and six months. Scale bars: whole organoids, 200 μm; others, 50 μm. FIG. 5B provides t-SNE plots of scRNA-seq data from individual one, three, and six months organoids from the GM08330 cell line. Top, t-SNE plots for the three organoids from each age after Harmony batch correction, colored by cell types, repeated from FIGS. 1A-1C for ease of comparison. FIG. 5C shows expression of selected marker genes used in cell-type identification. Violin plots show distribution of normalized expression in cells from organoids at one, three and six months (n=3 individual organoids per time point). PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells, IN: interneurons.

FIGS. 6A-6F demonstrate expression of selected ASD risk genes in cortical organoids cultured for one, three, and six months from two cell lines. FIG. 6A provides t-SNE plots of 58, 568 cells from 9 organoids from the GM08330 cell line, repeated from FIG. 1D. Cells are colored according to cell type (left) or time point (right). FIG. 6B shows gene set scores for a set of 102 genes associated with ASD risk⁸ across cell types, in cells from FIG. 6A. Repeated from FIG. 1E for ease of comparison. Scores above 0 indicate higher expression than similar modules of randomly chosen genes. FIG. 6C provides t-SNE plots showing normalized expression of selected ASD risk genes in cells from FIG. 6A. FIG. 6D provides t-SNE plots of 9 organoids from the Mito210 cell line, at one, three and six months, after Harmony batch correction. Cells are colored according to cell type (left) or timepoint (right). FIG. 6E shows gene set scores for the set of ASD risk genes as in FIG. 6B, in cells from FIG. 6D. Scores above 0 indicate higher expression than similar modules of randomly chosen genes. FIG. 6F provides t-SNE plots showing normalized expression of selected ASD risk genes in cells from FIG. 6D. PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells, IN: interneurons.

FIGS. 7A-7E demonstrate validation of SUV420H1^{+/-} edited and isogenic control cell lines and generation of brain organoids. FIG. 7A shows protein domain structure of SUV420H1. Bars indicate most common mutations in ASD patients. In this study, the Mito210 iPSC line was edited to induce a mutation in the N-domain (dashed bar) that resulted in a protein-truncating variant. FIG. 7B provides a Western blot analysis showing presence of H4K20me3, a target of SUV420H1 activity, in control and high reduction in SUV420H1^{+/-} mutant organoids, using an antibody directed against this modification. FIG. 7C shows immunohistochemistry for neuronal (MAP2), dorsal forebrain neural progenitors (EMX1, SOX2) and CFuPN (CTIP2) markers in one month organoids derived from the Mito210 SUV420H1^{+/-} mutant and isogenic control cell lines. Scale bar, 100 μm. FIG. 7D shows fraction of cells belonging to each cell type in individual organoids at 28 d.i.v., 35 d.i.v., and three months. P-values show significance of change between mutant and control organoids, based on logistic mixed models (see Methods). FIG. 7E provides enriched GO terms for DEPs between SUV420H1^{+/-} and control organoids cultured for 35 d.i.v. N-domain: N-terminal domain; H4K20me3, histone 4 lysine 20 trimethylation; d.i.v, days in vitro; PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cells; DEPs: differentially expressed proteins.

FIGS. 8A-8I demonstrate validation of PTEN^{+/-} edited and isogenic control cell lines and generation of cortical organoids. FIG. 8A shows protein domain structure of PTEN. Bars indicate most common mutations in ASD patients. In this study, the Mito210 iPSC line was edited to induce a mutation in the phosphatase domain (dashed bar) that resulted in a protein-truncating variant. FIG. 8B provides a Western blot analysis showing presence of PTEN and its targets in control, and reduction of PTEN along with increase in phosphorylation of PTEN targets in mutant organoids, using an antibody directed against these modifications. FIG. 8C shows immunohistochemistry for neuronal (MAP2), dorsal forebrain neural progenitors (EMX1, SOX2), and CFuPN (CTIP2) markers in one month organoids derived from the Mito210 PTEN mutant and isogenic control cell lines. Scale bar, 100 μm. FIGS. 8D-8E show scRNA-seq data from PTEN^{+/-} and control organoids cultured for 35 d.i.v., batch I (FIG. 8D) and II (FIG. 8E). Top, Combined t-SNE plots of control and mutant organoids (n=2 control and 3 mutant individual organoids). Cells are colored by cell type and the number of cells per plot is indicated. Right, t-SNE plots split into control and mutant organoids. Newborn deep layer projection neuron populations are highlighted in color. Bottom, barcharts showing the percentage of highlighted populations in each control and mutant organoid. Adjusted p values for a difference in cell type proportions between control and mutant, based on logistic mixed models (see Methods) are shown. FIG. 8F provides the fraction of cells belonging to each cell type in individual organoids after one month (top and middle) and three months (bottom) in vitro. P-values show significance of change between mutant and control organoids, based on logistic mixed models (see Methods). FIG. 8G provides enriched GO terms for DEPs between PTEN^{+/-} and control organoids cultured for 35 d.i.v. d.i.v.: days in vitro. FIG. 8H shows comparison of the level of protein expression change between 35 d.i.v. and 70 d.i.v. for control vs. mutant organoids, for all detected proteins. P value from a paired signed Wilcoxon rank test. FIG. 8I provides a correlation between protein expression differences between PTEN^{+/-} and control organoids at 35 d.i.v., and between control organoids at 35 d.i.v. and 70 d.i.v. Correlation is shown for proteins that significantly change between mutant and control organoids at 35 d.i.v. (middle), those that did not (right), and a comparison of their respective Pearson correlation coefficients (left). PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cell; DEPs: differentially expressed proteins.

FIGS. 9A-9E demonstrate validation of CHD8^{+/-} edited and isogenic control cell lines and generation of brain organoids. FIG. 9A shows protein domain structure of CHD8. Bars indicate most common mutations in ASD patients. The HUES66 line used in this study carries a mutation in the helicase-C domain (HELC domain, dashed bar) that resulted in a protein-truncating variant. FIG. 9B provides a Western blot analysis showing presence of CHD8 in control, and reduction in mutant brain organoids, using an antibody directed against this protein. FIG. 9C shows immunohistochemistry for neuronal (MAP2), dorsal forebrain progenitor (EMX1, SOX2), and CFuPN (CTIP2) markers in one month organoids derived from the HUESS66 CHD8 mutant and isogenic control cell lines. Scale bar, 100 μm. FIG. 9D provides enriched gene ontology terms for DEPs differentially expressed proteins between CHD8^{+/-} and control organoids cultured for 35 d.i.v. FIG. 9E shows fraction of cells belonging to each cell type in individual organoids. P-values show significance of change between mutant and control organoids, based on logistic mixed models (see Methods). d.i.v.: days in vitro; PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cell; IN, interneurons. DEPs: differentially expressed proteins.

FIGS. 10A-10B demonstrates full pseudotime trajectories and gene modules in SUV420H1^{+/-} and CHD8^{+/-} and their corresponding control organoids. FIG. 10A provides pseudotime trajectories from full datasets of SUV420H1^{+/-} 35 d.i.v. and control organoids, calculated with Monocle3. Partition highlighted in box was subsetted and the trajectory is shown in FIGS. 4A-4B. FIG. 10B provides module scores (top) and their distribution across mutant and control cells (bottom) for all modules resulting from WGCNA analysis of the partition of interest from SUV420H1^{+/-} and control organoids at 35 d.i.v. Cells were downsampled to have an equal number of cells per organoid. Names were assigned to each module based on the known functions of the genes included in each one. Horizontal bars show median scores, and dots show average score per organoid. P-values show differences between control and mutant based on linear mixed models (see Methods). d.i.v.: days in vitro; PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cell.

FIGS. 11A-11E demonstrate full gene modules in PTEN^{+/-} and CHD8^{+/-} and control organoids. FIGS. 11A-11B show pseudotime trajectories from full datasets of PTEN^{+/-} 3 months (FIG. 11A) and CHD8^{+/-} 3.5 months in vitro (FIG. 11B) and their control organoids, calculated with Monocle3. Partition highlighted in box (left box of FIG. 11A) was subsetted and the trajectory is shown in FIG. 4E. FIGS. 11C-11E provide module scores (top) and their distribution across mutant and control cells (bottom) for all modules resulting from WGCNA analysis of the partitions of interest. Cells were downsampled to have an equal number of cells per organoid. Names were assigned to each module based on the known functions of the genes included in each one. Horizontal bars show median scores, and dots show average score per organoid. P-values show differences between control and mutant based on linear mixed models (see Methods). d.i.v.: days in vitro; PN, projection neurons; aRG, apical radial glia; UL CPN, upper layer callosal projection neurons; oRG, outer radial glia; DL CFuPN, deep layer corticofugal projection neurons; DL, deep layers; IPC, intermediate progenitor cell; IN, interneurons.

FIGS. 12A-12C demonstrate biological processes affected in proteomics and scRNA-seq from the three mutations. FIG. 12A provides enriched GO terms for genes downregulated in mutant vs. control cells in each of FIGS. 4G-4I. Size of dot indicates the proportion of genes belonging to each term found in the list of upregulated genes. Color indicates adjusted enrichment p-value. FIG. 12B shows protein-protein interaction network using DEPs from all three sets of mutant organoids, created using the prize-collecting Steiner forest algorithm (see Methods). Protein nodes are colored by the mutant(s) that they were differentially expressed in. Lines between nodes indicate physical protein-protein interactions from the STRING database, where line thickness correlates with interaction confidence. Selected subclusters of the network and significantly enriched protein function terms for those subclusters are highlighted with gray rectangles and black text. FIG. 12C shows protein set distances between pairs of differentially expressed protein sets. For each pair of mutations, a PPI-weighted protein set distance⁵⁰ was calculated between DEPs—(pink diamond). In order to determine if this distance was smaller than would be expected by chance, size-matched sets were randomly chosen from the proteins detected in each experiment, and distance between these random sets was calculated 1000 times per pair. P-values were assigned by counting the number of times this random distance was less than the actual distance value between differential sets. DEPs: differentially expressed proteins.

FIGS. 13-28 re-present certain data from FIGS. 1-12 and provide additional data.

FIGS. 13A-13I demonstrate SUV420H1 haploinsufficiency induces asynchronous generation of both GABAergic neurons and deep layer excitatory neurons. FIG. 13A shows immunohistochemistry of Mito210 SUV420H1^{+/-} mutant and control organoids cultured for one month (35 d.i.v.). Optical section from the middle of whole-organoid dataset. Scale bars are 500 μm. SOX2, a marker of neuronal progenitors, is shown in red, and nuclei (Syto16) are shown in blue. FIG. 13B provides quantification of the size of control and SUV420H1^{+/-} organoids across lines at two weeks (17-18 d.i.v.) and one month (27-30 d.i.v.) in culture. The ratio of organoid size compared to the average of control organoids in each batch is plotted. Differentiation batch is indicated by the shade of the points. P-values from a two-sided t-test after Bonferroni adjustment. FIG. 13C shows immunohistochemistry for the post mitotic excitatory neuronal marker TBR1 and GABAergic marker DLX2 in Mito294 control and SUV420H1^{+/-} organoids at one month (35 d.i.v.). Scale bars: 200 μm. FIGS. 13D-13F provide scRNA-seq data from Mito294 35 d.i.v. (FIG. 13D), PGP1 35 d.i.v. (FIG. 13E), and Mito210 (batch I, see FIG. 21D for batch II) 28 d.i.v. (FIG. 13F) control and SUV420H1^{+/-} mutant organoids. Upper left shows combined t-SNE plots of control and mutant organoids (n=3 single organoids per genotype, n=2 for PGP1 SUV420H1^{+/-} mutant). Cells are colored by cell type, and the number of cells per plot is indicated. Upper right, bar charts showing the percentage of cells for the highlighted cell populations in each control and mutant organoid. Adjusted p-values for a difference in cell type proportions between control and mutant, based on logistic mixed models (see Methods) are shown. Below, t-SNE plots for control and mutant individual organoids. GABAergic neurons and immature deep layer projection neuron populations are highlighted in color. FIG. 13G provides scRNA-seq data from three month Mito294 89 d.i.v. control and SUV420H1^{+/-} mutant organoids, as in FIGS. 13D-13F. In individual organoid t-SNEs, GABAergic neurons, interneurons and their progenitors are highlighted in color. FIG. 13H shows pseudotime UMAPs for the 35 d.i.v. Mito210 SUV420H1^{+/-} (batch II) organoids, and their corresponding controls. Cells are colored according to cell type (left), pseudotime (blue, early to yellow, late; middle left), and genotype (control, blue; mutant, red; right). Middle left, the distribution of cells belonging to each genotype along the pseudotime trajectory. Cells were downsampled to an equal number between control and mutant. Insets show the cells belonging to the end of the trajectory from each genotype and correspond to the parts of the trajectory outlined by gray dotted boxes. n=3 single organoids per genotype. The part of the trajectory leading to immature deep layer projection neuron is shown. For full trajectory, see FIG. 22D. FIG. 13I shows a module of highly correlated genes identified through co-expression network analysis on the cells in FIG. 13G. Left, module scores per cell are shown on the associated UMAP. Right, violin plots show the distribution of module scores between controls and mutants. Horizontal bars show median scores, and dots show average score per organoid. Cells were downsampled to give an equal number in each organoid. Adjusted p-values show differences between control and mutant based on linear mixed models (see Methods). Additional modules are shown in FIG. 22E. aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons; GABA NP, GABAergic neuron progenitors; GABA N, GABAergic neurons; GABA INP, GABAergic interneuron progenitors; GABA IN, GABAergic interneurons.

FIGS. 14A-14H demonstrate SUV420H1 haploinsufficiency leads to early changes in neuronal gene regulation and later changes in circuit activity. FIG. 14A shows UMAPs of scATAC-seq data in Mito210 SUV420H1^{+/-} and control organoids at one month (31 d.i.v.) and three months (93 d.i.v.) (left), and coembedded UMAPs with scRNA-seq in Mito210 SUV420H1^{+/-} and control organoids at one month (28 d.i.v.) and three months (90 d.i.v.). FIG. 14B shows enriched gene ontology terms for the nearest genes to regions with increased and decreased accessibility in SUV420H1^{+/-} compared to control organoids. FIG. 14C provides genome tracks showing read coverage for representative regions with increased accessibility between SUV420H1^{+/-} and control organoids. Scales for the y axes (normalized counts) are displayed on the top right. FIGS. 14D-14H show calcium imaging in PGP1 SUV420H1^{+/-} and control organoids at four months (128 d.i.v.). FIG. 14D, Left, is a representative image of an organoid infected with SomaGCaMP6f2 (Scale bar: 100 μm). Insets: high-magnification images of numbered cells (Scale bar: 10 um). FIG. 14D, Right, shows individual traces of spontaneous calcium signal for each numbered cell are presented as DF/F (top) and pseudocolor heat map (bottom; see e for scale). FIG. 14E provides a heat map of calcium signal from individual cells (rows), showing the effect of 2 μM TTX. FIG. 14F shows the effect of NBQX on neuronal activity. Representative traces for individual cells were normalized (3 traces for SUV420H1^{+/-} are superimposed) and post-NBQX calcium transients are indicated by asterisks. FIG. 14G, Left, provides representative heat maps of calcium signal for each condition (control and SUV420H1^{+/-}). FIG. 14G, Right, shows spontaneous network burst frequency. Gray dots represent average values for individual organoids, and the bar shows the mean across the 5 organoids for each genotype. P-value from two-tailed t-test. FIG. 14H, Left, shows population-averaged calcium transients (top) and corresponding heat map for individual cells (bottom), displaying the difference in kinetics between control (top panel) and SUV420H1^{+/-} (bottom panel) organoids. FIG. 14H, Right, shows spontaneous network burst duration. Bar plot format as in FIG. 14G. P value from two-tailed t-test. aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons.

FIGS. 15A-15G demonstrate ARID1B haploinsufficiency induces asynchronous generation of both GABAergic neurons and deep layer projection neurons. FIG. 15A provides scRNA-seq data from Mito210 one month (35 d.i.v.) control and ARID1B^{+/-} mutant organoids, batch I. Top left shows combined t-SNE plots of control and mutant organoids (n=3 single organoids per genotype). Cells are colored by cell type, and the number of cells per plot is indicated. Top right, bar charts showing the percentage of cells for the highlighted cell populations in each control and mutant organoid. Adjusted p-values for a difference in cell type proportions between control and mutant, based on logistic mixed models (see Methods) are shown. Below, t-SNE plots for control and mutant individual organoids. GABAergic neurons and newborn deep layer projection neuron populations are highlighted in color. FIG. 15B shows immunohistochemistry for the postmitotic excitatory neuronal marker TBR1 (magenta) and GABAergic marker DLX2 (green) in Mito210 control and ARID1B^{+/-} mutant organoids at one month (35 d.i.v.). Scale bars: 200 μm. FIGS. 15C-15D provide scRNA-seq data from Mito210 one month (35 d.i.v.) control and ARID1B^{+/-} organoids, batch II (FIG. 15C), and from Mito210 three month (90 d.i.v.) control and ARID1B^{+/-} organoids (FIG. 15D), as in FIG. 15A. FIG. 15E shows immunohistochemistry for TBR1 (magenta) and DLX2 (green) in Mito210 control and ARID1B^{+/-} mutant organoids at three months (90 d.i.v.). Scale bars: 100 m. FIG. 15F provides pseudotime UMAPs for the 35 d.i.v. Mito210 ARID1B^{+/-} organoids (batch II), and their corresponding controls. Cells are colored according to cell type (left), pseudotime (blue, early to yellow, late; middle left), and genotype (control, blue; mutant, red; right). Middle left, the distribution of cells belonging to each genotype along the pseudotime trajectory. Cells were downsampled to an equal number between control and mutant. Insets show the cells belonging to the end of the trajectory from each genotype and correspond to the parts of the trajectory outlined by gray dotted boxes. n=3 single organoids per genotype. The part of the trajectory leading to immature deep layer projection neurons is shown. For full trajectory, see FIG. 25F. FIG. 15G shows a module of highly correlated genes identified through co-expression network analysis on the cells in FIG. 15F. Left, module scores per cell are shown on the associated UMAP. Right, violin plots show the distribution of module scores between controls and mutants. Horizontal bars show median scores, and dots show average score per organoid. Cells were downsampled to give an equal number in each organoid. Adjusted p-value shows the difference between control and mutant based on linear mixed models (see Methods). Additional modules are shown in FIG. 25G. aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons; GABA NP, GABAergic neuron progenitors; GABA N, GABAergic neurons; GABA INP, GABAergic interneuron progenitors; GABA IN, GABAergic interneurons.

FIGS. 16A-16G demonstrate CHD8 haploinsufficiency affects early neurodevelopment and leads to asynchronous generation of GABAergic interneurons. FIGS. 16A-16B provide scRNA-seq data from HUES66 3.5 months (109 d.i.v. (FIG. 16A) and 107 d.i.v. (FIG. 16B)) control and CHD8^{+/-} mutant organoids. Top left shows combined t-SNE plots of control and mutant organoids (n=3 or 2 single organoids per genotype). Cells are colored by cell type, and the number of cells per plot is indicated. Top right, bar charts showing the percentage of cells for the highlighted cell populations in each control and mutant organoid. Adjusted p-values for a difference in cell type proportions between control and mutant, based on logistic mixed models (see Methods) are shown. Bottom, t-SNE plots for control and mutant individual organoids. GABAergic interneurons and progenitors are highlighted in color. FIG. 16C shows immunohistochemistry for the postmitotic excitatory neuronal marker TBR1 (magenta) and GABAergic marker DLX2 (green) in HUES66 control and CHD8^{+/-} mutant organoids at 3.5 months (107 d.i.v.). Scale bars: 100 μm. FIG. 16D provides scRNA-seq data from HUES66 control and CHD8^{+/-} organoids at six months (190 d.i.v.), as in FIG. 16A. FIG. 16E shows immunohistochemistry for the post mitotic neuronal marker TBR1 (magenta) and GABAergic marker DLX2 (green) in HUES66 control and CHD8^{+/-} mutant organoids at six months (190 d.i.v.). Scale bars: 100 μm. FIG. 16F shows pseudotime UMAPs for the 109 d.i.v. HUES66 CHD8^{+/-} organoids, and their corresponding controls. Cells are colored according to cell type (left), pseudotime (blue, early to yellow, late; middle left), and genotype (control, blue; mutant, red; right). Middle left, the distribution of cells belonging to each genotype along the pseudotime trajectory. Cells were downsampled to an equal number between control and mutant. Insets show the cells belonging to the end of the trajectory from each genotype and correspond to the parts of the trajectory outlined by gray dotted boxes. n=3 single organoids per genotype. The part of the trajectory leading to GABAergic interneurons is shown. For full trajectory, see FIG. 26E. FIG. 16G provides a module of highly correlated genes identified through co-expression network analysis on the cells in FIG. 16F. Left, module scores per cell are shown on the associated UMAP. Right, violin plots show the distribution of module scores between controls and mutants. Horizontal bars show median scores, and dots show average score per organoid. Cells were downsampled to give an equal number in each organoid. Adjusted p-values show differences between control and mutant based on linear mixed models (see Methods). Additional modules are shown in FIG. 26F. aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons; GABA INP, GABAergic interneuron progenitors; GABA IN, GABAergic interneurons; GABA N, GABAergic neurons.

FIGS. 17A-17D demonstrate SUV420H1, ARID1B, and CHD8 haploinsufficiency affect similar developmental processes through distinct gene and protein targets. FIG. 17A shows log fold change of all genes which showed significant change (adjusted p<0.05) in all three of the 1 month datasets: Mito210 SUV420H1^{+/-} 35 d.i.v., Mito210 ARID1B^{+/-} 35 d.i.v., and HUES66 CHD8^{+/-} 35 d.i.v. Differentially expressed genes were calculated using all cells as a pseudobulk for Mito210 SUV420H1^{+/-} and Mito210ARID1B^{+/-}. FIG. 17B shows overlaps between differentially expressed genes in each cell type in the Mito210 SUV420H1^{+/-} 35 d.i.v., Mito210ARID1B^{+/-} 35 d.i.v. datasets (left), and the Mito210 SUV420H1^{+/-} 92 d.i.v., Mito210 ARID1B^{+/-} 90 d.i.v., and HUES66 CHD8^{+/-} 109 d.i.v. datasets (right). For each comparison of two gene lists, circles inside the box are colored and sized according to the significance of the number of overlapping genes in those two lists, reported as the Bonferroni-adjusted p value of a hypergeometric test. FIG. 17C provides proteomics data from Mito210 SUV420H1^{+/-}, Mito210 ARID1B^{+/-} and HUES66 CHD8^{+/-} 35 d.i.v. organoids. Protein-protein interaction network using the top 50 DEPs from all three sets of mutant organoids, created using the prize-collecting Steiner forest algorithm (see Methods). Protein nodes are colored by the mutant in which they were differentially expressed. Gray nodes indicate “Steiner nodes”, proteins that did not result from any screen but were included by the algorithm to connect DEPs. Lines between nodes indicate physical protein-protein interactions from the STRING database, where line thickness correlates with interaction confidence. Subclusters of the network and significantly enriched terms for those subclusters are highlighted with gray rectangles and black text. FIG. 17D provides protein set distances between pairs of differentially expressed protein sets. For each pair of mutations, a PPI-weighted protein set distance was calculated between all significant DEPs (FDR <0.1, pink diamond). To determine if this distance was smaller than would be expected by chance, size-matched sets were randomly chosen from the proteins detected in each experiment, and distance between these random sets was calculated 1000 times per pair. P-values were assigned by counting the fraction of times this random distance was less than the actual distance value between differential sets. aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons; GABA IN, GABAergic interneurons; CP/CH, Choroid Plexus/Cortical Hem; DEPs: differentially expressed proteins.

FIGS. 18A-18G demonstrate cortical organoids cultured for one, three and six months generate the cellular diversity of the human cerebral cortex with high organoid-to-organoid reproducibility. FIG. 18A provides scRNA-seq and immunohistochemistry analysis of GM08330 organoids cultured for one month (32 d.i.v.), three months (98 d.i.v.), and six months (190 d.i.v.). Left, t-SNE plots (n=3 organoids per timepoint, co-clustered). Cells are colored by cell type, and the number of cells per plot is indicated. Right, immunohistochemistry of specific markers. SOX2 (magenta), a marker of neural progenitors and TBR1 (green), a postmitotic neuronal marker, are shown at one month. SATB2 (magenta), a marker of CPNs, and CTIP2 (green), a marker of CFuPNs, are shown at three months. The astroglia markers S100B (magenta) and GFAP (green) are shown at six months. Below, schematic images of brain organoids in each timepoint. Scale bars are 100 μm. FIG. 18B shows immunohistochemistry for neuronal (MAP2), dorsal forebrain neural progenitor (EMX1, SOX2), CFuPN (CTIP2), and CPN (SATB2) markers in GM08330 organoids at one, three, and six months. Scale bars: whole organoids (leftmost column), 200 μm; others, 50 μm. FIG. 18C shows immunohistochemistry for cell type markers in Mito210 organoids, as in FIG. 18B. FIG. 18D top provides t-SNE plots of the scRNA-seq data from individual replicates from three organoids at one month, three organoids at three months, and three organoids at six months from the GM08330 cell line shown in FIG. 18A. FIG. 18D bottom provides bar charts showing the cell type composition of each individual organoid. On top of the bar charts, mutual information (MI) scores between cell type proportions and organoid identities are displayed. A MI score of 0 would indicate identical cell type proportions between organoids, while a score of 1 would indicate completely divergent profiles. In previous work, MI scores for endogenous brain datasets were reported to range from 0.008 to 0.064¹⁴. FIG. 18E provides scRNA-seq data of organoids from the Mito210 cell line at one month (35 d.i.v.), three months (92 d.i.v.), and six months (178 d.i.v.), as in FIG. 18D. Organoids for the one and three month timepoints are the same as the control organoids in FIG. 21D and FIG. 22B. FIG. 18F shows expression of selected marker genes used in cell-type identification. Violin plots show distribution of normalized expression in cells from GM08330 organoids at one, three and six months (n=3 individual organoids per time point). FIG. 18G shows expression of marker genes in Mito210 organoids, as in FIG. 18F. aRG, apical radial glia; DL, deep layers; UL, upper layer; PN, projection neurons; oRG, outer radial glia; IPC, intermediate progenitor cells; CPN, callosal projection neurons; CFuPN, corticofugal projection neurons; GABA INP, GABAergic interneuron progenitors; GABA IN, GABAergic interneurons.

FIGS. 19A-19H demonstrate expression of selected ASD risk genes in cortical organoids cultured for one, three, and six months from two cell lines. FIG. 19A provides t-SNE plots of 58,568 cells from nine organoids from the GM08330 cell line, data in FIGS. 18A, 18D, 18F, after Harmony batch correction. Cells are colored according to cell type (left) and timepoint (right). FIG. 19B provides gene set expression scores for a set of 102 genes associated with ASD risk⁶ across cell types, in cells from FIG. 19A. Scores above 0 indicate enriched expression over similar sets of randomly chosen genes. FIG. 19C provides t-SNE plots showing normalized expression of selected ASD risk genes in cells from FIG. 19A. FIG. 19D shows average expression of 102 genes associated with ASD risk across cell types and time points in GM08330 cell line. FIG. 19E provides t-SNE plots of nine organoids from the Mito210 cell line, data in FIGS. 18E, 18G, after Harmony batch correction. Cells are colored according to cell type (left) or timepoint (right). FIG. 19F shows gene set scores for the set of ASD risk genes as in FIG. 19B, in cells from FIG. 19E. Scores above 0 indicate higher expression than similar modules of randomly chosen genes. FIG. 19G provides t-SNE plots showing normalized expression of selected ASD risk genes in cells from FIG. 19E. FIG. 19H shows expression of 102 genes associated with ASD risk across cell types and time points in Mito210 cell line. RG, radial glia (aRG, oRG, and oRG/Astroglia), IPC, intermediate progenitor cells; CPN, callosal projection neurons; CFuPN, corticofugal projection neurons; EN, Excitatory neurons (UL CPN, DL CFuPN and PN); GABA IN, GABAergic interneurons.

FIGS. 20A-20J demonstrate generation and characterization of SUV420H1, ARID1B, and CHD8 mutant organoids. FIG. 20A shows protein domain structure of SUV420H1. The Mito210, PGP1 and Mito294 parental lines were edited to induce a mutation in the N-domain (arrow) that resulted in a protein-truncating variant. Bottom, characteristics of the cell lines in which a SUV420H1 mutation was introduced. FIG. 20B shows protein domain structure of ARID1B. The Mito210 and Mito294 parental lines were edited to induce a mutation before the ARID domain (arrow) that resulted in a protein-truncating variant. Bottom, characteristics of the cell lines in which a ARID1B mutation was introduced. FIG. 20C shows protein domain structure of CHD8. The HUES66, H1, GM08330, Mito294 and Mito210 lines were engineered to carry a mutation in the helicase C-terminal domain (HELC domain, arrow) that results in a protein-truncating variant. Bottom, characteristics of the cell lines in which a CHD8 mutation was introduced. FIGS. 20D-20F provide western blot analysis showing presence of SUV420H1 (FIG. 20D), ARID1B (FIG. 20E) and CHD8 (FIG. 20F) in control lines, and their reduction in the mutant lines. H4K20me3, a target of SUV420H1 activity, and total levels of histone H4 were also detected in control and in SUV420H1^{+/-} lines (FIG. 20D). ARID1B was not detectable in Mito294 even with a longer exposure of the blotted membrane (FIG. 20E, right). Asterisks indicate the bands used for quantification. Bottom, protein levels in control and mutant lines were quantified and normalized for housekeeping genes 3-Actin or GAPDH. FIG. 20G shows immunohistochemistry for neuronal (MAP2), dorsal forebrain neural progenitor (EMX1, SOX2) and CFuPN (CTIP2) markers in organoids at 35 d.i.v. derived from the Mito210 SUV420H1^{+/-}, Mito210 ARID1B^{+/-} and HUES66 CHD8^{+/-} mutant and isogenic control cell lines. Scale bar, 300 μm. h-j, FIGS. 20H-20J provide size quantification of control and SUV420H1^{+/-} (FIG. 20H), ARID1B1^{+/-} (FIG. 20I) and CHD8^{+/-} (FIG. 20J) organoids across lines and at different time points. FIG. 20H is repeated from FIG. 13B for ease of comparison. The ratio of organoid size compared to the average of control organoids in each batch is plotted. Differentiation batch (b.) is indicated by the shade of the points. Number of organoids and differentiations used for the measurement are summarized in FIG. 30. P-values from a two-sided t-test, after Bonferroni adjustment within each mutation.

FIGS. 21A-21E demonstrate cell type composition of SUV420H1^{+/-} and isogenic control organoids. FIGS. 21A-21C provide scRNA-seq data from one month (Mito294 35d.i.v. (FIG. 21A), PGP1 35 d.i.v. (FIG. 21B) and Mito210 28 d.i.v., batch I (FIG. 21C)) control and SUV420H1^{+/-} organoids. Bar charts show the percentage of cells for all the cell populations in each control and mutant organoid. Adjusted p-values for a difference in cell type proportions between control and mutant, based on logistic mixed models (see Methods) are shown. FIG. 21D provides scRNA-seq data from Mito210 35 d.i.v. (batch II) control and SUV420H1^{+/-} mutant organoids. Left, bar charts show the percentage of cells for all the cell populations in each control and mutant organoid, as in FIGS. 21A-21C. Right top shows combined t-SNE plots of control and mutant organoids (n=3 single organoids per genotype). Cells are colored by cell type, and the number of cells per plot is indicated. Right bottom, t-SNE plots for control and mutant individual organoids. Immature deep layer projection neuron populations are highlighted in color. FIG. 21E shows enriched gene ontology terms for genes upregulated and downregulated in SUV420H1^{+/-} vs. control across lines. Genes were calculated using cells from the partitions of interest. The top 5 most significant terms per dataset are shown. Size of dot indicates the proportion of genes belonging to each term found in the list of dysregulated genes (“GeneRatio”). Color indicates enrichment adjusted p value. Numbers in parentheses along the y axis indicate the number of differentially expressed genes in that dataset. As control, GO term enrichment was calculated for 100 random gene sets of the same size sampled from genes expressed in each dataset, and found no significant enrichment of these terms (see Methods). aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons; GABA N, GABAergic neurons.

FIGS. 22A-22E demonstrate cell type composition, full pseudotime trajectories, and gene modules in SUV420H1^{+/-} and isogenic control organoids. FIG. 22A provides scRNA-seq data from Mito294 three month (89 d.i.v.) control and SUV420H1^{+/-} organoids. Bar charts show the percentage of cells for all the cell populations in each control and mutant organoid. Adjusted p-values for a difference in cell type proportions between control and mutant, based on logistic mixed models (see Methods) are shown. FIGS. 22B-22C provide scRNA-seq data from three month Mito210 92 d.i.v. batch I (FIG. 22B) and 90 d.i.v. batch II (FIG. 22C) control and SUV420H1+/− mutant organoids. Left, bar charts show the percentage of cells for all the cell populations in each control and mutant organoid, as in FIG. 22A. Right top shows combined t-SNE plots of control and mutant organoids (n=3 single organoids per genotype). Cells are colored by cell type, and the number of cells per plot is indicated. Right bottom, t-SNE plots for control and mutant individual organoids. Deep layer CFuPN are highlighted in color. FIG. 22D shows pseudotime trajectory from the full dataset of Mito210 SUV420H1^{+/-} 35 d.i.v. (batch II) and control organoids, calculated with Monocle3. The partition highlighted by a box was subsetted and the trajectory is shown in FIG. 13H. FIG. 22E provides module scores (top) and their distribution across mutant and control cells (bottom) for all modules resulting from WGCNA analysis of the partition of interest from Mito210 SUV420H1^{+/-} and control organoids at 35 d.i.v. (batch II). Cells were downsampled to have an equal number of cells per organoid. Names were assigned to each module based on the known functions of the genes included in each one. Horizontal bars show median scores, and dots show average score per organoid. Adjusted p-values show differences between control and mutant based on linear mixed models (see Methods). aRG, apical radial glia; DL, deep layer; UL, upper layer; PN, projection neurons; CP/CH, Choroid Plexus/Cortical Hem; oRG, outer radial glia; IPC, intermediate progenitor cells; CPN, callosal projection neurons; CFuPN, corticofugal projection neurons; GABA INP, GABAergic interneuron progenitors; GABA IN, GABAergic interneurons; GABA N, GABAergic neurons.

FIGS. 23A-23B demonstrate scATAC-seq analysis in SUV420H1^{+/-} and isogenic control organoids. FIG. 23A provides genome tracks showing read coverage for representative regions with increased accessibility between Mito210 SUV420H1^{+/-} and control organoids, split by cell type. Scales for the y axes (normalized counts) are displayed on the top right. FIG. 23B top shows 5 de novo motifs enriched in the regions with altered accessibility in Mito210 SUV420H1^{+/-} compared to control organoids at one month (31 d.i.v.) and three months (93 d.i.v.), as calculated with HOMER (see Methods). Regions that showed increased accessibility in mutant compared to control organoids are in green and purple, while those with decreased accessibility are in red and blue. Transcription factors with known binding sites resembling the discovered motifs are shown.

FIGS. 24A-24H demonstrate neuronal spontaneous activity in SUV420H1^{+/-} and isogenic control organoids. FIG. 24A left provides representative image of a PGP1 SUV420H1 organoid infected with SomaGCaMP6f2. FIG. 24A right, DF/F signal at the peak of a network burst. Scale bar: 100 μm. FIG. 24B top, representative trace of spontaneous calcium signal (corresponding to cell #3 in FIG. 14D). FIG. 24B, bottom, high magnification traces of calcium transients, displaying the difference in amplitude between the isolated event and the network burst (top), and normalized traces (bottom) showing their kinetics and the multiple peaks of the burst signal. FIGS. 24C-24D provide synchronous network activity in human brain organoids. Heatmap of cross-correlation index (FIG. 24C) and cross-correlogram against a reference signal (cell #135) for a representative recording. As a control, the signal of 10 cells were circularly shifted by a random phase and the cross-correlation was then calculated. In FIG. 24D, the average value was plotted, and the synchronous activations as well as the periodic bursting can be seen (“All cells” in red). FIG. 24E provides MEA recordings. Top, Spatial configuration of recording electrodes. Middle, Example raw traces for the numbered electrodes shown at the top, and the effect upon 2 μM TTX application. Yellow columns indicate the network bursts. Right, Individual (grey) and average (color) spike waveforms for each electrode. High magnification of the trace #4 showing the individual spikes (asterisk) during a burst event. Bottom, Average spike waveform (right) in a unit of electrodes (left), extracted at the time points determined by the spikes in electrode #4. FIG. 24F shows effect of NBQX on calcium signal. Heatmap of DF/F signal for 15 representative cells in control (top) and SUV420H1^{+/-} (bottom) organoids. FIGS. 24G-24H show inter-spike interval (ISI) analysis for the network bursting. Relative frequency (FIG. 24G) and cumulative frequency distribution (FIG. 24H) of ISI for control and SUV420H1^{+/-} organoids. In FIG. 24H, Kolmogorov-Smirnov test (n=5 organoids per genotype).

FIGS. 25A-25G demonstrate cell type composition, full pseudotime trajectories, and gene modules of ARID1B^{+/-} and isogenic control organoids. FIGS. 25A-25C provide scRNA-seq data from Mito210 one month (35 d.i.v. batch I in FIG. 25A, batch II in FIG. 25B) and Mito210 three months (90 d.i.v., in FIG. 25C) control and ARID1B^{+/-} organoids. Bar charts show the percentage of cells for all the cell populations in each control and mutant organoid. Adjusted p-values for a difference in cell type proportions between control and mutant, based on logistic mixed models (see Methods) are shown. FIG. 25D shows immunohistochemistry for the postmitotic excitatory neuronal marker TBR1 (magenta) and GABAergic marker DLX2 (green) in Mito210 control and ARID1B^{+/-} organoids at three months (90 d.i.v.). Scale bars: 500 μm. FIG. 25E provides scRNA-seq data from Mito294 one month (35 d.i.v.) ARID1B^{+/-} and control organoids. Right top shows combined t-SNE plots of control and mutant organoids (n=3 single organoids per genotype). Cells are colored by cell type, and the number of cells per plot is indicated. Bar charts show the percentage of cells for all the cell populations in each control and mutant organoid, as in FIGS. 25B-25C. Right bottom, t-SNE plots for control and mutant individual organoids. GABAergic neurons, newborn deep layer projection neurons and immature deep layer projection neuron populations are highlighted in color. FIG. 25F shows pseudotime trajectories from the full dataset of Mito210 ARID1B^{+/-} 35 d.i.v. batch II and control organoids, calculated with Monocle3. The partition highlighted by a box was subsetted and the trajectory is shown in FIG. 15F. FIG. 25G shows module scores (top) and their distribution across mutant and control cells (bottom) for all modules resulting from WGCNA analysis of the partition of interest from Mito210 ARID1B1^{+/-} and control organoids at 35 d.i.v. Cells were downsampled to have an equal number of cells per organoid. Names were assigned to each module based on the known functions of the genes included in each one. Horizontal bars show median scores, and dots show average score per organoid. Adjusted p-values show differences between control and mutant based on linear mixed models (see Methods). aRG, apical radial glia; DL, deep layer; UL, upper layer; PN, projection neurons; CP/CH, Choroid Plexus/Cortical Hem; oRG, outer radial glia; IPC, intermediate progenitor cells; CPN, callosal projection neurons; CFuPN, corticofugal projection neurons; GABA NP, GABAergic neuron progenitors; GABA N, GABAergic neurons; GABA INP; GABAergic interneuron progenitors; GABA IN, GABAergic interneurons.

FIGS. 26A-26F demonstrate cell type composition, immunohistochemistry, and full pseudotime trajectories and gene modules of CHD8^{+/-} and isogenic control HUES66 organoids. FIGS. 26A-26B provide scRNA-seq data from HUES66 3.5-month (109 d.i.v. (FIG. 26A), batch I and 107 d.i.v. (FIG. 26B). batch II) CHD8^{+/-} and control organoids. Bar charts show the percentage of cells for all the cell populations in each control and mutant organoid. Adjusted p-values for a difference in cell type proportions between control and mutant, based on logistic mixed models (see Methods) are shown. FIG. 26C shows immunohistochemistry for neuronal (MAP2), dorsal forebrain neural progenitor (EMX1, SOX2) and CFuPN (CTIP2) markers in HUES66 CHD8^{+/-} and control organoids at 3.5 months (107 d.i.v., top), and six months (190 d.i.v., bottom). Scale bars: whole organoids, 500 μm; others, 100 μm. FIG. 26D provides scRNA-seq data from HUES66 CHD8^{+/-} and control organoids at six months (190 d.i.v.). Bar charts show the percentage of cells for all the cell populations in each control and mutant organoid as in FIGS. 26A-26B. FIG. 26E show pseudotime trajectories from the full dataset of HUES66 batch I CHD8^{+/-} and control organoids at 109 d.i.v., calculated with Monocle3. The partition highlighted by a box was subsetted and the trajectory is shown in FIG. 16F. FIG. 26F shows module scores (top) and their distribution across mutant and control cells (bottom) for all modules resulting from WGCNA analysis of the partition of interest from HUES66 CHD8^{+/-} and control organoids at 109 d.i.v. Cells were downsampled to have an equal number of cells per organoid. Names were assigned to each module based on the known functions of the genes included in each one. Horizontal bars show median scores, and dots show average score per organoid. Adjusted p-values show differences between control and mutant based on linear mixed models (see Methods). aRG, apical radial glia; DL, deep layer; UL, upper layer; PN, projection neurons; oRG, outer radial glia; IPC, intermediate progenitor cells; CPN, callosal projection neurons; CFuPN, corticofugal projection neurons; GABA INP, GABAergic interneuron progenitors; GABA IN, GABAergic interneurons.

FIGS. 27A-27D demonstrate bulk RNA-seq and scRNA-seq of CHD8^{+/-} and isogenic control organoids from multiple cell lines. FIG. 27A provides bulk RNA-seq data from HUES66, GM83330 and H1 35 d.i.v. organoids. Enriched gene ontology terms for genes differentially expressed in CHD8^{+/-} vs. control organoids. The top 5 most significant terms per dataset are shown. Size of dot indicates the proportion of genes belonging to each term found in the list of dysregulated genes (“GeneRatio”). Color indicates enrichment adjusted p-value. Numbers in parentheses along the x axis indicate the number of differentially expressed genes in that dataset. FIGS. 27B-27D provide scRNA-seq data from control and CHD8^{+/-} organoids at 3.5 months (GM83330 108 d.i.v., batch I (FIG. 27B), GM83330 108 d.i.v., batch II (FIG. 27C) and H1 105 d.i.v. (FIG. 27D)). Bar charts show the percentage of cells for all the cell populations in each control and mutant organoid. Adjusted p-values for a difference in cell type proportions between control and mutant, based on logistic mixed models (see Methods) are shown. Right top shows combined t-SNE plots of control and mutant organoids (n=3 single organoids per genotype). Cells are colored by cell type, and the number of cells per plot is indicated. Right bottom, t-SNE plots for control and mutant individual organoids. aRG, apical radial glia; DL, deep layer; UL, upper layer; PN, projection neurons; CP/CH, Choroid Plexus/Cortical Hem; oRG, outer radial glia; IPC, intermediate progenitor cells; CPN, callosal projection neurons; CFuPN, corticofugal projection neurons; GABA INP, GABAergic interneuron progenitors; GABA IN, GABAergic interneurons; GABA N, GABAergic neurons.

FIGS. 28A-28J demonstrate convergent differential genes and differentially expressed proteins for the three mutations. FIG. 28A shows differential expression of all 102 genes associated with ASD risk⁶ in the three datasets Mito210 SUV420H1^{+/-} 35 d.i.v., Mito210 ARID1B^{+/-} 35 d.i.v. and in HUES66 CHD8^{+/-} 35 d.i.v. compared to relative controls. Expression of risk genes was calculated using all cells (pseudobulk) for Mito210 SUV420H1^{+/-} and Mito210 ARID1B^{+/-}. Boxes are colored according to−log₁₀(adjusted p value) according to whether they are upregulated (purple), or downregulated (turquoise) in mutant vs. control. Genes are ordered according to hierarchical clustering (using Euclidean distance) of those values. FIGS. 28B-28C show enriched gene ontology terms for genes upregulated (FIG. 28B) and downregulated (FIG. 28C) in mutant vs. control. Genes were calculated using the cells as in FIG. 28A. The top 5 most significant terms per dataset are shown. Size of dot indicates the proportion of genes belonging to each term found in the list of dysregulated genes (“GeneRatio”). Color indicates enrichment adjusted p-value. Numbers in parentheses along the x axis indicate the number of differentially expressed genes in that dataset. FIGS. 28D-28F provide volcano plot showing fold change versus adjusted p-value of measured proteins in MS experiments on Mito210 SUV420H1^{+/-} (FIG. 28D), Mito210 ARID1B^{+/-} (FIG. 28E), and HUES66 CHD8^{+/-} (FIG. 28F) vs. control organoids at 35 d.i.v. (n=4 single organoids per genotype for SUV420H1, 4 controls and 5 mutants for ARID1B, and n=3 single organoids per genotype for CHD8). Significant DEPs are shown in red (FDR <0.1). FIGS. 28G-28I provide selected enriched gene ontology terms for DEPs in Mito210 SUV420H1^{+/-} (FIG. 28G), Mito210 ARID1B^{+/-} (FIG. 28H), and HUES66 CHD8^{+/-} (FIG. 28I) vs. control organoids cultured for 35 d.i.v. FIG. 28J shows protein set distances between the top 50 DEPs per mutation. For each pair of mutations, a PPI-weighted protein set distance was calculated between DEPs (pink diamond). To determine if this distance was smaller than would be expected by chance, size-matched sets were randomly chosen from the proteins detected in each experiment, and distance between these random sets was calculated 1000 times per pair. P-values were assigned by counting the fraction of times this random distance was less than the actual distance value between differential sets. DEPs: differentially expressed proteins. MS: mass spectrometry.

FIG. 29 provides information on mutations induced in pluripotent stem cell lines.

FIG. 30 provides the number of individual organoids measured faor each comparison of organoid size. See FIGS. 20H-20J.

### DETAILED DESCRIPTION OF THE INVENTION

As described herein, reproducible organoid models of the cerebral cortex were combined with single cell RNA-seq, single cell ATAC-seq, proteomics, and electrophysiological analysis to investigate the role of ASD risk genes in human cortical development, across multiple human cell lines. The focus was on chromatin modifiers expressed at early stages of brain development, and genes that have emerged repeatedly as top hits in studies of ASD genetic risk were selected: SUV420H1, PTEN, ARID1B, and CHD8. All of the genes are associated with severe neurodevelopmental abnormalities in patients, including shared phenotypes of high frequency of macrocephaly, suggesting some degree of convergence in effect. However, their broad functions and widespread expression make it difficult to predict how their mutation leads to disease and whether shared mechanisms may play a role. It is shown herein that mutations in these genes converge on a shared phenotype of asynchronous neurogenesis that selectively affects specific classes of cortical neurons. Notably, although each mutation caused similar developmental abnormalities, they acted through divergent molecular mechanisms. Moreover, while these phenotypes were broadly consistent across cell lines from different donors, it was demonstrated that the degree of expressivity varies depending on the risk gene and for different aspects of the phenotype, highlighting the nuanced interactions of risk genes and genomic contexts that produce the phenotypic manifestation of ASD.

Described herein are methods of screening for pathological mechanisms in neurodevelopmental and/or neuropsychiatric disorders. The neurodevelopmental and/or neuropsychiatric disorder or condition may be any disorder affecting synaptic function, neuronal network activity and/or stimulation. A neurodevelopmental and/or neuropsychiatric disease or condition may be a disease or condition involving neural loss mediated or characterized at least partially by at least one of deterioration of neural stem cells and/or progenitor cells.

“Neuropsychiatric disorder” encompasses mental disorders attributable to diseases of the nervous system. Non-limiting examples of neuropsychiatric disorders include addictions, childhood developmental disorders, eating disorders, degenerative diseases, mood disorders, neurotic disorders, psychosis, sleep disorders, depression, obsessive-compulsive disorder, schizophrenia, visual hallucination, auditory hallucination, eating disorder, bipolar disorder, epilepsy, autism spectrum disorder (ASD), and amyotrophic lateral sclerosis (ALS).

“Neurodevelopmental disorder” encompasses mental disorders attributable to impairments of the growth and development of the brain and/or nervous system. Non-limiting examples of neurodevelopmental disorders include attention deficit hyperactivity disorder (ADHD), developmental language disorder (DLD), communication or speech disorder, autism spectrum disorder (ASD), intellectual disabilities, motor disorders (e.g., developmental coordination disorder, stereotypic movement disorder, tic disorder), neurogenetic disorder (e.g., Fragile X syndrome, Down syndrome, Rett syndrome, hypogonadotropic hypogonadal syndromes), learning disorders (e.g., dyslexia or dyscalculia), traumatic brain injury and/or congenital brain injury, and fetal alcohol spectrum disorder.

In some embodiments, the methods of screening for pathological mechanisms in neurodevelopmental and/or neuropsychiatric disorders comprise introducing one or more genetic variations to an organoid. In some embodiments, the organoid is a reproducible organoid. In some embodiments, the organoid is a cortical organoid. The organoid may be derived from cells of any suitable species. In some embodiments, the organoid is derived from mammalian cells. In some embodiments, the organoid is derived from human or rodent (e.g., mouse) cells (e.g., human or rodent stem cell or pluripotent cell). In some embodiments, the organoid is derived from human cells. In some embodiments, the organoid is derived from cells (e.g., human cells or rodent cells, human or rodent stem cells or progenitor cells) comprising a mutation associated with a neurological disease or condition, e.g., a neurodevelopmental or neuropsychiatric disorder or condition.

Stem cells and progenitor cells used to derive the organoids described herein can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

In some embodiments, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

Embryonic stem (ES) cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subject's adipose tissue, for example using the CELUTION™ SYSTEM from Cytori, as disclosed in U.S. Pat. Nos. 7,390,484 and 7,429,488 which are incorporated herein in their entirety by reference.

In some embodiments, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the organoid is derived from PGP1 (Personal Genome Project 1) iPSC (induced pluripotent stem cells); HUES66 hESC (human embryonic stem cells); H1 hESC (also known as WA01); GM08330 iPSC (also known as GM8330-8); Mito 210 iPSC; Mito 294 iPSC. Additional examples of reproducible organoids that may be used herein are described in WO 2020/243657, the entire contents of which is incorporated herein by reference.

In some embodiments, one or more genetic variations are introduced to an organoid. In some embodiments, the stem cell from which the organoid is derived is altered. The alteration to the stem cell may occur in any manner which is available to the skilled artisan, for example, utilizing a TALEN, ZFN, or a CRISPR/Cas system. Such CRISPR/Cas systems can employ a variety of Cas proteins (Haft et al. PLoS Comput Biol. 2005; 1(6)e60). In some embodiments, the CRISPR/Cas system is a CRISPR type I system. In some embodiments, the CRISPR/Cas system is a CRISPR type II system. In some embodiments, the CRISPR/Cas system is a CRISPR type V system. It should be understood that although examples of methods utilizing CRISPR/Cas (e.g., Cas9 and Cpf1) and TALEN are described in detail herein, the invention is not limited to the use of these methods/systems.

In some embodiments, the alteration to the stem cell is a modification or cleavage of a target sequence. In other embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target sequence.

In some embodiments, the alteration, e.g., an indel, is made to a gene, e.g., a gene associated with a neurodevelopmental or neuropsychiatric disorder. For example, an alteration, e.g., indel, may target a protein domain that is mutated in a subject suffering from a neurodevelopmental or neuropsychiatric disorder. In one embodiment, the indel is a protein-truncating indel. In some embodiments, the gene is an autism spectrum disorder (ASD) risk gene. In some embodiments, the gene is expressed early in brain development. In some embodiments, the gene is a chromatin modifier. In some embodiments, the gene is selected from the group consisting of SUV420H1 (KMT5B), ARID1B, PTEN, and CHD8. In some embodiments, the gene is selected from the group consisting of SUV420H1, ARID1B, and CHD8. In one embodiment, the gene is SUV420H1. In one embodiment, the gene is ARID1B. In one embodiment, the gene is CHD8. In one embodiment, the gene is PTEN.

In some embodiments, an organoid is derived from the altered stem cell, e.g., the stem cell comprising the indel. In some aspects, one or more genetic variations (e.g., a mutation or alteration) is introduced into an organoid via the altered stem cell. In some aspects, an indel alteration targeting a protein domain is introduced into the organoid. In some embodiments, the genetic variation introduced to the organoid causes haploinsufficiency in a gene. In some embodiments, the one or more genetic variations introduced to the organoid causes haploinsufficiency in SUV420H1, ARID1B, PTEN, and/or CHD8. In some embodiments, the one or more genetic variations introduced to the organoid cause haploinsufficiency in SUV420H1, ARID1B, and/or CHD8. In some embodiments, the organoids comprise haploinsufficient mutations in SUV420H1, ARID1B, and/or CHD8.

In some embodiments, the methods further comprise profiling the mutant organoids using single-cell RNA sequencing. In some embodiments, the mutant organoids are further profiled using one or more of immunohistochemistry, whole proteome analysis, single cell assay for transposase-accessible chromatin sequencing (ATAC-seq), and electrophysiological analysis. The organoids may additionally be assessed for changes in expression of genes and/or gene products over a period of time by any method known in the art including immunohistochemistry, immunofluorescence, flow cytometry, polymerase chain reaction (PCR), quantitative PCR, real-time PCR, gene expression array, mRNA sequencing, high-throughput sequencing, Western blot, Northern blot, and ELISA.

The mutant organoids may be screened or profiled at one or more time points, e.g., after introduction of a genetic variation. In some embodiments, the mutant organoids are screened at least 1, 5, 10, 15, 20, 25, 30, 40, 60, 90, or 120 days after introduction of the genetic variation. In some embodiments, the mutant organoids are screened at least 1, 2, 3, 4, 5, 6, 7, 8, 9, 10, 11, or 12 months after introduction of the genetic variation. In one embodiment, the mutant organoids are screened at least 1, 3, and/or 6 months after introduction of the genetic variation.

In some embodiments, the profiling of the mutant organoids can be used to identify phenotypic alterations that occur as a result of the genetic variation. In some embodiments, the phenotypic alteration comprises the accelerated development of cortical neurons. In some embodiments, the phenotypic alteration comprises the overproduction of cortical neuronal lineages, e.g., neuronal overgrowth. In some embodiments, the phenotypic alteration comprises asynchronous cortical neurogenesis. In one embodiment, the phenotypic alteration comprises asynchronous development of deep layer projection neuron lineage. In one embodiment, the phenotypic alteration comprises premature expansion of GABAergic neuron lineage. In one embodiment, the phenotypic alteration comprises an imbalance of production of cortical excitatory neurons. In some embodiments, early stage developmental changes in the organoid (e.g., the accelerated development of cortical neurons) are related to later abnormal circuit activity of the organoid.

In some embodiments, the mutant organoids exhibit differential expression of one or more genes or gene products associated with synaptic transmission, neuronal maturation, neuronal connectivity, neuronal processes (e.g., axon guidance, synaptogenesis), synapse formation, cytoskeleton remodeling, and/or neuronal development. The differential expression of the proteins may vary based on the genetic variation introduced into the organoid, as well as based on the underlying stem cell from which the organoid is derived. In some embodiments, the mutant organoids exhibit reduced spontaneous neuronal activity and/or delayed neuronal differentiation, e.g., as compared to a control organoid. In some embodiments, the mutant organoids exhibit differentially expressed proteins associated with neuronal maturation and neuronal function. In some embodiments, the mutant organoids exhibit differential expression of one or more genes or gene products selected from the group consisting of CRH, GAD2, SLC32A1, POU3F2, ETV1, GAD1, RBFOX1, JAG1, ZFHX4, FBN2, ZNF385D, SHD, RCAN2, GPC3, ZNF439, APITD1, GNG3, ARPC1B, DDIT3, C14orfl42, NKAIN3, ODC1, CD99, MYO10, TXNDC9, NETO2, TCEAL7, TMEM163, PNO1, PHF19, GCSH, HYAL2, C1QBP, TMEM14C, NPM3, TRIAP1, MRPL3, TJP1, PSMD12, EHBP1, C7orf50, GTF3C6, WIZ, BACH2, GLCCI1, NEDD4L, RAB3A, IER5, GPM6A, CHODL, GUCY1B3, ZNF37A, DPP10, CCSAP, NMRAL1, GSE1, CPM, IER3, INSIG1, AUTS2, NELL2, TUBB4A, GPR85, MYT1L, CALCOCO1, CACNA1A, RAP1GAP, TYW3, EFNA1, KIAA1456, STRA6, COL6A2, BCL11B, SNX32, TPM2, SEMA3A, IGFB5, CRYZ, ABTB1, ISLR2, RBM24, SYN3, SYNDIG1, ZEB2, NRSN1, MEF2C, PALMD, KCNG1, GRIK3, FAM50B, SLA, and CARTPT. In some embodiments, the mutant organoids exhibit differential expression of one or more genes or gene products selected from the group consisting of APITD1, C14orfl42, ODC1, TXNDC9, PNO1, GCSH, HYAL2, C1QBP, TMEM14C, NPM3, TRIAP1, MRPL3, TJP1, PSMD12, C7orf50, GTF3C6, GLCCI1, NEDD4L, RAB3A, GUCY1B3, ZNF37A, CCSAP, INSIG1, TUBB4A, GPR85, CALCOCO1, CACNA1A, RAP1GAP, KIAA1456, BCL11B, SNX32, SEMA3A, ISLR2, SYN3, ZEB2, NRSN1, MEF2C, GRIK3, FAM50B, SLA, and CARTPT. In some embodiments, the mutant organoids exhibit upregulated expression of one or more genes or gene products selected from the group consisting of GLCCI1, NEDD4L, RAB3A, GUCY1B3, ZNF37A, CCSAP, INSIG1, TUBB4A, GPR85, CALCOCO1, CACNA1A, RAP1GAP, KIAA1456, BCL11B, SNX32, SEMA3A, ISLR2, SYN3, ZEB2, NRSN1, MEF2C, GRIK3, FAM50B, SLA, and CARTPT.

Also described herein are methods of screening test agents to identify candidate therapeutic agents. In some aspects, organoids (e.g., mutant organoids) described herein or generated as described by the methods of the invention are used. In some embodiments, organoids are generated from cells derived from a subject having a neurodevelopmental or neuropsychological disease or disorder (e.g., autism spectrum disorder (ASD)).

In some embodiments, a method of screening for a candidate therapeutic agent comprises contacting an organoid having one or more genetic variations as described herein with a test agent, and testing the effect of the one or more candidate agents on the organoid. For example, testing the effect of the one or more candidate agents on asynchronous development of a cortical neuronal lineage (e.g., asynchronous development of deep layer projection neuron lineage and/or premature expansion of GABAergic neuron lineage).

In some embodiments, the effect of the one or more candidate agents on the differential expression of one or more genes or gene products is assessed. Differential expression may be assessed for one or more genes or gene products selected from the group consisting of CRH, GAD2, SLC32A1, POU3F2, ETV1, GAD1, RBFOX1, JAG1, ZFHX4, FBN2, ZNF385D, SHD, RCAN2, GPC3, ZNF439, APITD1, GNG3, ARPC1B, DDIT3, C14orfl42, NKAIN3, ODC1, CD99, MYO10, TXNDC9, NETO2, TCEAL7, TMEM163, PNO1, PHF19, GCSH, HYAL2, C1QBP, TMEM14C, NPM3, TRIAP1, MRPL3, TJP1, PSMD12, EHBP1, C7orf50, GTF3C6, WIZ, BACH2, GLCCI1, NEDD4L, RAB3A, IER5, GPM6A, CHODL, GUCY1B3, ZNF37A, DPP10, CCSAP, NMRAL1, GSE1, CPM, IER3, INSIG1, AUTS2, NELL2, TUBB4A, GPR85, MYT1L, CALCOCO1, CACNA1A, RAP1GAP, TYW3, EFNA1, KIAA1456, STRA6, COL6A2, BCL11B, SNX32, TPM2, SEMA3A, IGFB5, CRYZ, ABTB1, ISLR2, RBM24, SYN3, SYNDIG1, ZEB2, NRSN1, MEF2C, PALMD, KCNG1, GRIK3, FAM50B, SLA, and CARTPT. In some embodiments, differential expression may be assessed for one or more genes or gene products selected from the group consisting of APITD1, C14orfl42, ODC1, TXNDC9, PNO1, GCSH, HYAL2, C1QBP, TMEM14C, NPM3, TRIAP1, MRPL3, TJP1, PSMD12, C7orf50, GTF3C6, GLCCI1, NEDD4L, RAB3A, GUCY1B3, ZNF37A, CCSAP, INSIG1, TUBB4A, GPR85, CALCOCO1, CACNA1A, RAP1GAP, KIAA1456, BCL11B, SNX32, SEMA3A, ISLR2, SYN3, ZEB2, NRSN1, MEF2C, GRIK3, FAM50B, SLA, and CARTPT. In one embodiment, downregulation of one or more genes or gene products selected from the group consisting of GLCCI1, NEDD4L, RAB3A, GUCY1B3, ZNF37A, CCSAP, INSIG1, TUBB4A, GPR85, CALCOCO1, CACNA1A, RAP1GAP, KIAA1456, BCL11B, SNX32, SEMA3A, ISLR2, SYN3, ZEB2, NRSN1, MEF2C, GRIK3, FAM50B, SLA, and CARTPT is assessed.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting the organoid with an agent) is intended to include incubating the agent and organoid together in vitro. It is understood that the organoid contacted with an agent can also be simultaneously or subsequently contacted with another agent. In some embodiments, the organoid is contacted with at least two, at least three, at least four, at least five, at least six, at least seven, at least eight, at least nine, or at least ten agents.

In some embodiments, one or more parameters of the organoid are measured in response to contact with the test agent. In certain aspects, the one or more parameters include: the accelerated development of cortical neurons, asynchronous cortical neurogenesis, asynchronous development of deep layer projection neuron lineage, and/or premature expansion of GABAergic neuron lineage. In one aspect, differential gene or gene product expression is measured in response to contact with the test agent. In certain aspects, the one or more parameters include: spontaneous neuronal activity and/or delayed neuronal differentiation.

In some embodiments, the control organoid is an organoid not contacted with the test agent. In some embodiments, the test organoid is derived from a subject having a neurodevelopmental or neuropsychological disorder and the control organoid is from a subject without the neurodevelopmental or neuropsychological disorder. Thus, in certain embodiments, if contact of the test organoid with the test agent results in the test organoid exhibiting a property more similar to the control organoid, then the agent can be identified as a beneficial agent for the neurodevelopmental or neuropsychological disorder.

In some embodiments, a high throughput screen (HTS) is performed. A high throughput screen can utilize the highly reproducible organoids described herein. High throughput screens often involve testing large numbers of compounds with high efficiency, e.g., in parallel. Often such screening is performed in multiwell plates other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrön R & Hertzberg R P. Design and implementation of high-throughput screening assays. Methods Mol Biol., 565:1-32, 2009 and/or An WF & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hvser.

The articles “a”, “an” and “the” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. It should also be understood that any embodiment of the invention, e.g., any embodiment found within the prior art, can be explicitly excluded from the claims, regardless of whether the specific exclusion is recited in the specification.

As used herein the term “comprising” or “comprises” is used in reference to compositions, methods, and respective component(s) thereof, that are essential to the invention, yet open to the inclusion of unspecified elements, whether essential or not. The term “consisting essentially of” refers to those elements required for a given embodiment. The term permits the presence of additional elements that do not materially affect the basic and novel or functional characteristic(s) of that embodiment of the invention. The term “consisting of” refers to compositions, methods, and respective components thereof as described herein, which are exclusive of any element not recited in that description of the embodiment.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group.

Certain claims are presented in dependent form for the sake of convenience, but any dependent claim may be rewritten in independent format to include the limitations of the independent claim and any other claim(s) on which such claim depends, and such rewritten claim is to be considered equivalent in all respects to the dependent claim (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited. It is contemplated that all aspects described above are applicable to all different embodiments of the invention. It is also contemplated that any of the above embodiments can be freely combined with one or more other such embodiments whenever appropriate.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### EXEMPLIFICATION

### Example 1—Human Brain Organoids Reveal Accelerated Development of Cortical Neuron Classes as a Shared Feature of Autism Risk Genes

### Results

### Single-Cell Map of Developing Cortical Organoids Reveals Cell Type-Specific Enrichment of ASD Risk Genes

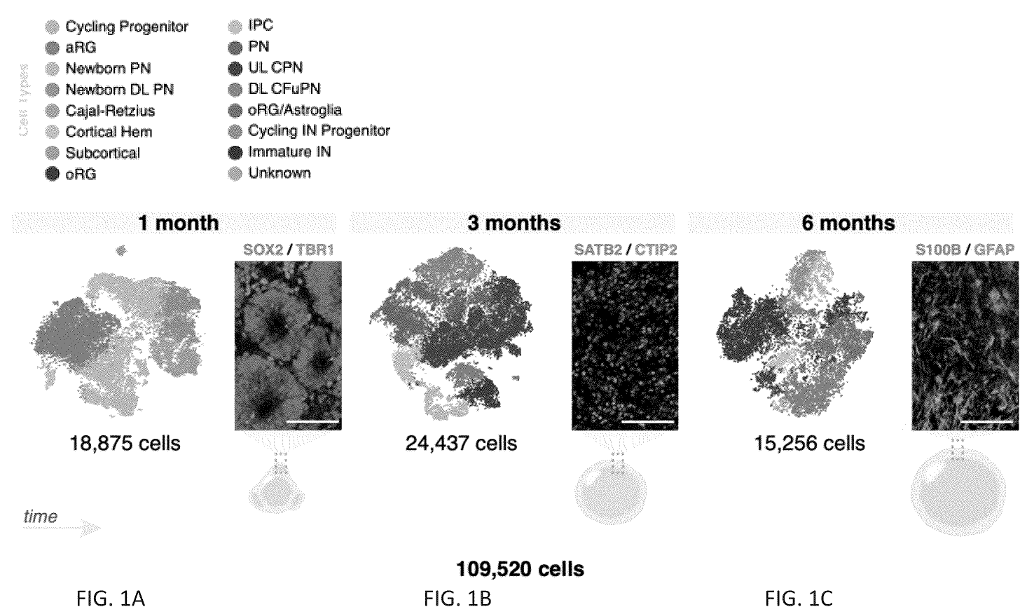

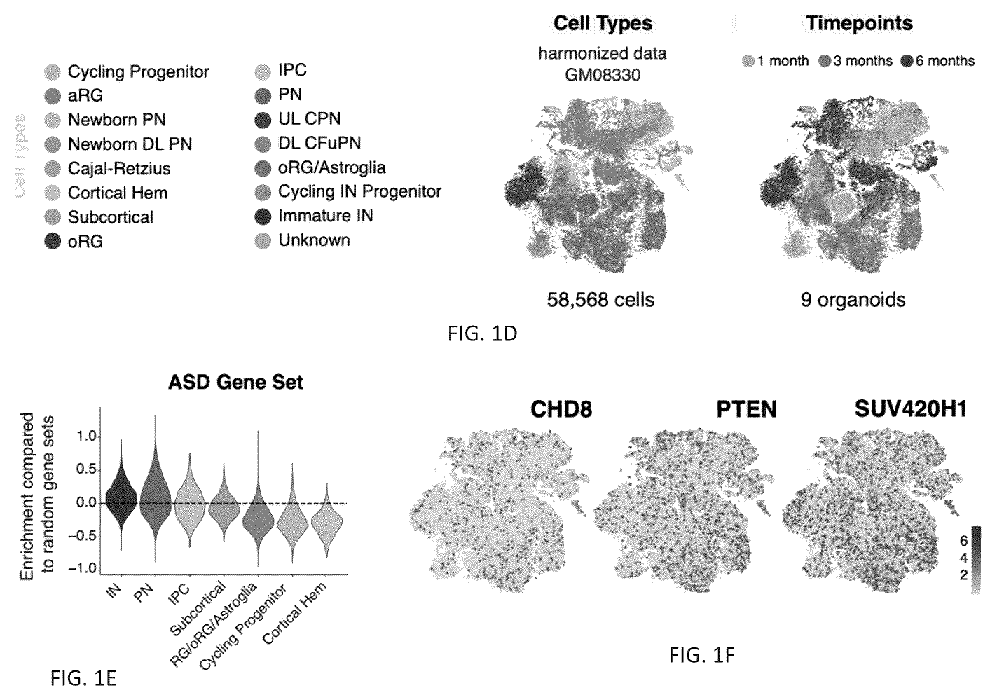

In order to determine whether human brain organoids could be used as models to understand the roles of ASD risk genes in human brain development, it was first sought to verify expression of a large set of known risk genes in this cellular system across a developmental timeline. A large dataset of scRNA-seq data was generated from individual organoids produced from two different human induced pluripotent stem cell (iPSC) lines, GM08330 and Mito210, using a recently-developed cortical organoid model (FIG. 1 and FIGS. 5-6)¹³.

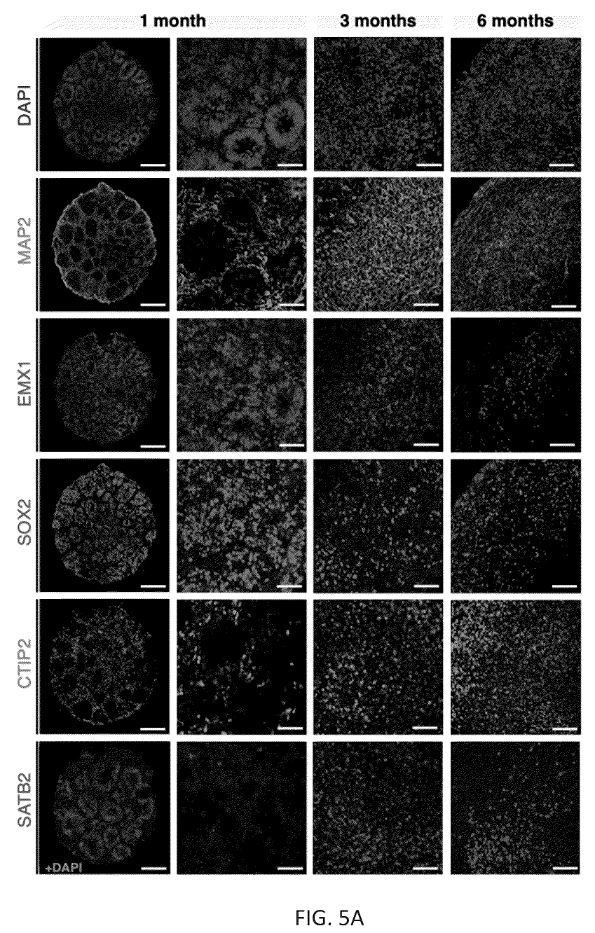

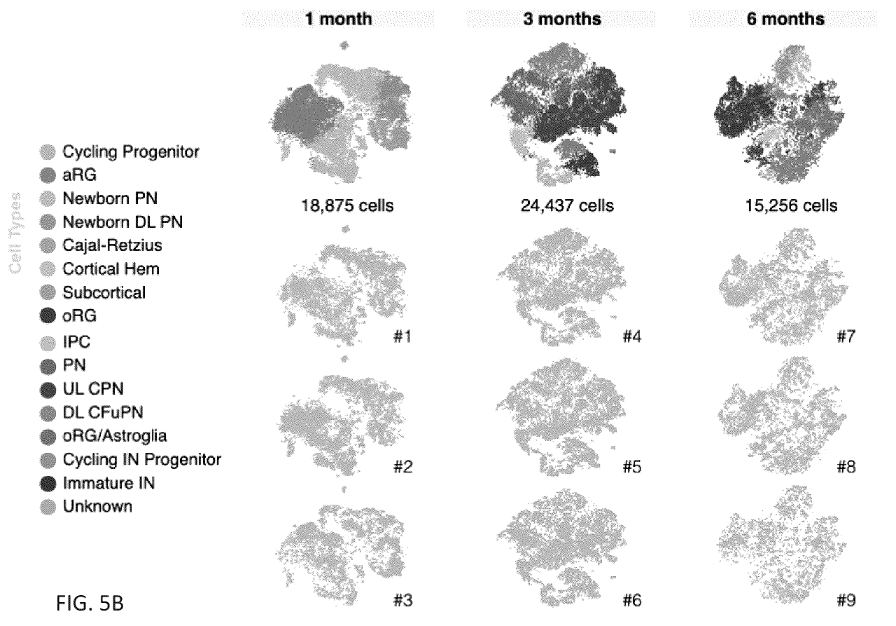

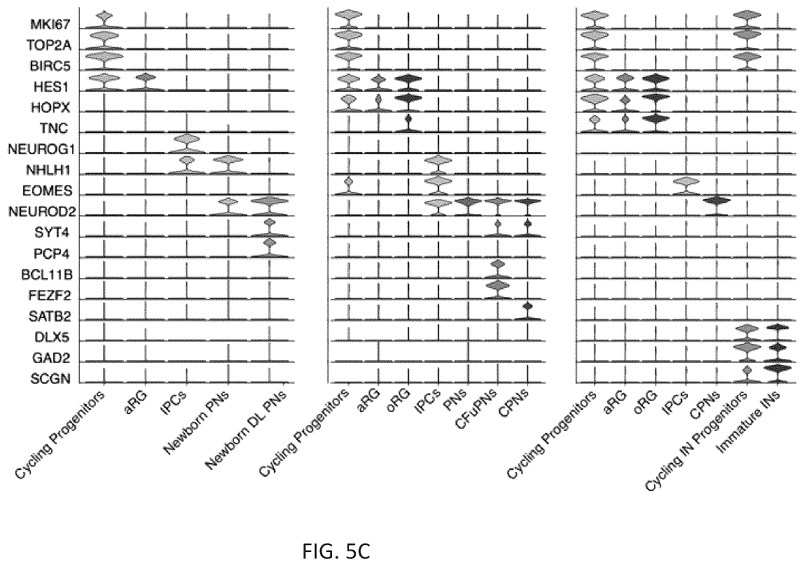

Individual organoids were profiled by immunohistochemistry and scRNA-seq across three stages of in vitro development: one month, when organoids contain multiple classes of progenitors and neurogenesis is beginning; three months, when progenitor diversity increases, including the appearance of outer radial glia, and multiple subtypes of projection neurons emerge; and six months, when astroglia and interneurons are present (n=3 single organoids per timepoint and line; FIGS. 1A-1C and FIG. 5). At all ages, the cell type composition was highly reproducible between individual organoids, irrespective of batch and stem cell line (FIG. 5B)¹³.

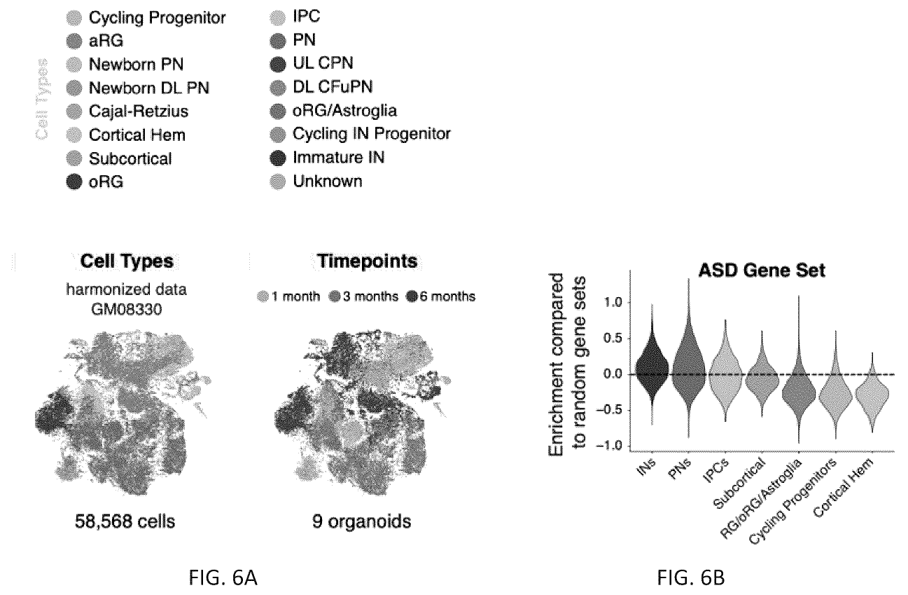

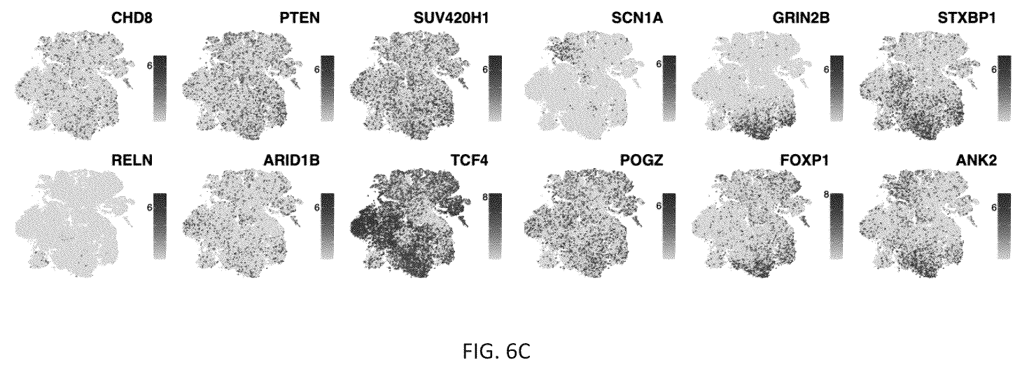

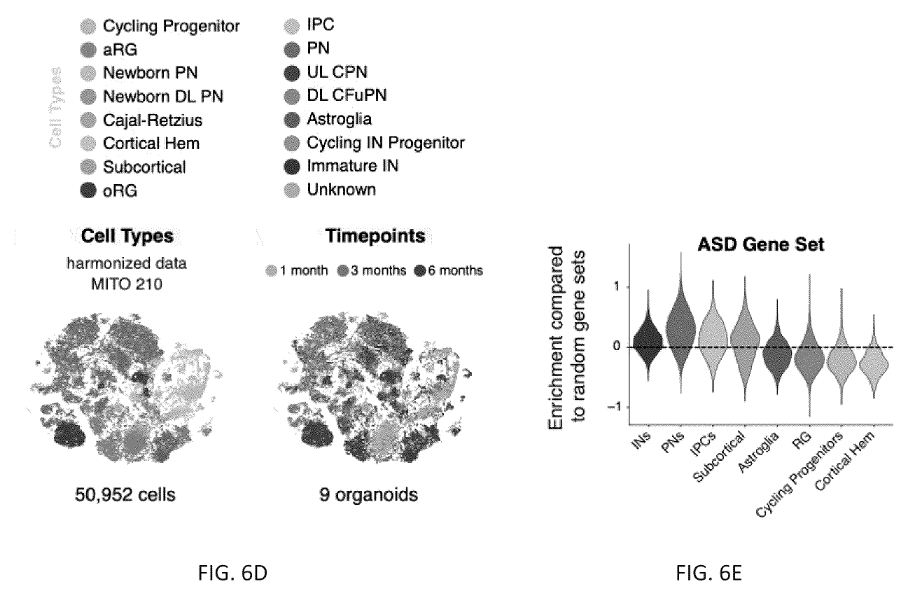

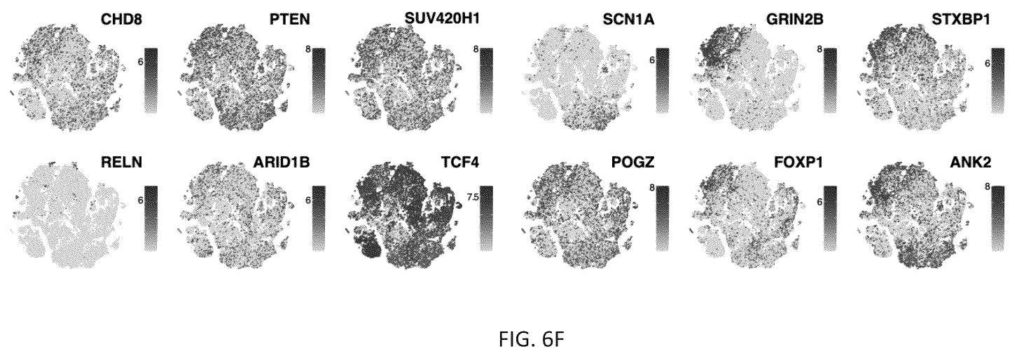

The cell type-specific expression of ASD risk genes in this organoid model was then examined using a set of 102 genes identified as contributing to ASD risk from the largest-to-date ASD exome sequencing study⁸. The expression of these genes was first evaluated as a set in each cell type of the combined scRNA-seq datasets (FIG. 1D and FIGS. 6A, 6D). Consistent with published data for the fetal human brains, it was found that as a set, ASD genes were highly enriched in excitatory projection neurons and interneurons, compared to the other cell types present in the organoids, in both stem cell lines (FIG. 1E and FIGS. 6B, 6E). Next, expression of individual genes was examined; detectable expression of each of the 102 genes was found in patterns that varied by cell type and developmental stage (FIG. 1F and FIGS. 6C, 6F). These results validated these organoids as reproducible models to investigate cortical cell types and their developmental trajectories, and indicated that organoids might be used to discover neurodevelopmental phenotypes of ASD risk genes.

In order to begin to understand the molecular effects of ASD risk genes and whether they show convergent effects in human brain development, three genes were chosen for further study. The above panel of ASD risk genes⁸ was started with, and the focus was on those that were: a) more strongly associated with ASD than intellectual disability/developmental delay and other neurodevelopmental abnormalities^{8,21}, b) predominantly found as de novo protein-truncating variants, which are more likely to be associated with increased ASD liability⁴⁸, and c) expressed early in brain development, and thus likely to reflect early developmental events that can be modeled in organoids⁸.

Based on these criteria, SUV420H1, CHD8, and PTEN were chosen, which span different degrees of ASD risk, with CHD8 having the highest risk association⁸. In the scRNA-seq dataset, each of these three genes was expressed across multiple ages and cell types, precluding a priori prediction of cell type-specific effects (FIG. 1F and FIGS. 6C, 6F). These genes were selected for comprehensive unbiased profiling using the organoid model system.

### SUV420H1 Haploinsufficiency Causes Increased Production of Deep-Layer Cortical Neurons

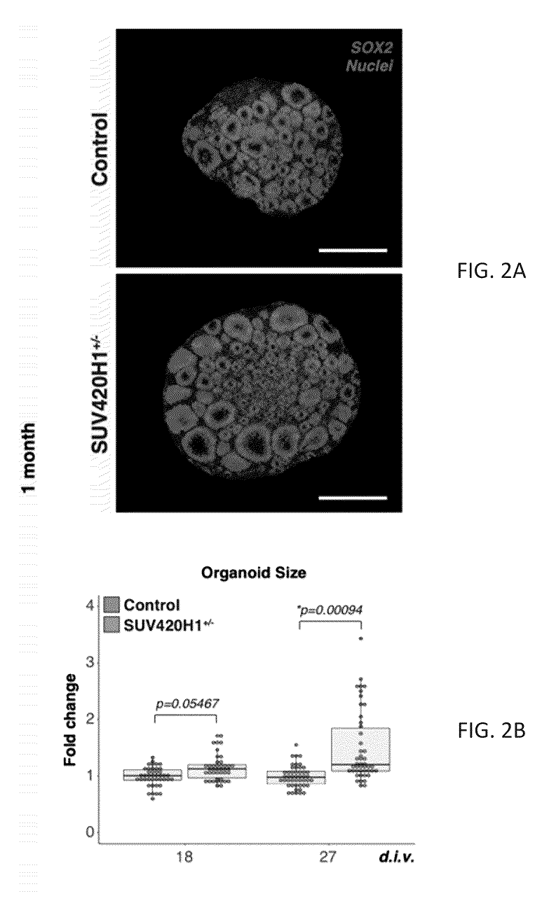

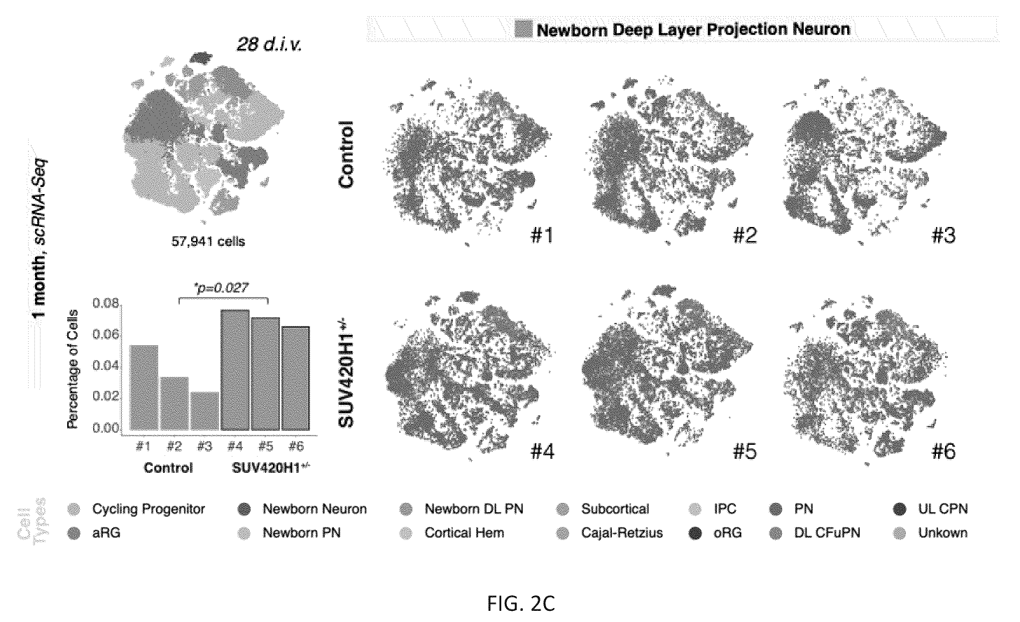

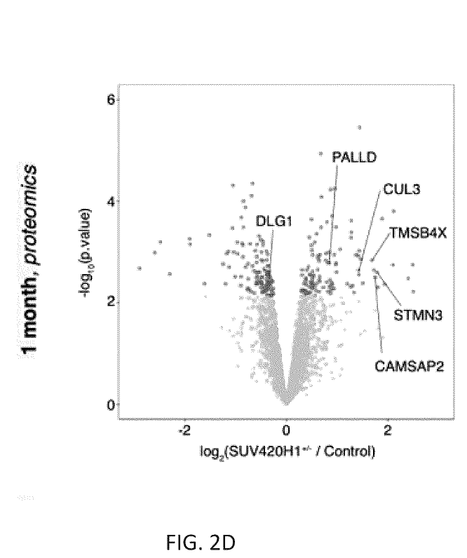

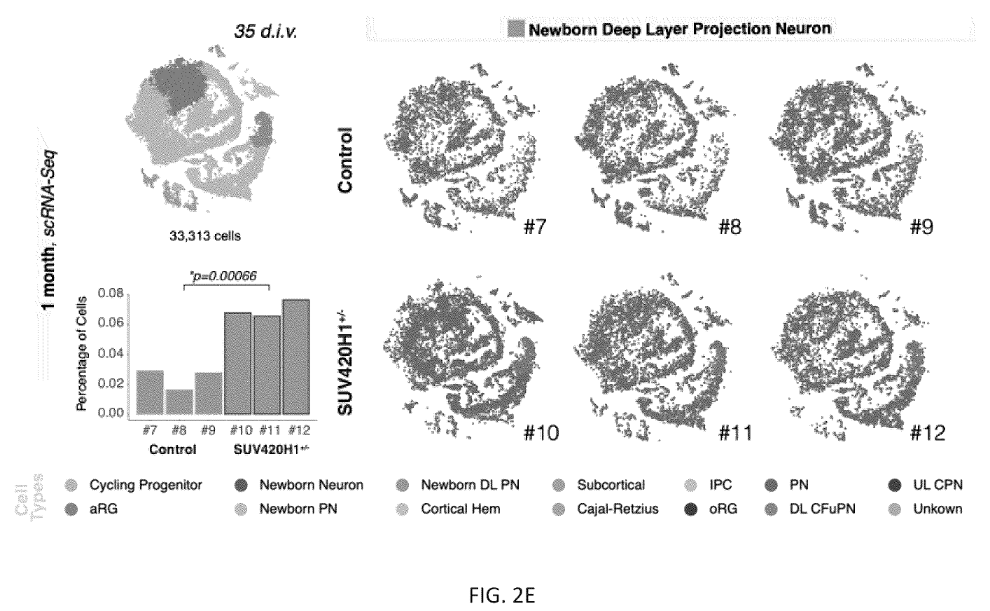

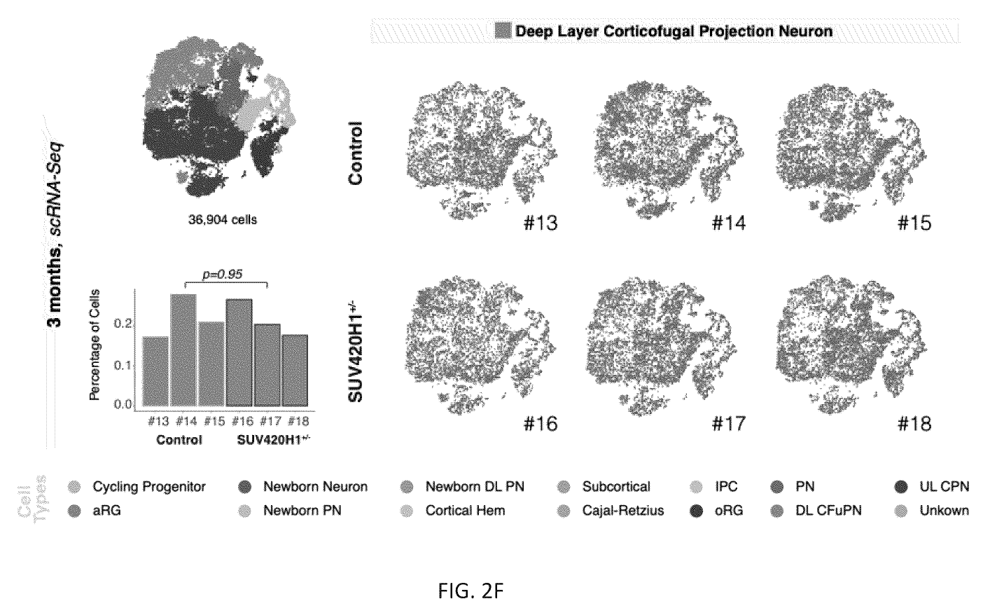

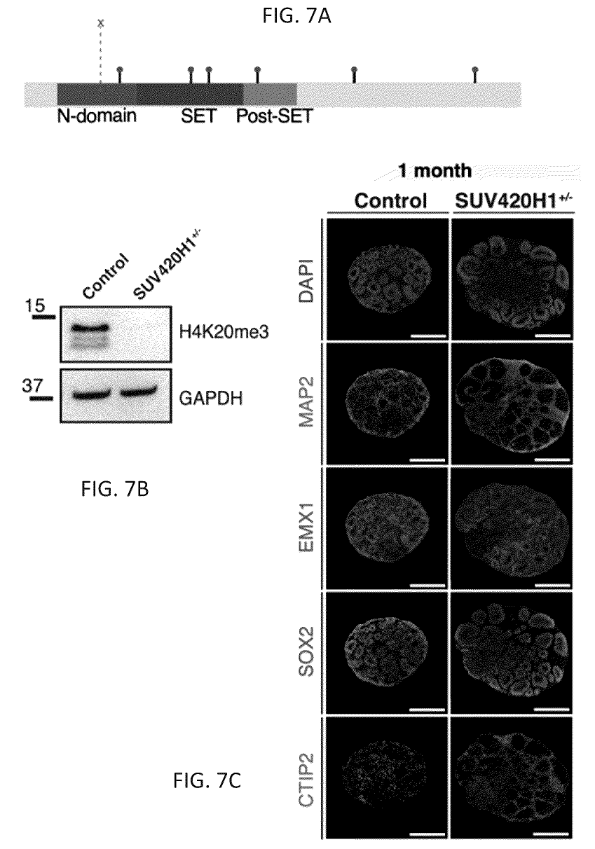

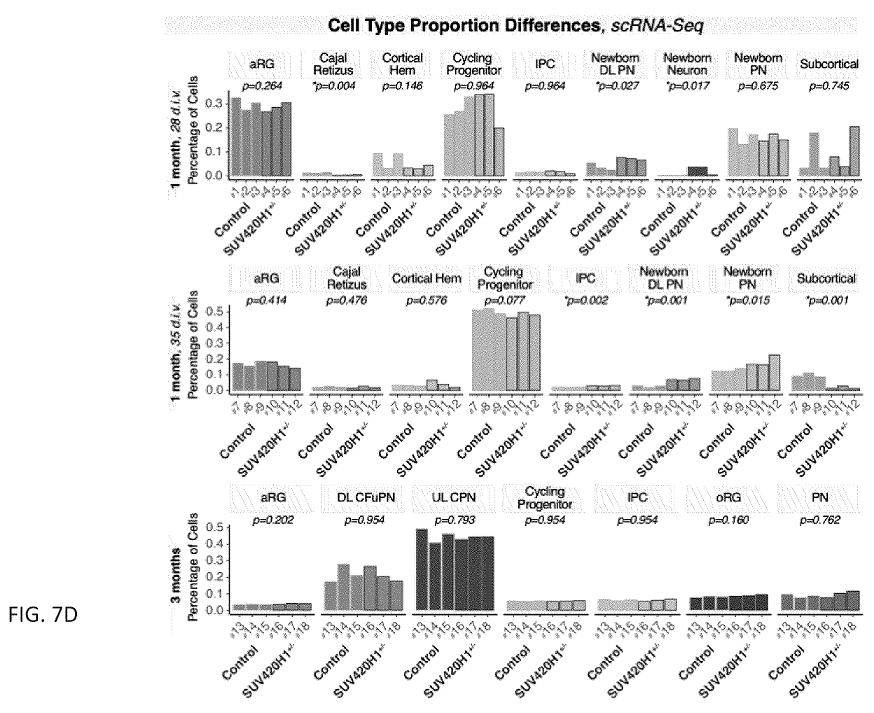

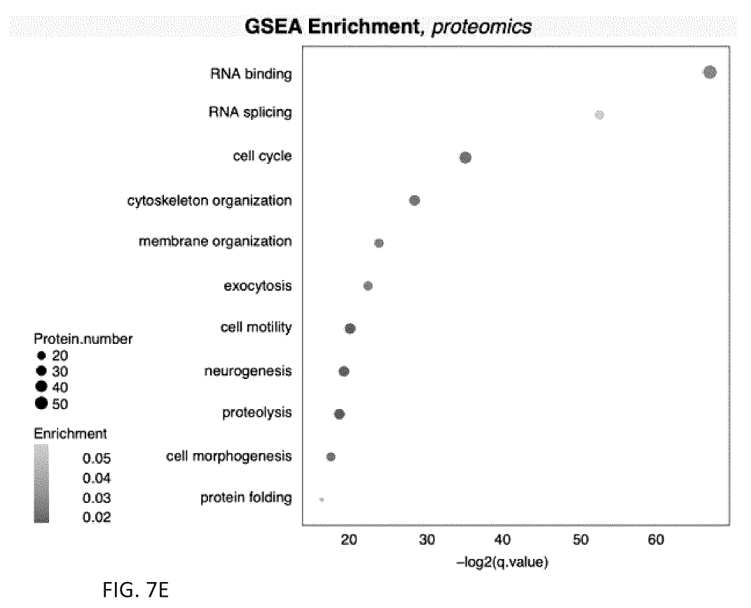

To investigate the effects induced by SUV420H1 haploinsufficiency on human cortical development, a human iPSC line bearing a protein-truncating frameshift in the N-domain of the SUV420H1 protein (see Methods) was generated and validated, and cortical organoids were generated from isogenic control and SUV420H1 heterozygous mutant cell lines (FIG. 2 and FIGS. 7A-7C). Immunohistochemistry for the progenitor markers EMX1 and SOX2, for the early pan-neuronal marker MAP2, and for the cortical pyramidal neuron subtype marker CTIP2 at one month in culture showed that mutant organoids and their respective controls were able to efficiently begin differentiation (FIG. 2A and FIG. 7C). Notably, at 27 days in vitro (d.i.v.), SUV420H1 mutant organoids were already substantially larger compared to controls, mimicking the macrocephaly phenotype observed in individuals carrying de novo mutations in the SUV420H1 gene (n=93 organoids, p=0.0009, t-test; FIG. 2B)^{21,22,27}.

The analysis was first focused on one-month organoids. Transcriptional profiling was performed by scRNA-seq of 57,941 single cells from individual control and mutant organoids at 28 d.i.v. (n=3 single organoids per genotype; FIG. 2C). This identified ten main transcriptionally distinct cell types, including different progenitor cell types (apical radial glia, intermediate progenitors, and cycling progenitors) and newborn neuronal cell types. The cellular composition of both control and mutant SUV420H1 organoids at 28 d.i.v. was highly reproducible within genotypes. However, comparison between the genotypes showed a consistent increase in the cluster containing newborn deep layer projection neurons (the first-born neurons of the cortical plate^{29,30}), accompanied by a decrease in a population of Cajal-Retzius cells (earlier-born pioneering neurons of the pre-plate³¹) in the mutant (FIG. 2C, FIG. 7D). The newborn deep-layer neurons, which expressed TBR1, FOXP2, and TLE4, were consistently increased in every organoid analyzed (n=3 single organoids per genotype; p=0.027, logistic mixed models; FIG. 2C).

To investigate the effect of SUV420H1 haploinsufficiency on protein expression at one month in culture, whole-proteome analysis was performed on single organoids from SUV420H1 mutant and control lines, from a separate differentiation (n=3 single organoids per genotype), using isobaric tandem mass tag (TMT) multiplexed quantitative mass spectroscopy (MS). 233 proteins determined to be significantly differentially expressed were identified (FDR<0.1, moderated t-test; >4,000 proteins detected per sample, FIG. 2D). Using Gene Set Enrichment Analysis (GSEA), it was found that differentially expressed proteins (DEPs) were enriched for the term “neurogenesis”, as well as other broader processes such as “RNA binding” and “cell cycle” (FIG. 7E). DEPs included proteins associated with neuronal processes such as axon guidance (e.g., PALLD, TMSB4X, CAMSAP2), synaptogenesis (e.g., DLG1, CUL3), and neuronal development (STMN3) (FIG. 2D), suggesting an effect on neuronal differentiation in the mutant.

In order to determine whether phenotypic abnormalities induced by SUV420H1 heterozygosity persisted in more mature developmental stages, scRNA-seq was performed on two later timepoints, 35 d.i.v. and three months, using additional batches of organoids from two additional differentiations (FIGS. 2E-2F). At 35 d.i.v, scRNA-seq on 33,313 single cells from SUV420H1 control and mutant organoids revealed an increase in newborn deep layer projection neurons, as well as smaller changes in intermediate progenitor cells, newborn projection neurons, and the subcortical clusters (FIG. 7D). In agreement with the relative increase in newborn deep layer projection neurons observed at 28 d.i.v., a larger increase was detected in this same population of neurons in a separate batch of organoids differentiated to 35 d.i.v. The phenotype was more pronounced at this later developmental stage, and remained consistent in every individual organoid analyzed (n=3 single organoids per genotype; p=0.00066, logistic mixed models; FIG. 2E).

These data indicate that despite widespread SUV420H1 expression (FIG. 1F and FIGS. 6C, 6F), its mutation results in a cell type-specific phenotype affecting a defined population of cortical neurons. Interestingly, this phenotype was rescued over developmental time in organoids; scRNA-seq on 36,904 cells from control and mutant organoids after three months in vitro showed that the percentage of deep layer corticofugal projection neurons was no longer significantly different between SUV420H1 mutant and control (n=3 single organoids per genotype; p=0.95, logistic mixed models; FIG. 2F), nor were any other cell types (FIG. 7D). Altogether, this data indicates that deep layer excitatory neurons are generated earlier in SUV420H1 mutant organoids compared to isogenic controls, and that this neuronal overgrowth phenotype is restricted to a defined early developmental period.

### PTEN Haploinsufficiency Leads to Accelerated Production of Upper Layer Callosal Projection Neurons

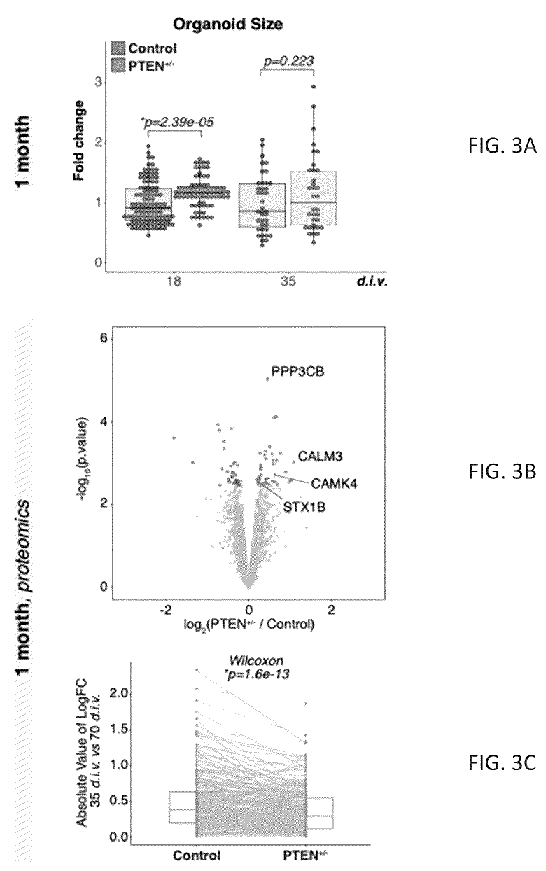

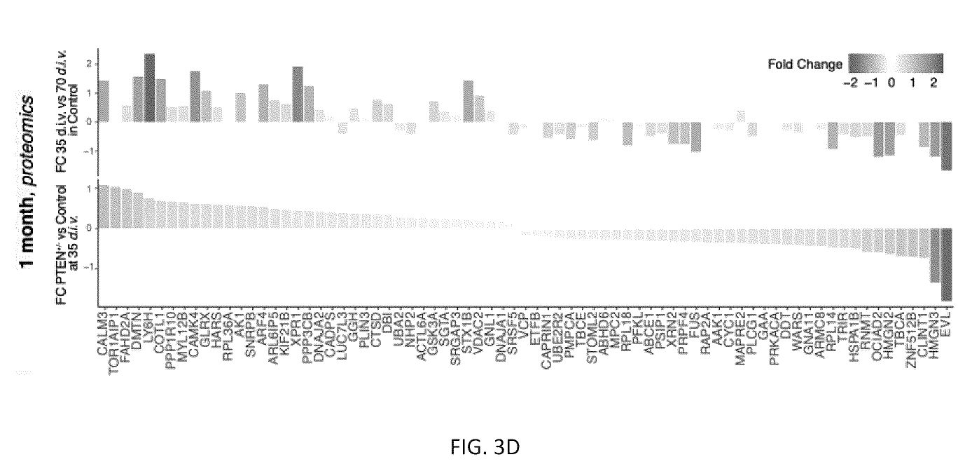

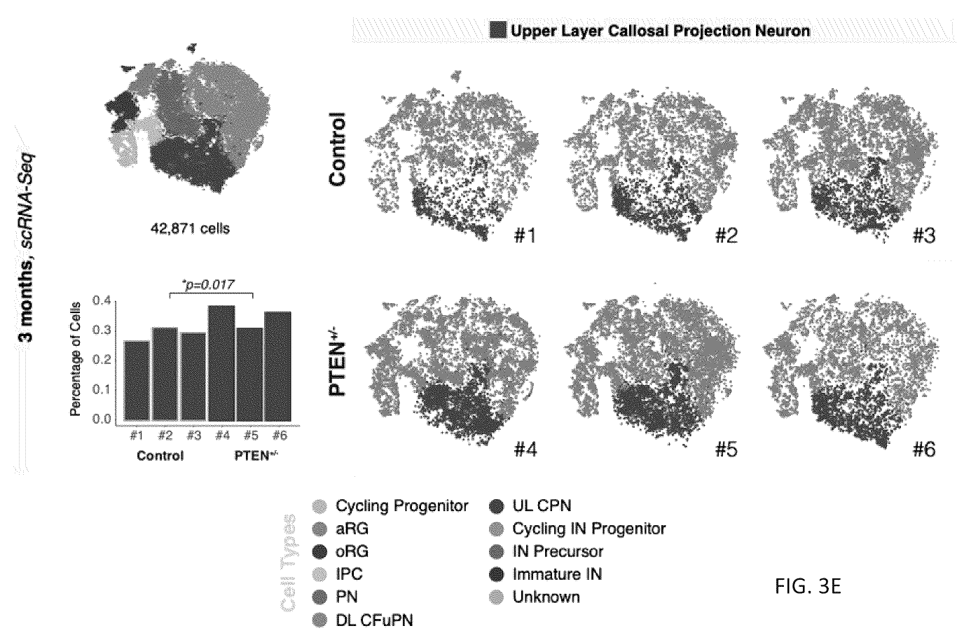

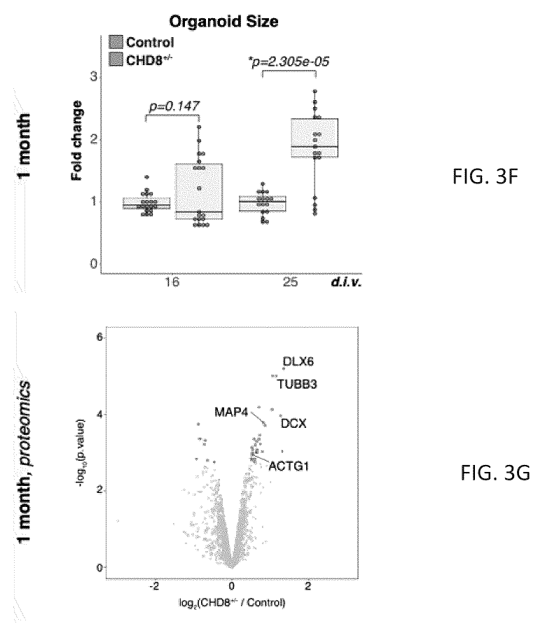

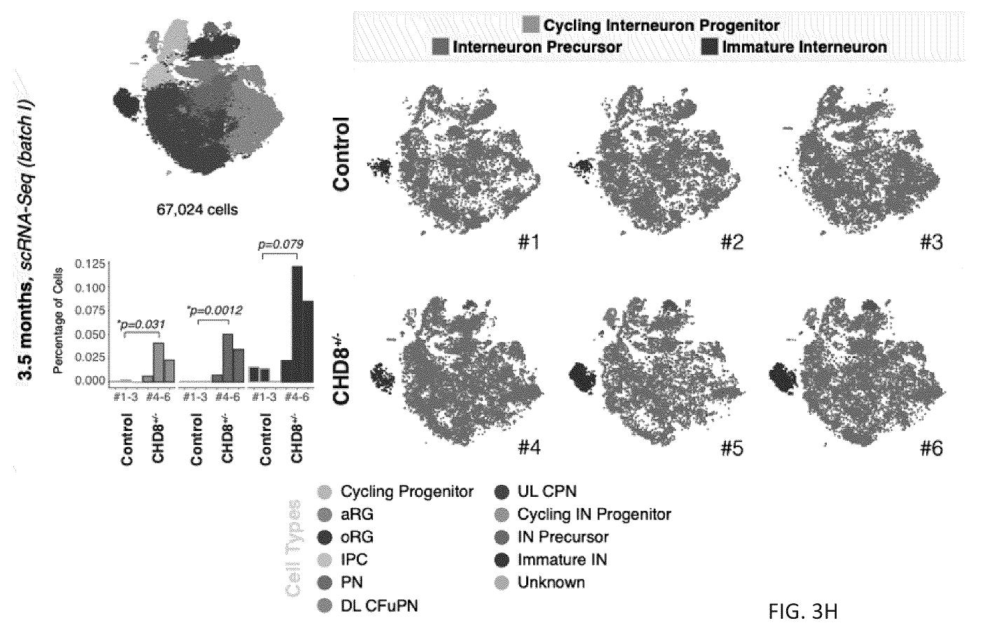

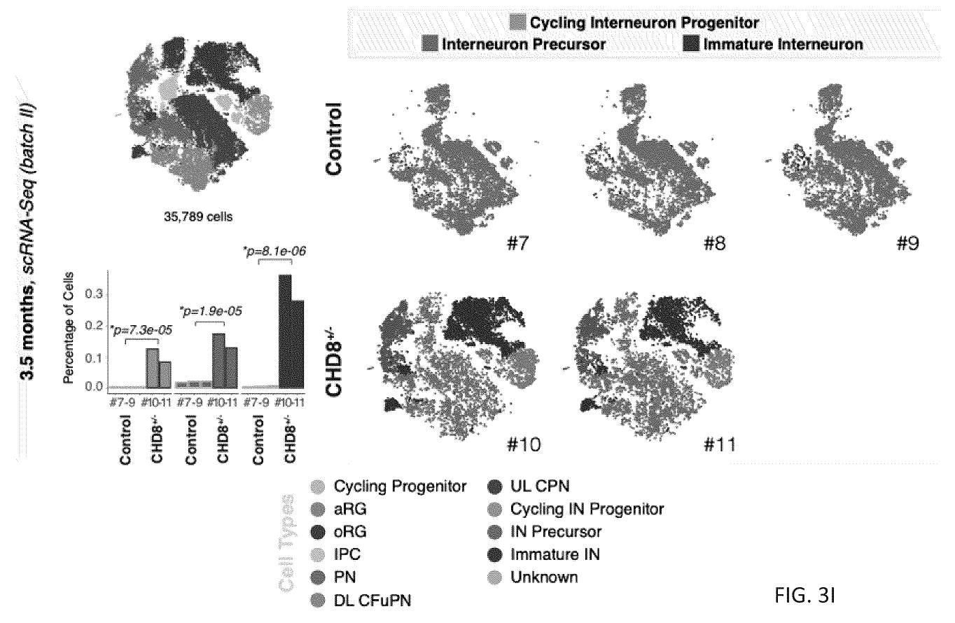

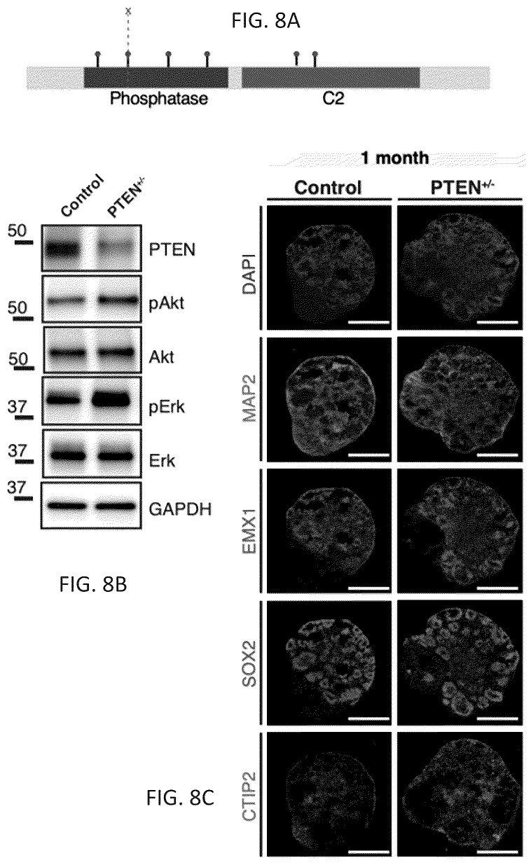

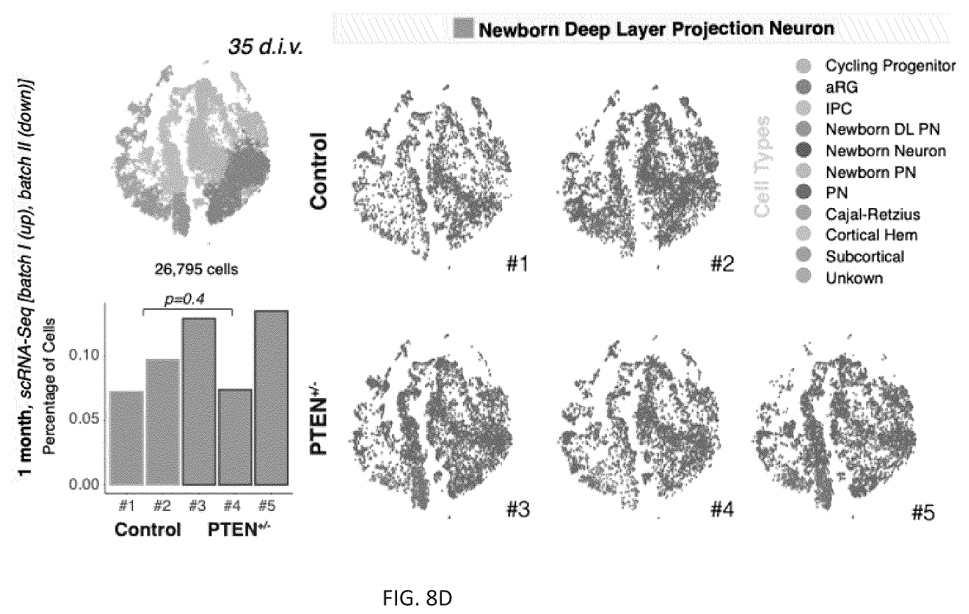

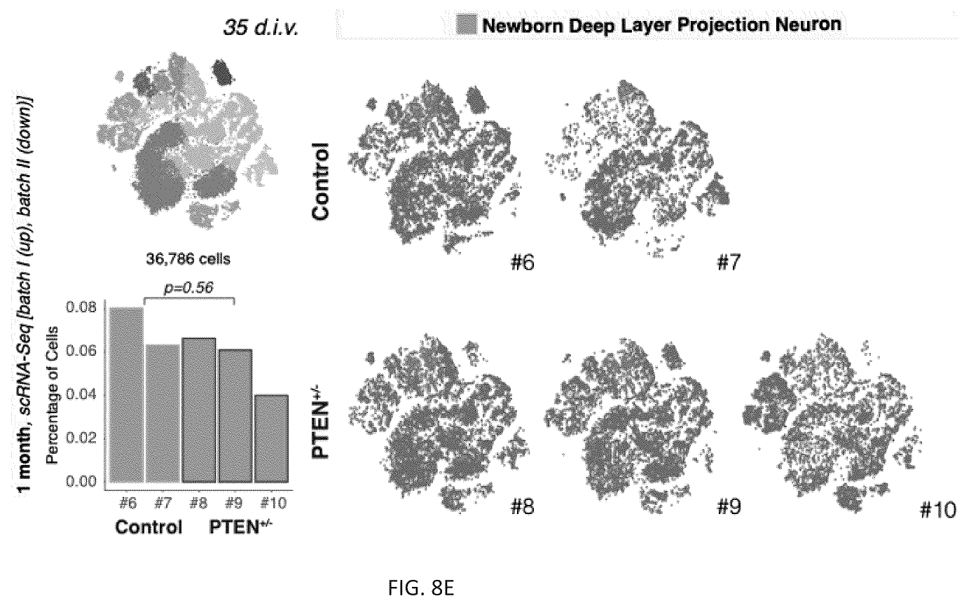

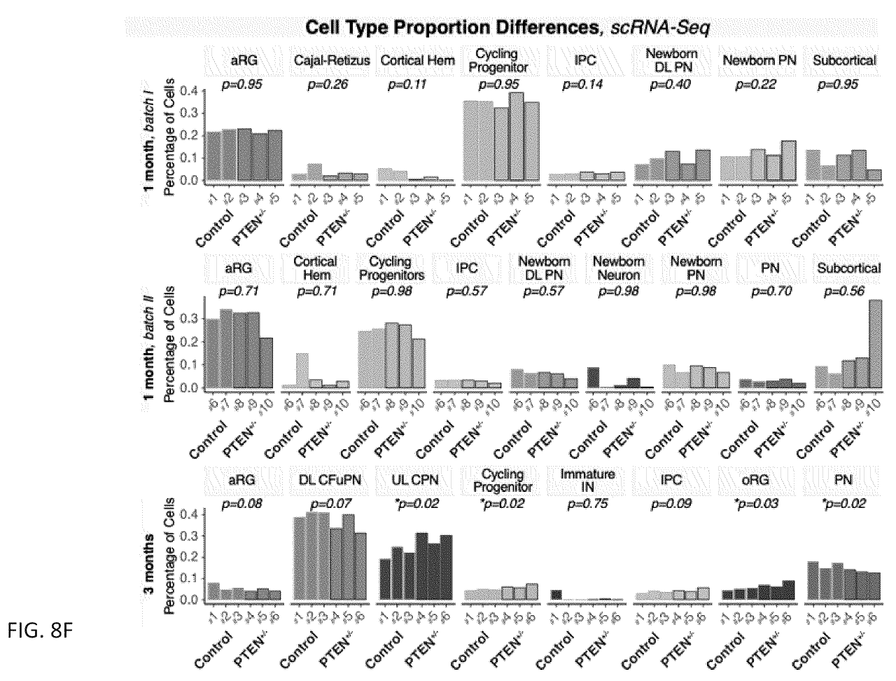

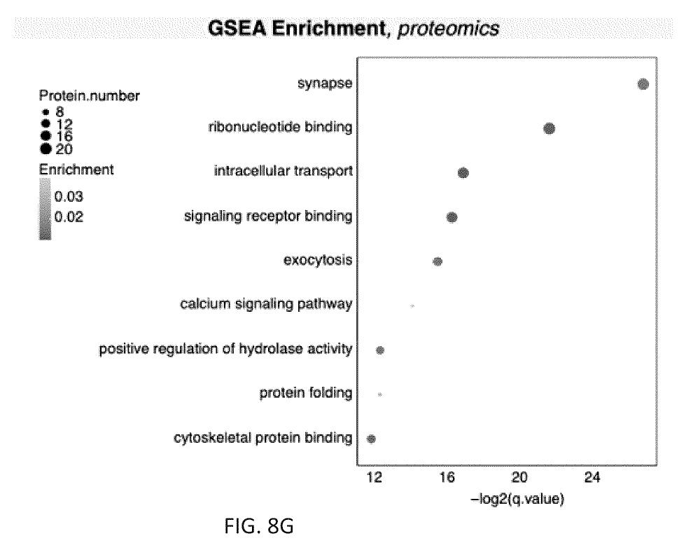

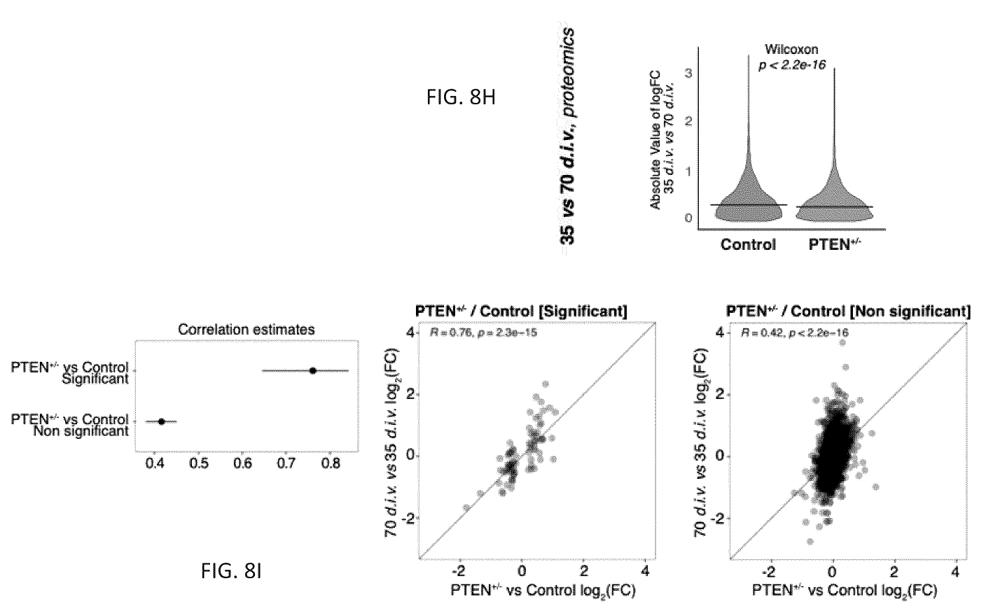

To understand whether mutations in other ASD risk genes also result in altered production of neurons of the cortical plate, the molecular phenotype of PTEN haploinsufficiency was examined in the organoid model. A PTEN mutant iPSC line was created with a protein-truncating frameshift mutation in the phosphatase domain, a region found mutated in patients (³², see Methods), and generated mutant and isogenic control organoids (FIGS. 3A-3E and FIGS. 8A-8C). Immunohistochemistry on one-month organoids indicated the presence of appropriate progenitor and neuronal populations, confirming appropriate initiation of organoid development (FIG. 8C). Like the SUV420H1 mutant organoids, PTEN mutant organoids showed increased size compared to their isogenic controls at 18 d.i.v. (n=84 organoids, p=2.39e−05, t-test; FIG. 3A). This resembles the macrocephaly phenotype reported in patients with PTEN mutations²⁸.

To investigate the cellular composition of PTEN mutant and isogenic control organoids, scRNA-seq and proteomic analysis was performed on individual organoids at one month in culture (FIG. 3B and FIG. 8D). Analysis of 26,795 single cells at 35 d.i.v. showed that, in contrast to SUV420H1, in the PTEN mutant organoids there was no difference in the proportion of newborn deep layer neurons (n=3 single organoids per genotype, p=0.4, logistic mixed models; FIGS. 8D, 8F) or any other cell type (FIG. 8F). In order to validate this result, the scRNA-seq analysis was repeated and proteomic analysis was included using two new differentiations. scRNA-seq of an additional 36,786 cells from a second batch of organoids grown for 35 d.i.v confirmed the first result, revealing no difference between genotypes in this replicate experiment (n=3 single organoids per genotype, p=0.56, logistic mixed models; FIGS. 8E-8F). However, whole-proteome MS on single PTEN mutant and isogenic control organoids at one month in culture (n=4 single organoids per genotype), showed significant differential expression of 75 proteins between genotypes (p<0.1, moderated t-test; FIG. 3B). GSEA identified “synapse” as the most enriched term for PTEN DEPs (FIG. 8G), along with exocytosis (e.g., STX1B) and the calcium signaling pathway (e.g., PPP3CB, CALM3, and CAMK4) (FIG. 3B), pointing to an effect of PTEN mutation on neuronal development.

To better understand the developmental relevance of these changes, whole proteome analysis was performed of PTEN mutant and isogenic control organoids at a later timepoint, 70 d.i.v. (n=3 single organoids per genotype). While there were no differences in protein abundance between mutant and controls, when the proteins that were differentially expressed between ages (35 vs 70 d.i.v.) in each genotype were examined, the mutant organoids showed significantly smaller expression changes between ages than controls (p=1.6e−13, Wilcoxin rank test, FIG. 3C and FIG. 8H), indicating that the mutant 35 d.i.v. organoids resembled their older counterparts more closely than the control 35 d.i.v. organoids. Furthermore, the mutation-driven changes (i.e., the set of proteins that were differentially expressed between genotypes in the 35 d.i.v. organoids) were correlated with age-driven changes (i.e., the set of proteins that changed in both the control and mutant proteomes through time, 35 d.i.v. vs 70 d.i.v.), indicating that PTEN mutation affects expression of developmentally-regulated proteins (R=0.76, p<1e−15, Pearson correlation; FIG. 3D and FIG. 8I). This result points to accelerated differentiation of PTEN mutant organoids that may occur later than one month in culture.

scRNA-seq profiling was therefore performed of 42,871 cells from PTEN mutant and control organoids at three months in culture (n=3 separate organoids per genotype). Mutant organoids at this stage showed a small increase in cycling progenitors and oRG (FIG. 8F), consistent with the overgrowth phenotype observed in mutant organoids (FIG. 3A) and with a prior study reporting that PTEN homozygous loss-of-function human cerebral organoids showed overgrowth coupled with an expansion of neural progenitors³³. Strikingly, at this stage, the PTEN mutant organoids also showed overproduction of a specific population of excitatory neurons: callosal projection neurons (p-val=0.017, logistic mixed models; FIG. 3E and FIG. 8F).

Although affecting a different population of excitatory neurons then the SUV420H1 mutation, both SUV420H1 and PTEN converged on a share phenotype of imbalance of production of cortical excitatory neurons.

### CHD8 Haploinsufficiency Causes Overproduction of Cortical Interneurons

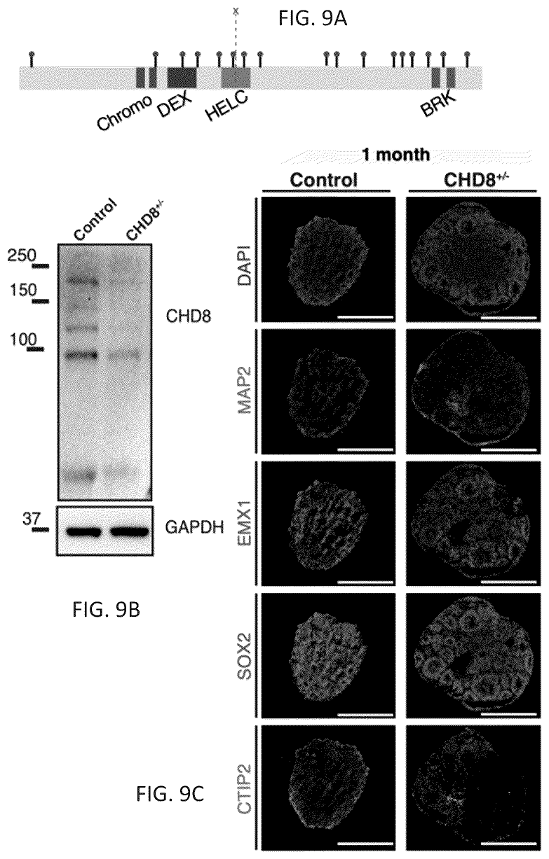

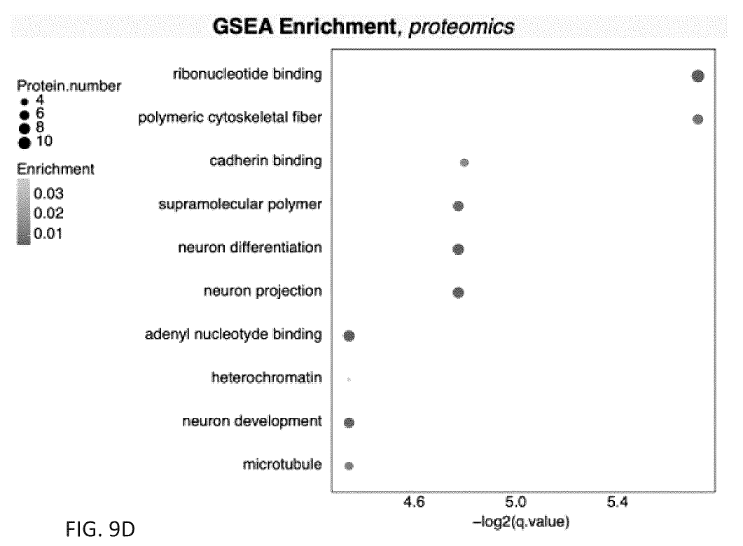

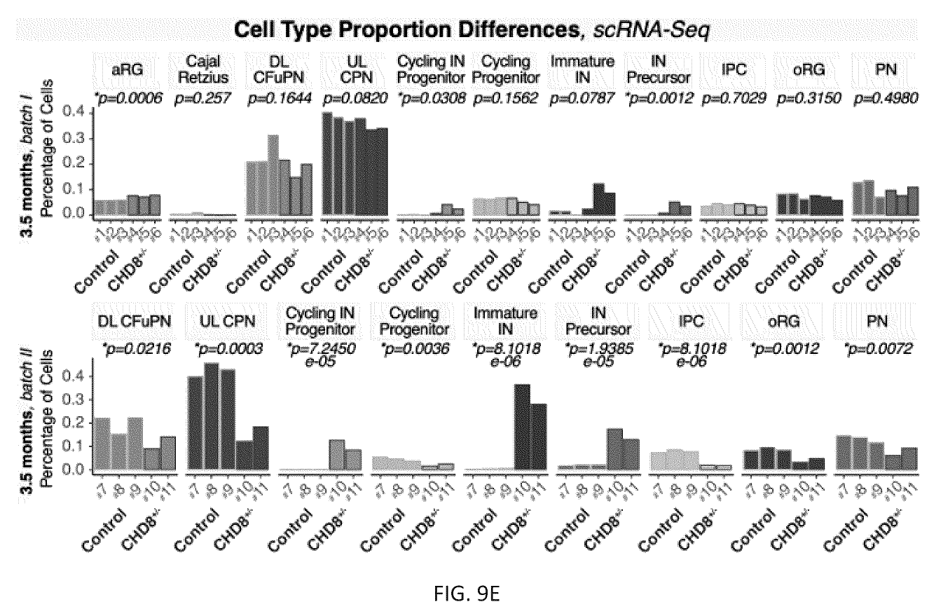

In order to determine if alterations in the speed of neurogenesis of cortical neurons was shared by a third mutation, a human ESC line heterozygote was obtained for a protein-truncating frameshift in the helicase-C domain of the CHD8 gene (see Methods), and generated mutant and isogenic control cortical organoids (FIGS. 3F-3I and FIGS. 9A-9C). Immunohistochemistry at one months in vitro indicated the presence of dorsal forebrain progenitors (SOX2 and EMX1) and neuronal populations (MAP2 and CTIP2), indicating that haploinsufficiency in CHD8 does not disrupt early stages of neurodevelopment using this organoid protocol (FIG. 9C). Notably, as with SUV420H1 and PTEN, CHD8 mutant organoids showed increased size at 25 d.i.v. (n=33 organoids, p=2.305e−05, t-test; FIG. 3F), consistent with macrocephalic phenotypes reported in 80% of patients with mutations in CHD8²⁶.

Whole-proteome MS was performed on one-month CHD8 organoids (n=3 for each of mutant and control), and significant differential expression of 35 proteins was found between genotypes. The interneuron fate regulator DLX6³⁴ was found among the most upregulated proteins in the mutant (FIG. 3G), suggesting an earlier commitment of CHD8 mutant progenitors towards an interneuron fate. Additionally, other proteins associated with neuronal differentiation and cytoskeleton remodeling (TUBB3, DCX, MAP4, ACTG1) were upregulated in mutant organoids (FIG. 3G). Enriched terms in GSEA included “neuron differentiation”, “neuron projection”, and “neuron development” (FIG. 9D). These data suggest that cortical neurons might also be affected in CHD8 haploinsufficient organoids, similarly to what observed for SUV420H1 and PTEN.

These results prompted the performance of scRNA-seq analysis on six CHD8^{+/-} and control organoids cultured for 3.5 months, a stage when interneurons begin to appear (67,024 cells, n=3 single organoids per genotype, 109 d.i.v.). At this stage, the analysis revealed a dramatically increased number of cycling interneuron progenitors, interneuron precursors, and immature interneurons in mutant organoids (n=3 single organoids per genotype; cycling interneuron progenitors: p=0.031, interneuron precursors: p=0.0012, immature interneurons: p=0.079, logistic mixed models; FIG. 3H). A slight increase was observed in the number of apical radial glia (FIG. 9E), suggesting that the increased number of progenitors might not be restricted to the ones committed to an interneuron fate.

This experiment was repeated using an independent batch of organoids from a separate differentiation, profiling 35,789 single cells of control and mutant organoids collected at 3.5 months. An even more dramatic increase in the numbers of immature interneurons was observed, as well as in the numbers of interneuron precursors and cycling interneuron progenitors (n=3 single organoids per genotype; immature interneurons: p=8.1e−06, interneuron precursors: p=1.9e−05, cycling interneuron progenitors: p=7.2e−05, logistic mixed models; FIG. 3I). The increase in interneurons was so large that a decrease in the represented proportion of all the other cell types was observed in the mutant organoids (FIG. 9E). The data demonstrate that, as observed for the other two mutations, CHD8 haploinsufficiency is associated with overgrowth of a specific neuronal lineages of the cortical microcircuit: cortical interneurons.

Altogether, the data indicate that despite the widespread expression and broad functions of each of the three ASD risk genes analyzed here, all converge on overproduction of cortical neuronal lineages. Interestingly, each mutation affects different subsets of neuronal populations. These findings indicate that ASD pathology in these three genes may converge on a broad phenotype (neuronal overgrowth), rather than on a shared cell type or molecular pathway.

### Developmental Trajectories of SUV420H1, PTEN, and CHD8 Show Accelerated Neurogenesis of Specific Neuronal Subtypes

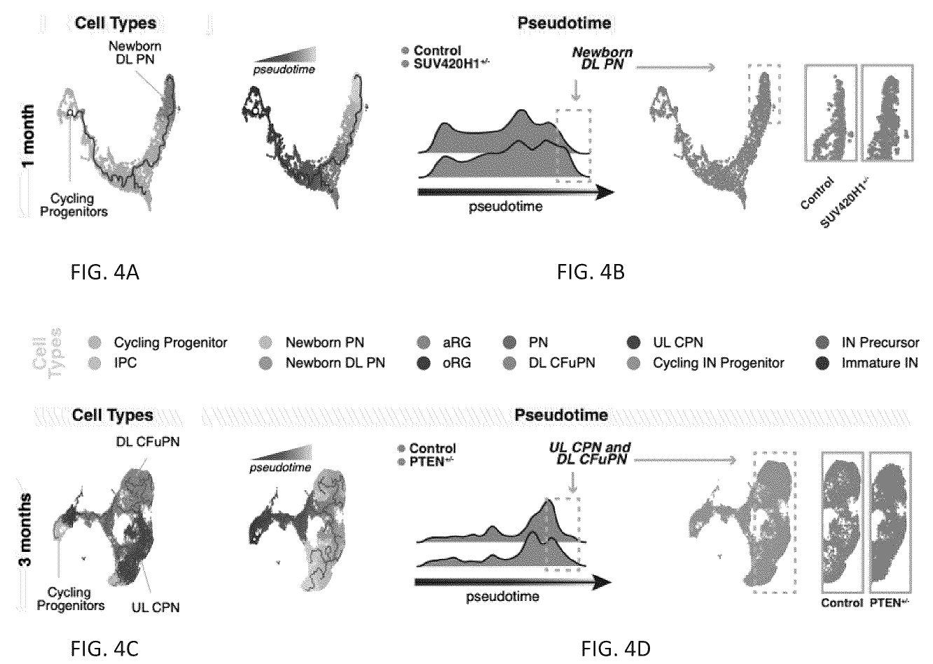

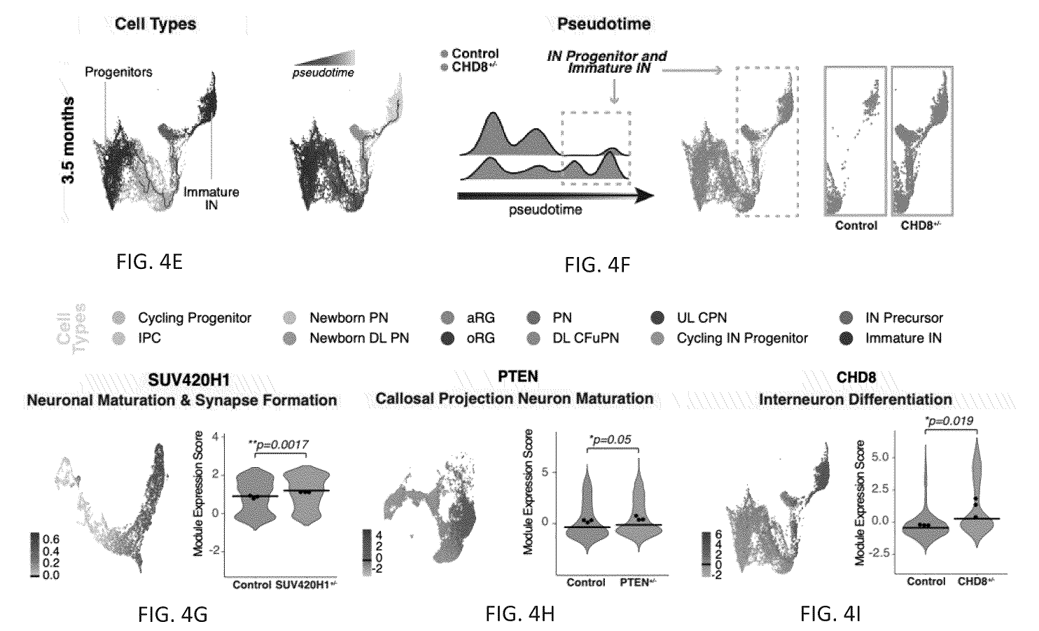

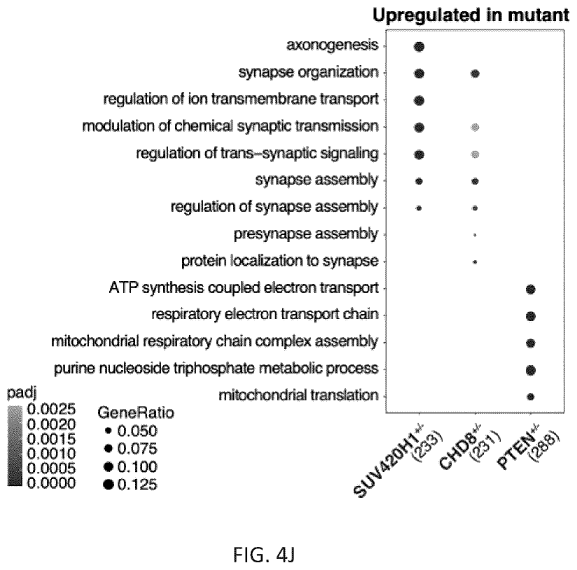

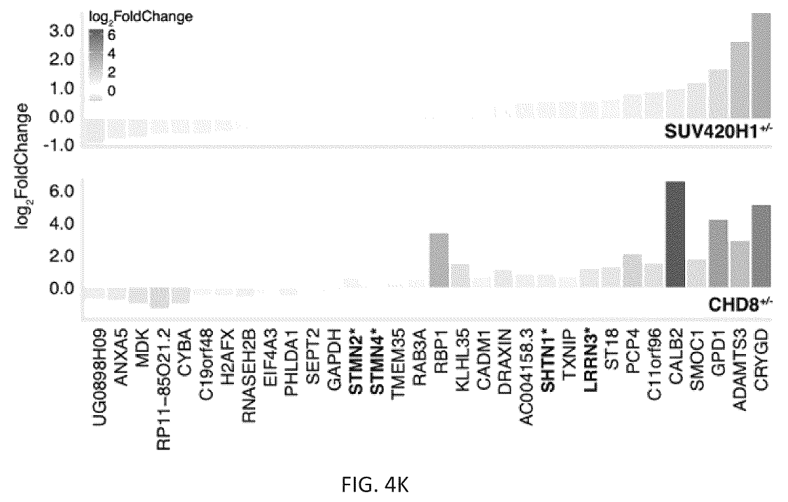

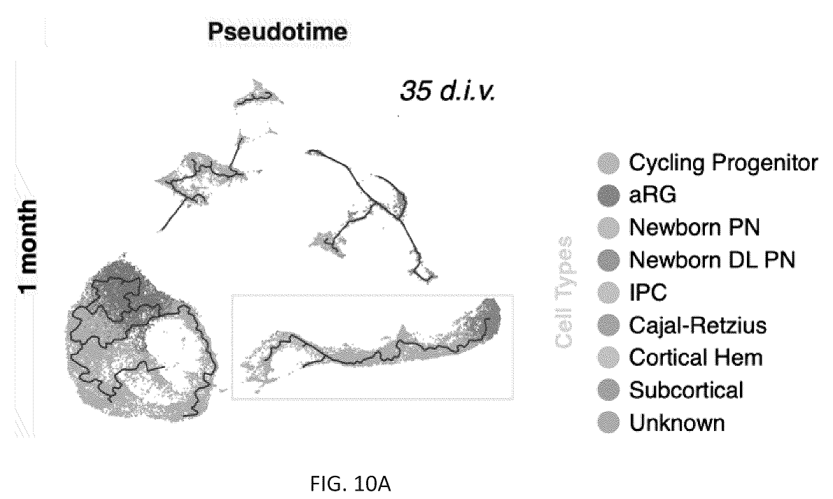

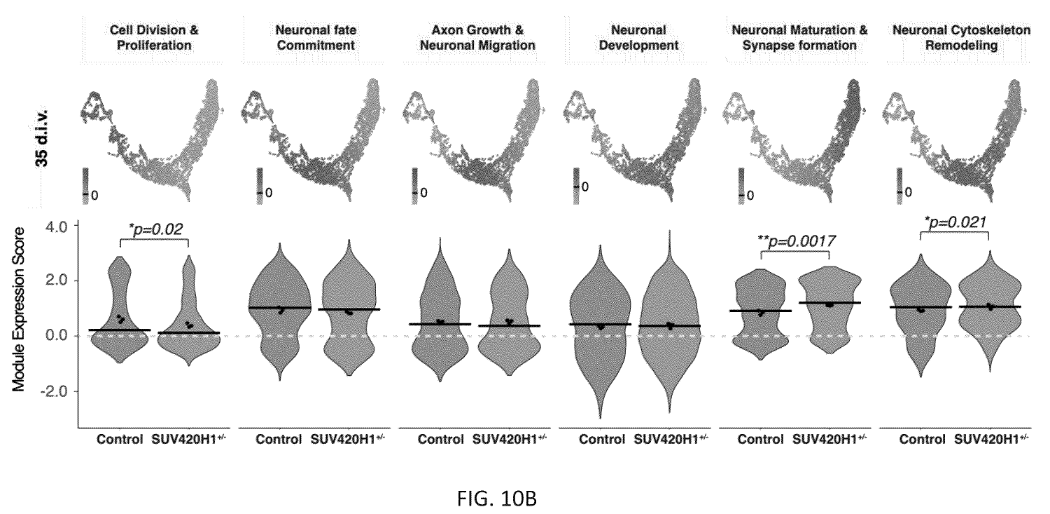

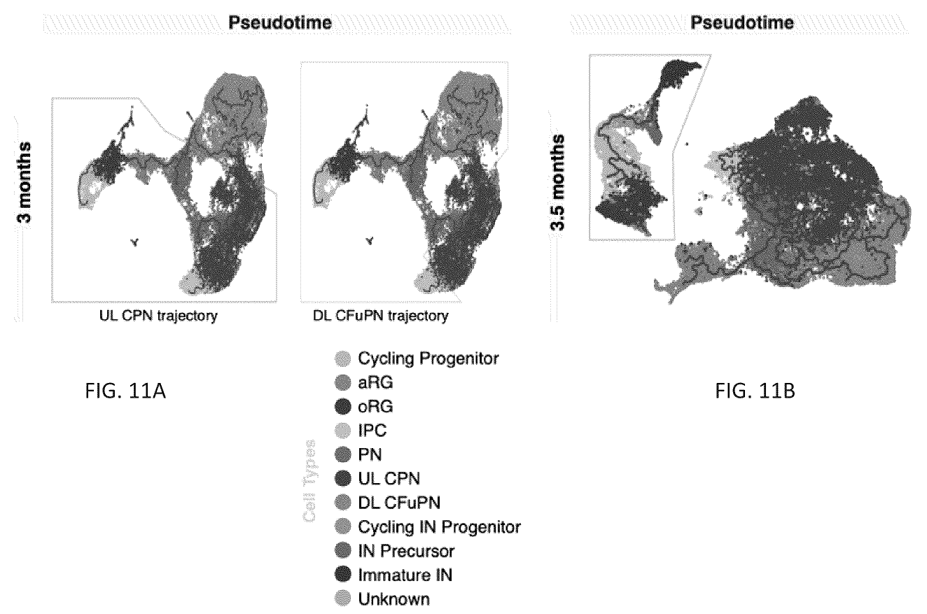

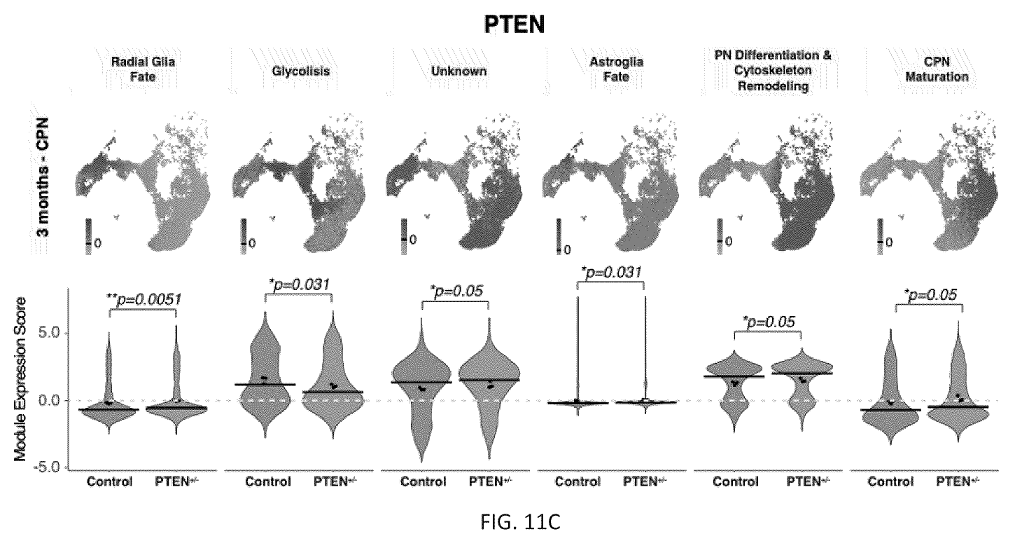

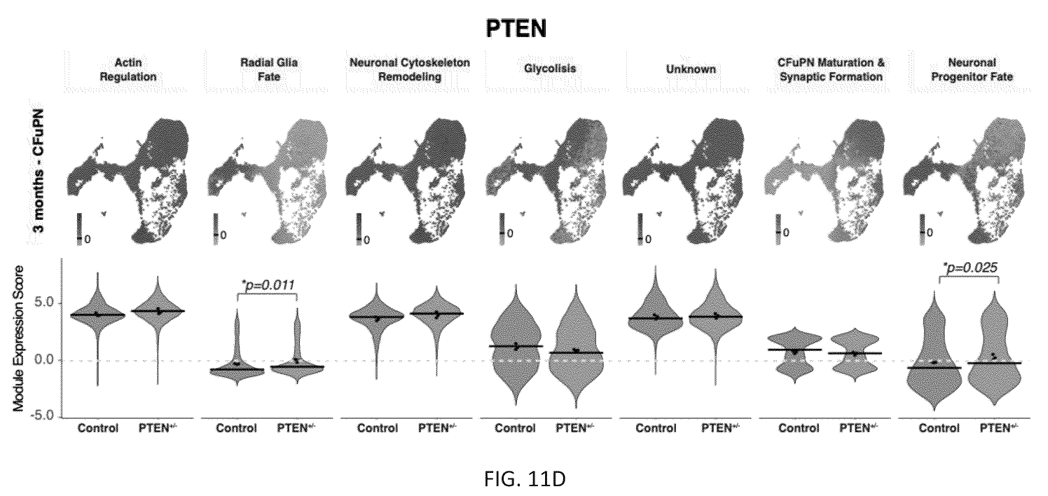

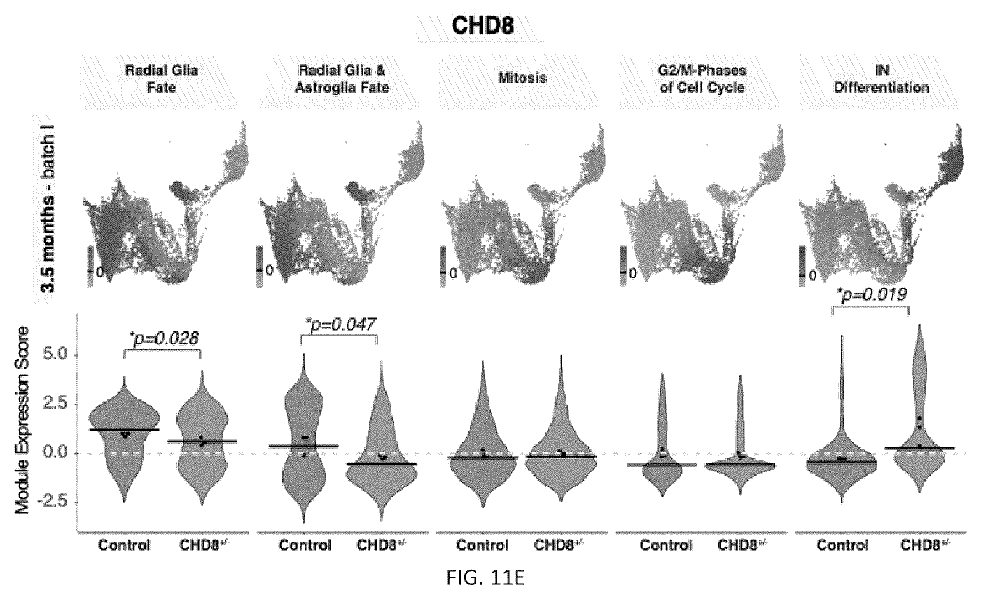

In order to determine if similarly altered developmental progression was observed between the three mutations, it was next sought to identify where each mutant population of neurons was located along the organoid developmental trajectory. Pseudotime trajectories were calculated for each scRNA-seq dataset using Monocle3³⁵, after downsampling to have an equal number of control and mutant cells. For all three mutants, the pseudotemporal ordering of cell types approximated that of in vivo human development, following a trajectory that progressed from progenitors to neuronal cell types (FIGS. 4A-4F, FIG. 10A, and FIGS. 11A-11B). For SUV420H1 at 35 d.i.v., the cells split into two partitions, one containing progenitors traversing the cell cycle, and one containing cycling progenitors maturing towards newborn deep layer neurons (FIG. 10A). The latter contained the neuronal populations most consistently affected by the mutation, and was thus selected as the partition of interest. For PTEN, the cells were all assigned to one trajectory, with progenitors at the beginning, and a branch point between cells progressing to callosal projection neurons versus corticofugal projection neurons (FIG. 11A). Finally, for CHD8, the partition of interest showed cells progressing from radial glia to interneurons, while another contained the progression from radial glia to projection neurons (FIG. 11B1).

Strikingly, for each of the three genes, mutant organoids showed an increased distribution of cells toward the end point of these developmental trajectories, i.e., at a more advanced stage of differentiation, compared to control (FIGS. 4B, 4D, 4F). Combined with the increased number of neurons in the mutant organoids, these results suggest an accelerated neurogenesis phenotype in each of these mutants.

In order to explore molecular differences that may underlie these phenotypes, gene expression regulation was examined across the partitions of interest using a co-expression network analysis. Using Weighted Correlation Network Analysis (WGCNA,³⁶), modules of genes with highly correlated expression across cells in each dataset were found. Full lists of modules and their associated genes can be found in FIGS. 10-11. For each mutation, at least one module was enriched towards the end of the trajectory and was found to be significantly increased in expression in mutant organoids. In the SUV420H1 35 d.i.v. dataset, several modules were identified (FIG. 10B), one of which was significantly enriched in mutant organoids compared to control (p=0.0017, linear mixed models, FIG. 4G). This module contained multiple genes known to be involved in neuronal maturation and synapse formation (such as GRIA2, CAMK2B, SNAP25, CNTNAP2, NRCAM, and NRNX2). For the three-month PTEN mutant organoids, modules were separately calculated for each branch of the pseudotime (FIG. 11A), leading to upper layer callosal projection neurons and deep layer corticofugal projection neurons, respectively (FIGS. 11C-11D). In the trajectory leading to upper layer callosal projection neurons, several modules were found to be differentially expressed between control and mutant (FIG. 11C); in particular, a module containing genes related to callosal projection neurons and neuronal maturation (such as SATB2, CAMKV, NELL2, SNX7, and SYBU) showed increased expression in mutant cells (p=0.05, linear mixed models, FIG. 4H). Finally, in 3.5-month CHD8 mutant organoids, a module containing genes related to interneuron differentiation (such as DLX1, DLX2, DLX6-AS1, DLX5, SCGN, and GAD2) was upregulated in the mutant (p=0.019, linear mixed models, FIG. 4I), while modules related to progenitor fate were downregulated (FIG. 11E). These modules all demonstrate altered gene regulation consistent with accelerated neuronal maturation.

Collectively, these results demonstrate that mutations in SUV420H1, PTEN, and CHD8 each lead to an acceleration of the developmental trajectory of specific neuronal subtypes in human cortical organoids.

### CHD8 and SUV420H1 Haploinsufficiency Share Signatures of Synaptic Processes

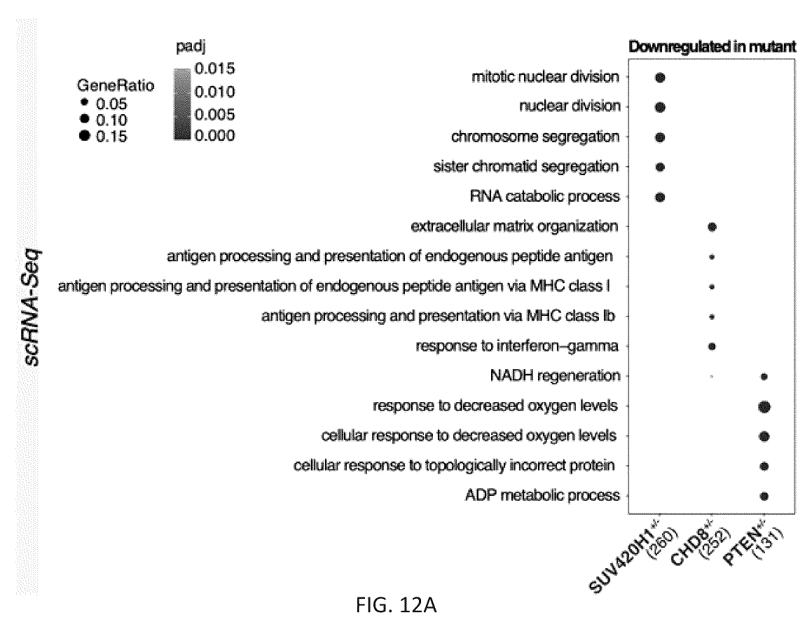

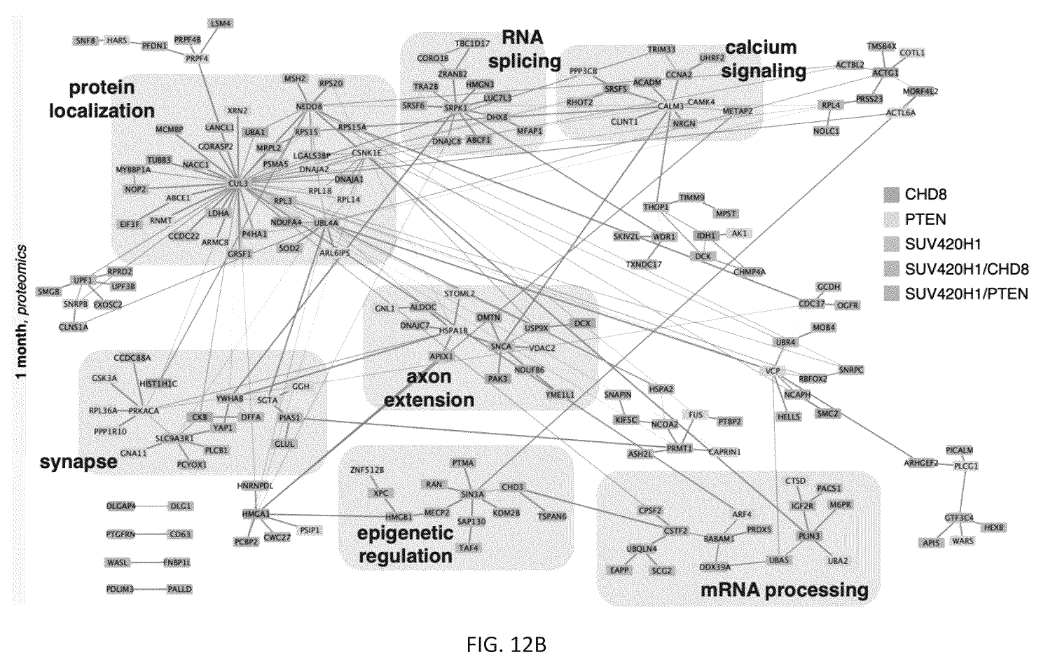

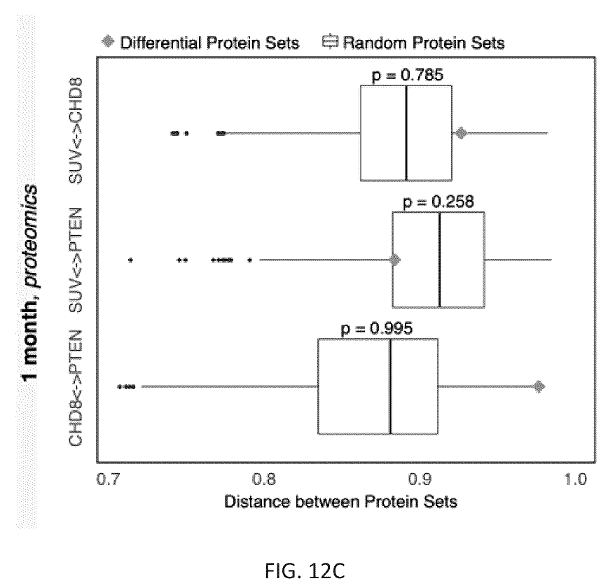

Finally, it was sought to determine whether molecular phenotypes of these three genes converged at the level of specific biological processes or protein networks. First, differential gene regulation changes across the three mutations were compared. Reads from the cells in each selected partition of interest were summed within each organoid, and DESeq2 was used to calculate differentially expressed genes (DEGs) between mutant and control in each dataset (a technique which explicitly controls for sample-to-sample variation). No notable overlap of GO terms for genes downregulated in any of the three mutations was observed (FIG. 12A). Remarkably, however, for both the genes upregulated in the excitatory lineage of 35 d.i.v. SUV420H1 organoids (FIG. 4A), and the genes upregulated in the interneuron lineage of 3.5-month CHD8 organoids (FIG. 4E), synaptic gene ontology (GO) terms such as “synapse organization” and “regulation of trans-synaptic signaling” were significantly enriched (p<0.0025 in all cases, over-representation test; FIG. 4J). Although these GO terms were not significantly enriched for genes dysregulated in the PTEN dataset, some of these genes were upregulated, or trending towards upregulation, in the trajectory branch containing callosal projection neurons of those organoids as well (marked with a * in FIG. 4K). The consistent upregulation of these gene sets across these disparate cell types and timepoints indicates convergent dysregulation in molecular pathways relating to synapse formation and function between CHD8 and SUV420H1 (FIGS. 4J-4K).

Lastly, an integrated protein interaction network was built using the DEPs across mutations, and tested the hypothesis of a convergence of altered networks. It was found that DEPs from all three mutations populated multiple sub-clusters of the network enriched for terms including “synapse”, “axon extension”, and “calcium signaling” (p<0.01 in all cases; FIG. 12B). However, protein-protein interactome weighted distance between the protein sets was calculated, it was found that the protein sets were not closer than would be expected by chance (p >0.25 in all cases; FIG. 12C). This suggests that although, in one-month organoids, SUV420H1, CHD8, and PTEN mutations each affect broad molecular processes related to neuronal function, they do not preferentially act through the same protein effectors.

Collectively, these data show that SUV420H1, CHD8, and PTEN heterozygous organoids converge on a phenotype of accelerated neurogenesis and overexpression of synaptic genes, and show cell type-specific effects on individual neuronal populations.

### Discussion

The process by which mutations in ASD-related genes converge on the neurobiology of this multifaceted disorder remains unclear. Studies have been made difficult by the complexity of ASD genetics, limited experimental access to the human brain, and the lack of experimental models for human brain development. By leveraging reproducible human cortical organoids and single cell genomics, it was found that mutations in three ASD risk genes, SUV420H1, PTEN, and CHD8 converge on a shared phenotype of accelerated early cortical neurogenesis, and define three neuronal classes of the cortical microcircuit as the specific cell types affected. These findings are interesting in light of prior work indicating that ASD genetics affects neurogenesis³⁷⁻³⁹, and it underscores the value of organoids to identify both the developmental processes affected and the target cell types involved.

The finding that different ASD risk genes converge over accelerated neuronal production but diverge one level of complexity below it, with each mutation affecting distinct neuronal types, suggests that the shared clinical pathology of these genes may derive from high-order processes of neuronal differentiation and circuit wiring, as opposed to convergent use of the same cellular and molecular mechanisms. Indeed, for the three ASD risk genes analyzed minimal sharing of molecular pathways was found. These results inform a framework for therapeutic approaches that might be better tailored to the modulation of common, dysfunctional circuit properties instead of common molecular pathways.

Excitatory/inhibitory imbalance of the cortical microcircuit is a major hypothesis for the etiology of ASD⁴⁰⁻⁴⁵. The imbalance could derive from a change in the numerical proportions of cell types within the circuit, incorrect wiring, or altered properties of the neurons and glial cells involved, among others. The increased number of neurons that were observed in mutant organoids points at an early defect in circuit development whereby a small number of neuron classes develop asynchronously with the remaining cell types of the circuit. Studies in vivo have shown that this kind of relatively subtle developmental alterations, even if resolved later in development, can result in disproportionally large functional effects on the mature circuit⁴⁶⁻⁴⁸. Future work to increase the fidelity of circuit and tissue architecture in organoids will be crucial to investigate the implications of ASD risk alterations and potential interventions on circuit physiology and function.

Reproducible brain organoid models, combined with high-throughput single cell genomic methods, are suitable systems for unbiased identification of pathogenic mechanisms in neurodevelopmental disorders. The work paves the way for larger efforts to multiplex perturbation and analysis of large spectra of ASD risk genes⁴⁹ in organoids to understand whether synergistic mechanisms are at play across this vast number of variants and possibly across disease manifestations.

### Materials and Methods

### Data Reporting

The experiments were not randomized. Investigators were not blinded during experiments and analysis of results.

### Pluripotent Stem Cell Culture

The HUES66 human ES cell line and the edited CHD8^{+/-} line (also known as HUES66 AC2—this clone has a heterozygous 13 nucleotide deletion which resulted in a stop codon at amino acid 1248 [CHD8 gRNA: 5′-TTCTTACTGTGTACCCGGGC-3′ (TGG)] (SEQ ID NO: 1)) was from the laboratory of F. Zhang (MIT/BROAD Institute); the Mito210 human iPSC line was from the laboratory of B. Cohen (McLean Hospital); the GM08330 human iPSC line (also known as GM8330-8) was from the laboratory of M. Talkowski (MGH Hospital). Cell lines were cultured in feeder-free conditions on 1% Geltrex (Gibco) coated cell culture dishes (Corning) using either mTESR1 medium (StemCell Technologies) or mTESR+ medium (StemCell Technologies) with 100 U/mL penicillin and 100 μg/ml streptomycin, at 37° C. in 5% CO2. PSC cultures and colonies were dissociated with the Gentle Cell Dissociation Reagent (StemCell Technologies) or ReLeSR (StemCell Technologies). All human PSC cultures were maintained below passage 50, were negative for mycoplasma, and karyotypically normal (analysis performed by WiCell Research Institute). The HUES66 line was authenticated using STR analysis completed by GlobalStem (2008). The Mito210 line was authenticated by genotyping analysis (Fluidigm FPV5 chip) performed by the Broad Institute Genomics Platform (2017). The GM08330 line was not authenticated. All experiments involving human cells were approved by the Harvard University IRB and ESCRO committees.

### CRISPR Guide Design for SUV420H1 and PTEN

The CRISPR guides for SUV420H1 and PTEN were designed using the Benchling CRISPR Guide Design Tool (Benchling Biology Software (2017). Retrieved from https://benchling.com). The guide was designed to maximize on-target efficiency and minimize off-target sites in intragenic regions^{51,52}. For SUV420H1, a guide was designed to target the beginning of the first exon to create a protein truncation in all known protein coding transcripts (SUV420H1 gRNA: 5′-CAAGAACCAAACTGGTTGCT-3′ (AGG) (SEQ ID NO: 2)). The PTEN guide was chosen to induce a stop codon in the catalytic phosphatase domain (PTEN gRNA: 5′-CCAAATTTAATTGCAGAGGT-3′ (AGG) (SEQ ID NO: 3)).

### CRISPR-Mediated Gene Editing of SUV420H1 and PTEN

Mutations in SUV420H1 and PTEN were induced in the Mito210 iPSC line. For SUV420H1, Nanoblades were generated as previously described⁵³. Parental iPSC in mTeSR1 were plated in 12-well plates pre-coated with Geltrex. When cells reached about 80% confluency, 300 μl of mTeSR1 with nanoblades (10 μl) and 4 μg/ml of Polybrene were added. Cells were incubated for 6 hours at 37° C., after which fresh mTeSR1 was added. For selection of edited clones, cells were enzymatically detached and plated onto Geltrex and mTeSR1 in a ratio of ˜5,000 cells per 60 mm dish. 10 μM of Y-27632 was added to increase single cell survival after splitting. When the colonies started to appear, each clone was manually collected and split into a single well of a 96-well plate coated with Geltrex and mTeSR1. During colony picking, some cells were reserved for DNA extraction and clonal screening.

The PTEN mutation was generated by the Broad Institute Stem Cell Facility. The transfections were performed with the NEON transfection system (using settings of 1050V, 30 ms, 2 pulses and 2.5×10\hat{ }5 cells); locus specific guides plus Cas9 (EnGen Cas9 NLS from NEB) were used; cells were cultured either in mTeSR or StemFlex (Gibco) media on Geltrex coated plates.

### Sequence Confirmation of SUV420H1 and PTEN Edits

Insertions/deletions in individual clones were screened via PCR amplification using primers flanking the guide and a second round of amplification using unique bar codes (Kappa HiFi Master Mix, Roche) with an annealing temperature of 60° C. and sequenced using Miseq (Illumina). For SUV420H1 the clone chosen for this study had a heterozygous 10 nucleotide deletion which resulted in a stop codon at amino acid 90 of the full-length transcript, resulting in protein truncation in all known protein coding transcripts. For PTEN the clone chosen had a heterozygous 1 nucleotide insertion which resulted in a stop codon at amino acid 91 in the phosphatase domain of the full-length transcript.

### Organoid Differentiation

Dorsal forebrain-patterned organoids were generated as previously described^{13,54}.

### Immunohistochemistry

Samples were fixed in 4% paraformaldehyde (PFA) (Electron Microscopy Services). Samples were washed with 1× phosphate buffered saline (PBS) (Gibco), cryoprotected in a 30% sucrose solution, embedded in optimum cutting temperature (OCT) compound (Tissue Tek) and cryosectioned at 12-18 μm thickness. Sections were washed with 0.1% Tween-20 (Sigma) in PBS, blocked for 1 hour at room temperature (RT) with 6% donkey serum (Sigma)+0.3% Triton-X-100 (Sigma) in PBS and incubated with primary antibodies O/N diluted with 2.5% donkey serum+0.1% Triton-X-100 in PBS. After washing, sections were incubated at RT with secondary antibodies diluted in the same solution as with primary antibodies for 2 hours, washed, and incubated with DAPI staining (1:10,000 in PBS+0.1% TWEEN-20) for 15 minutes to visualize cell nuclei.

### Whole-Organoid Imaging

Organoids were processed using the SHIELD protocol⁵⁵. Briefly, organoids were fixed for 30 minutes in 4% PFA at RT. The tissues were then treated with 3% (wt/v) polyglycerol-3-polyglycidyl ether (P3PE) for 48 h in ice cold 0.1M phosphate buffer (pH7.2) at 4° C. then transferred to 0.3% P3PE in 0.1M sodium carbonate (pH10) for 24 h at 37° C. Samples were rinsed and cleared in 0.2M SDS in 50 μmM phosphate buffer (pH 7.3) for 48 h at 55° C. Organoids were stained with Syto16 (ThermoFischer #S7578), anti-SOX2 (R&D #AF2018) and anti-TBR1 (CST #45664S) using the SmartLabel system (Lifecanvas). Tissues were washed extensively for 24 h in PBS+0.1% Triton-X-100 and antibodies were fixed to the tissue using a 4% PFA solution overnight at RT. Tissues were refractive index-matched in PROTOS solution (RI=1.519) and imaged using a SmartSPIM axially-swept light-sheet microscope (LifeCanvas Technologies). 3D image datasets were acquired using a 15×0.4 NA objective (ASI-Special Optics, #54-10-12).

### Microscopy and Organoid Size Analysis

Images of organoids in culture were taken with an EVOS FL microscope (Invitrogen), Lionheart™ FX Automated Microscope (BioTek Instruments), or with a Zeiss Axio Imager.Z2. Immunofluorescence images were acquired with the latter two and analyzed with the Gen5 (BioTek Instruments) or Zen Blue (Zeiss) image processing software, respectively. ImageJ⁵⁶ was used to quantify organoid area. Area values were obtained by manually drawing a continuous line around the perimeter of the organoid, and the software counts pixels in the indicated area. Measurements were plotted as a ratio to the average value for control organoids of each differentiation batch.

### Western Blotting

iPSC were washed with 1× phosphate buffered saline (PBS) (Gibco) and protein were extracted incubating with N-PER™ Neuronal Protein Extraction Reagent (ThermoFisher) supplemented with protease (cOmplete™ Mini Protease Inhibitor Cocktail, Roche) and phosphatase inhibitor (PhosSTOP, Sigma) for 15 minutes on ice. After 5-7 minutes of lysis, organoids were dissociated using a pipette tip. Lysates were transferred to microcentrifuge tubes and centrifuged for 10 minutes at 13,500 rpm at 4° C. The supernatants were transferred to new tubes and stored at −20° C. Protein concentration was quantified using the Pierce™ BCA Protein Assay Kit (ThermoFisher). Proteins were separated on a NuPAGE™ 4-12%, Bis-Tris Gel (Invitrogen) or Mini-PROTEAN 4-15% Gels (Biorad) and transferred onto PVDF membrane via the iBlot 2 Dry Blotting System (ThermoFisher) or traditional dry transfer system. Blots were then blocked in 5% nonfat dry milk (Bio-Rad) for 1 hour at RT and incubated with primary antibodies with gentle agitation on a shaker overnight. Afterward, the blots were washed 3× for 10 minutes in TBST and incubated at RT with secondary horseradish peroxidase-conjugated antibodies for 1 hour. The blots were washed in TBST again and briefly incubated in SuperSignal™ West Femto Maximum Sensitivity Substrate (ThermoFisher). The blots were immediately imaged on the ChemiDoc XRS+ System (Bio-Rad).

### Cell Lysis and FASP Digestion for Mass Spectrometry

For SUV420H1 and PTEN 35 d.i.v., four mutant and four control organoids were used. For CHD8 35 d.i.v., and for PTEN 70 d.i.v., three mutant and three control organoids were used. Cells were placed into microTUBE-15 (Covaris) microtubes with TPP buffer (Covaris) and lysed using a Covaris S220 Focused-ultrasonicator instrument with 125 W power over 180 s at 10% max peak power. Upon precipitation with chloroform/methanol, extracted proteins were weighed and digested according to FASP protocol (100 μg for PTEN and CHD8; 150 g for SUV420H1). Briefly, the 10K filter was washed with 100 μL of triethylammonium bicarbonate (TEAB). Each sample was added, spun, and the supernatant discarded. Then, 100 μL of Tris(2-carboxyethyl) phosphine (TCEP) 37 was added for one hour, spun, and the supernatant discarded. While shielding from light, 100 μL IAcNH2 was added for 1 hour followed by spinning and discarding the supernatant. Next, 150 μL 50 μmM TEAB +Sequencing Grade Trypsin (Promega) was added and left overnight at 38° C., upon which the samples were spun and supernatant collected. Lastly, 50 μL 50 μmM TEAB was added to the samples, followed by spinning and supernatant collection. The samples were then transferred to HPLC.

### TMT Mass Tagging Protocol Peptide Labeling

The TMT (Tandem Mass Tag) Label Reagents were equilibrated to RT and resuspended in anhydrous acetonitrile or ethanol (for the 0.8 mg vials 41 μL were added, for the 5 mg vials 256 μL were added). The reagent was dissolved for 5 minutes with occasional vortexing. 41 μL of the TMT Label Reagent (the equivalent of 0.8 mg) was added to each 100-150 g sample. The reaction was incubated for 1 hour at RT. 8 μL of 5% hydroxylamine was added to the sample and incubated for 15 minutes to quench the reaction. Samples were combined, dry in speedvac (Eppendorf, Germany) and stored at −80° C.

### Hi pH Separation and Mass Spectrometry Analysis

Before submission to LC-MS/MS (Liquid Chromatography with tandem mass spectrometry), each experiment sample was separated on a Hi-pH column (Thermo, CA) according to the vendor's instructions. After separation into 40 fractions, each fraction was submitted for a single LC-MS/MS experiment performed on a Lumos Tribrid (Thermo, CA) equipped with 3000 Ultima Dual nanoHPLC pump (Thermo, CA). Peptides were separated onto a 150 μm inner diameter microcapillary trapping column packed first with approximately 3 cm of C18 Reprosil resin (5 μm, 100 Å, Dr. Maisch GmbH, Germany) followed by PharmaFluidics (Belgium) micropack analytical column 50 cm. Separation was achieved through applying a gradient from 5-27% ACN in 0.1% formic acid over 90 min at 200 nl min−1. Electrospray ionization was enabled through applying a voltage of 1.8 kV using a home-made electrode junction at the end of the microcapillary column and sprayed from stainless-steel tips (PepSep, Denmark). The Lumos Orbitrap was operated in data-dependent mode for the MS methods. The MS survey scan was performed in the Orbitrap in the range of 400-1,800 m/z at a resolution of 6×104, followed by the selection of the twenty most intense ions (TOP20) for CTD-MS2 fragmentation in the Ton trap using a precursor isolation width window of 2 m/z, AGC setting of 10,000, and a maximum ion accumulation of 50 ms. Singly-charged ion species were not subjected to CID fragmentation. Normalized collision energy was set to 35 V and an activation time of 10 ms. Ions in a 10 ppm m/z window around ions selected for MS2 were excluded from further selection for fragmentation for 90 s. The same TOP20 ions were subjected to HCD MS2 event in Orbitrap part of the instrument. The fragment ion isolation width was set to 0.8 m/z, AGC was set to 50,000, the maximum ion time was 150 μms, normalized collision energy was set to 34V and an activation time of 1 ms for each HCD MS2 scan.

### Mass Spectrometry Data Generation

Raw data were submitted for analysis in Proteome Discoverer 2.4 (Thermo Scientific) software. Assignment of MS/MS spectra was performed using the Sequest HT algorithm by searching the data against a protein sequence database including all entries from the Human Uniprot database (SwissProt 19,768 2019) and other known contaminants such as human keratins and common lab contaminants. Sequest HT searches were performed using a 10 ppm precursor ion tolerance and requiring each peptides N-/C termini to adhere with Trypsin protease specificity, while allowing up to two missed cleavages. 16-plex TMTpro tags on peptide N termini and lysine residues (+304.207 Da) was set as static modifications while methionine oxidation (+15.99492 Da) was set as variable modification. A MS2 spectra assignment false discovery rate (FDR) of 1% on protein level was achieved by applying the target-decoy database search. Filtering was performed using a Percolator (64 bit version,⁵⁷). For quantification, a 0.02 m/z window centered on the theoretical m/z value of each of the six reporter ions and the intensity of the signal closest to the theoretical m/z value was recorded. Reporter ion intensities were exported in the result file of Proteome Discoverer 2.4 search engine as Excel tables. The total signal intensity across all peptides quantified was summed for each TMT channel, and all intensity values were normalized to account for potentially uneven TMT labeling and/or sample handling variance for each labeled channel.

### Mass Spectrometry Data Analysis

Potential contaminants were filtered out and proteins supported by at least two unique peptides were used for further analysis. Proteins that were missing were kept in at most one sample per condition. Data were transformed and normalized using variance stabilizing normalization using the DEP package of Bioconductor⁵⁸. To perform statistical analysis, data were imputed for missing values using random draws from a Gaussian distribution with width 0.3 and a mean that is down-shifted from the sample mean by 1.8. To detect statistically significant differential protein abundance between conditions a moderated t-test was performed using the LIMMA package of Bioconductor⁵⁹, employing an FDR threshold of 0.1. GSEA was performed using the GSEA software⁶⁰. Gene Ontology (GO) and KEGG pathway annotation were utilized to perform functional annotation of the significantly regulated proteins. GO terms and KEGG pathways with FDR q-values <0.05 were considered statistically significant. For PTEN datasets, correlation between mutant effect (PTEN+/− vs control at 35 d.i.v.) and time effect (35 d.i.v. vs. 70 d.i.v. in control organoids) was calculated using Pearson correlation. Between PTEN 35 d.i.v. and 70 d.i.v., changes in protein levels in mutant and control organoids were compared to one another with a signed paired Wilcoxin rank test, using stat_compare_means from the ggpubr R package (available at rpkgs.datanovia.com/ggpubr/).

To build protein interaction networks, the prize-collecting Steiner forest algorithm was used^{61,62} using all three sets of DEPs as terminals, with the absolute value of their log fold change as prizes. This algorithm optimizes the network to include high-confidence protein interactions between protein nodes with large prizes. The PCSF R package v0.99.1⁶³ was used to calculate networks, with the STRING database as a background protein-protein interactome⁶⁴, using parameters r=0.1, w=0.8, b=15, and mu=0.01. As is default in that package, the network was subclustered using the edge betweenness clustering algorithm from the igraph package, and functional enrichment is performed on each cluster using the ENRICHR API. The Cytoscape software version 3.6.1 was used for network visualization⁶⁵. To assess relationships between the three sets of differential proteins, a PPI-weighted gene distance⁵⁰, was calculated between each pair of protein sets. A background distribution was calculated by drawing size-matched random lists of proteins from all detected proteins in each dataset and calculating the pMM between these sets. This was repeated 1000 times, and a p value was calculated by evaluating the amount of times randomized pMMs were lower than the value calculated using differential proteins. DEPs: differentially expressed proteins.

### Dissociation of Brain Organoids and Single-Cell RNA-Seq

Individual brain organoids were dissociated into a single-cell suspension using the Worthington Papain Dissociation System kit (Worthington Biochemical). A detailed description of the dissociation protocol is available at Protocol Exchange, with adaptations depending on age and size⁶⁶. Dissociated cells were resuspended in ice-cold PBS containing 0.04% BSA (Sigma, PN-B8667), counted them with Countess II (ThermoFisher), and then adjusted the volume to the final concentration of 1000 cells/μl. Approximately 17,400, 9600 or 8000 cells were used per channel (to give an estimated recovery of 10,000, 6000 or 5000 cells per channel), loaded them onto a Chromium™ Single Cell 3′ Chip (10× Genomics, PN-120236), and processed them through the Chromium Controller to generate single cell GEMs (Gel Beads in Emulsion). Single-cell RNA-seq libraries with the Chromium™ Single Cell 3′ Library & Gel Bead Kit v3 (10× Genomics, PN-1000075), with the exception of a few libraries in the earlier experiments that were prepared with a v2 kit (10× Genomics, PN-120236). Libraries from different samples were pooled based on molar concentrations and sequenced on a NextSeq 500 or NovaSeq S1 instrument (Illumina) with 28 bases for read 1 (26 bases for v2 libraries), 55 bases for read 2 (57 bases for v2 libraries) and 8 bases for Index 1. If necessary, after the first round of sequencing, libraries were re-pooled based on the actual number of cells in each and re-sequenced with goal of producing an equal number of reads per cell for each sample and to reach a sequencing saturation of at least 20% for v3 libraries (50% for v2 libraries).

### Single Cell RNA-Seq Data Analysis

Reads from RNA-seq were aligned to the GRCh38 human reference genome and the cell-by-gene count matrices were produced with the Cell Ranger pipeline (10× Genomics)⁶⁷. Cellranger version 2.0.1 was used for GM08330 experiments and for HUES66 3.5 months batch I, while version 3.0.2 was used for all other experiments. Default parameters were used, except for the ‘-cells’ argument. Data was analyzed using the Seurat R package v3.1.5⁶⁸ using R v3.6. Cells expressing a minimum of 500 genes were kept, and UMI counts were normalized for each cell by the total expression, multiplied by 106, and log-transformed. Variable genes were found using the “mean.var.plot” method, and the ScaleData function was used to regress out variation due to differences in total UMIs per cell. Principal component analysis (PCA) was performed on the scaled data for the variable genes, and top principal components were chosen based on Seurat's ElbowPlots (at least 15 PCs were used in all cases). Cells were clustered in PCA space using Seurat's FindNeighbors on top principal components, followed by FindClusters with resolution=1.0. Variation in the cells was visualized by t-SNE (t-distributed stochastic neighbor embedding) on the top principal components.

In the case of GM08330 one month organoids, cells were demultiplexed using genotype clustering from cells from a different experiment that were sequenced in the same lane. To demultiplex, SNPs were called from CellRanger BAM files with the cellSNP tool v0.1.5, and then the vireo function was used with default parameters and n_donor-2, from the cardelino R library v0.4.0⁶⁹ to assign cells to each genotype.

In three cases, one out of six organoids were excluded from the analysis as outliers. See the Statistics & Reproducibility section for details.

For each dataset, upregulated genes in each cluster were identified using the VeniceMarker tool from the Signac package v0.0.7 from BioTuring (available at github.com/bioturing/signac). Cell types were assigned to each cluster by looking at the top most significant upregulated genes. In a few cases, clusters were further subclustered to assign identities at higher resolution.

To assess gene expression of ASD risk genes in GM08330 and Mito210 control organoids across timepoints, datasets from one, three, and six months were merged using Seurat, then batch corrected using Harmony v1.0 with default parameters⁷⁰. Since the one month data is dominated by cell cycle signal, the ScaleData function was used to regress out variation due to both total UMI count per cell and to cell cycle stage differences, calculated using Seurat's CellCycleScore. Variation was visualized using t-SNE on the first 30 Harmony dimensions. Expression of the 102 ASD-linked genes identified in the Satterstrom et. al. study⁸, compared to control gene lists with similar average expression, was evaluated using Seurat's AddModuleScore function. The distribution of module scores between broad cell types was compared using Seurat's violin plot.

To compare cell type proportions between control and mutant organoids, count tables were prepared from dataset metadata. For each cell type present in a dataset, a column was created that indicated whether a cell belonged to that cell type or not. Using the R package lme4 v1.1-23⁷¹, the glmer function was used to estimate a mixed-effect logistic regression model with this binary cell type column as output, the control or mutant state of the cell as a fixed predictor, and the organoid that the cell belonged to as a random intercept. In order to calculate the significance of the contribution of control-versus-mutant state to the model, another model was fit without that predictor, and the anova function was used to compare the two model fits. P values for each cell type were then adjusted for multiple hypothesis testing using the Benjamini-Hochberg correction.

### Pseudotime, Gene Module, and Differential Expression Analysis

Pseudotime trajectories were created for each dataset using the Monocle3 v. 0.2.0 software package³⁵. Briefly, a CellDataSet was created from the raw counts and metadata in the corresponding Seurat object, and then cells were subsetted to contain an equal amount from control and mutant. Data was processed as default in Monocle3. A starting point for the trajectory was chosen manually by finding an endpoint of the tree located in the earliest developmental cell type (generally, cycling progenitors). Where the cells were split into more than one partition, the starting point was chosen within the partition containing the mature cell types of interest (PN for one month SUV40H1 organoids, IN for 3.5 month CHD8 organoids), and then a new UMAP was calculated using just these cells. Results were then visualized on the resulting UMAP, and by plotting the density of pseudotime values assigned to control and mutant cells.

In order to learn patterns of coordinated gene regulation across the cells, WGCNA³⁶ was applied to each dataset. Where cells were split into partitions in the above pseudotime analysis, only cells belonging to the partition of interest were used. For the PTEN three months dataset, cells were split into two partitions based on the branching of the pseudotime into two subtypes of projection neurons: one excluding callosal projection neurons, and the other excluding corticofugal neurons. Normalized gene expression data was further filtered to remove outlying genes, mitochondrial and ribosomal genes. Outliers were identified by setting the upper (>9) and lower (<0.15) thresholds to the average normalized expression per gene. After processing, blockwiseModules function from the WGCNA v1.69 library was performed in R with the parameters networkType=“signed”, minModuleSize=4, corType=“Bicor”, maxPOutliers=0.1, deepSplit=3,trapErrors=T, and randomSeed=59069. Other than power, remaining parameters were left as the default setting. The power parameter that determines the resolution of gene module output was chosen independently for each dataset. To pick an adequate power, the pickSoftThreshold function from WGCNA was used to test values from 1 to 30. Final resolution was determined by studying the output modules and choosing the resolution that captured most variation in the fewest total number of modules—this resulted in a power of 18 for SUV40H1 28 d.i.v., 3 for SUV40H1 35 d.i.v., and 12 for both partitions of PTEN 3 months and HUES66 3.5 months.

In order to calculate significance of differential expression of modules in the data, Seurat objects were downsampled to have an equal number of cells per organoid, and then the AddModuleScore function was used to assign a score to each cell. For each module, linear mixed-effect models were fit to the data, with the modules scores as the output, the organoid the cell belongs to as a random intercept, and with or without the control-versus-mutant state as a predictor. The anova function was used to compare the models and evaluate the use of control-versus-mutant as a predictor, and p values were then adjusted across modules using the Benjamini-Hochberg correction.

Differential genes between control and mutant organoids were assessed using a method to control for variability organoid-to-organoid. Datasets were subsetted to the cells from the “partition of interest” in the above pseudotime analysis, in order to isolate the cells predicted to be in the developmental trajectory of the affected neurons for each mutant. Reads were then summed across cells in each organoid. Genes with less than 10 total reads were excluded, and then DESeq2⁷² was used to calculate DEGs, with each organoid as a sample. The clusterProfiler⁷³ R package was used to find enriched biological processes in these gene sets, with the enrichGO function and the compareCluster function to highlight processes the gene sets might have in common. Redundant GO terms were removed with the simplify function, with cutoff=0.7. DEGs: differentially expressed genes.

### Statistics and Reproducibility

For organoid analysis, SUV420H1^{+/-} organoids: n=42 18 d.i.v. control organoids, 42 18 d.i.v. mutant organoids, 47 27-d.i.v. control organoids, and 46 27-d.i.v. mutant organoids, from 4 differentiation batches. For PTEN^{+/-} organoids: n=114 18-d.i.v. control organoids, 77 18-d.i.v. mutant organoids, 39 35-d.i.v. control organoids, and 34 35-d.i.v. mutant organoids, from 3 differentiation batches. For CHD8^{+/-} organoids: n=19 16 d.i.v. control organoids, 19 16 d.i.v. mutant organoids, 16 25 d.i.v. control organoids, and 17 25 d.i.v. mutant organoids, from 2 differentiation batches.

For proteomic analysis, four mutant and four control organoids were used in the case of SUV420H1 and PTEN 35 d.i.v. For CHD8 35 d.i.v., and for PTEN 70 d.i.v., three mutant and three control organoids were used.

Finally, in each scRNA-seq dataset, three organoids per genotype were initially sequenced. In three cases, one out of six organoids were excluded from the analysis as outliers. In PTEN^{+/-} 35 d.i.v. batch I, a control organoid was excluded because not enough cells could be recovered from that sequencing lane, and in the second batch, a control organoid was excluded because it clustered entirely separately from the other 5 organoids, indicating a potential differentiation issue. Finally, in CHD8^{+/+} 3.5 month batch II, a mutant organoid was excluded due to mostly containing inhibitor-lineage cells, with very few projection neuron cells. Although an increase in interneuron-lineage cells was seen in all mutant organoids in this experiment, this extreme example was excluded to be conservative. This leaves us with a total of 57 organoids considered in downstream analysis, with a total of 416,051 cells.

### REFERENCES

1 American Psychiatric Association. in Diagnostic and Statistical Manual of Mental Disorders (2013).
2 Lord, C. et al. Autism spectrum disorder. Nat Rev Dis Primers 6, 5, doi:10.1038/s41572-019-0138-4 (2020).
3 Rosenberg, R. E. et al. Characteristics and concordance of autism spectrum disorders among 277 twin pairs. Arch Pediatr Adolesc Med 163, 907-914, doi:10.1001/archpediatrics.2009.98 (2009).
4 Iossifov, I. et al. De novo gene disruptions in children on the autistic spectrum. Neuron 74, 285-299, doi:10.1016/j.neuron.2012.04.009 (2012).
5 O'Roak, B. J. et al. Sporadic autism exomes reveal a highly interconnected protein network of de novo mutations. Nature 485, 246-250, doi:10.1038/nature10989 (2012).
6 Sanders, S. J. et al. Insights into Autism Spectrum Disorder Genomic Architecture and Biology from 71 Risk Loci. Neuron 87, 1215-1233, doi:10.1016/j.neuron.2015.09.016 (2015).
7 De Rubeis, S. et al. Synaptic, transcriptional and chromatin genes disrupted in autism. Nature 515, 209-215, doi:10.1038/nature13772 (2014).
8 Satterstrom, F. K. et al. Large-Scale Exome Sequencing Study Implicates Both Developmental and Functional Changes in the Neurobiology of Autism. Cell 180, 568-584.e523, doi: doi.org/10.1016/j.cell.2019.12.036 (2020).
9 Ruzzo, E. K. et al. Inherited and De Novo Genetic Risk for Autism Impacts Shared Networks. Cell 178, 850-866.e826, doi:10.1016/j.cell.2019.07.015 (2019).
10 Amin, N. D. & Pa ca, S. P. Building Models of Brain Disorders with Three-Dimensional Organoids. Neuron 100, 389-405, doi:10.1016/j.neuron.2018.10.007 (2018).
11 Lancaster, M. A. & Knoblich, J. A. Organogenesis in a dish: Modeling development and disease using organoid technologies. Science 345, 1247125, doi:10.1126/science.1247125 (2014).
12 Velasco, S., Paulsen, B. & Arlotta, P. 3D Brain Organoids: Studying Brain Development and Disease Outside the Embryo. Annu Rev Neurosci 43, 375-389, doi:10.1146/annurev-neuro-070918-050154 (2020).
13 Velasco, S. et al. Individual brain organoids reproducibly form cell diversity of the human cerebral cortex. Nature 570, 523-527, doi:10.1038/s41586-019-1289-x (2019).
14 Jprgensen, S., Schotta, G. & Sprensen, C. S. Histone H4 Lysine 20 methylation: key player in epigenetic regulation of genomic integrity. Nucleic Acids Research 41, 2797-2806, doi:10.1093/nar/gkt012 (2013).
15 Wickramasekara, R. N. & Stessman, H. A. F. Histone 4 Lysine 20 Methylation: A Case for Neurodevelopmental Disease. Biology (Basel) 8, 11, doi:10.3390/biology8010011 (2019).
16 Nicetto, D. et al. Suv4-20h Histone Methyltransferases Promote Neuroectodermal Differentiation by Silencing the Pluripotency-Associated Oct-25 Gene. PLOS Genetics 9, e1003188, doi:10.1371/journal.pgen.1003188 (2013).
17 Rhodes, C. T. et al. Cross-species analyses unravel the complexity of H3K27me3 and H4K20me3 in the context of neural stem progenitor cells. Neuroepigenetics 6, 10-25, doi: doi.org/10.1016/j.nepig.2016.04.001 (2016).
18 Barnard, R. A., Pomaville, M. B. & O'Roak, B. J. Mutations and Modeling of the Chromatin Remodeler CHD8 Define an Emerging Autism Etiology. Front Neurosci 9, 477-477, doi:10.3389/fnins.2015.00477 (2015).
19 Lee, Y.-R., Chen, M. & Pandolfi, P. P. The functions and regulation of the PTEN tumour suppressor: new modes and prospects. Nature Reviews Molecular Cell Biology 19, 547-562, doi:10.1038/s41580-018-0015-0 (2018).
20 Skelton, P. D., Stan, R. V. & Luikart, B. W. The Role of PTEN in Neurodevelopment. Complex Psychiatry 5, 60-71, doi:10.1159/000504782 (2019).
21 Stessman, H. A. F. et al. Targeted sequencing identifies 91 neurodevelopmental-disorder risk genes with autism and developmental-disability biases. Nature genetics 49, 515-526, doi:10.1038/ng.3792 (2017).
22 C Yuen, R. K. et al. Whole genome sequencing resource identifies 18 new candidate genes for autism spectrum disorder. Nature neuroscience 20, 602-611, doi:10.1038/nn.4524 (2017).
23 Cristofano, A. D., Pesce, B., Cordon-Cardo, C. & Pandolfi, P. P. Pten is essential for embryonic development and tumour suppression. Nature Genetics 19, 348-355, doi:10.1038/1235 (1998).
24 Nishiyama, M. et al. Early embryonic death in mice lacking the beta-catenin-binding protein Duplin. Mol Cell Biol 24, 8386-8394, doi:10.1128/mcb.24.19.8386-8394.2004 (2004).
25 Schotta, G. et al. A chromatin-wide transition to H4K20 monomethylation impairs genome integrity and programmed DNA rearrangements in the mouse. Genes Dev 22, 2048-2061, doi:10.1101/gad.476008 (2008).
26 Bernier, R. et al. Disruptive CHD8 mutations define a subtype of autism early in development. Cell 158, 263-276, doi:10.1016/j.cell.2014.06.017 (2014).
27 Faundes, V. et al. Histone Lysine Methylases and Demethylases in the Landscape of Human Developmental Disorders. Am J Hum Genet 102, 175-187, doi:10.1016/j.ajhg.2017.11.013 (2018).
28 Yehia, L., Keel, E. & Eng, C. The Clinical Spectrum of PTEN Mutations. Annu Rev Med 71, 103-116, doi:10.1146/annurev-med-052218-125823 (2020).
29 Lodato, S. & Arlotta, P. Generating neuronal diversity in the mammalian cerebral cortex. Annu Rev Cell Dev Biol 31, 699-720, doi:10.1146/annurev-cellbio-100814-125353 (2015).
30 Greig, L. C., Woodworth, M. B., Galazo, M. J., Padmanabhan, H. & Macklis, J. D. Molecular logic of neocortical projection neuron specification, development and diversity. Nat Rev Neurosci 14, 755-769, doi:10.1038/nrn3586 (2013).
31 Marín-Padilla, M. Human cerebral cortex Cajal-Retzius neuron: development, structure and function. A Golgi study. Front Neuroanat 9, 21, doi:10.3389/fnana.2015.00021 (2015).
32 Orrico, A. et al. Novel PTEN mutations in neurodevelopmental disorders and macrocephaly. Clinical Genetics 75, 195-198, doi:10.1111/j.1399-0004.2008.01074.x (2009).
33 Li, Y. et al. Induction of Expansion and Folding in Human Cerebral Organoids. Cell Stem Cell 20, 385-396.e383, doi:10.1016/j.stem.2016.11.017 (2017).
34 Lim, L., Mi, D., Llorca, A. & Marín, O. Development and Functional Diversification of Cortical Interneurons. Neuron 100, 294-313, doi:10.1016/j.neuron.2018.10.009 (2018).
35 Cao, J. et al. The single-cell transcriptional landscape of mammalian organogenesis. Nature 566, 496-502, doi:10.1038/s41586-019-0969-x (2019).
36 Langfelder, P. & Horvath, S. WGCNA: an R package for weighted correlation network analysis. BMC Bioinformatics 9, 559, doi:10.1186/1471-2105-9-559 (2008).
37 Lalli, M. A., Avey, D., Dougherty, J. D., Milbrandt, J. & Mitra, R. D. High-throughput single-cell functional elucidation of neurodevelopmental disease-associated genes reveals convergent mechanisms altering neuronal differentiation. Genome Res 30, 1317-1331, doi:10.1101/gr.262295.120 (2020).
38 Cederquist, G. Y. et al. A Multiplex Human Pluripotent Stem Cell Platform Defines Molecular and Functional Subclasses of Autism-Related Genes. Cell Stem Cell 27, 35-49.e36, doi:10.1016/j.stem.2020.06.004 (2020).
39 Mariani, J. et al. FOXG1-Dependent Dysregulation of GABA/Glutamate Neuron Differentiation in Autism Spectrum Disorders. Cell 162, 375-390, doi:10.1016/j.cell.2015.06.034 (2015).
40 Rubenstein, J. L. R. & Merzenich, M. M. Model of autism: increased ratio of excitation/inhibition in key neural systems. Genes, Brain and Behavior 2, 255-267, doi:10.1034/j.1601-183X.2003.00037.x (2003).
41 Coghlan, S. et al. GABA system dysfunction in autism and related disorders: from synapse to symptoms. Neurosci Biobehav Rev 36, 2044-2055, doi:10.1016/j.neubiorev.2012.07.005 (2012).
42 Pizzarelli, R. & Cherubini, E. Alterations of GABAergic signaling in autism spectrum disorders. Neural Plast 2011, 297153, doi:10.1155/2011/297153 (2011).
43 Gogolla, N. et al. Common circuit defect of excitatory-inhibitory balance in mouse models of autism. J Neurodev Disord 1, 172-181, doi:10.1007/s11689-009-9023-x (2009).
44 Rubenstein, J. L. R. The generation of cortical interneurons. Epilepsia 51, 68-68, doi:10.1111/j.1528-1167.2010.02854.x (2010).
45 Zikopoulos, B. & Barbas, H. Altered neural connectivity in excitatory and inhibitory cortical circuits in autism. Front Hum Neurosci 7, 609-609, doi:10.3389/fnhum.2013.00609 (2013).
46 Bocchi, R. et al. Perturbed Wnt signaling leads to neuronal migration delay, altered interhemispheric connections and impaired social behavior. Nature Communications 8, 1158, doi:10.1038/s41467-017-01046-w (2017).
47 Gao, P., Sultan, K. T., Zhang, X. J. & Shi, S. H. Lineage-dependent circuit assembly in the neocortex. Development 140, 2645-2655, doi:10.1242/dev.087668 (2013).
48 Lodato, S., Shetty, A. S. & Arlotta, P. Cerebral cortex assembly: generating and reprogramming projection neuron diversity. Trends in Neurosciences 38, 117-125, doi:https://doi.org/10.1016/j.tins.2014.11.003 (2015).
49 Jin, X. et al. <em>In vivo</em>Perturb-Seq reveals neuronal and glial abnormalities associated with Autism risk genes. bioRxiv, 791525, doi:10.1101/791525 (2019).
50 Yoon, S. et al. GScluster: network-weighted gene-set clustering analysis. BMC Genomics 20, 352, doi:10.1186/s12864-019-5738-6 (2019).
51 Doench, J. G. et al. Rational design of highly active sgRNAs for CRISPR-Cas9-mediated gene inactivation. Nature Biotechnology 32, 1262-1267, doi:10.1038/nbt.3026 (2014).
52 Hsu, P. D. et al. DNA targeting specificity of RNA-guided Cas9 nucleases. Nature Biotechnology 31, 827-832, doi:10.1038/nbt.2647 (2013).
53 Mangeot, P. E. et al. Genome editing in primary cells and in vivo using viral-derived Nanoblades loaded with Cas9-sgRNA ribonucleoproteins. Nature Communications 10, 45, doi:10.1038/s41467-018-07845-z (2019).
54 Silvia, V., Bruna, P. & Paola, A. Protocol Exchange, doi:10.21203/rs.2.9542/v1 (2020).
55 Park, Y.-G. et al. Protection of tissue physicochemical properties using polyfunctional crosslinkers. Nature Biotechnology 37, 73-83, doi:10.1038/nbt.4281 (2019).
56 Schneider, C. A., Rasband, W. S. & Eliceiri, K. W. NIH Image to ImageJ: 25 years of image analysis. Nat Methods 9, 671-675, doi:10.1038/nmeth.2089 (2012).
57 Kall, L., Storey, J. D., MacCoss, M. J. & Noble, W. S. Posterior error probabilities and false discovery rates: two sides of the same coin. J Proteome Res 7, 40-44, doi:10.1021/pr700739d (2008).
58 Zhang, X. et al. Proteome-wide identification of ubiquitin interactions using UbIA-MS. Nature Protocols 13, 530-550, doi:10.1038/nprot.2017.147 (2018).
59 Ritchie, M. E. et al. limma powers differential expression analyses for RNA-sequencing and microarray studies. Nucleic Acids Res 43, e47, doi:10.1093/nar/gkv007 (2015).
60 Subramanian, A. et al. Gene set enrichment analysis: A knowledge-based approach for interpreting genome-wide expression profiles. Proceedings of the National Academy of Sciences 102, 15545-15550, doi:10.1073/pnas.0506580102 (2005).
61 Tuncbag, N. et al. Simultaneous reconstruction of multiple signaling pathways via the prize-collecting steiner forest problem. J Comput Biol 20, 124-136, doi:10.1089/cmb.2012.0092 (2013).
62 Tuncbag, N. et al. Network-Based Interpretation of Diverse High-Throughput Datasets through the Omics Integrator Software Package. PLoS Comput Biol 12, e1004879, doi:10.1371/journal.pcbi.1004879 (2016).
63 Akhmedov, M. et al. PCSF: An R-package for network-based interpretation of high-throughput data. PLoS Comput Biol 13, e1005694, doi:10.1371/journal.pcbi.1005694 (2017).
64 Szklarczyk, D. et al. STRING v11: protein-protein association networks with increased coverage, supporting functional discovery in genome-wide experimental datasets. Nucleic Acids Res 47, D607-d613, doi:10.1093/nar/gky1131 (2019).
65 Shannon, P. et al. Cytoscape: a software environment for integrated models of biomolecular interaction networks. Genome research 13, 2498-2504, doi:10.1101/gr.1239303 (2003).
66 Paola, A., Giorgia, Q. & John Lawrence, S. Protocol Exchange, doi:10.1038/protex.2017.049 (2020).
67 Zheng, G. X. Y. et al. Massively parallel digital transcriptional profiling of single cells. Nature Communications 8, 14049, doi:10.1038/ncomms14049 (2017).
68 Stuart, T. et al. Comprehensive Integration of Single-Cell Data. Cell 177, 1888-1902.e1821, doi:10.1016/j.cell.2019.05.031 (2019).
69 Huang, Y., McCarthy, D. J. & Stegle, O. Vireo: Bayesian demultiplexing of pooled single-cell RNA-seq data without genotype reference. Genome Biology 20, 273, doi:10.1186/s13059-019-1865-2 (2019).
70 Korsunsky, I. et al. Fast, sensitive and accurate integration of single-cell data with Harmony. Nature Methods 16, 1289-1296, doi:10.1038/s41592-019-0619-0 (2019).
71 Bates, D., Machler, M., Bolker, B. & Walker, S. Fitting Linear Mixed-Effects Models Using lme4. 2015 67, 48, doi:10.18637/jss.v067.i01 (2015).
72 Love, M. I., Huber, W. & Anders, S. Moderated estimation of fold change and dispersion for RNA-seq data with DESeq2. Genome Biology 15, 550, doi:10.1186/s13059-014-0550-8 (2014).
73 Yu, G., Wang, L.-G., Han, Y. & He, Q.-Y. clusterProfiler: an R package for comparing biological themes among gene clusters. OMICS 16, 284-287, doi:10.1089/omi.2011.0118 (2012).

### Example 2—Human Brain Organoids Reveal Asynchronous Development of Cortical Neuron Classes as a Shared Feature of Autism Risk Genes

This Example both re-presents certain data from Example 1 and provides additional data.

### Results

### Single-Cell Map of Developing Cortical Organoids Reveals Cell Type-Specific Enrichment of ASD Risk Genes

To experimentally investigate whether mutations in different ASD risk genes converge on shared molecular, cellular, or developmental phenotypes, cortical organoids, an in vitro model of human cortical development, was leveraged using a robust protocol that was previously developed¹⁴. This model has been validated to undergo reproducible generation of cortical cellular diversity, along consistent developmental trajectories, in each individual organoid.

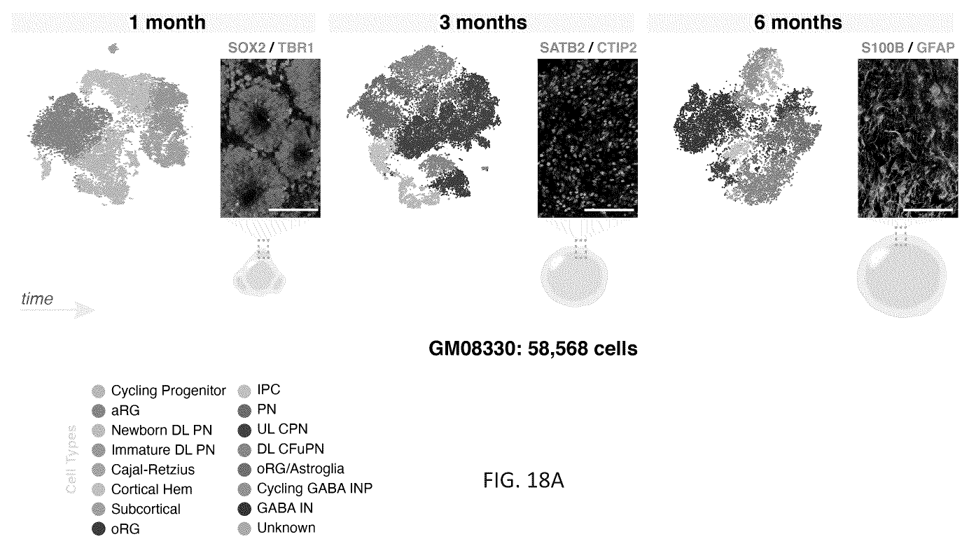

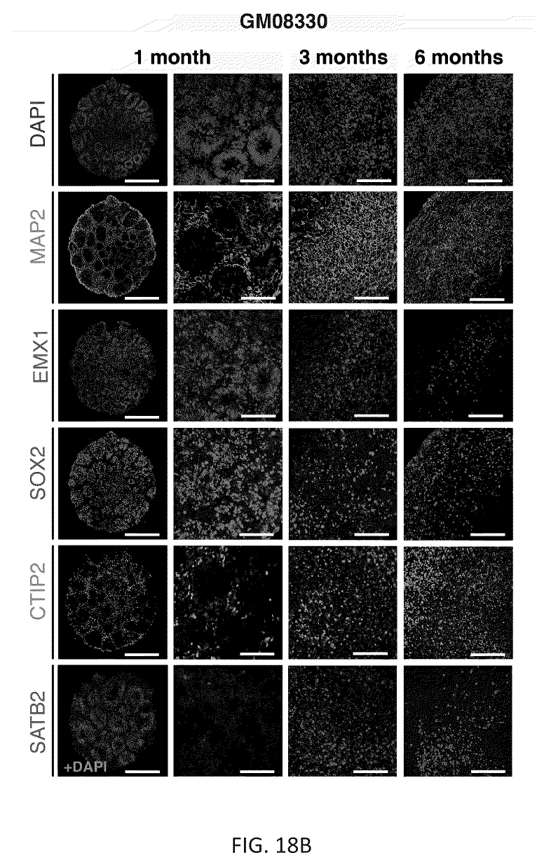

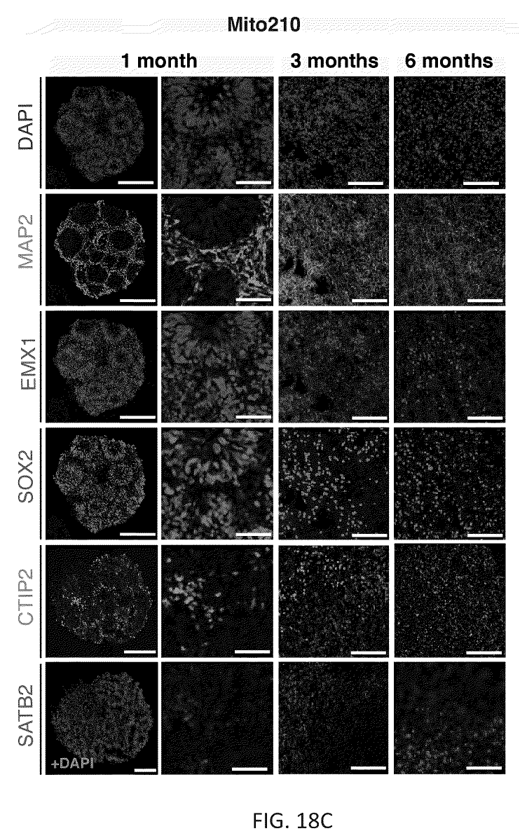

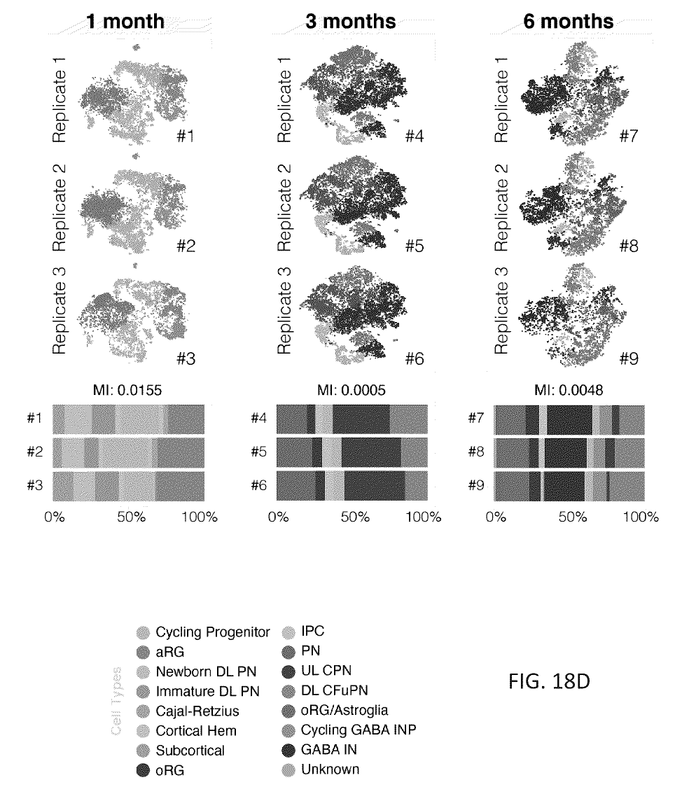

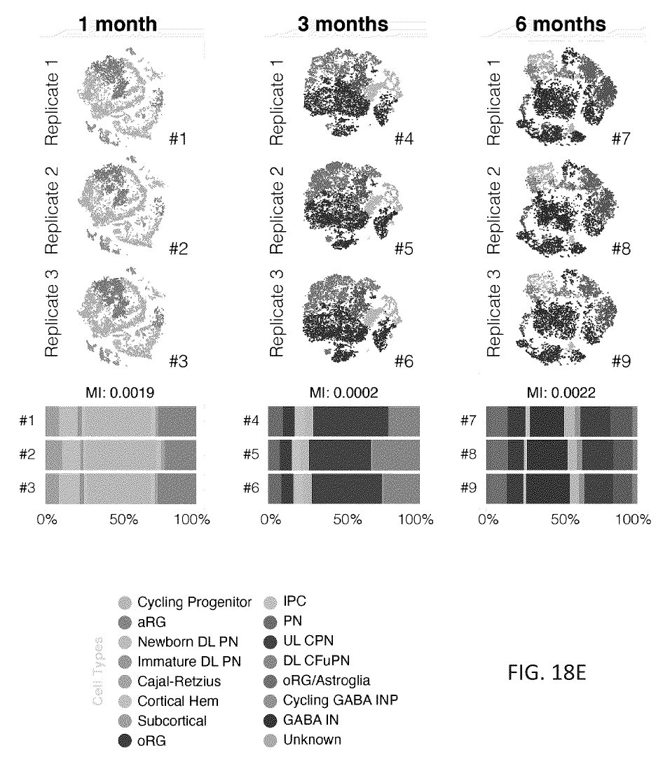

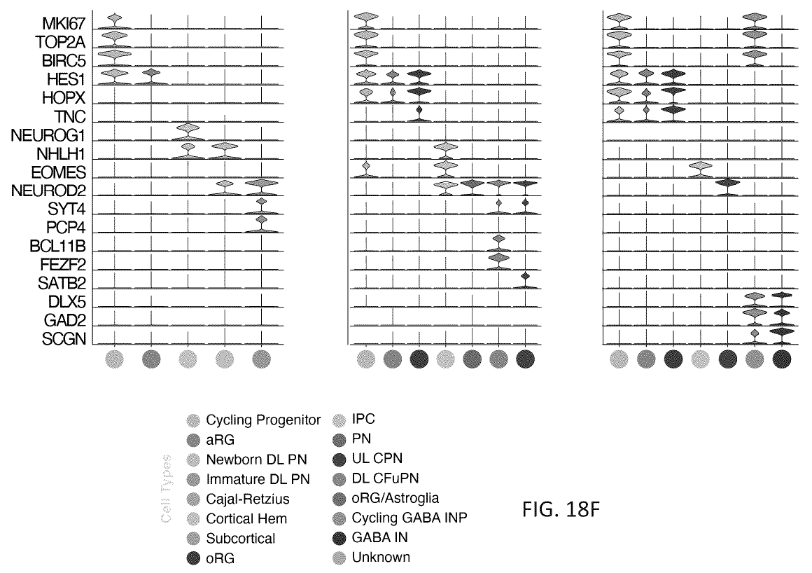

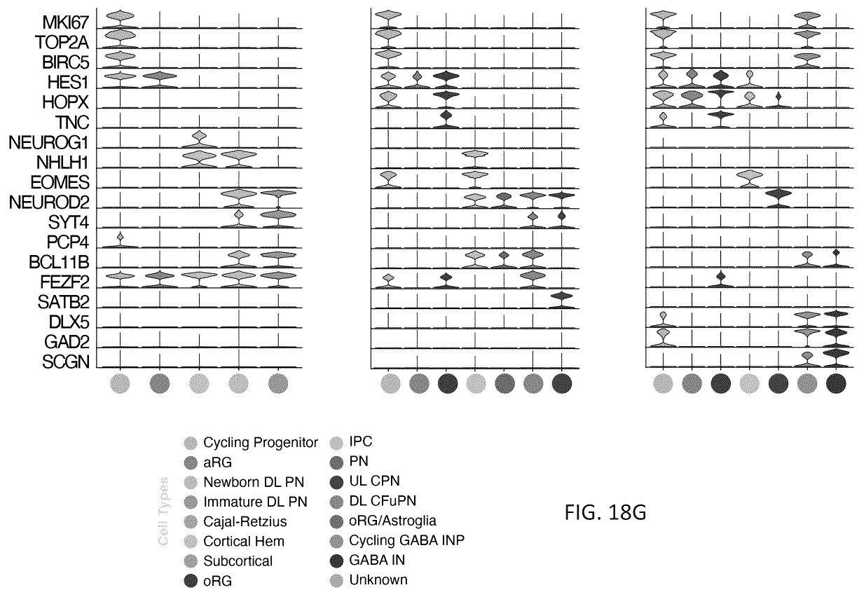

First, organoids were generated from two different human induced pluripotent stem cell (iPSC) lines, GM08330 and Mito210 (Methods, FIG. 18). Individual organoids were profiled by immunohistochemistry and single-cell RNA-seq (scRNA-seq) (GM08330: 9 organoids, 58,568 single cells; Mito210: 9 organoids, 50,952 single cells) across three stages of in vitro development: one month, when organoids contain a large proportion of progenitors and neurogenesis is beginning; three months, when progenitor diversity increases, including production of outer radial glia, and multiple subtypes of cortical excitatory neurons emerge; and six months, when interneurons and astroglia are present (FIGS. 18A-18C). At each timepoint, the cell type composition was highly reproducible between individual organoids, irrespective of batch and stem cell line (FIGS. 18D-18G)¹⁴.

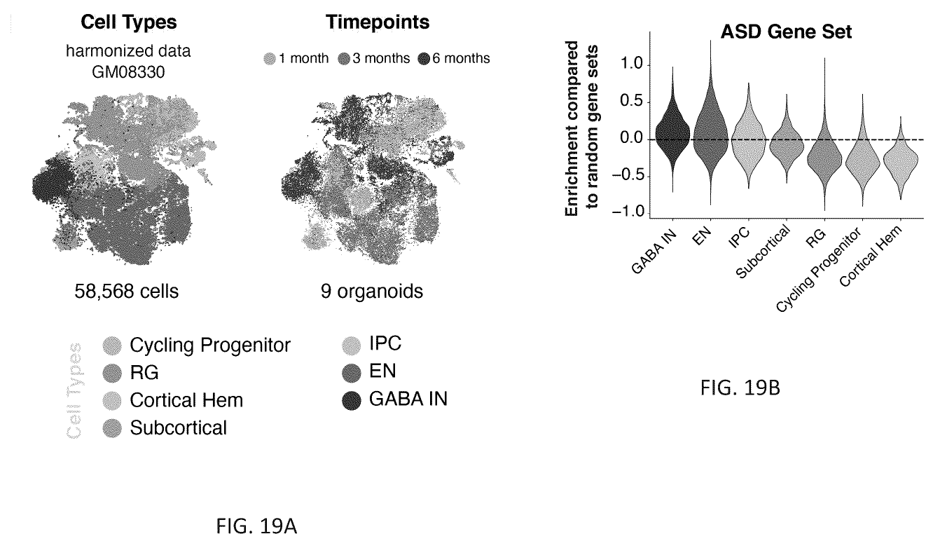

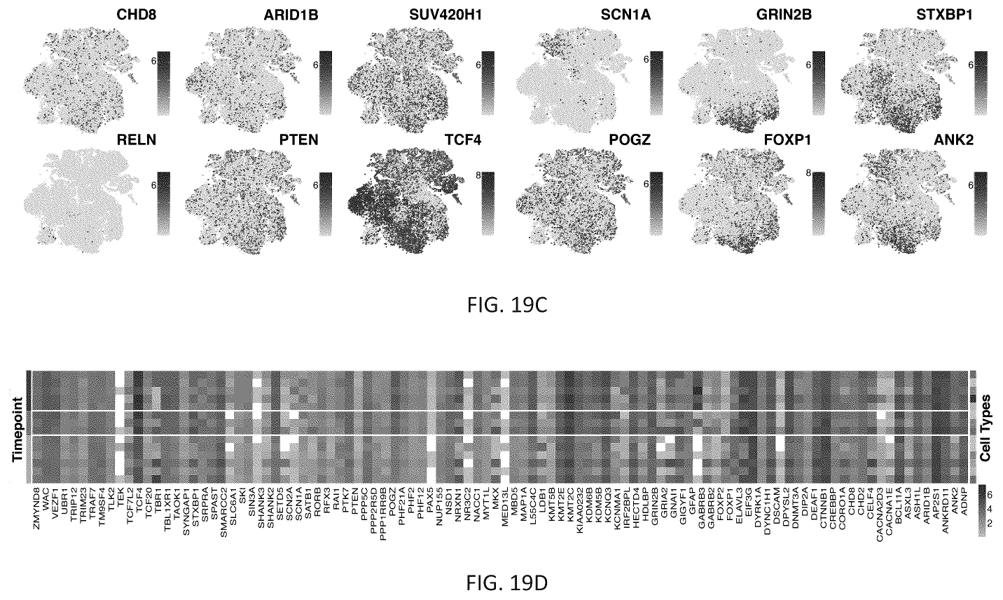

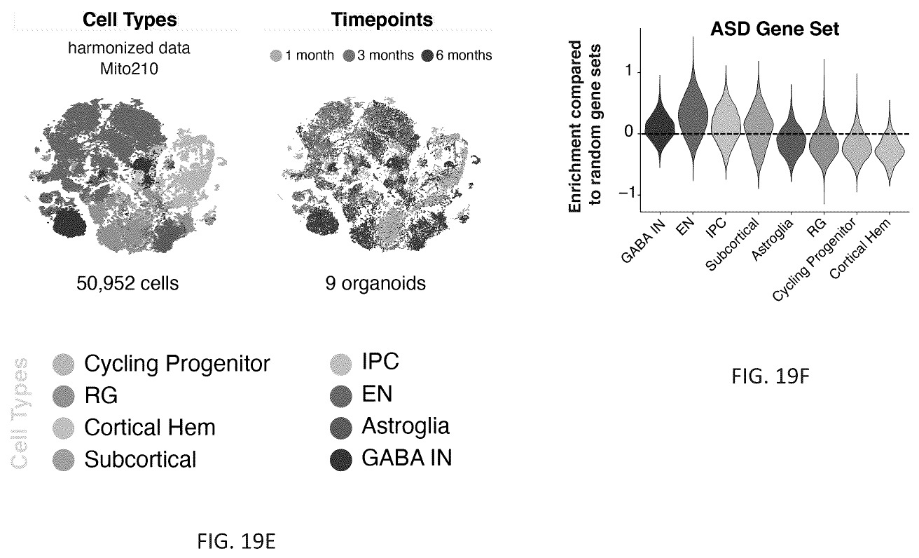

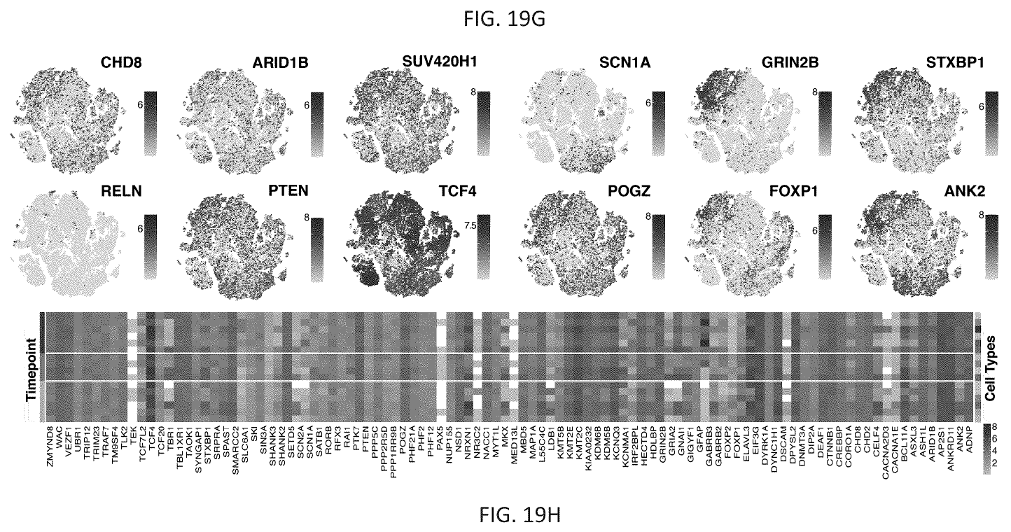

Next, 102 genes identified as contributing to ASD risk from the largest-to-date ASD exome sequencing study⁶ were taken and their expression was verified in these organoids across this developmental timeline (FIG. 19). All 102 genes were widely expressed, in patterns that varied by cell type and developmental stage (FIGS. 19C-19D, FIGS. 19G-19H). Moreover, while a combined signature of all 102 ASD risk genes was likewise widely expressed, this signature was expressed at higher levels (median enrichment score >0) in cortical excitatory projection neurons and GABAergic interneurons, compared to the other cell types in the organoids (e.g., progenitor populations and glia) (FIGS. 19A-19B, FIGS. 19E-19F), consistent with reports in the fetal human brain⁶. This result was consistent across both stem cell lines (FIGS. 19B, 19F). These data indicate that this organoid model may be used to discover neurodevelopmental phenotypes associated with ASD risk genes.

Organoids with Haploinsufficient Mutations in SUV420H1, ARID1B, and CHD8 Recapitulate Brain Size Abnormalities Observed in Patients

To begin to understand the phenotypic manifestation of ASD risk genes and whether they show convergent effects on human cortical development, three genes were chosen for further study. The 102 ASD risk genes⁶ were filtered for genes that were a) significantly implicated in risk for ASD (familywise error rate ≤0.05)⁶, b) predominantly found as de novo protein-truncating variants, which are more likely to be associated with increased ASD liability^{6,22}, and c) expressed early in brain development⁶, and thus likely to cause developmental abnormalities that can be modeled in organoids. Finally, genes were selected from a single functional category, focusing on chromatin modifiers, a class of proteins often affected in ASD and likely to influence brain development.

From the candidates meeting these criteria, SUV420H1, ARID1B, and CHD8 were focused on. SUV420H1 is a histone methyltransferase^{23,24} whose function in brain development is largely unknown but has been linked to neuroectodermal differentiation²⁵ and regulation of adult neural stem and progenitor cell pools²⁶. ARID1B is a core component of the BAF complex²⁷, which regulates cell cycle progression and acquisition of neuronal specific traits²⁸. CHD8 is an ATP-dependent chromatin-remodeling factor associated with regulation of cell cycle and neuronal differentiation²⁹⁻³¹. All three were expressed in the single-cell organoid atlas across multiple timepoints and cell types, precluding a priori prediction of cell type-specific effects (FIGS. 19C-19D, FIGS. 19G-19H).

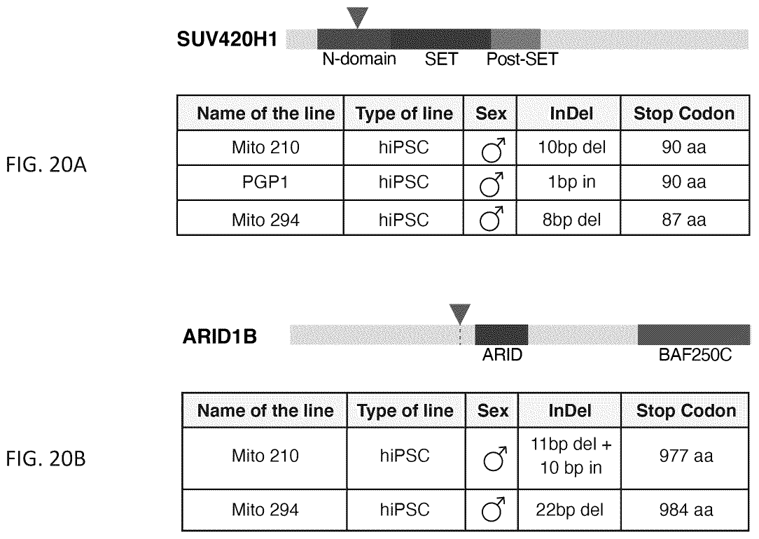

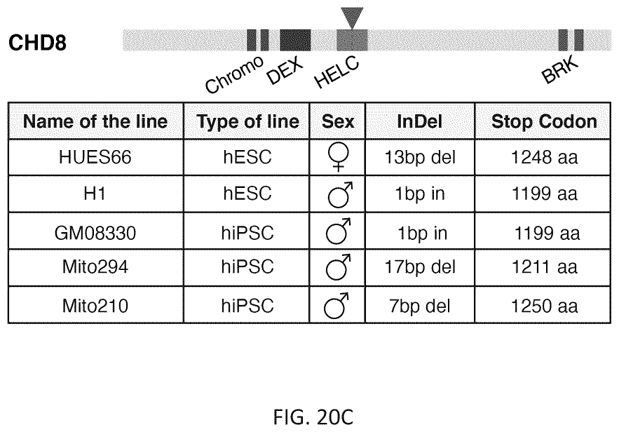

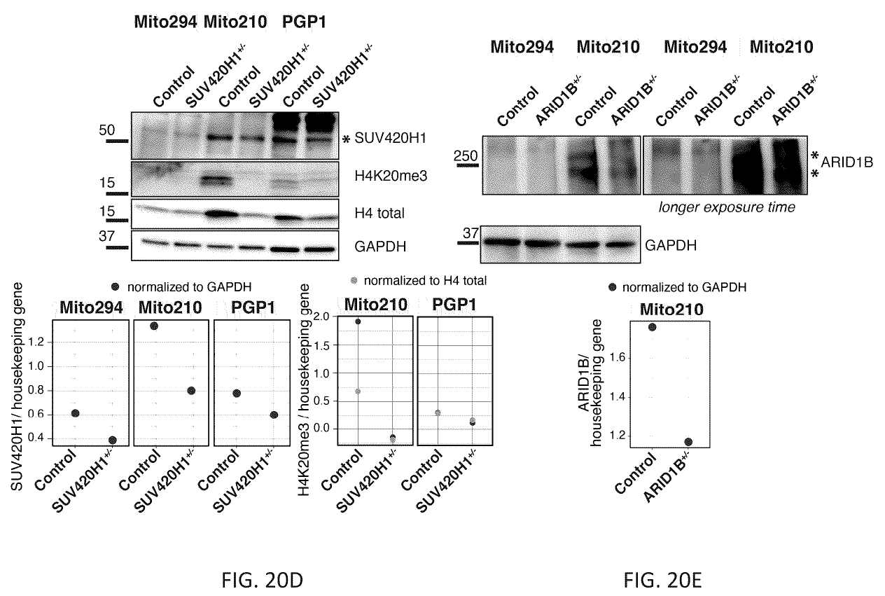

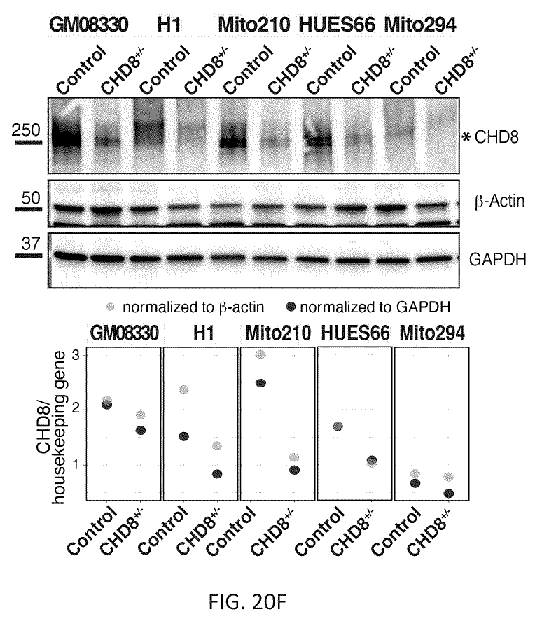

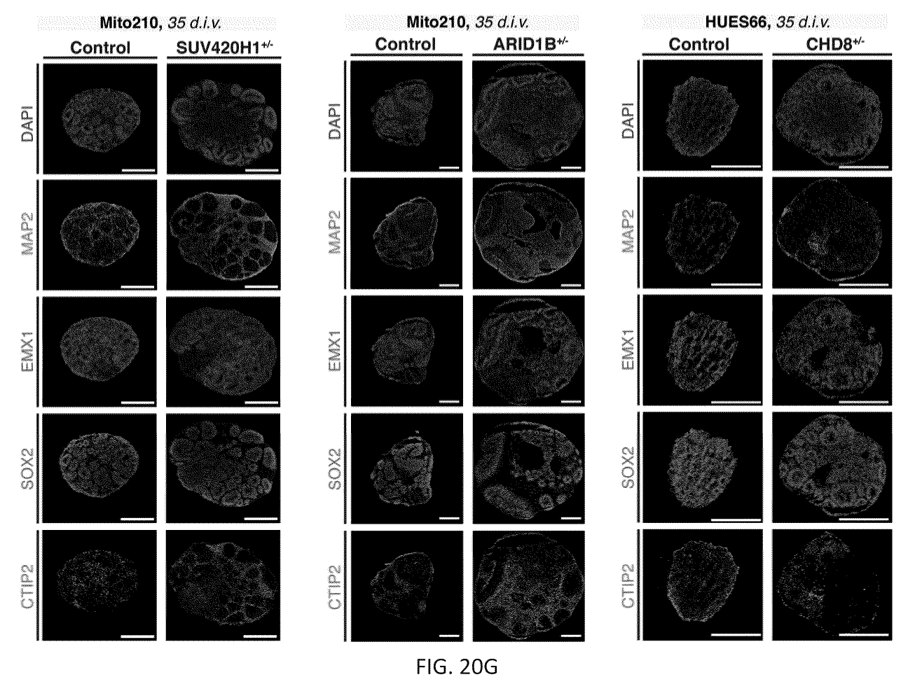

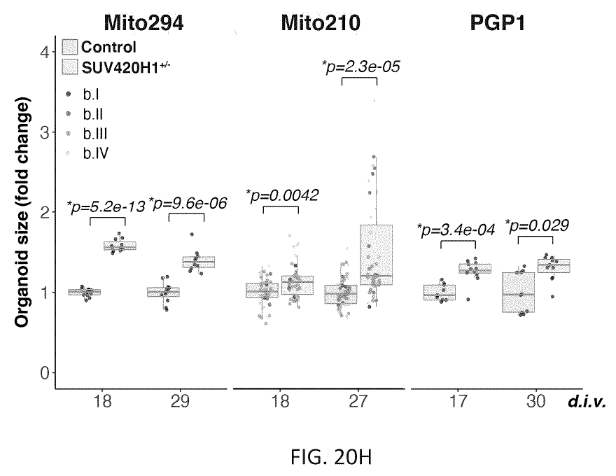

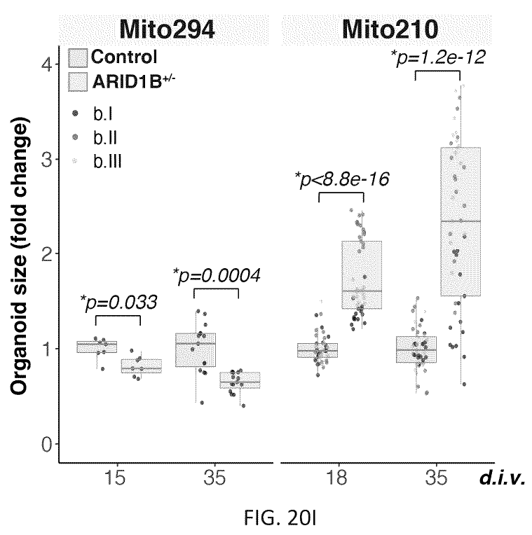

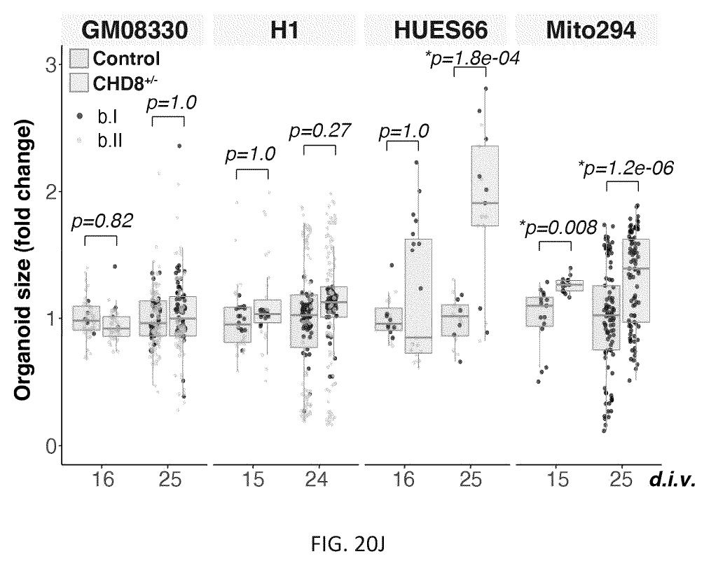

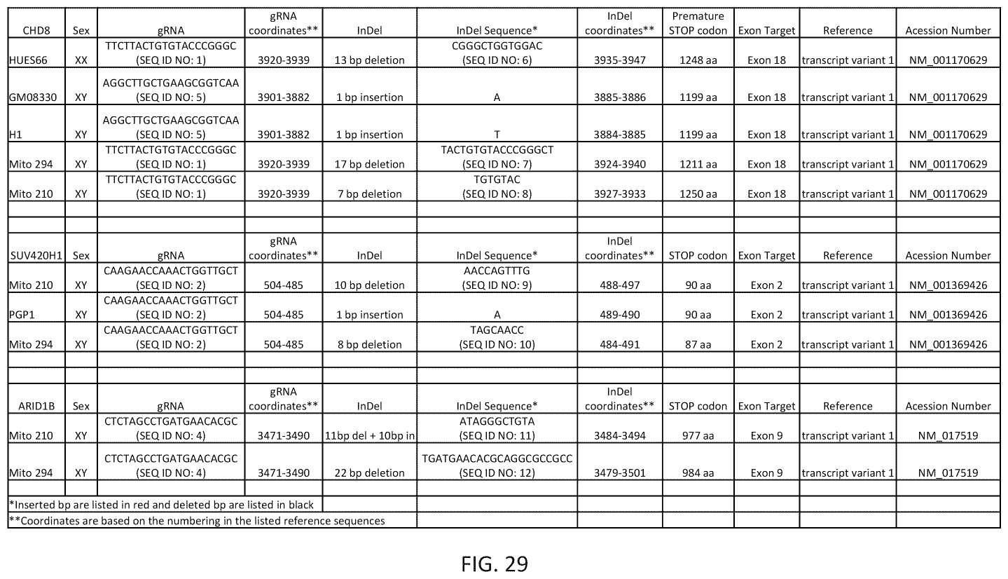

For each of the three genes, heterozygous protein-truncating indel mutations were engineered using a CRISPR/Cas9 strategy, in each case targeting a protein domain that is mutated in patients (FIGS. 20A-20C). Mutations were engineered into multiple genomic contexts for each gene, using guide RNAs targeting the same region of each gene for each stem cell line, and expanded single heterozygote clones (Methods). It was confirmed that each mutant line had indels expected to result in loss-of-function truncations (FIG. 29).

For SUV420H1, a protein-truncating frameshift was introduced in the N-terminal domain, a region affected in patients²⁴, in three parental lines from different donors (Mito294, PGP1, and Mito210; Methods and FIG. 20A). For ARID1B, two parental lines were used (Mito294 and Mito210), targeting exon 9, in a region recently found mutated in patients³² (Methods and FIG. 20B). For CHD8, five parental lines were used, two human embryonic stem cell (hESC) lines (HUES66, H1) and three iPSC lines (GM08330, Mito294, and Mito210), targeting the helicase C-terminal domain, a region mutated in patients¹⁹ (Methods and FIG. 20C).

Notably, for all three risk genes there was substantial variation in endogenous levels of each protein, as well as in the degree of reduction in the mutants, as determined by Western blot of undifferentiated stem cells from each mutant and isogenic control line. In each case, different genomic contexts (parental lines) showed substantial difference in endogenous expression of the risk proteins, such that in a line with high endogenous expression in the control, the level of the protein in the corresponding mutant line, even though significantly reduced, may resemble the level in another control line with lower endogenous expression (e.g., compare Mito210 and PGP1 for SUV420H1; FIGS. 20D-20F). Specifically, for SUV420H1, two lines (Mito210 and PGP1) had robust SUV420H1 protein levels in the controls and showed a clear reduction of protein in the mutants (FIG. 20D). As SUV420H1 is a histone H4 methyltransferase²⁴, a functional impairment was also validated by quantifying the levels of H4K20me3 methylation. Both lines showed reduction in H4K20me3 methylation in the mutant, corroborating a decrease in SUV420H1 protein function (FIG. 20D). As with SUV420H1 protein levels, the degree of reduction varied between lines, with Mito210 showing a larger decrease. By contrast, the Mito294 SUV420H1 control cell line had lower endogenous levels of SUV420H1 and H4K20me3 (FIG. 20D). For ARID1B, the level of ARID1B protein was undetectable in both the control and mutant Mito294 ARID1B lines, while the Mito210 control had higher endogenous expression, and the corresponding mutant retained an appreciable level of protein despite a sizeable decrease (FIG. 20E). For CHD8, the HUES66, Mito210, and H1 cell lines had robust protein expression in the controls, and the corresponding mutants had the largest decreases in CHD8 levels, while the GM08830 and Mito294 had relatively modest decreases in the mutant lines (FIG. 20F). Overall, for all three mutations, variation in the baseline level of each protein in the control parental lines was found, which in turn influenced the absolute amount of protein remaining in the heterozygote derivative. These data underscored the importance of investigating risk genes across multiple genomic contexts, as different pluripotent stem cell lines might vary in phenotypic expressivity due to variation in endogenous risk gene expression, among other differences.

Cortical organoids were produced from each isogenic control and heterozygous mutant line using the previously-established protocol¹⁴. Organoids from all lines initiated appropriate neural development (FIG. 20G), with the exception of the Mito210 CHD8 mutant line, which was therefore excluded from this study.

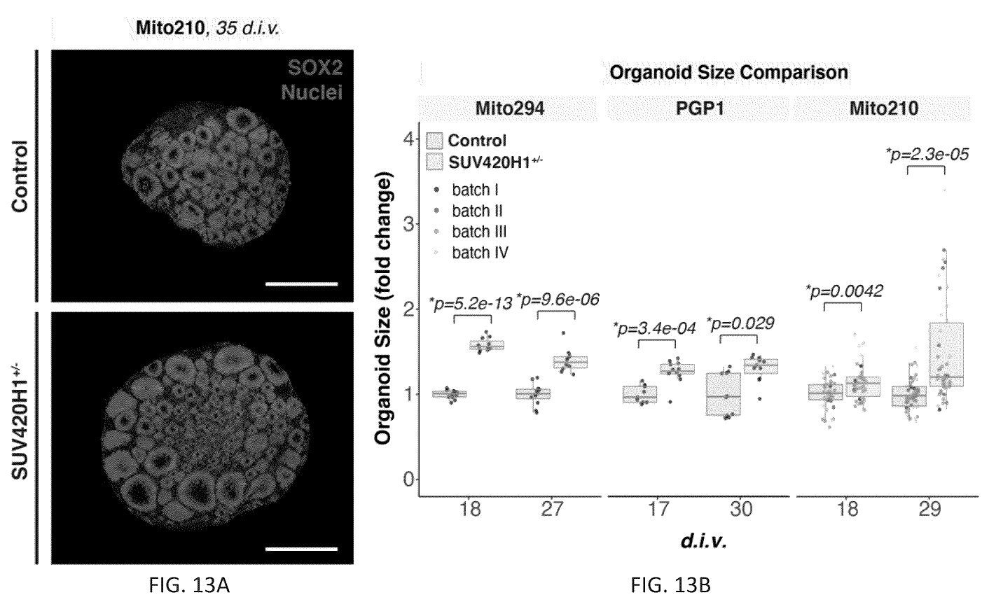

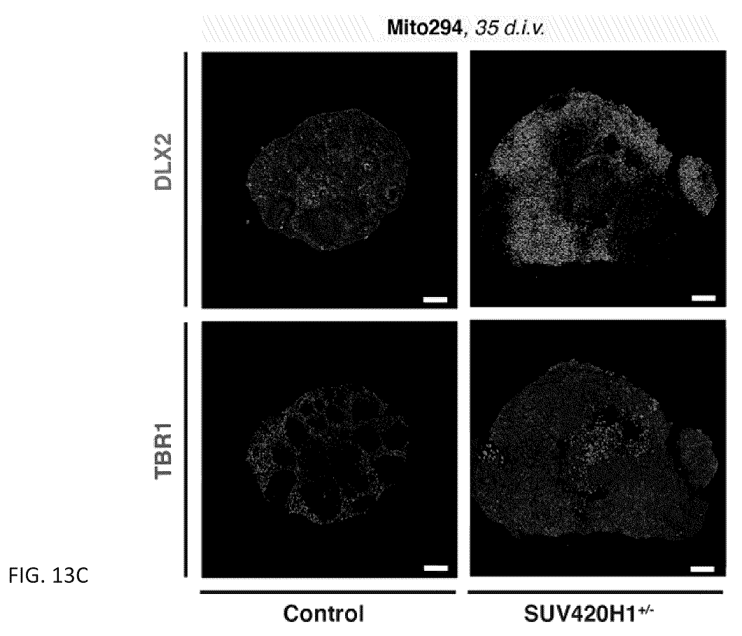

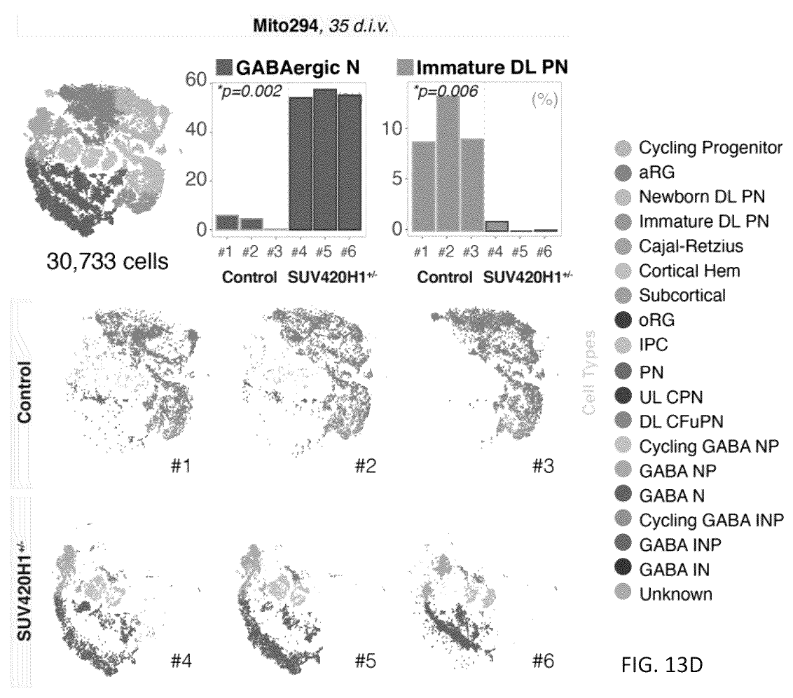

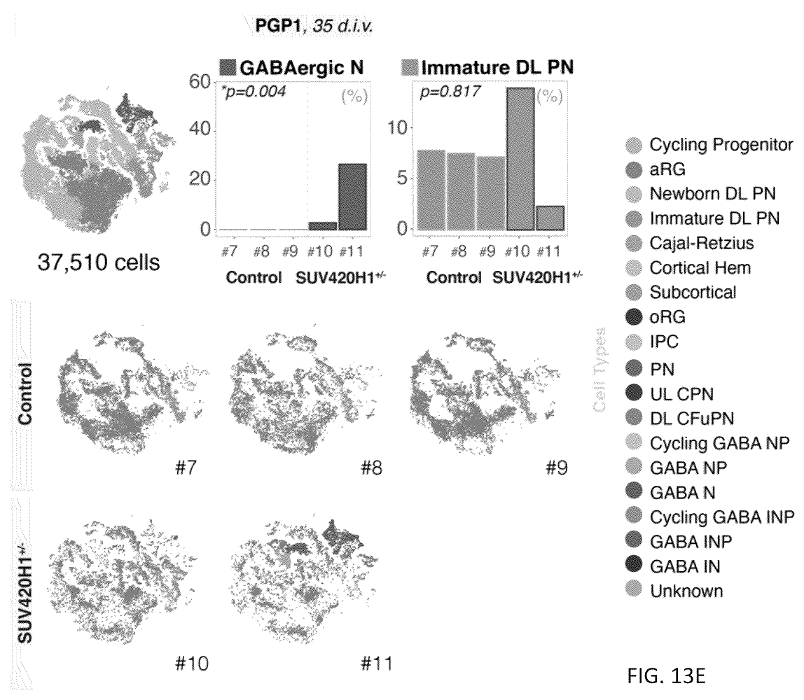

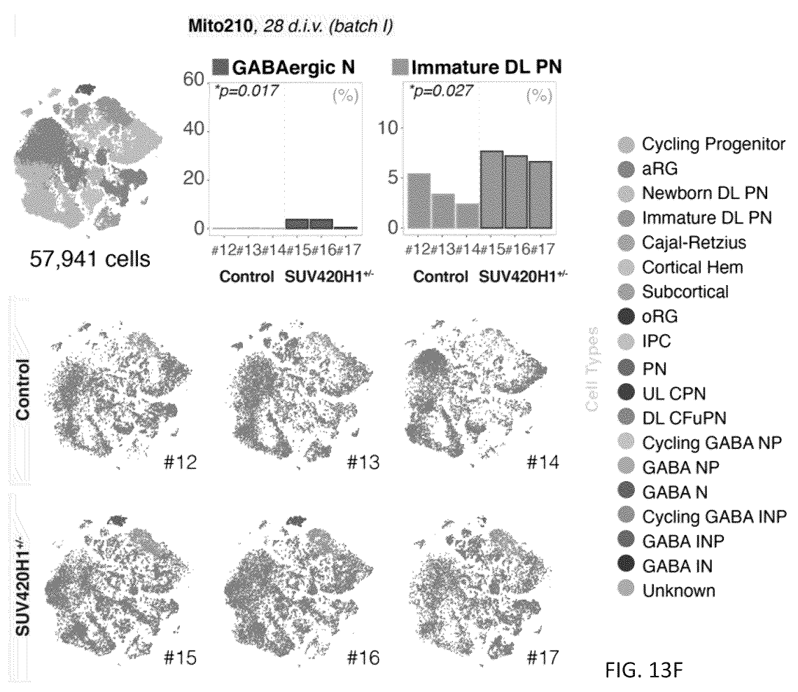

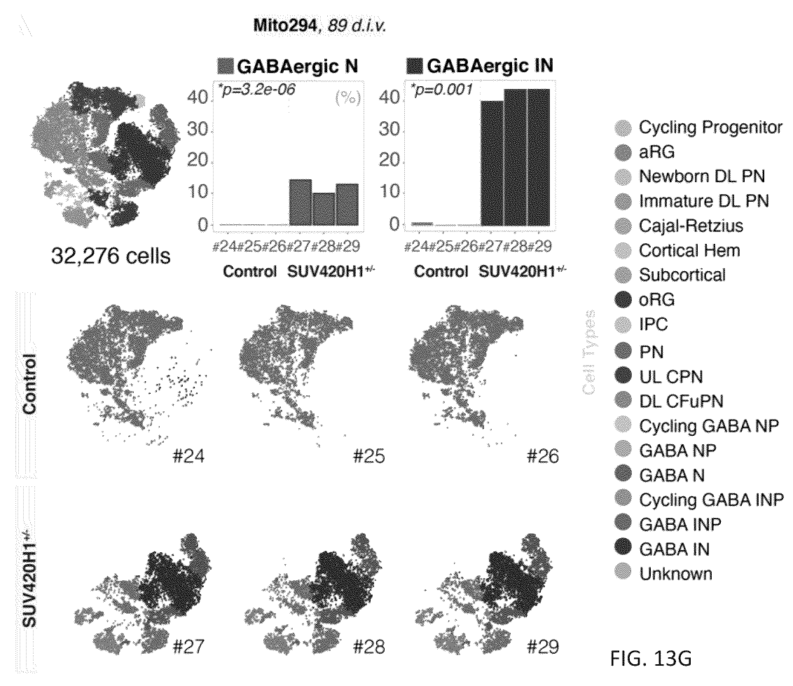

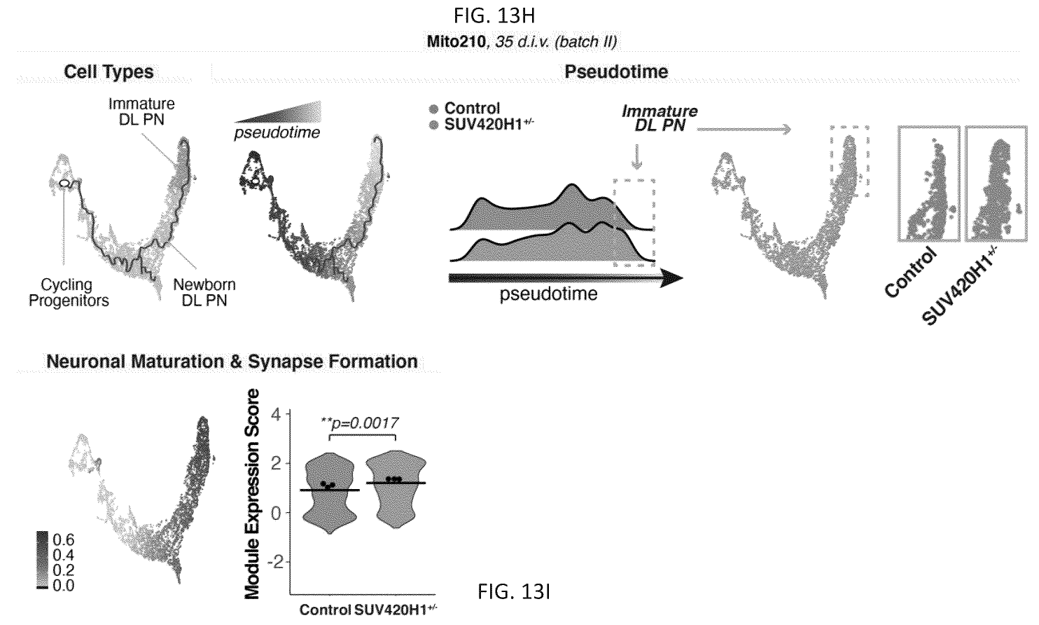

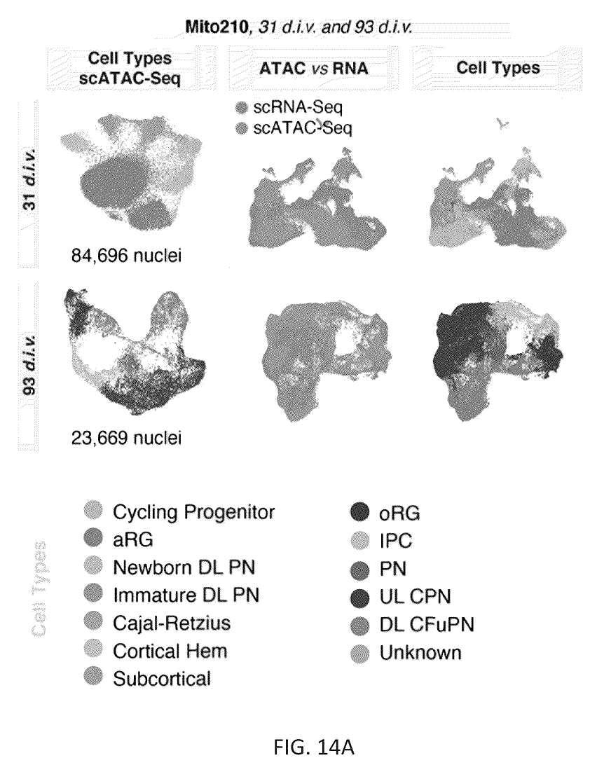

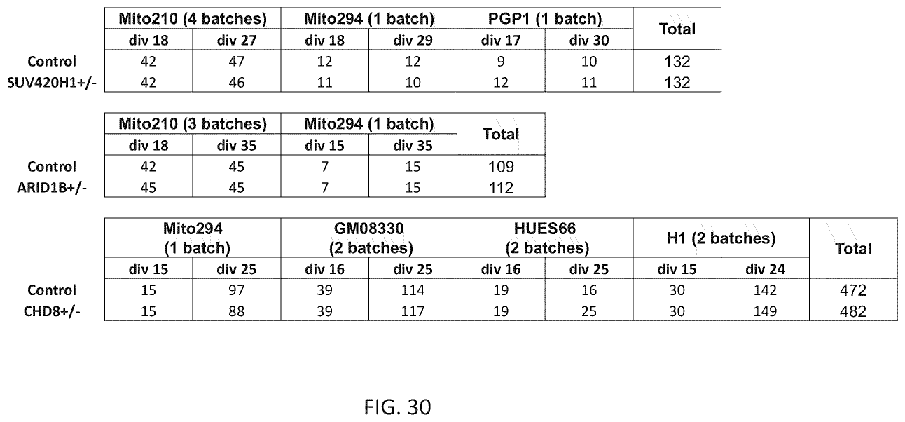

As all three genes are linked to macrocephaly and/or microcephaly in patients, organoid size was quantified for each mutant and control line (see FIG. 30 for n of each experiment). For SUV420H1, mutant organoids from all genomic contexts were significantly larger than controls at both two weeks (17-18 d.i.v.) and one month (27-30 d.i.v.), reminiscent of the macrocephalic phenotype observed in individuals carrying de novo mutations in the SUV420H1 gene²⁴ (p<0.05 in all cases, t-test; FIGS. 13A-13B, FIG. 20H).

For ARID1B, haploinsufficient organoids showed an increase in size in the Mito210 background at both two weeks and one month (adjusted p<8.8×10⁻¹⁶ at 18 d.i.v. and adjusted p=1.2×10⁻¹² at 35 d.i.v., t-test; FIG. 20I), but an opposite effect in the Mito294 background (adjusted p=0.033 at 15 d.i.v. and p=0.0004 at 35 d.i.v., t-test; FIG. 20I). This is notable, given that in patients, haploinsufficient mutations in ARID1B have been associated with both macrocephaly and microcephaly^{21,33}, suggesting that distinct phenotypes might result from interactions of ARID1B mutation with distinct genomic contexts.

For CHD8, at one month (24-25 d.i.v.), mutant organoids were significantly larger compared to controls in two out of the four lines tested, across multiple batches (adjusted p=1.8×10⁻⁴ in HUES66, p=1.2×10⁻⁶ in Mito294, t-test; FIG. 20J). This is consistent with macrocephalic phenotypes reported in ˜80% of patients with mutations in CHD8¹⁹.

These data indicate that human cortical organoids bearing haploinsufficient mutations in SUV420H1, ARID1B, and CHD8 replicate size defects related to the macrocephalic and microcephalic abnormalities seen in patients carrying these mutations, suggesting that organoids can capture clinically-relevant features of ASD pathology and justifying further investigation using this model.

### SUV420H1 Haploinsufficiency Induces Asynchronous Generation of Both GABAergic Neurons and Deep Layer Excitatory Projection Neurons

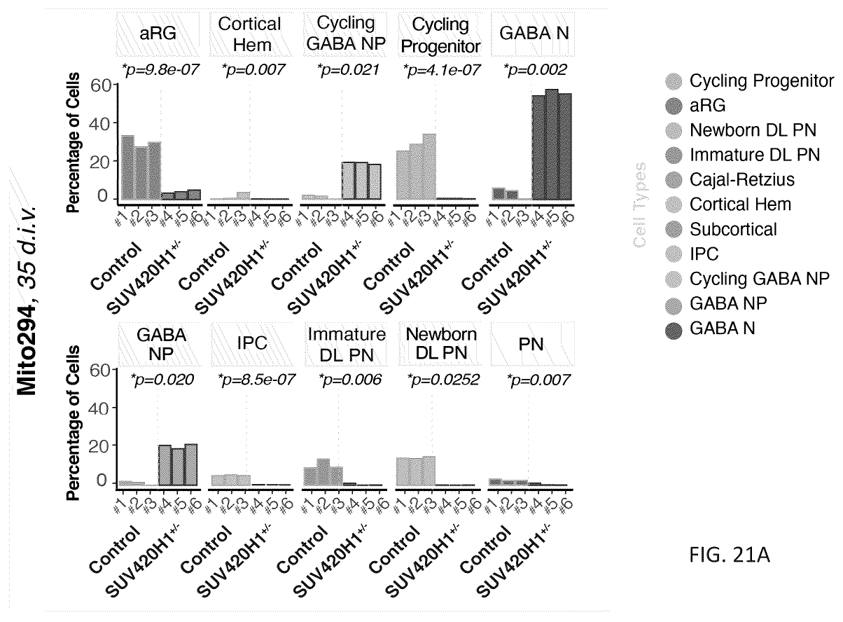

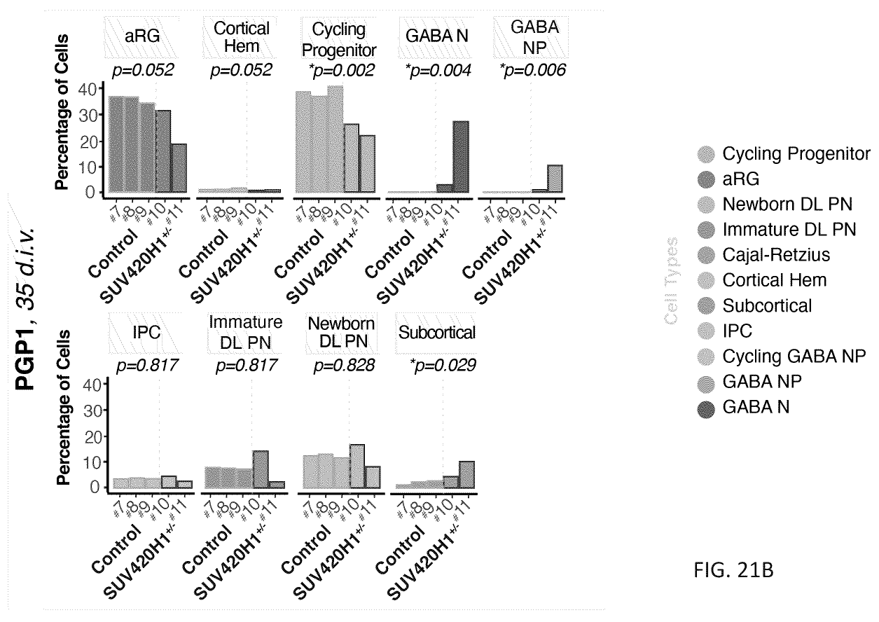

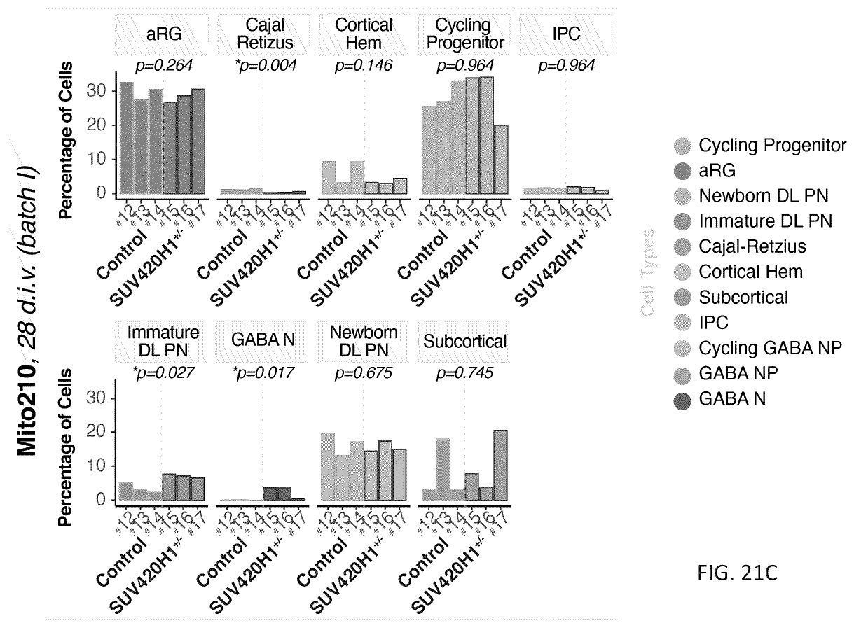

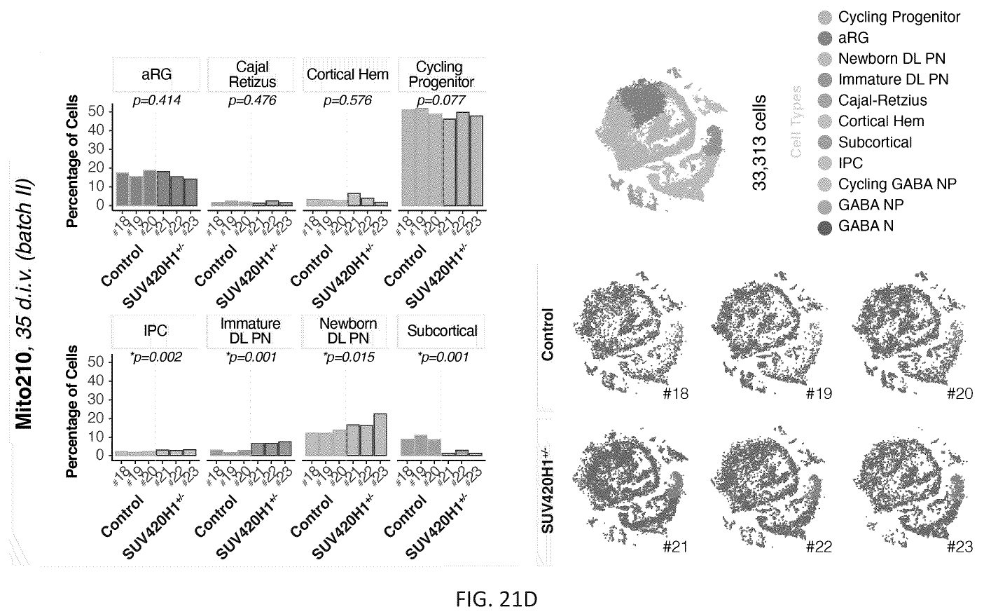

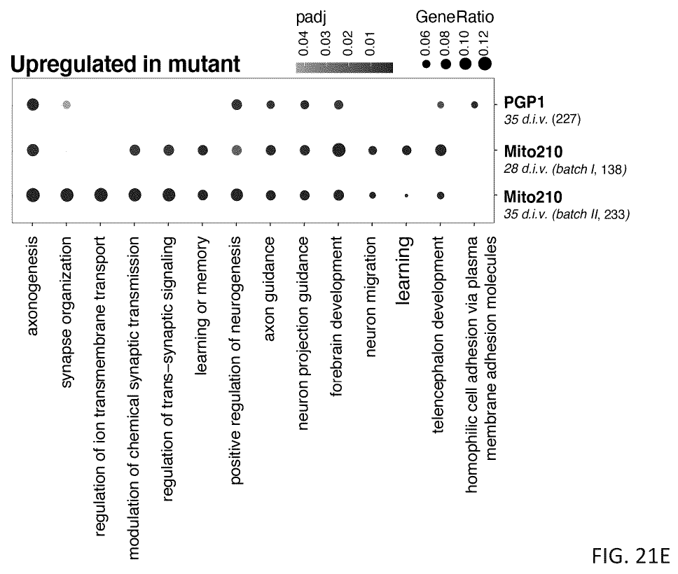

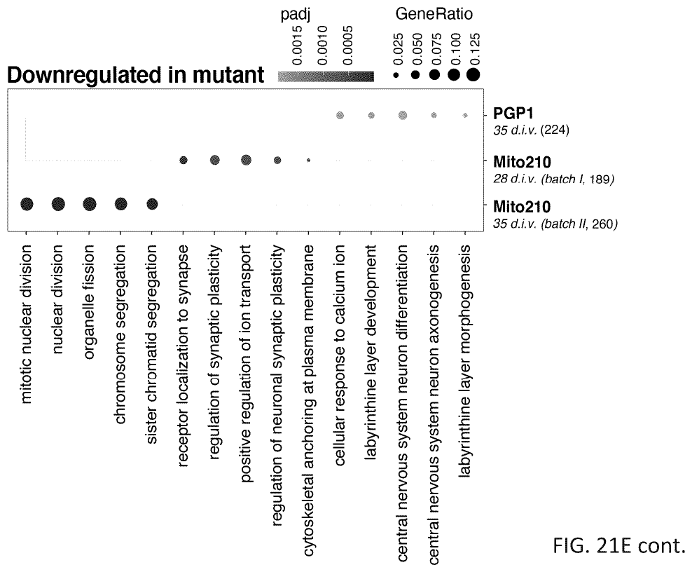

In order to investigate developmental abnormalities in the SUV420H1 mutant, single-cell RNA-seq was performed on mutant and control organoids at different stages of in vitro development. Striking developmental defects were found in specific populations of cortical neurons in mutant organoids. Early stages of mutant and control organoids were profiled for Mito294 SUV420H1 (30,733 single cells at 35 d.i.v.), PGP1 SUV420H1 (37,510 single cells at 35 d.i.v.), and Mito210 SUV420H1 (57,941 single cells at 28 d.i.v., and 33,313 single cells at 35 d.i.v., from two separate differentiations). At this early time point, mutant organoids showed a consistent presence of GABAergic neurons in all the genomic contexts profiled (FIGS. 13C-13F and FIGS. 21A-21D). This is striking because GABAergic neurons are rare or absent in control organoids until approximately 3.5 months¹⁴. The GABAergic population in mutant organoids at one month expressed broad markers of GABAergic identity (e.g., DLX2, DLX6, GAD1, GAD2; this population is henceforth referred to as “GABAergic neurons”).

Despite the consistency of this phenotype across lines and differentiations, there were noticeable differences in phenotypic severity (expressivity) across genomic contexts. Specifically, the Mito294 SUV420H1 line showed the most dramatic increase in GABAergic neurons, with over 50% of the cells in all mutant organoids belonging to the GABAergic lineage, and <5% belonging to the excitatory projection neuron lineage (n=3 organoids per genotype, adjusted p=0.002, logistic mixed models; FIGS. 13C-13D and FIG. 21A). PGP1 SUV420H1 showed a phenotype of intermediate severity with up to 35% of cells belonging to the GABAergic lineage (n =2-3 organoids per genotype, adjusted p=0.004, logistic mixed models, FIG. 13E and FIG. 21B). Finally, the Mito210 SUV420H1 showed the mildest phenotype, with no more than 5% of cells belonging to the GABAergic lineage in one batch (28 d.i.v., n=3 organoids per genotype, adjusted p=0.017, logistic mixed models; FIG. 13F and FIG. 21C) and no appearance of GABAergic neurons in a second differentiation batch at 35 d.i.v. (FIG. 21D). This suggests that while converging on the same phenotype of premature generation of GABAergic neurons, the broader genetic and epigenetic context of each cell line can modulate the phenotypic expressivity of the SUV420H1 mutation.

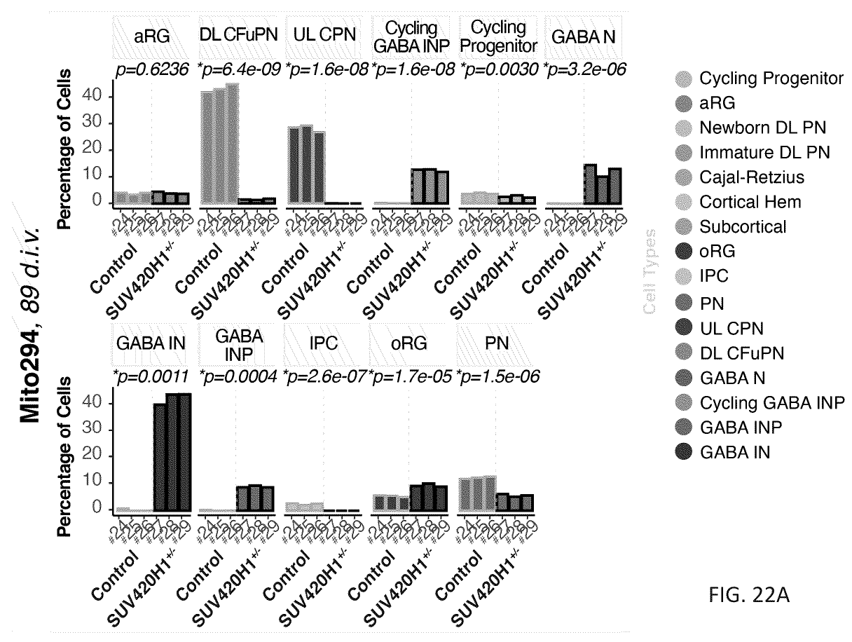

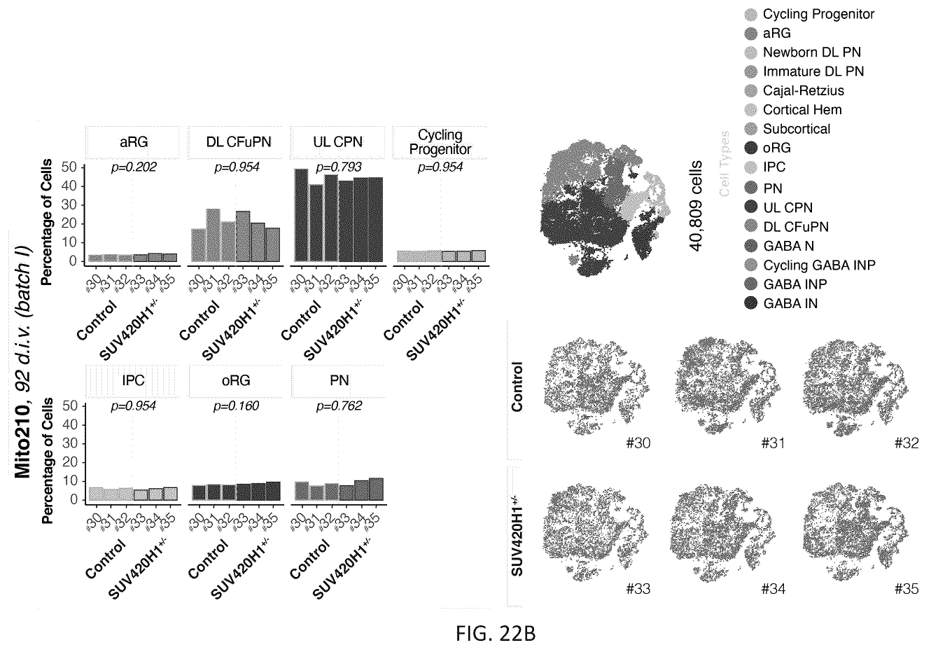

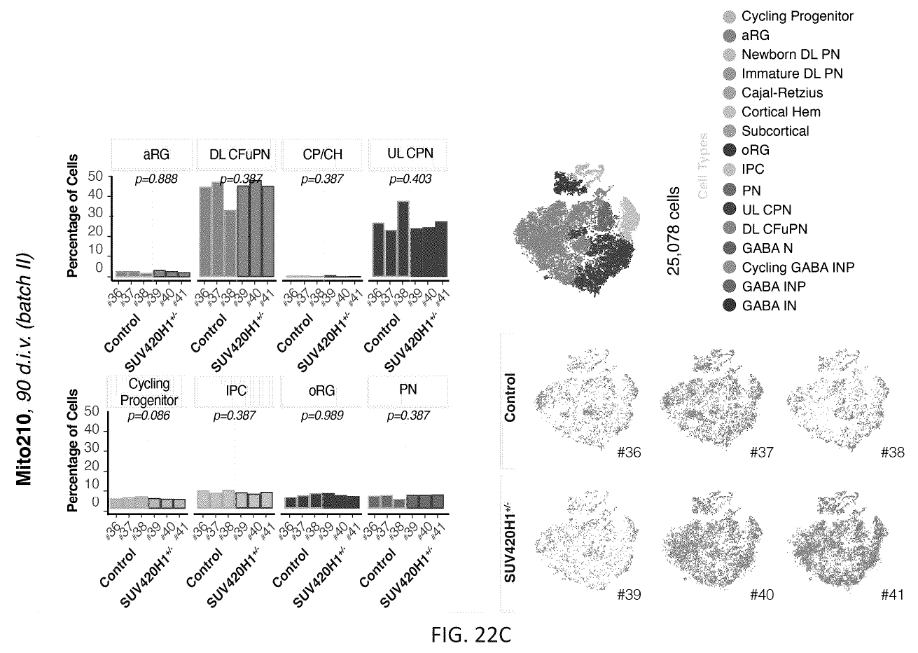

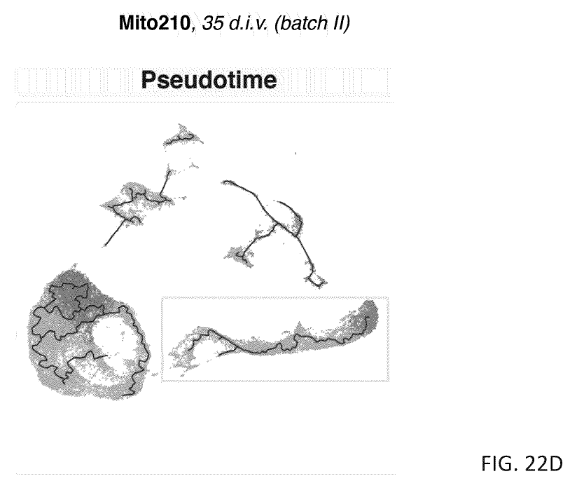

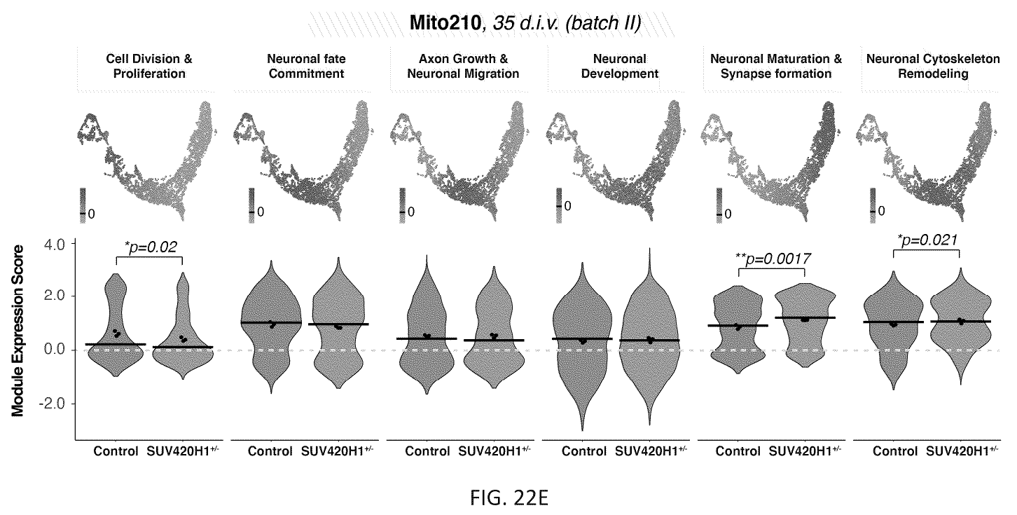

Whether the increase in GABAergic neurons induced by SUV420H1 haploinsufficiency persisted at later stages of development was then investigated. Organoids from the two lines that showed the greatest difference in phenotypic severity (Mito294 and Mito210) were profiled. At three months (89 d.i.v.), scRNA-seq of the Mito294 SUV420H1 mutant organoids, which showed the most dramatic phenotype at one month (FIGS. 13C-13D), still showed a nearly complete switch to a GABAergic population compared to control organoids, which had only cells of the cortical excitatory lineage (32,276 single cells, n=3 single organoids per genotype; FIG. 13G and FIG. 22A). At three months and beyond, GABAergic populations express clear molecular features of cortical interneurons in addition to the general GABAergic markers expressed at one month (e.g., NR2F2, SP8, PROX1; therefore indicated as “GABAergic interneurons”). However, scRNA-seq on two distinct batches from Mito210 SUV420H1 control and mutant organoids after three months in vitro (batch I at 92 d.i.v.: 40,809 single cells, n=3 single organoids per genotype; batch II at 90 d.i.v.: 25,078 single cells, n=3 single organoids per genotype), showed no GABAergic interneurons in the mutant or the control at this stage (FIGS. 22B-22C). This indicates that depending on its expressivity, the GABAergic phenotype can be transient and resolve over development (Mito210) or persist (Mito294) in mutant organoids.

It was then sought to examine possible changes in other cell types. Due to the pronounced overgrowth of the GABAergic lineage in the Mito294 SUV420H1 mutant organoids, most other cell types had reduced proportions (FIGS. 13C-13D, FIG. 13G, FIG. 21A, FIG. 22A). However, in the Mito210 SUV420H1 mutant organoids at one month, the milder GABAergic phenotype allowed us to quantify an increase in immature deep layer projection neurons, the first-born neurons of the cortical plate^{34,35}, in two differentiation batches (FIG. 13F and FIGS. 21C-21D). In this line at 28 d.i.v. (batch I), the immature deep layer projection neurons were consistently increased in every organoid analyzed (n=3 single organoids per genotype, adjusted p =0.027, logistic mixed models; FIG. 13F and FIG. 21C). Analysis of the second differentiation batch of the Mito210 SUV420H1 control and mutant organoids sampled at 35 d.i.v confirmed an increase in this population of immature deep layer projection neurons (n=3 organoids per genotype, adjusted p=0.001, logistic mixed models; FIG. 21D). In addition, the proportions of earlier cell types of the deep layer projection neuron lineage (intermediate progenitor cells and early-postmitotic newborn deep layer neurons) were also increased (FIG. 21D). Similar to the transient nature of the GABAergic neuron phenotype in this line, the deep layer projection neuron phenotype was rescued after three months in vitro; equivalent proportions of deep layer corticofugal projection neurons (the more mature state of the deep layer population affected at one month) were found in mutant and control organoids from two different differentiation batches (92 and 90 d.i.v., FIGS. 22B-22C).

In the PGP1 background, although the GABAergic phenotype was consistently seen, an increase in the number of deep layer projection neurons in the PGP1 SUV420H1 mutant organoids was not seen at one month (35 d.i.v., FIG. 13E, FIG. 21B). To evaluate whether there were molecular differences indicative of changes in maturation state, gene expression changes in the developmental lineage leading to deep layer projection neurons were examined. The cells in this developmental lineage were extracted from the scRNA-seq datasets (see below) and differentially expressed genes between SUV420H1 mutant and control in both the PGP1 and Mito210 datasets were calculated. Genes upregulated in mutant organoids from both lines were enriched in gene ontology (GO) terms related to neuronal differentiation and maturation (FIG. 21E; Methods), pointing towards a more advanced developmental state of deep layer projection neurons in SUV420H1 mutants, in both genomic contexts (Mito210 and PGP1). This indicates that in the PGP1 background, the SUV420H1 mutation similarly affects deep layer projection neuron development, causing a molecular profile indicative of more advanced maturation but without a consistent change in cell proportions. Interestingly, although the Mito210 line showed a more severe phenotype for the deep layer projection neurons than the PGP1 line, the latter showed a more severe phenotype for the GABAergic neurons, indicating that different features of the mutant phenotype can be differentially modulated by the same genomic context.

### Increase in Deep Layer Projection Neurons in SUV420H1 Mutants Reflects Accelerated Differentiation

To better understand the mechanisms behind the increased number of neurons in the SUV420H1 mutants, the developmental dynamics of the affected cell types were examined along a developmental continuum of cells within a specific lineage. As the very low numbers of GABAergic neurons in the control lines at one month precluded accurate computation of developmental trajectories, the deep layer projection neuron phenotype was focused on, and pseudotime trajectories were calculated using Monocle3³⁶. As pseudotime analysis results in multiple developmental trajectories leading to different end states (for example, astrocytes or projection neurons), the portion of the trajectory corresponding to the development of the affected cell types (the “partition of interest”) was first identified. Initial Monocle analysis of the combined 35 d.i.v. Mito210 SUV420H1 mutant and control organoids (batch II) split the cells into two major partitions, one containing progenitors traversing the cell cycle, and another containing cycling progenitors maturing towards deep layer projection neurons (FIG. 22D). After re-computing trajectories on only the cells in the latter partition, Mito210 SUV420H1 mutant organoids showed an increased distribution of cells toward the end point of these developmental trajectory compared to controls (p<2.2×10⁻¹⁶, one-sided Kolmogorov-Smirnov test, FIG. 13H), supporting an acceleration in the developmental trajectory of these neurons. Co-expression analysis by WGCNA³⁷ (Methods) identified six co-varying gene modules across mutant and control cells in the partition of interest (FIG. 22E), which were labeled by known genes. A module containing multiple genes known to be involved in neuronal maturation and synapse formation (e.g., GRIA2, CAMK2B, SNAP25, CNTNAP2, NRCAM, and NRNX2), was positively correlated with pseudotime progression (FIG. 13I; Pearson correlation r=0.94, p<2.2×10⁻¹⁶). This module was significantly upregulated in mutant organoids compared to controls (adjusted p=0.0017, linear mixed models, FIG. 13I). Together, these results support an accelerated differentiation phenotype in this class of neurons induced by SUV420H1 haploinsufficiency.

It was then sought to explore possible mechanisms for the premature expression of maturation-associated genes in this mutant. SUV420H1 is a Histone-Lysine N-Methyltransferase responsible for catalyzing the methylation of H4K20me1 to H4K20me2 and H4K20me3, ultimately leading to chromatin compaction and transcriptional silencing²⁴. Therefore, changes in chromatin accessibility were examine in these mutants.

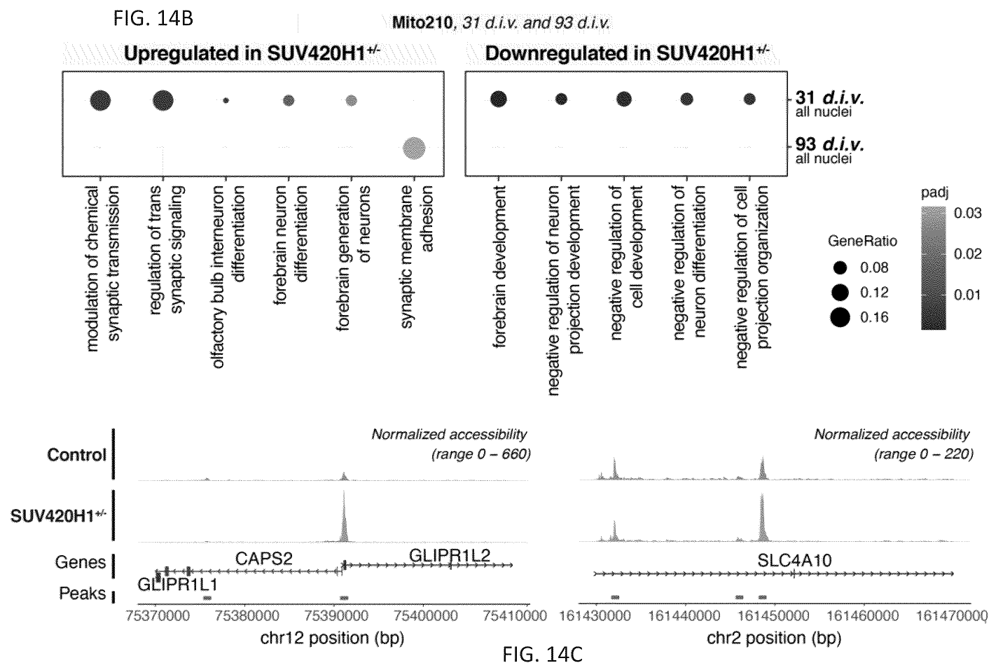

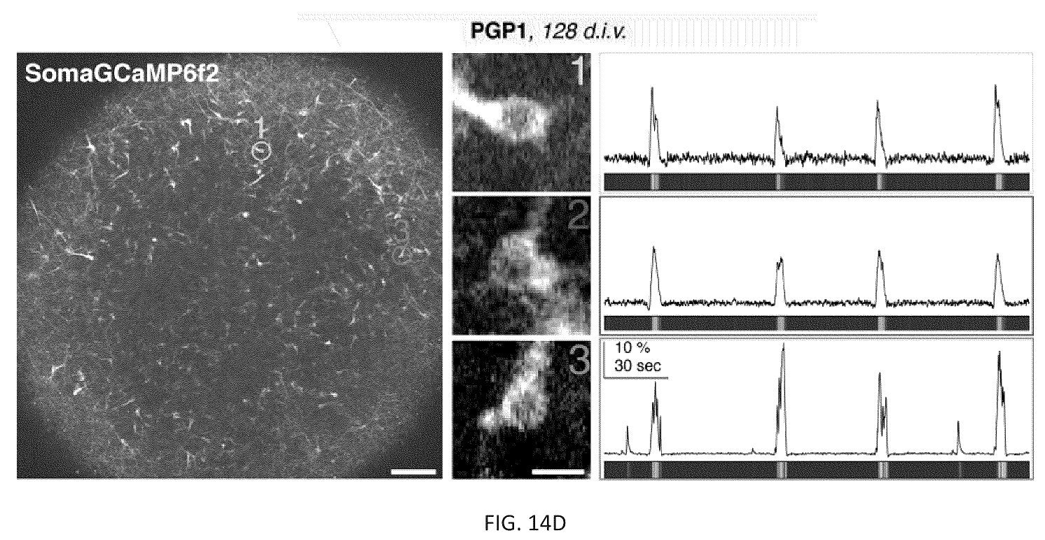

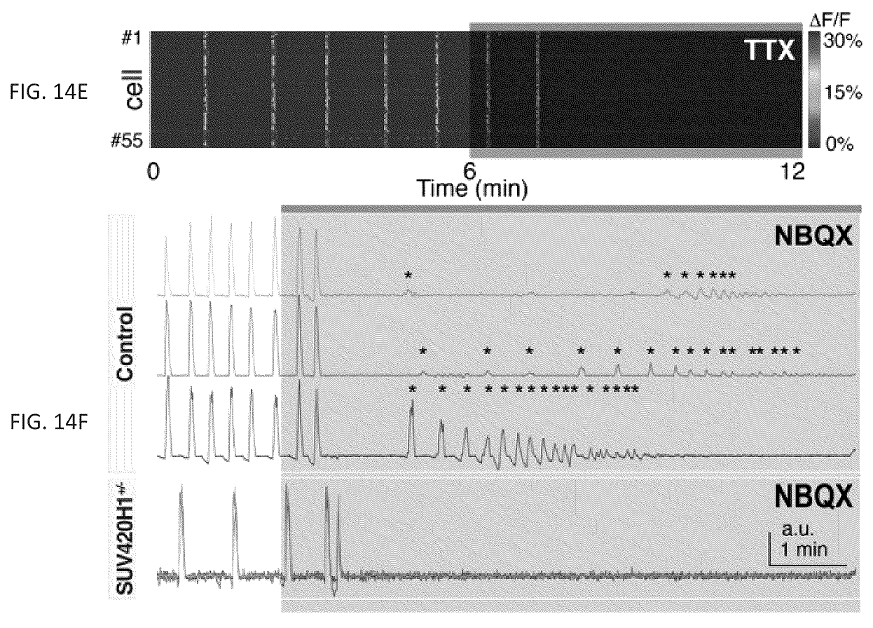

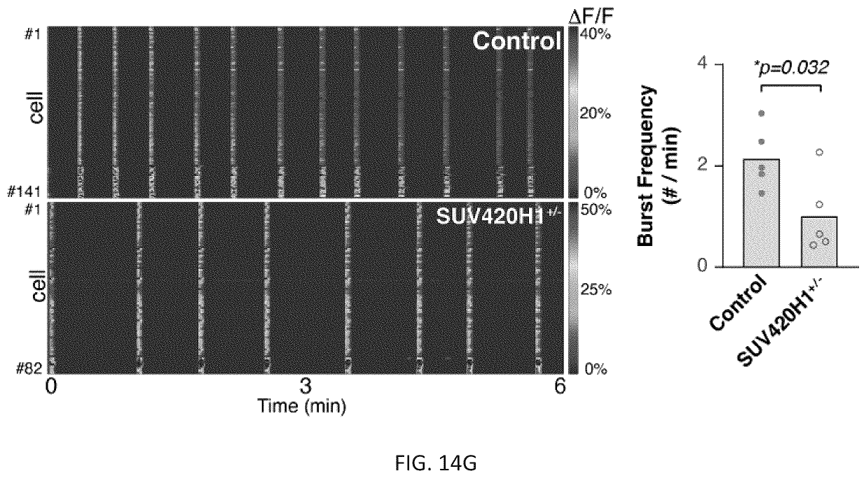

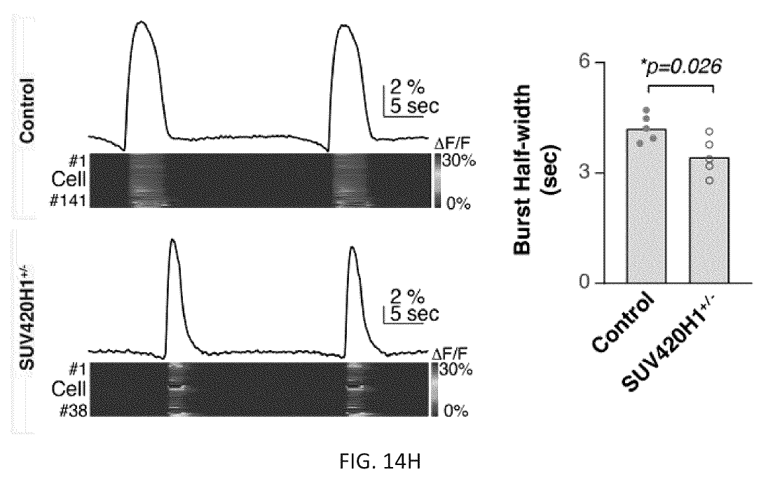

Single-cell Assay for Transposase Accessible Chromatin was performed using sequencing (scATAC-seq) on Mito210 SUV420H1 organoids at both one and three months (84,696 single nuclei at 31 d.i.v., n=3 single organoids per genotype; 23,669 single nuclei at 93 d.i.v., n=3 single organoids per genotype). Cell types in both datasets were annotated by projecting scRNA-seq annotations. Co-embedding scATAC-seq and scRNA-seq data showed that chromatin accessibility capture most of the cell types identified by gene expression, and further confirmed the overall consistency between expression and chromatin accessibility profiles (FIG. 14A).

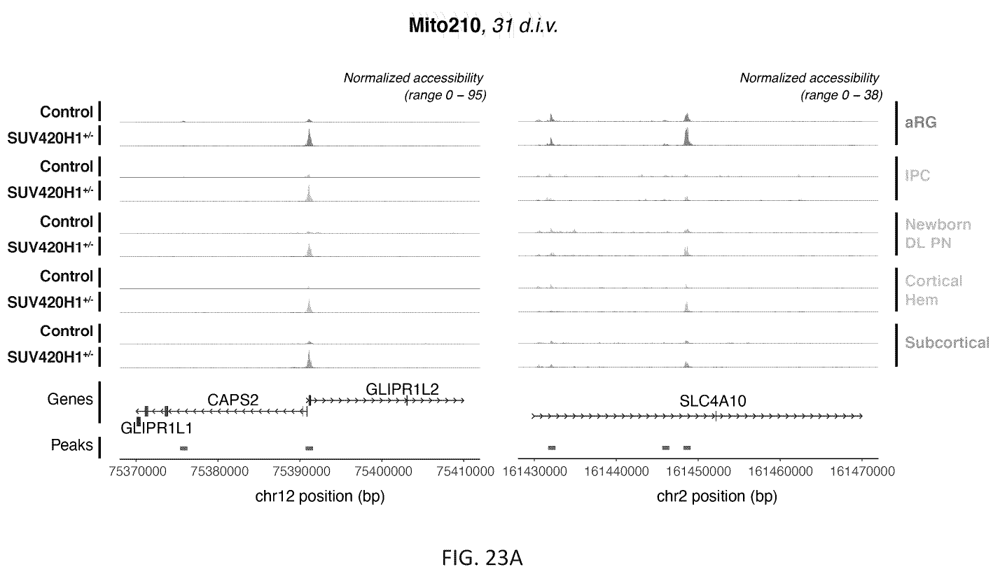

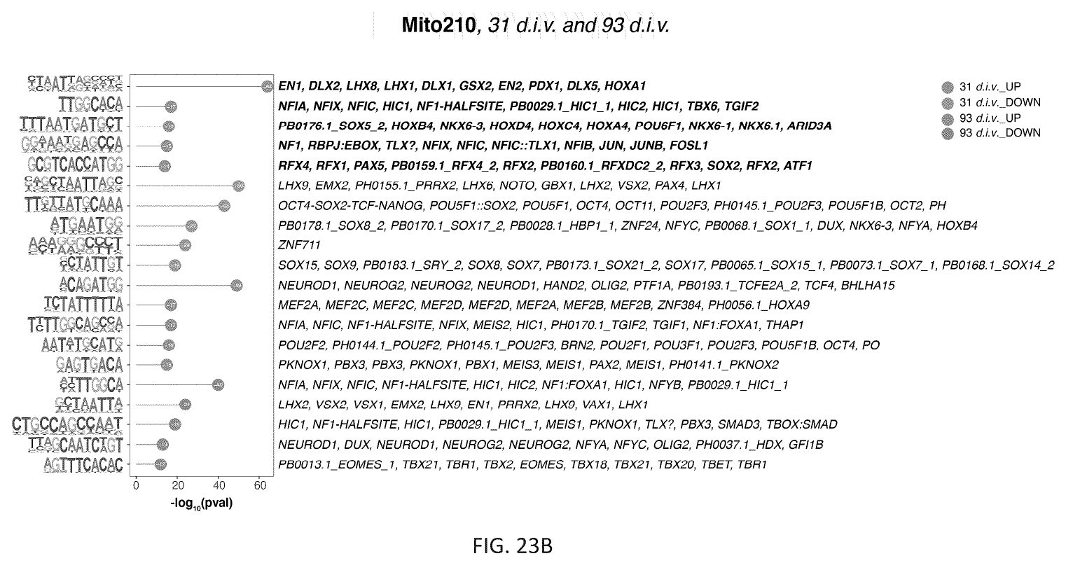

At one month (31 d.i.v.), a modest number of regions were differentially accessible (adjusted p<0.1, logistic regression) between mutant and control for most cell types (range 6-34), except for apical radial glia cells, the most prevalent (and thus most powered) cell type at this time point, which had 515 differentially accessible regions (DARs). Because most of the significant DARs overlapped across cell types, all cells were combined and DARs were recalculated to identify a final set of 414 DARs. The genes nearest (within 10 Kb) to DARs with increased accessibility in mutant organoids were enriched for GO terms associated with synaptic transmission and neuronal maturation, whereas genes nearest to DARs with reduced accessibility were enriched for negative regulation of neuronal maturation, connectivity, and development (FIG. 14B). Thus, haploinsufficient mutation in SUV420H1 leads to changes in chromatin accessibility linked to regulation of genes involved in neurodevelopment and neuronal maturation (e.g., CAPS2 and SLC4A10, FIG. 14C, FIG. 23A), consistent with the phenotypes observed by scRNA-seq.

At a later developmental stage, three months in vitro (93 d.i.v.), only 43 significant DARs (adjusted p<0.1) were detected across all cells in both mutant and control organoids, suggesting that many of the differences detected at one month resolve as development progresses, in agreement with the scRNA-seq data. Of interest, although differences in neuron type proportions resolve in this genomic context (Mito210 SUV420H1, scRNA-seq analysis, FIGS. 22B-22C), regions that were more accessible in the mutant at three months were enriched for GO terms linked to synaptic function (FIG. 14B), suggesting that some level of differential regulation of genes important to neuronal maturation and function remained.

Next, motif enrichment in the top 400 DARs was examined at each time point (31 d.i.v. and 93 d.i.v., ranked by adjusted p value). Regions with increased accessibility in the mutant were enriched for Transcription factor (TF) binding sites for regulators of neurogenesis and patterning of the developing nervous system, including EN1, DLX2, LHX8, LHX1, DLX1, GSX2, EN2, PDX1, DLX5, HOXA1 at one month, and NEUROD1, NEUROG2, HAND2, OLIG2, PTF1A, PB0193.1, TCFE2A, TCF4, BHLHA15 at three months (FIG. 23B). Several of these genes are also involved in development of the GABAergic lineage (DLX2, DLX1, DLX5, GSX2), consistent with the phenotypic abnormalities observed in GABAergic neurons in SUV420H1 mutants.

Altogether, the results show that in SUV420H1 mutant organoids, both GABAergic neurons and deep layer projection neurons undergo accelerated asynchronous development relative to isogenic control lines, in a defined temporal window during early corticogenesis. Although both phenotypes were present in multiple human pluripotent stem cell lines, the data indicates that genomic context can differentially modulate phenotypic abnormalities affecting different cell types.

### SUV420H1 Mutants Display Reduced Spontaneous Neuronal Activity

It was hypothesized that the developmental abnormalities in the GABAergic and deep layer projection neuron populations seen in the SUV420H1 mutant organoids may affect cortical local circuits composed of these neuron classes, a possibility supported by the changes in the expression and accessibility of synapse-associated genes.

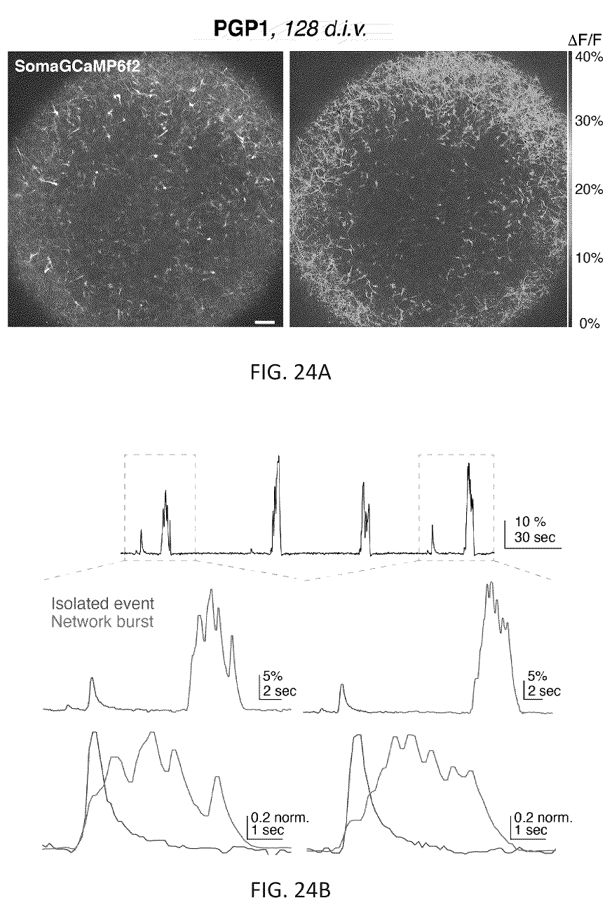

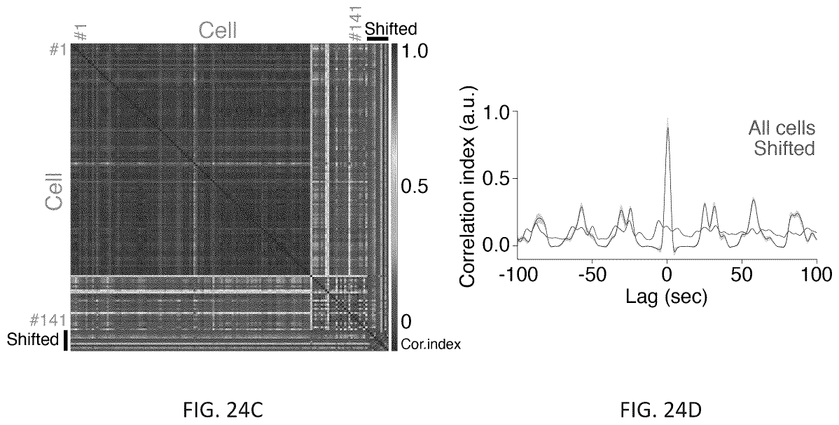

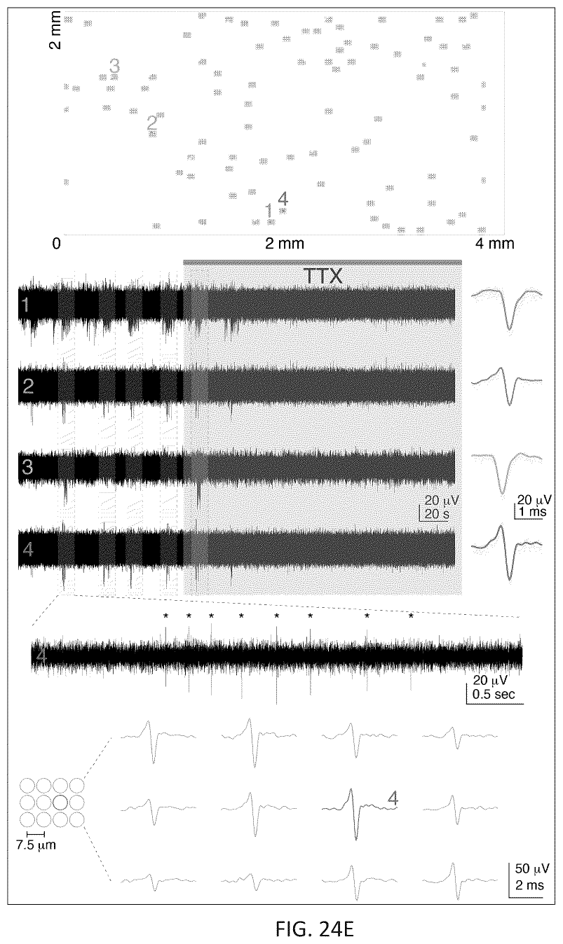

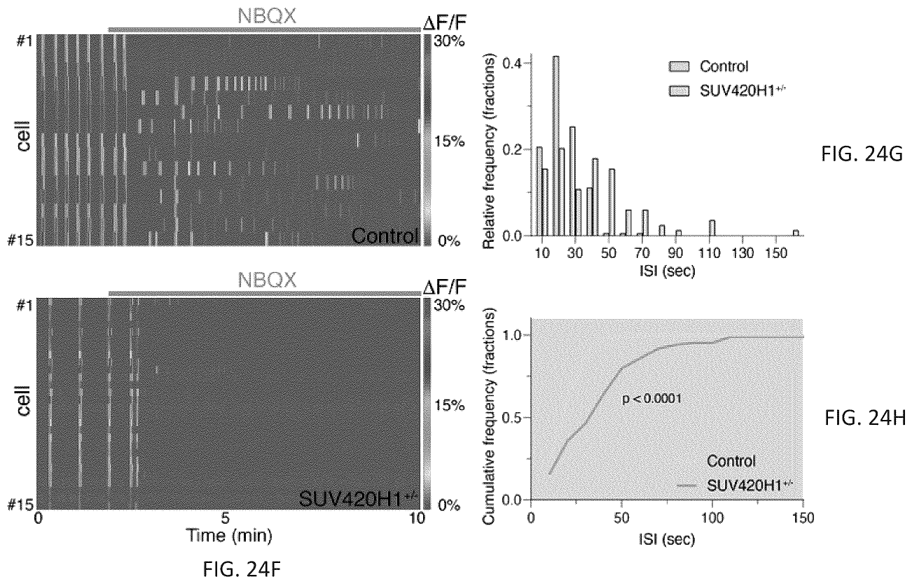

To test this hypothesis, calcium imaging analysis was performed in intact preparations of SUV420H1 control and mutant organoids. Spontaneous neuronal activity of organoids from the cell line that displayed an intermediate severity of phenotype was analyzed (PGP1 SUV420H1, FIG. 13E) by transducing control and mutant organoids with CAG::SomaGCaMP6f2³⁸ adeno-associated viruses (Methods) and performing acute recordings of intracellular calcium dynamics in intact four month organoids (128 d.i.v.) with a confocal microscope (FIG. 14D, FIG. 24A). The predominant form of activity observed was a tetrodotoxin (TTX)-sensitive calcium signal (n=10/10 organoids, FIG. 14E), whose large amplitude, slow kinetics, and multi-peak structure suggested that it was mediated by trains of action potentials (FIG. 24B). These large calcium spikes occurred in periodic, synchronized bursts across the majority of labeled neurons (FIGS. 24C-24D), resembling early network activity observed in the developing brain^{39,40}. Extracellular single-unit recordings using a multi-electrode array (MEA) confirmed that network bursting was composed of TTX-sensitive trains of action potentials across multiple neurons (FIG. 24E). Network bursting was abolished upon bath application of 20 μM NBQX (2,3-dioxo-6-nitro-1,2,3,4-tetrahydrobenzo[f]quinoxaline-7-sulfonamide), an antagonist of non-NMDA glutamate receptors (FIG. 14F), suggesting that coordinated network activity was being driven by excitatory synaptic transmission.

Notably, after blockade of excitatory synapses with NBQX, only control organoids showed residual uncorrelated activity, while mutant organoids displayed no calcium transients under the same conditions (FIG. 14F and FIG. 24F), suggesting that the cells in the control organoids were more excitable, a canonical feature of more immature neurons⁴¹. This is consistent with the accelerated differentiation in the mutant shown by the molecular data.

Moreover, comparing the frequency (p=0.032, t-test, FIG. 14G) and duration (p=0.026, t-test, FIG. 14H) of network bursts across conditions showed a relative reduction in activity in mutant organoids (FIGS. 24G-24H), indicating that haploinsufficiency of the SUV420H1 gene reduced spontaneous activity in human brain organoids. This finding is in line with data from a Suv420h1 knockdown mouse model, in which neurons from the prefrontal cortex showed reduced frequency of spontaneous excitatory postsynaptic currents⁴². Altogether, these data suggest that beyond the molecular and cellular changes observed in the SUV420H1 mutant organoids, haploinsufficiency of this gene can also induce long-term abnormalities in circuit activity.

### ARID1B and SUV420H1 Mutants Converge on Asynchronous Development of the Same Neuronal Classes

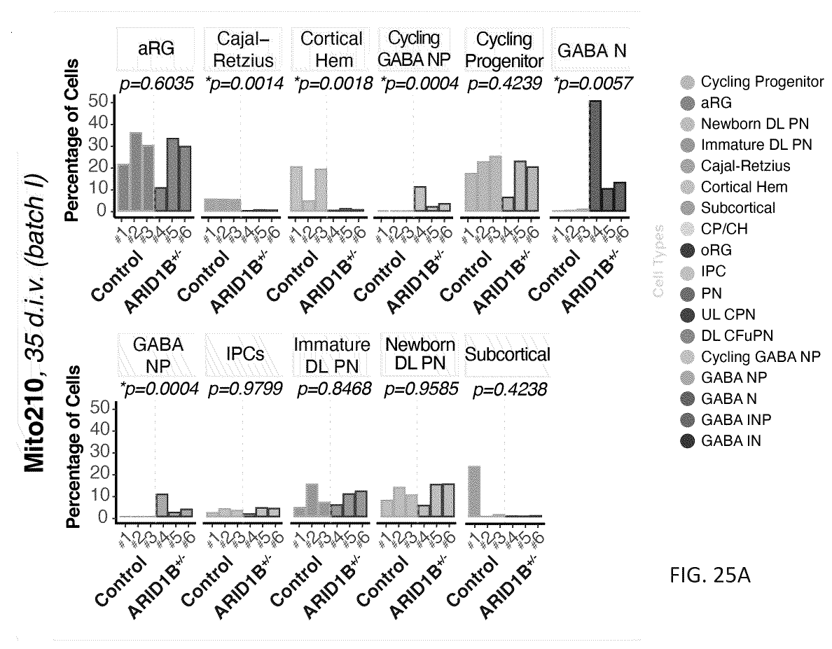

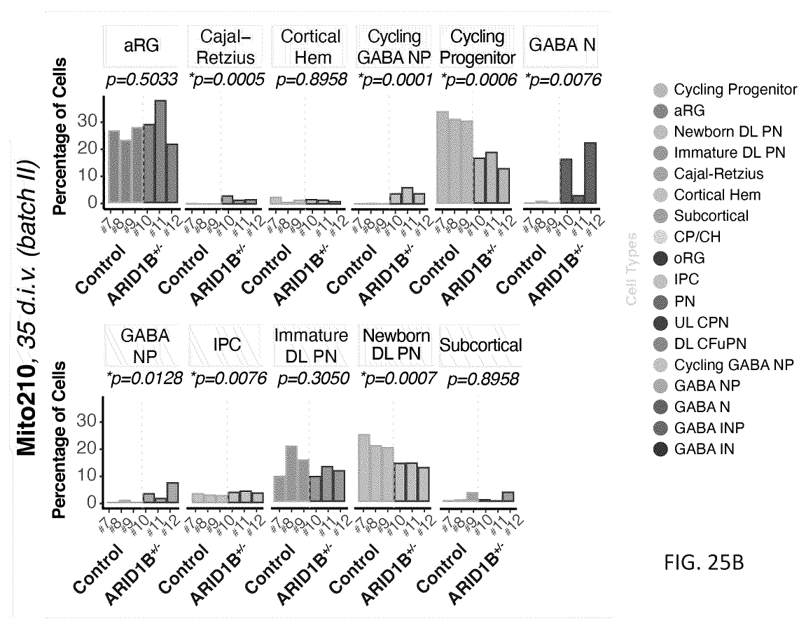

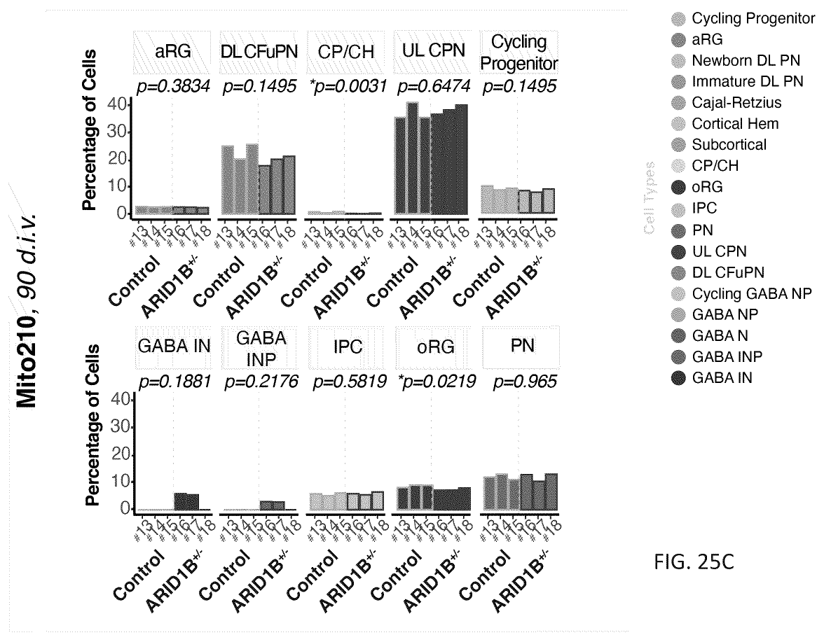

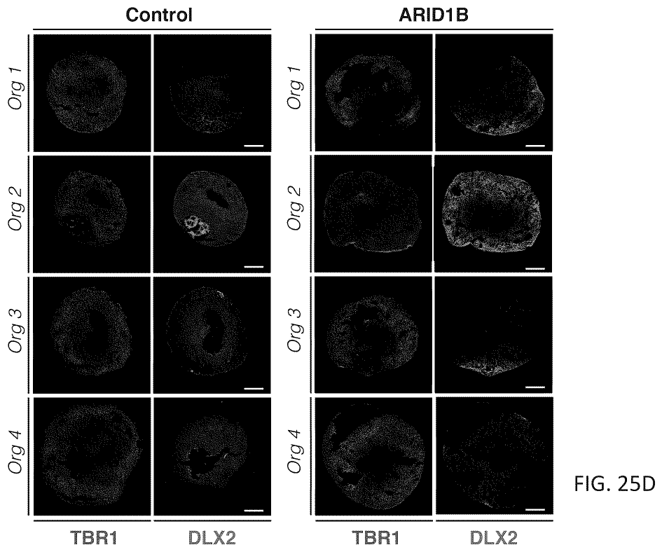

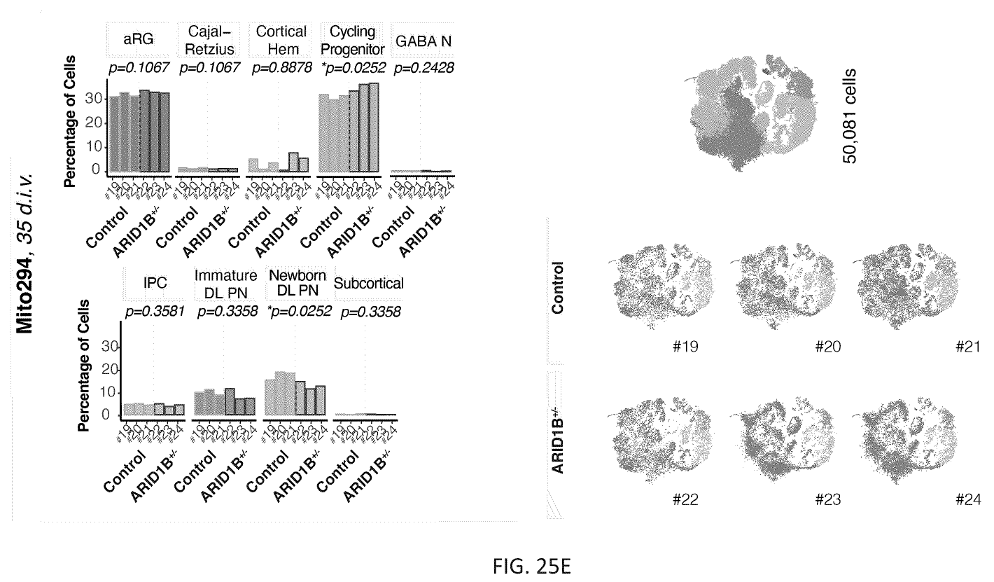

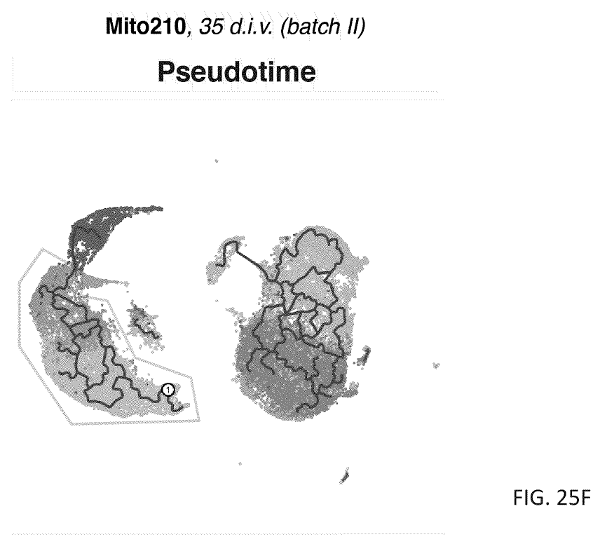

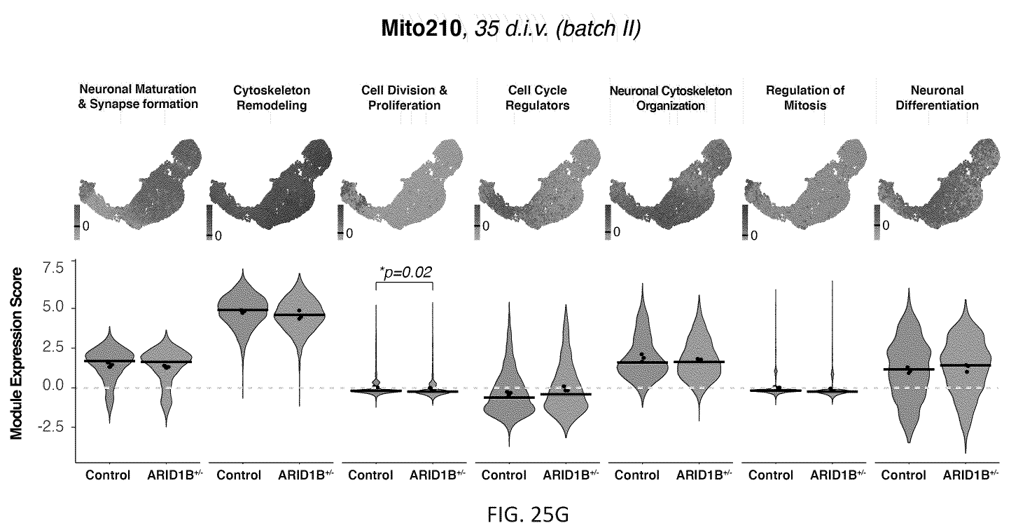

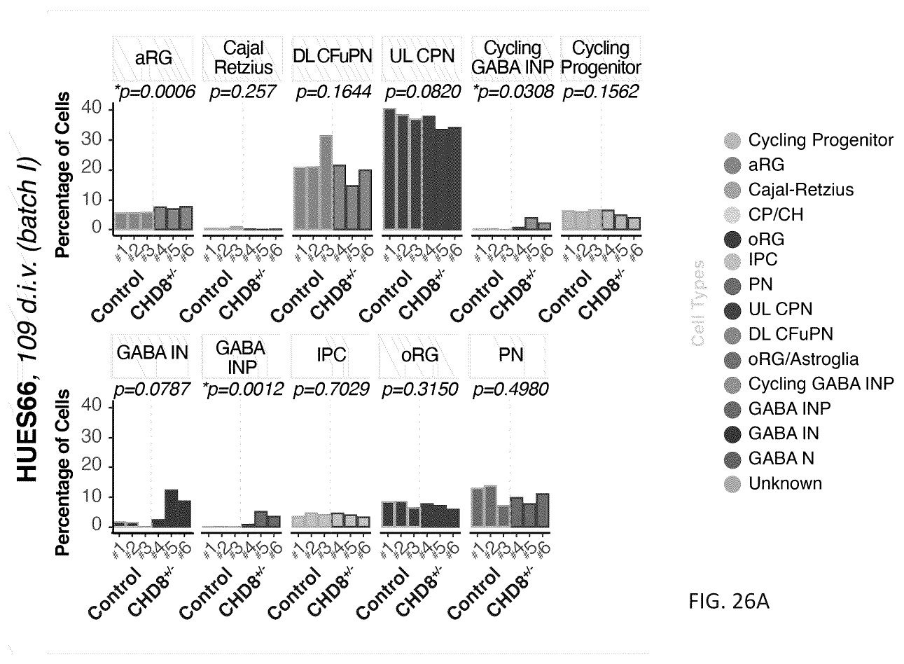

As SUV420H1 haploinsufficiency altered the rate of production of GABAergic neurons and immature deep layer projection cells, next it was investigated whether similar changes in the rate of production of neuronal classes were observed with mutations in other ASD risk genes. Individual Mito210 ARID1B mutant and isogenic control organoids were profiled at one month (35 d.i.v.) by scRNA-seq to compare changes in their cellular composition (FIGS. 3A-3C and FIGS. 25A-25B), profiling 78,556 single cells from two independent differentiation batches at 35 d.i.v. (batch I: 43,556 single cells; batch II: 35,000 single cells).

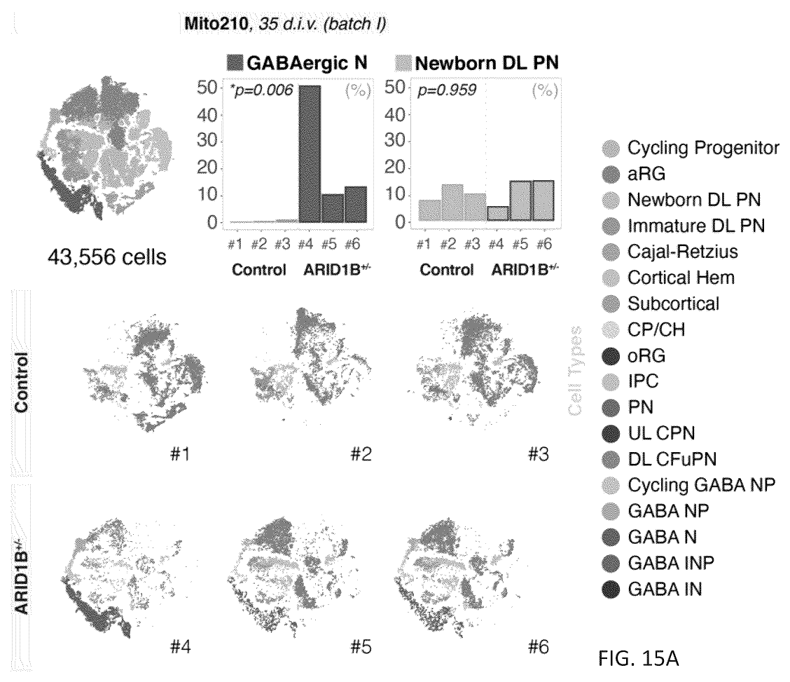

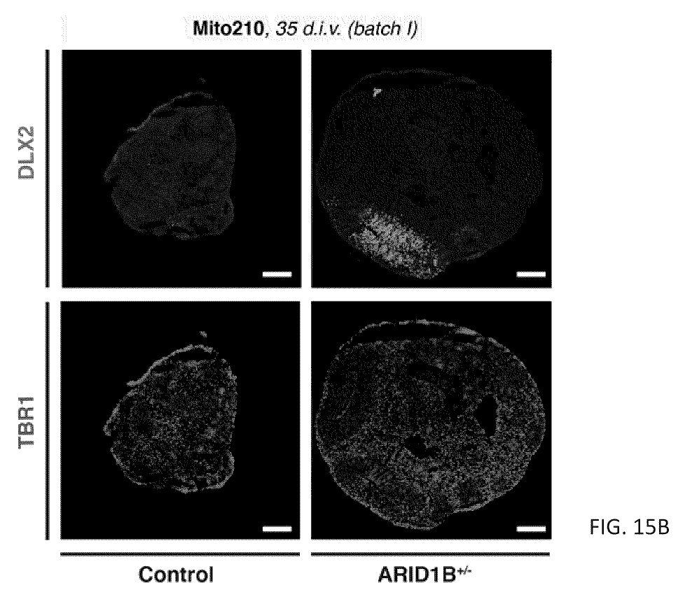

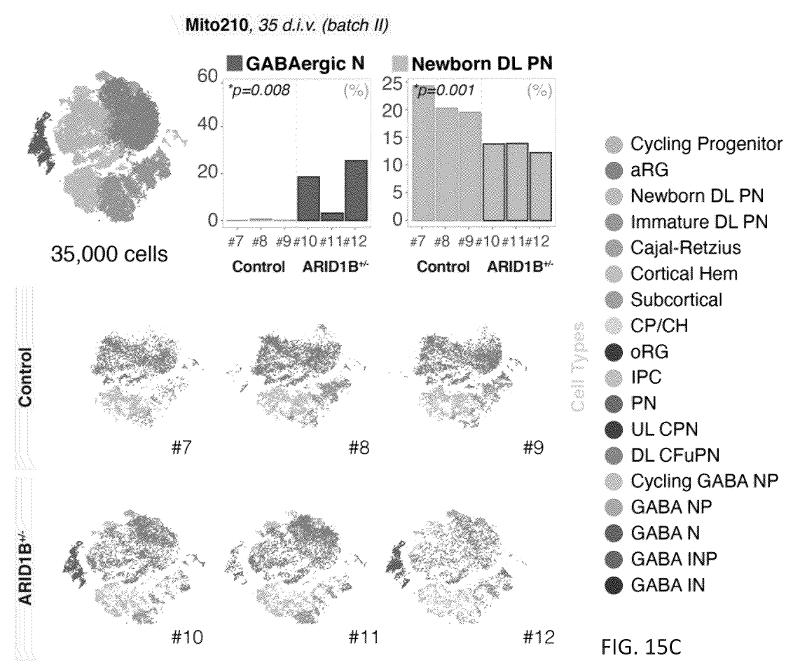

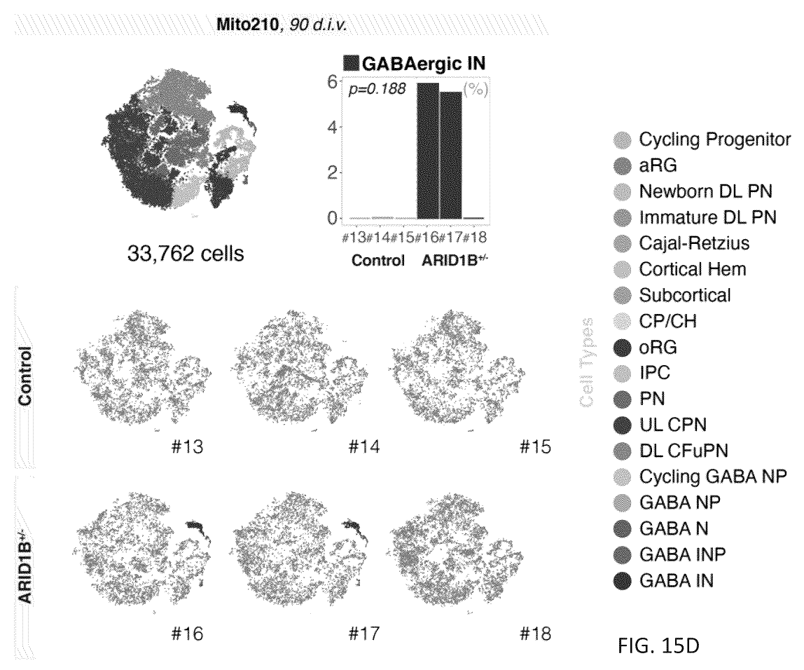

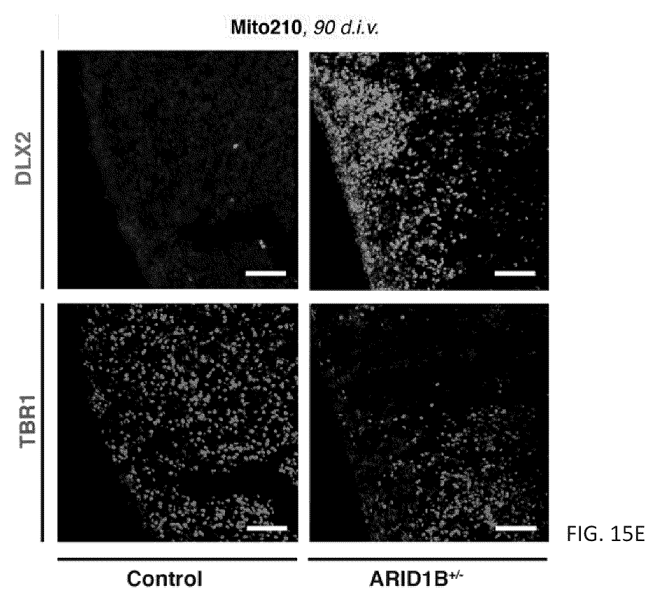

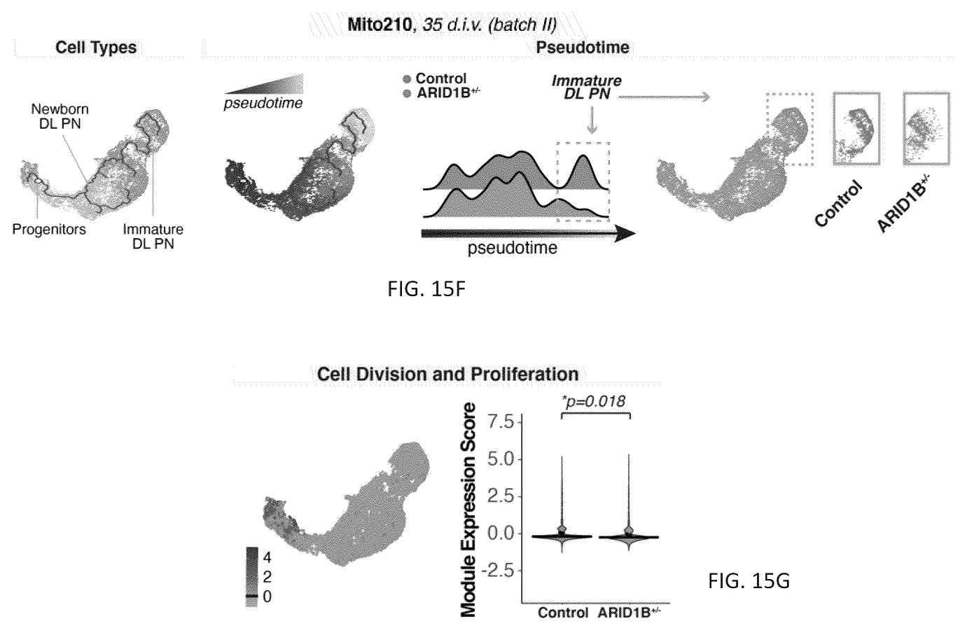

While control organoids had few or no GABAergic lineage cells at this age, the Mito210 ARID1B mutant organoids showed a consistent increase in the proportions of GABAergic neurons and their progenitors (n=3 single organoids per genotype, GABAergic neurons: adjusted p=0.0057 in batch I and adjusted p=0.0076 in batch II; GABAergic neuron progenitors: adjusted p=0.0004 in batch I and adjusted p=0.013 in batch II; cycling GABAergic neuron progenitors: p=0.0004 in batch I and p=0.0001 in batch II; logistic mixed models; FIGS. 15A-15C and FIGS. 25A-25B). In the first batch, GABAergic neurons constituted up to 50% of the profiled cells, making it difficult to draw conclusions about changes in any other cell types (FIG. 15A and FIG. 25A). In the second batch, the increase in cells of GABAergic origin was more modest, allowing for the detection of a significant reduction in the number of newborn deep layer projection neurons. These neurons were consistently decreased in every organoid analyzed from this batch (batch II: n=3 single organoids per genotype; adjusted p=0.001, logistic mixed models; FIG. 15C and FIG. 25B). These data indicate that heterozygous loss of ARID1B in the Mito210 background results in two phenotypes, affecting both the GABAergic populations and the deep layer projection neuron lineage. These are the same neuron populations whose development was affected in organoids haploinsufficient for SUV420H1. While ARID1B had an opposite effect on the deep layer projection neuron lineage, the data shows convergence of two previously unrelated risk genes in the classes of cells that they affect during cortical development.

At three months, scRNA-seq of Mito210 ARID1B mutant and control organoids (90 d.i.v., 33,762 total single cells; n=3 individual organoids per genotype) also showed an increase in GABAergic lineage cells (GABAergic interneurons) in two of three mutant organoids (vs. 0 of 3 controls; adjusted p=0.19, logistic mixed models; FIG. 15D and FIG. 25C). This was further supported by immunostaining, in which three of four mutant organoids contained DLX2-positive cells, while no DLX2 expression was detected in the four controls (FIG. 15E and FIG. 25D). Thus, while the phenotype of increased GABAergic populations was still detectable at a later developmental stage, it attenuated over time.

As was found for SUV420H1, these phenotypes were affected by the genomic context in which the mutation occurs. Control and ARID1B mutant organoids were generated in the Mito294 parental line, and 50,081 cells were profiled at 35 d.i.v. (n=3 per genotype). Consistent with the Mito210 ARID1B phenotype, there was a decreased number of newborn deep layer projection neurons in the mutant organoids compared to control (p=0.025, logistic mixed models; FIG. 25E). However, there was no significant increase in the GABAergic population in this background (p=0.25, logistic mixed models; FIG. 25E). This line, Mito294, had the most pronounced increase in GABAergic neurons after SUV420H1 mutation, showing that distinct aspects of the mutant phenotype may be differentially modulated by the genomic context in which the mutation acts, and that the genomic context modifies the expressivity of each risk gene mutation differently.

Pseudotime analysis of the developmental trajectory corresponding to the deep layer projection neuron lineage (the neurons altered in both genomic contexts) supported a model of delayed differentiation (FIG. 15F and FIG. 25F). In particular, examining the component of cells progressing from progenitors to deep layer projection neurons from Mito210 ARID1B organoids at one month (35 d.i.v., batch II) showed a decreased distribution of cells toward the endpoint of this trajectory (p<2.2×10⁻¹⁶, one-sided Kolmogorov-Smirnov test, FIG. 15F), supporting a phenotype of delayed neuronal differentiation affecting deep layer projection neurons. Of the seven gene modules co-varying across both mutant and control (FIG. 25G), the module “Cell Division and Proliferation” was enriched in progenitor cells and contained multiple genes involved in DNA replication and the cell cycle (e.g., CENPU, MCM3, MCM5, E2F1). This module was significantly upregulated in mutant organoids (adjusted p=0.018, linear mixed models, FIG. 15G), consistent with delayed differentiation of deep layer projection neurons in ARID1B mutant organoids.

In sum, like the SUV420H1 mutants, ARID1B mutant organoids exhibit both a phenotype of premature expansion of the GABAergic neuron lineage, and asynchronous development of the deep layer projection neuron lineage. Notably, as in the SUV420H1 mutants, these two phenotypes were differentially modulated in distinct parental lines in the ARID1B mutant organoids, further supporting the finding that genomic context modulates the expressivity of developmental defects in a phenotype-specific manner.

### CHD8 Haploinsufficiency Leads to Asynchronous Generation of GABAergic Interneurons in a Background-Dependent Manner

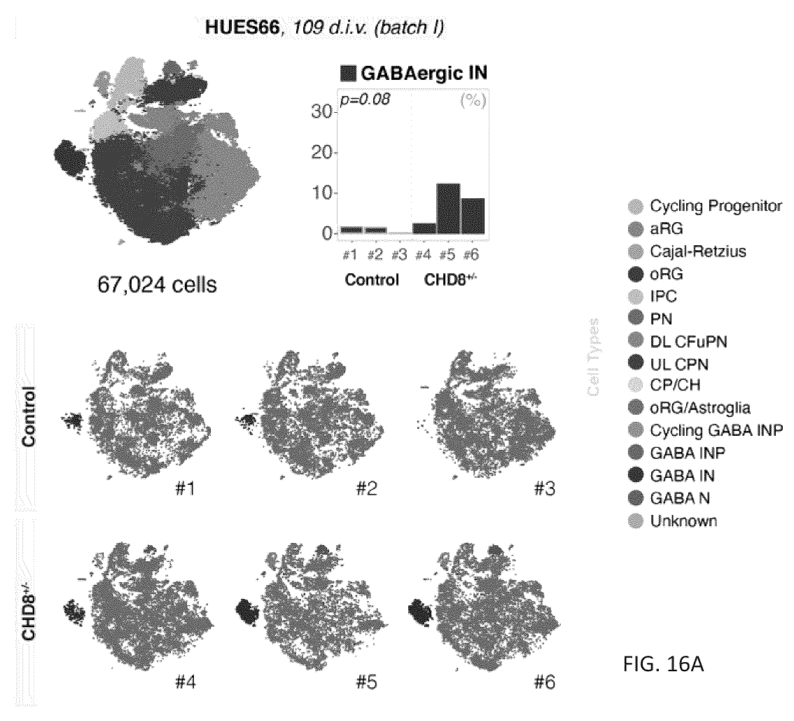

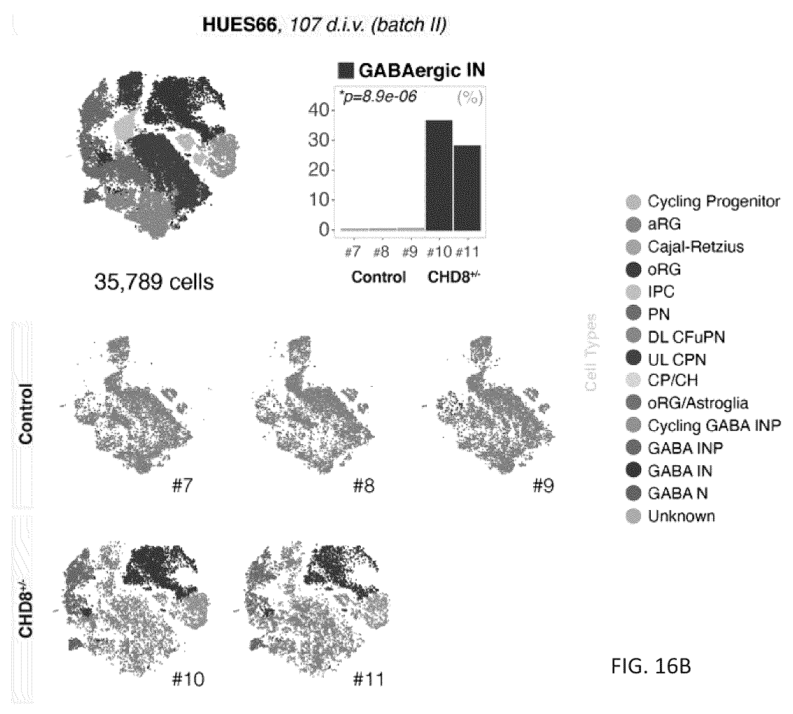

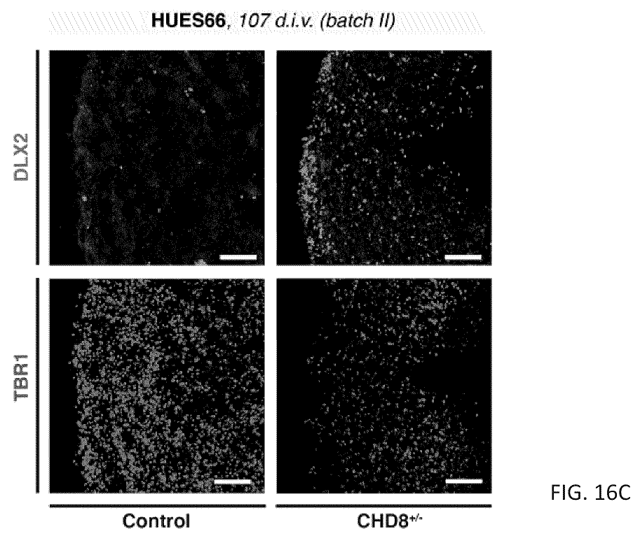

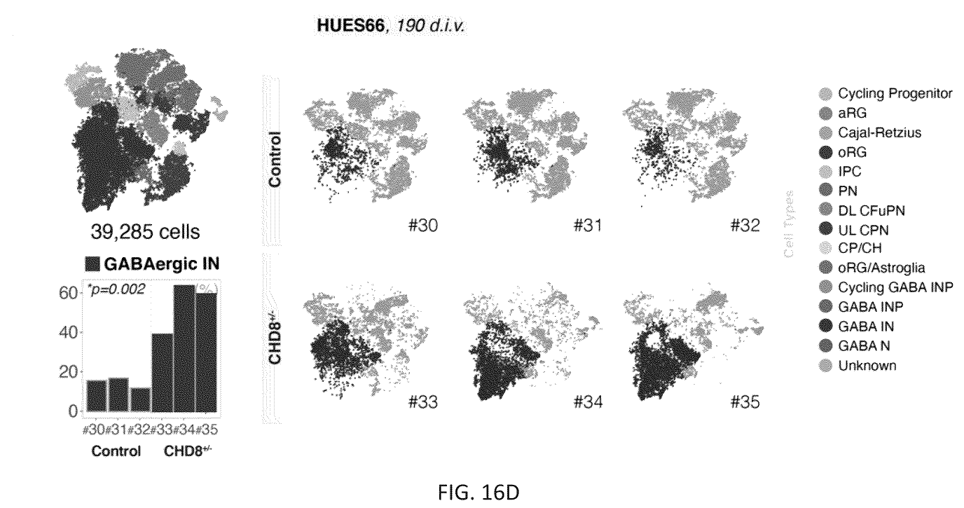

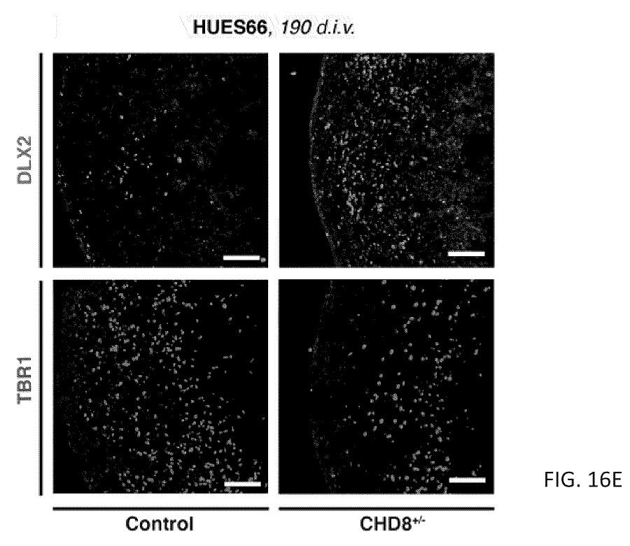

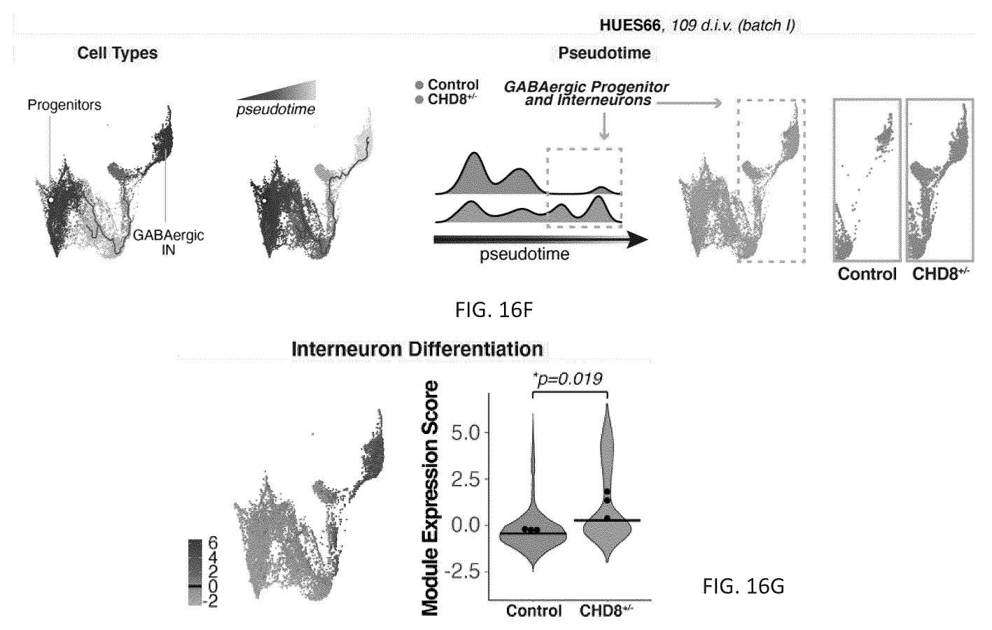

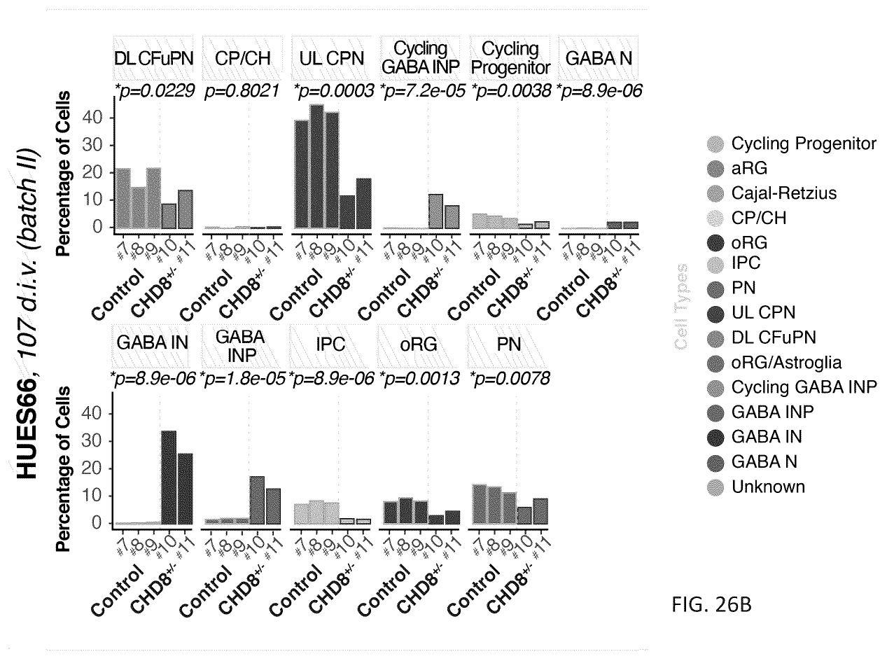

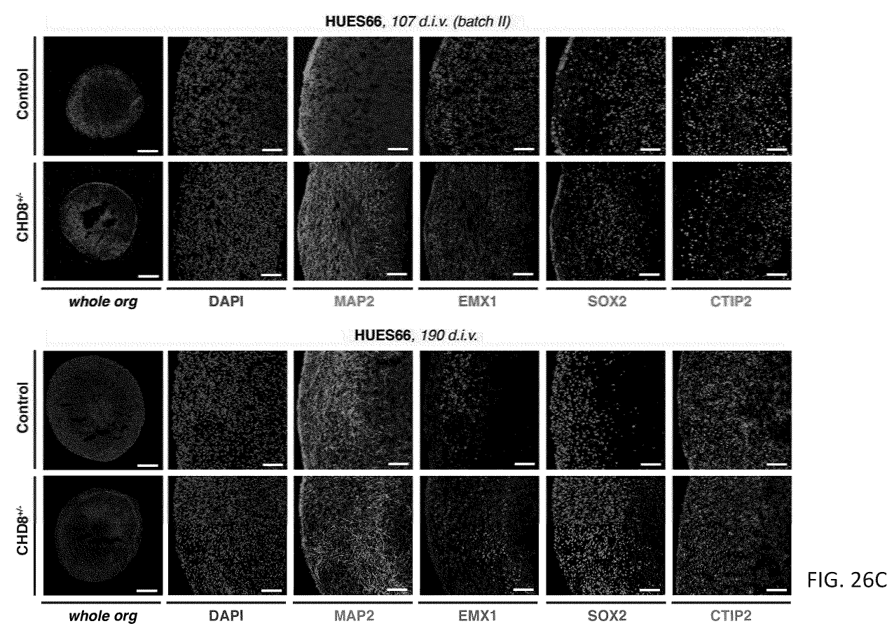

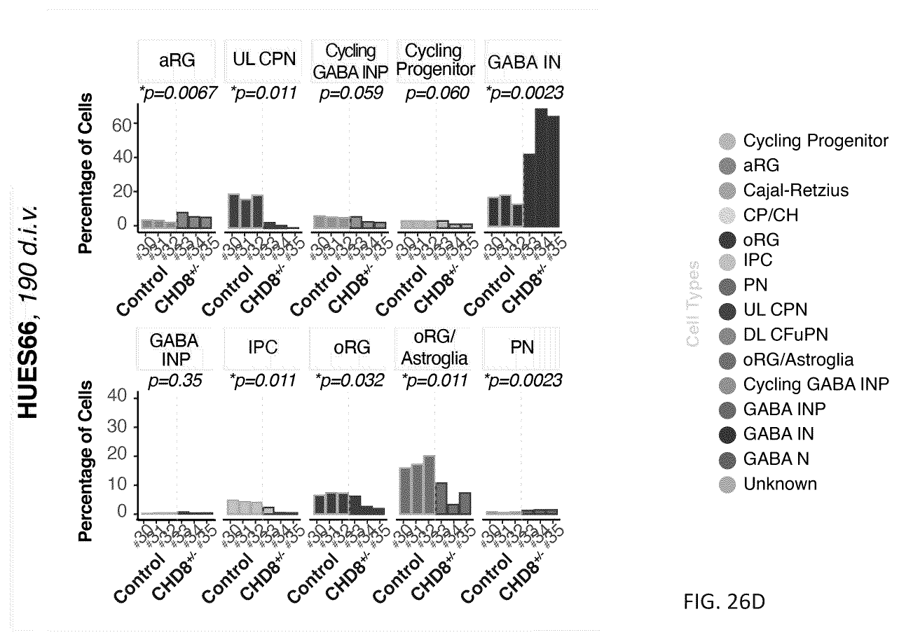

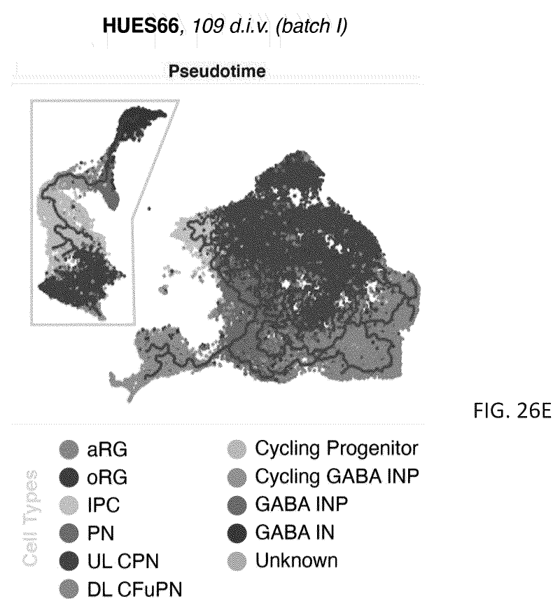

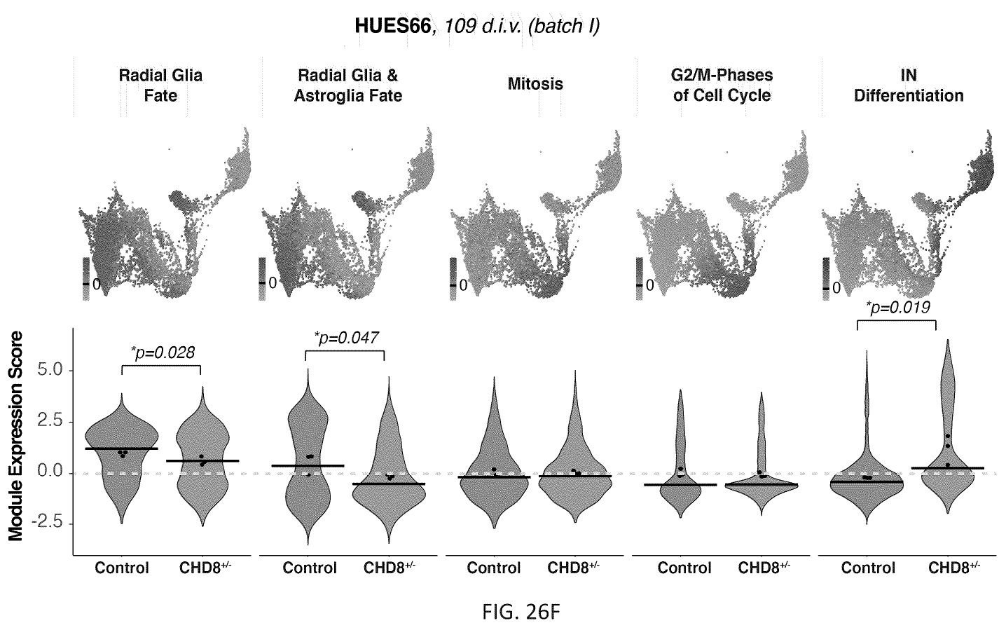

To further explore the hypothesis of convergent phenotypes among ASD risk genes, mutant and control HUES66 CHD8 organoids were profiled at 3.5 months (109 d.i.v., 67,024 single cells, n=3 single organoids per genotype; FIG. 16A and FIG. 26A). An increased number of GABAergic interneurons and their progenitors were found in the HUES66 CHD8 mutant organoids (GABAergic interneurons: adjusted p =0.079, cycling GABAergic interneuron progenitors: adjusted p=0.031, GABAergic interneuron progenitors: adjusted p=0.0012, logistic mixed models; FIG. 16A and FIG. 26A). A second independent batch of HUES66 CHD8 organoids showed an even more dramatic increase in the numbers of GABAergic interneurons and their progenitors (n=2-3 single organoids per genotype; cycling GABAergic interneuron progenitors: adjusted p=7.2×10⁻⁵, GABAergic interneuron progenitors: adjusted p=1.8×10⁻⁵, GABAergic interneurons: adjusted p=8.9×10⁻⁶, logistic mixed models; FIGS. 16B-16C and FIGS. 26B-26C). The increase in GABAergic interneurons in this batch was so pronounced that the represented proportion of all other cell types were reduced in the mutant organoids (FIG. 26B).

Notably, scRNA-seq on the HUES66 CHD8 organoids after six months in culture showed that the phenotypic abnormalities induced by CHD8 haploinsufficiency persisted (190 d.i.v., n=3 individual organoids per genotype, 39,285 cells), with a consistent increase in GABAergic interneurons in mutant vs. control organoids (adjusted p=0.002, logistic mixed models; FIGS. 16D-16E and FIGS. 26C-26D).

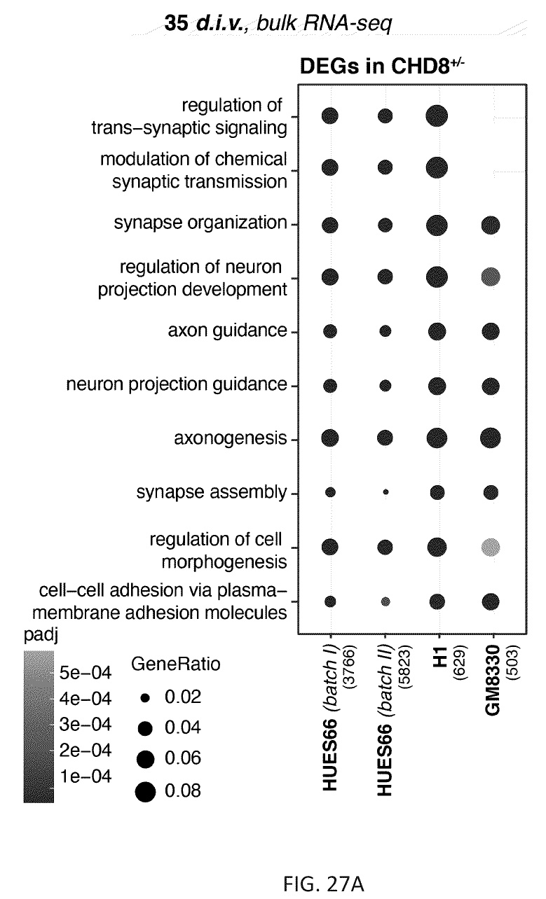

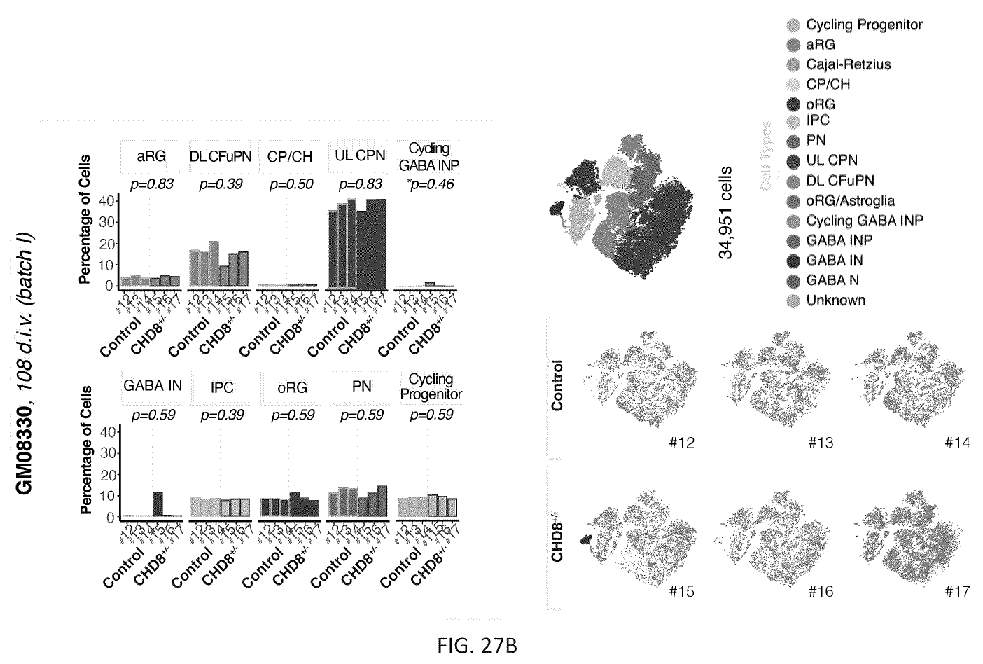

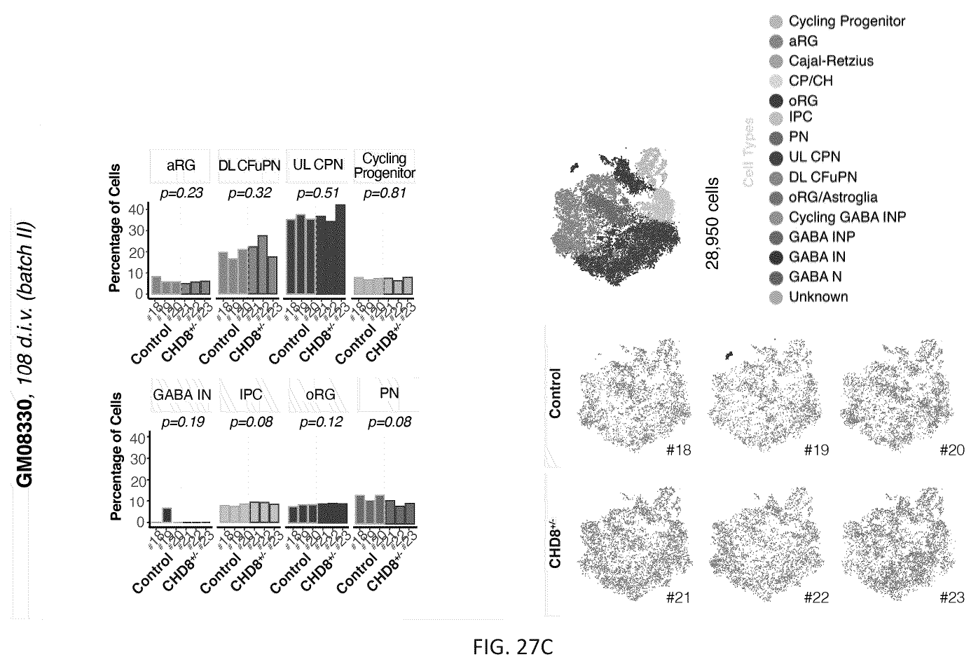

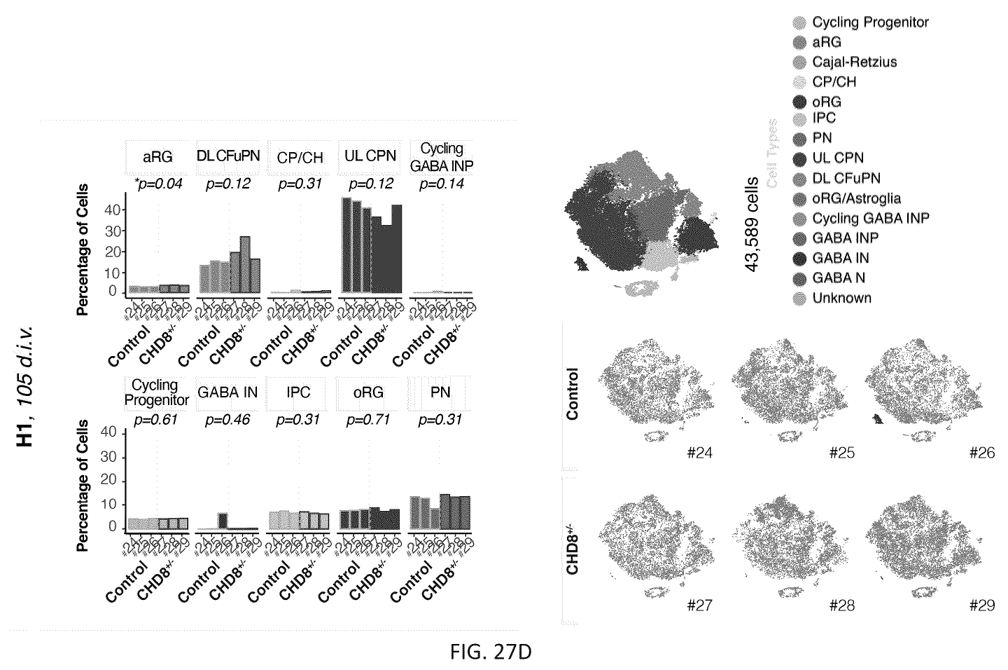

This increase in GABAergic populations is consistent with two recent reports that CHD8 haploinsufficiency affects expression of GABAergic interneuron marker genes, using in vitro models derived from two additional human genomic contexts, a control iPSC line and a hESC line, engineered to carry mutations in CHD8^{43,44}. As both SUV420H1 and ARID1B had shown significant variation in expressivity between lines, it was sought to determine if there were likewise genomic contexts that could modulate the phenotypic manifestation of CHD8. Therefore, mutant and control CHD8 organoids generated from four different parental lines spanning different basal levels of CHD8 protein expression were compared (FIGS. 20C, 20F), all organoids were engineered to carry mutations in the same gene region (FIG. 29). Initial assessment by bulk RNA-seq of 35 d.i.v. organoids showed that while differentially expressed genes (DEG) between mutant and control did not significantly overlap between lines, DEGs from three of the four lines (HUES66, GM08330, and H1) were enriched in overlapping GO terms, related to neurodevelopment and neuronal maturation (FIG. 27A). However, scRNA-seq on CHD8 mutant and control organoids from GM08330 and H1 at 3.5 months (105-108 d.i.v., n=3 individual organoids per genotype, 107,490 cells) showed no significant difference between mutant and control, such that neither had a large population of GABAergic interneurons (FIGS. 27B-27D). Thus, as for ARID1B and SUV420H1 mutants, the GABAergic phenotype did not manifest with the same severity in all backgrounds, suggesting that there are genomic contexts capable of buffering these developmental abnormalities.

Trajectory analysis on the 3.5 months HUES66 CHD8 control and mutant organoids supported a model where the increase in GABAergic interneurons is due to accelerated differentiation of GABAergic neurons in the mutant. The partition containing the GABAergic lineage was extracted, progressing from radial glia to GABAergic interneurons (FIG. 16F and FIG. 26E). HUES66 CHD8 mutant organoids showed an increased distribution of cells toward the end point of the developmental trajectory, i.e., at a more advanced stage of development, compared to controls (p<2.2*10⁻¹⁶, one-sided Kolmogorov-Smirnov test, FIG. 16F). Combined with the increased number of neurons in the mutant organoids, these results further support a phenotype of accelerated differentiation of GABAergic interneurons in this mutant. WGCNA analysis identified five co-varying gene modules across the cells in the GABAergic lineage (FIG. 26F), which included a module of interneuron differentiation genes (e.g., DLX1, DLX2, DLX6-AS1, DLX5, SCGN, and GAD2) that was upregulated in the HUES66 CHD8 mutant (adjusted p=0.019, linear mixed models, FIG. 16G), and two modules related to progenitor biology that were downregulated in the mutant (FIG. 26F).

Thus, similarly to SUV420H1 and ARID1B mutants, CHD8 haploinsufficiency leads to accelerated development of the GABAergic lineage, which in the case of CHD8 leads to a persistent increase in the proportion of these cell types through cortical development. For all three of these risk genes, this phenotype can be observed in multiple parental lines, but, notably, different donor lines vary in their degree of phenotypic expressivity.

### SUV420H1, ARID1B, and CHD8 Haploinsufficiency Affect Similar Developmental Processes Through Distinct Gene and Protein Targets

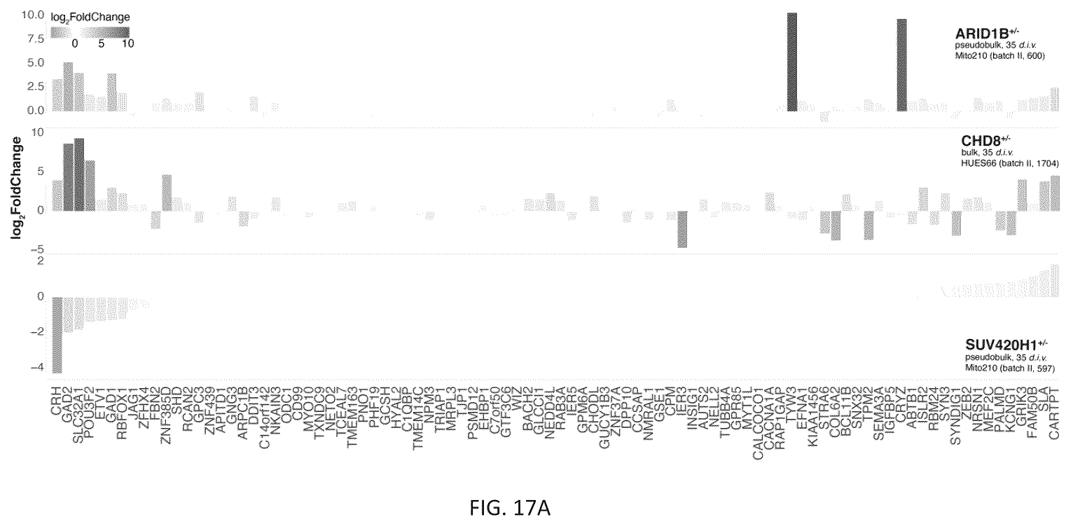

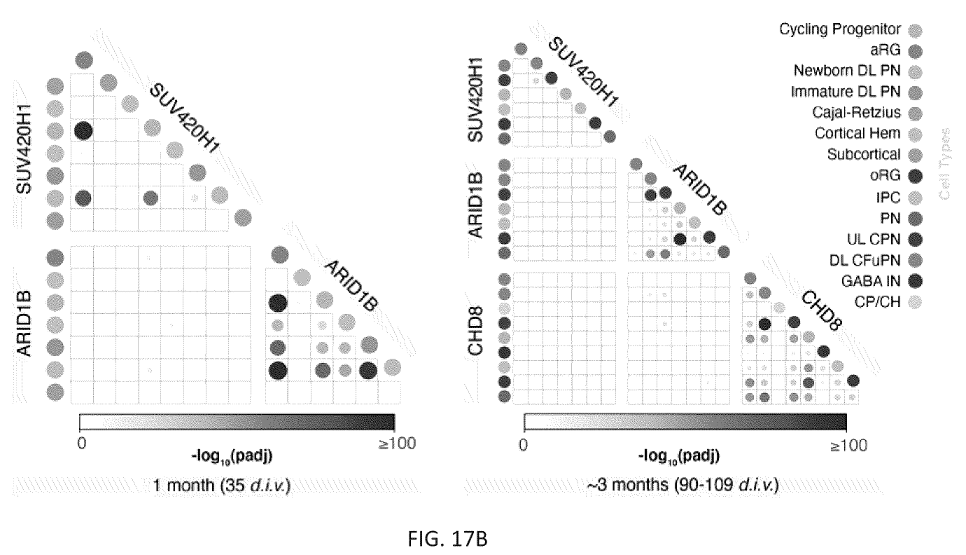

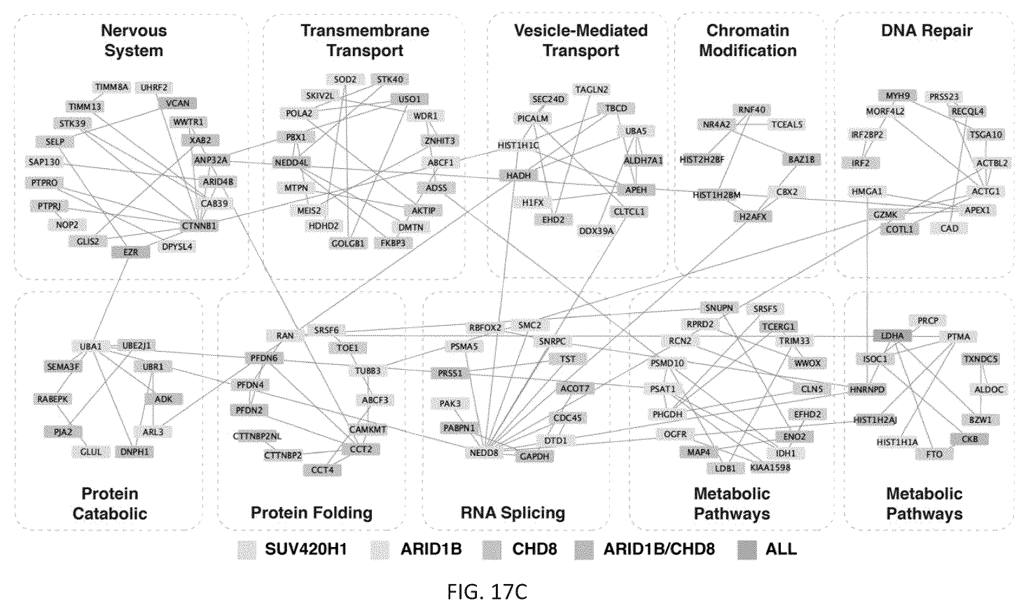

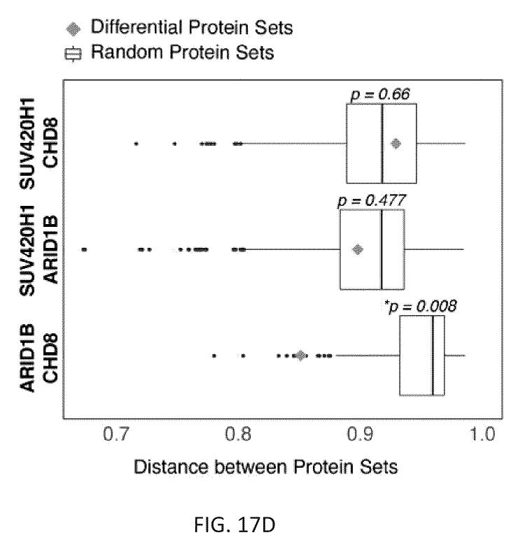

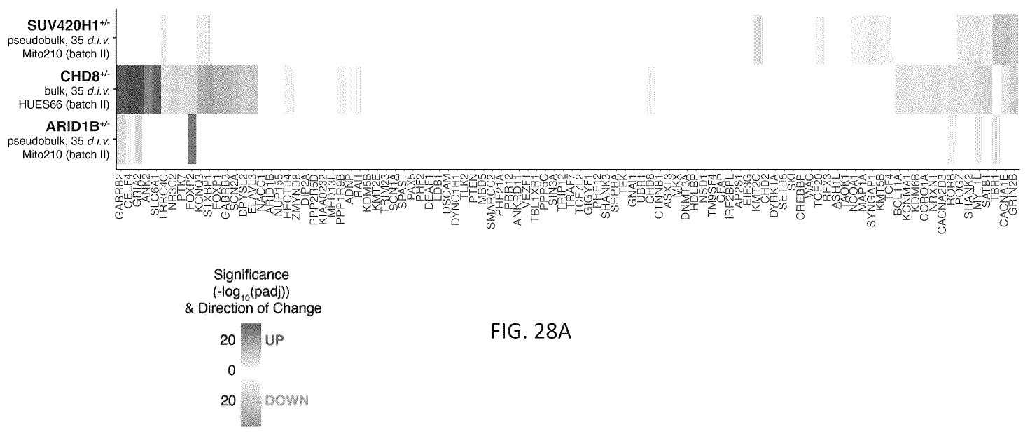

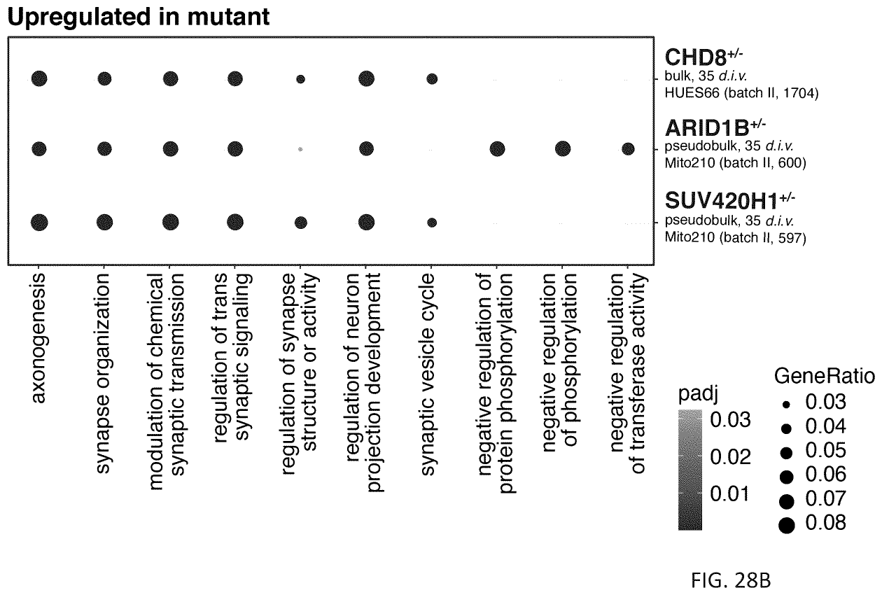

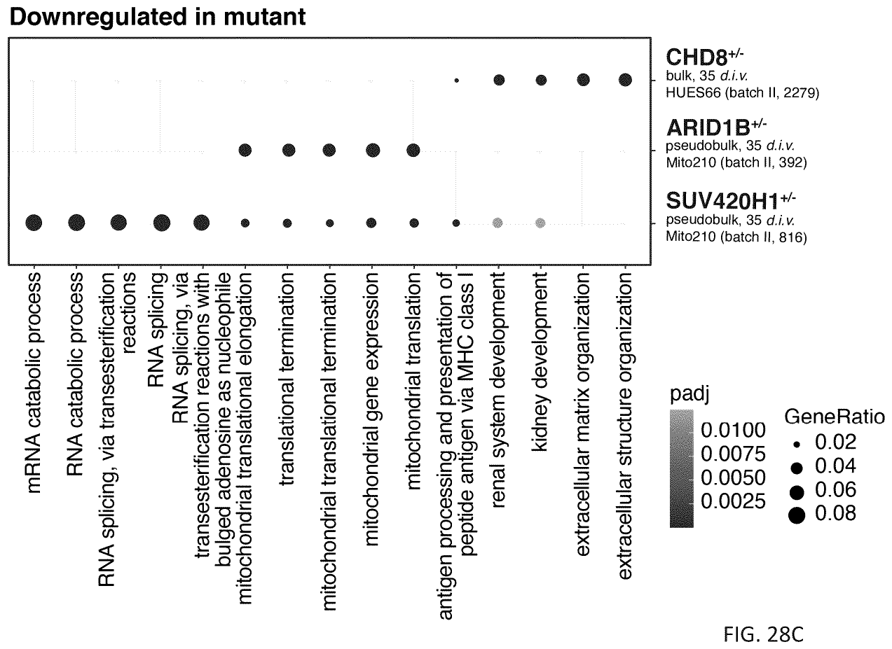

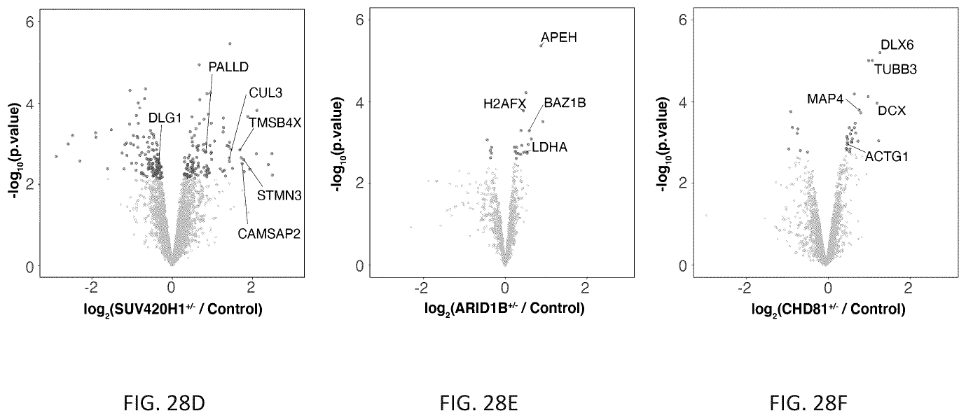

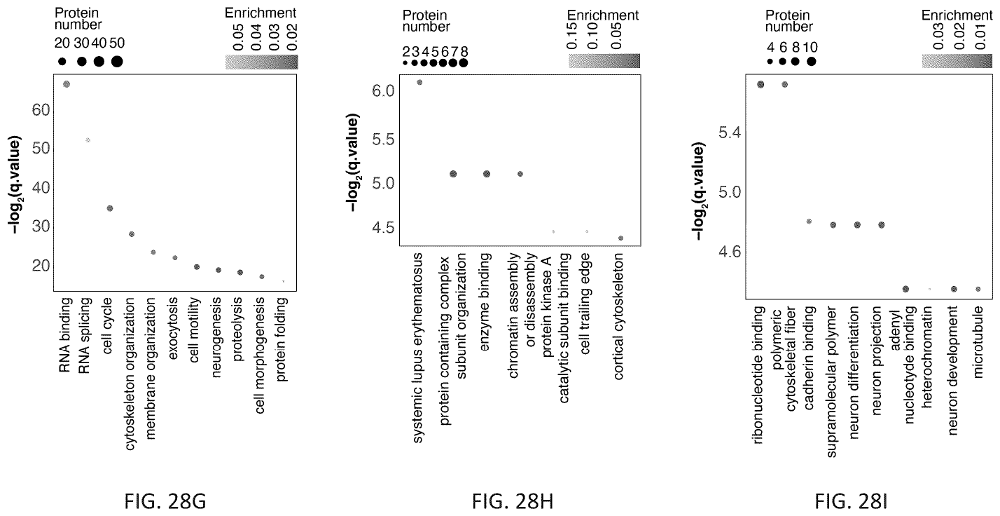

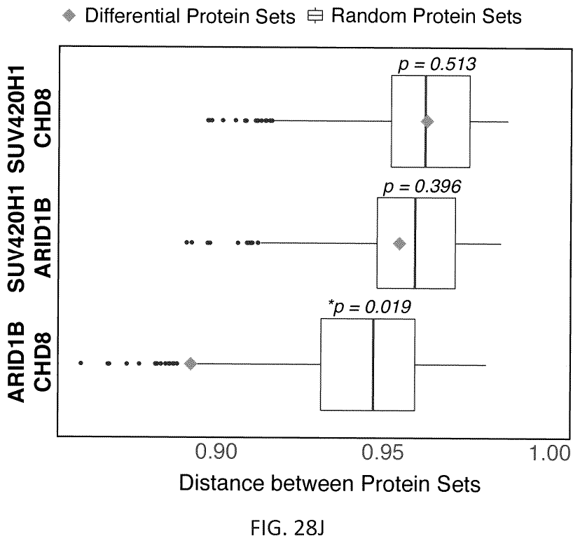

The three ASD risk genes investigated, SUV420H1, ARID1B, and CHD8, may converge on asynchronous development of cortical neuronal lineages by acting through common molecular pathways, or by using distinct mechanisms. To investigate these possibilities, gene expression changes across the three ASD risk genes was compared. Cell lines that showed a strong phenotype (Mito210 SUV420H1, Mito210 ARID1B, and HUES66 CHD8) at one timepoint (one month, 35 d.i.v.) were focused on. First, DESeq2⁴⁵ (using a model which explicitly controls for sample-to-sample variation⁴⁶) was applied to calculate DEGs between mutant and control in each of the following datasets: pseudo-bulk from Mito210 SUV420H1 (batch II), pseudo-bulk from Mito210 ARID1B (batch II), and bulk RNA-seq from HUES66 CHD8 (batch I and II combined), all collected at 35 d.i.v. 1,689 DEGs in SUV420H1, 1,241 DEGs in ARID1B, and 4,864 DEGs in CHD8 were found; of these, only 92 were shared in all three mutants, 41 of which were changed in the same direction for all mutations (FIG. 17A). Of these 41, 26 genes were upregulated, including genes related to neuronal subtype specification and maturation, such as SLA, GRIK3, MEF2C, ZEB2, SEMA3A, BCL11B, CACNA1A, and RAB3A. The DEGs also included several ASD risk genes (FIG. 28A, overlap of 102 ASD risk genes with the DEG sets, p=0.00054 for SUV420H1, p=0.23 for ARID1B, and p=3.5×10⁻⁵ for CHD8, hypergeometric test), indicating that SUV420H1 and CHD8 influence the expression of other genes that affect ASD risk. Despite the low degree of overlap in DEGs, the upregulated genes in each shared enrichment for multiple terms related to neuron development and synaptic structure and function (FIG. 28B); such an overlap was not observed for the downregulated genes (FIG. 28C).

The single-cell data was leveraged to compare cell-type-specific changes in expression in each mutant. DEGs were calculated between mutant and control for each cell type present at one or three months in each dataset, and the overlap was quantified between each of these gene sets within matching time points (SUV420H1 Mito210 35 d.i.v. versus ARID1B Mito210 35 d.i.v. batch II, and SUV420H1 Mito210 92 d.i.v. versus ARID1B Mito210 90 d.i.v. versus CHD8 HUES66 109 d.i.v.). While similar cell types within the same mutation often exhibited highly-overlapping DEG sets, DEGs caused by different mutations rarely overlapped, even for identical or closely-related cell types (FIG. 17B). Thus, although these three mutants share a degree of convergence in altered neurodevelopmental processes, they affect largely distinct genes.

Similar findings were obtained at the level of protein expression, as assessed by whole-proteome mass spectrometry of mutant and control single organoids. Isobaric tandem mass tag (TMT) multiplexed quantitative mass spectroscopy was performed on Mito210 SUV420H1 (n=4 single organoids per genotype), Mito210 ARID1B (n=4-5 single organoids per genotype), and HUES66 CHD8 (n=3 single organoids per genotype) organoids collected at one month (35 d.i.v.). 233 proteins were identified that were significantly differentially expressed for SUV420H1 (≥4,000 proteins detected per sample, FDR <0.1, FIG. 28D), 24 proteins for ARID1B (≥900 proteins detected per sample, FDR <0.1 FIG. 28E), and 34 proteins for CHD8 (≥2,800 proteins detected per sample, FDR <0.1, FIG. 28F). As with differential gene expression, there was very low overlap of differentially expressed proteins (DEPs) between mutations, with only five proteins significantly dysregulated in at least two mutations. DEPs and enriched biological processes (by gene set enrichment) for all mutations resembled the gene modules previously identified by scRNA-seq (FIGS. 28G-28I). SUV420H1 and CHD8 DEPs were enriched for neuronal differentiation, consistent with their accelerated neuronal development. DEPs between SUV420H1 mutant and control organoids included proteins associated with neuronal processes such as axon guidance (e.g., PALLD, TMSB4X, CAMSAP2), synaptogenesis (e.g., DLG1, CUL3), and neuronal development (STMN3) (FIG. 28D), further supporting an effect of SUV420H1 haploinsufficiency on neuronal maturation. The most differentially upregulated proteins in CHD8 mutant organoids included proteins associated with neuronal differentiation and cytoskeleton remodeling (e.g., TUBB3, DCX, MAP4, ACTG1), as well as the GABAergic neuron fate regulator DLX6⁴⁷ (FIG. 28F), suggesting an earlier commitment of mutant progenitors to a GABAergic neuron fate. By contrast, in the ARID1B mutant organoids, DEPs included proteins such as H2AX and BAZ1B, known to play a critical role in controlling repair of DNA damage and cell cycle regulation^{48,49} (FIG. 28E), consistent with the differential gene module highlighted in the scRNA-seq data for this gene (FIG. 15G).

It was then sought to evaluate whether the affected proteins in the three mutants were predicted to interact with one another, or with shared target proteins. The top 50 DEPs (ranked by adjusted p value) for each of the three mutations were used as input to create a network of interacting proteins^{50,51}, followed by clustering to identify subnetworks (Methods). These subnetworks were enriched for terms including “nervous system”, “vesicle-mediated transport”, “chromatin modification” and “metabolism” (adjusted p≤0.1 in all cases; FIG. 17C). Each subnetwork contained DEPs from multiple mutations, indicating that all three risk genes alter proteins involved in shared processes, albeit by dysregulating different proteins. Finally, it was sought to assess how closely related the dysregulated proteins were across mutations, by calculating a weighted distance between the three sets of DEPs, based on known protein interactions⁵². Only the protein sets for ARID1B vs. CHD8 were closer than expected by chance (p=0.008, FIG. 17D and FIG. 28J). This indicates that, at this stage of development, CHD8 and ARID1B mutant organoids may act through related protein networks, but via the dysregulation of distinct sets of proteins.

Altogether, the results suggest that despite the widespread expression and broad functions of these three risk genes, SUV420H1, ARID1B, and CHD8, all cause phenotypes of asynchronous development of specific human cortical neuronal lineages (GABAergic cells and deep layer projection neurons), but do not affect the same genes and proteins. Moreover, these phenotypic abnormalities can be modified by the genomic context in which each mutation acts, affecting expressivity in a gene- and phenotype-specific manner.

### Discussion

The process by which mutations in ASD risk genes converge on the neurobiology of this multifaceted disorder remains unclear. Progress in this field has been difficult because of the complexity of ASD genetics, limited experimental access to the human brain, and the lack of experimental models for human brain development¹¹. By leveraging reproducible human cortical organoids, proteomics, and single cell genomics (scATAC-seq and scRNA-seq), it was found that mutations in three ASD risk genes, SUV420H1, ARID1B, and CHD8 converge on a shared phenotype of asynchronous cortical neurogenesis, and define two main neuronal classes of the local cortical circuit (GABAergic neurons and deep layer projection neurons) as the specific cell populations affected. This is intriguing, as prior studies of postmortem ASD brains⁵³ and human PSC lines derived from idiopathic ASD patients⁵⁴⁻⁵⁶ have pointed at dysregulation of GABAergic neuron development. In addition, studies in mouse models and human neural cultures have linked several ASD risk genes to abnormalities in neuronal development,^{31,57} including upregulation of GABAergic neuron marker genes in CHD8 mutant cultures⁴⁴. The new findings indicate that despite widespread expression of ASD risk genes in multiple cell types and developmental stages, their resulting developmental abnormalities are specific for selected populations of cells. Identification of GABAergic neurons and deep layer projection neurons as the cells affected by mutation in these genes now provides a focus for investigation of disease mechanisms affecting these exact cell types. Notably, the development of these neurons is affected through largely non-overlapping molecular targets in each mutation. This encourages future mechanistic investigation focused on shared developmental processes rather than shared molecular pathways, a hypothesis that can now be tested across many more ASD mutations.

It is shown that cell lines from different donors affect the same cell types, but differ in the severity of the individual phenotypes. This is consistent with the known phenotypic variability associated with ASD risk gene mutations in human populations^{7,8,13,19} and highlights the importance of studying risk genes across multiple human genomic contexts. Notably, the data indicates that different genomic contexts differentially modulate expressivity based on both the risk gene mutated and the nature of the specific phenotype caused by each mutation. For example, the Mito294 cell line shows a pronounced phenotype in the SUV420H1 mutation, but is resilient to the same phenotype caused by mutation in ARID1B. Furthermore, the effects of the broader genetic context are highly nuanced with relation to the phenotype; in SUV420H1 mutant, it was found that although the Mito210 line showed a more severe phenotype for the deep layer projection neurons than the PGP1 line, the latter showed a more severe phenotype for the GABAergic neurons. One possible mechanism underlying this variability may be idiosyncratic differences in the baseline expression and function of each risk gene between individuals (FIGS. 20D-20F), as has been noted in humans and other animals^{7,8}. However much remains to be studied on how genomic context modulates the phenotypic manifestation of disease.

The finding that different ASD risk genes converge on a phenotype of asynchronous neuronal development but mostly diverge one level of complexity below it (i.e., at the level of molecular targets), suggests that the shared clinical pathology of these genes may derive from high-order processes of neuronal differentiation and circuit wiring, as opposed to convergent use of the same molecular effectors. These results suggest the value of investigating therapeutic approaches aimed at the modulation of shared dysfunctional circuit properties in addition to shared molecular pathways.

Excitatory/inhibitory imbalance of the cortical microcircuit is a major hypothesis for the etiology of ASD⁵⁸⁻⁶⁰. Such an imbalance could result from a change in the numerical proportions of cell types within the circuit, incorrect wiring, and/or altered properties of the neurons and glial cells involved, among others. The altered overall proportion of neurons that were observed in mutant organoids points to an early defect in circuit development whereby a small number of neuron classes develop asynchronously with the remaining cell types of the circuit. Studies in vivo suggest that these types of relatively subtle developmental alterations, even if resolved later in development, could result in disproportionally large functional effects on the mature circuit⁶¹⁻⁶³. This hypothesis is supported by the observation of changes in spontaneous activity in SUV420H1 mutant organoids at four months in the calcium imaging analysis. As organoid models become more sophisticated in reproducing wiring maps of the endogenous brain, incorporating additional cell types (e.g., microglia, pericytes, endothelium), and developing stereotypical structure¹¹, they will empower investigation of the long-term effects of these early developmental abnormalities on circuit physiology and function.

Reproducible brain organoid models, combined with high-throughput single cell genomic methods, are suitable systems for unbiased identification of pathogenic mechanisms in neurodevelopmental disorders. This work paves the way for future efforts to multiplex perturbation and analysis of large panels of ASD risk genes in organoids to understand whether convergent mechanisms are at play across the multitudinous implicated genes, and for a deeper analysis of the effect of different genomic contexts on ASD outcome.

### Materials and Methods

### Pluripotent Stem Cell Culture

The HUES66 CHD8 parental hESC line⁶⁴ and CHD8 mutant line (HUES66 AC2), a clone which has a heterozygous 13 nucleotide deletion, resulting in a stop codon at amino acid 1248 (CHD8 gRNA: 5′-TTCTTACTGTGTACCCGGGC-3′ (TGG) (SEQ ID NO: 1)), were kindly provided by N. Sanjana, X. Shi, J. Pan, and F. Zhang (Broad Institute of MIT and Harvard). The psychiatric control Mito210 and Mito294 parental iPSC lines were provided by B. Cohen (McLean Hospital); the parental PGP1 iPSC line by G. Church (Harvard University)⁶⁵; the GM08330 iPSC line (a.k.a. GM8330-8) by M. Talkowski (MGH) and was originally from Coriell Institute and the H1 parental hESC line (a.k.a. WA01) was purchased from WiCell. Cell lines were cultured as previously described^{14,66}. Among these cell lines iPSC lines from individuals with no known history of ASD or other psychiatric condition (Mito210 and Mito294 confirmed by structured psychiatric interview, PGP1 with publicly available records) were included. All human pluripotent stem cell lines were maintained below passage 50, were negative for mycoplasma (assayed with MycoAlert PLUS Mycoplasma Detection Kit, Lonza), and karyotypically normal (G-banded karyotype test performed by WiCell Research Institute). The HUES66 and PGP1 lines were authenticated using STR analysis completed by GlobalStem (in 2008) and TRIPath (in 2018), respectively. The Mito210 and Mito294 lines were authenticated by genotyping analysis (Fluidigm FPV5 chip) performed by the Broad Institute Genomics Platform (in 2017). The H1 and GM08330 lines were authenticated using STR analysis completed by WiCell (in 2021). In Mito294 ARID1B control line a CNV smaller than 0.5 Mb on Ch19 was detected via SNP array analysis. The GM08330 parental line and modified lines have all an interstitial duplication in the long (q) arm of chromosome 20. All experiments involving human cells were performed according to ISSCR 2021 guidelines⁶⁷, and approved by the Harvard University IRB and ESCRO committees.

### CRISPR Guide Design

The CRISPR guides for SUV420H1 and ARID1B were designed using the Benchling CRISPR Guide Design Tool (Benchling Biology Software, 2017). The guides were designed to maximize on-target efficiency and minimize off-target sites in intragenic regions^{68,69}. For SUV420H1, a guide was designed to target the N-terminal domain to create a protein truncation early in the translated protein in all known protein coding transcripts (SUV420H1 gRNA: 5′-CAAGAACCAAACTGGTTGCT-3′ (AGG) (SEQ ID NO: 2)). The ARID1B guide was chosen to induce a stop codon immediately before the ARID domain (ARID1B gRNA: 5′-CTCTAGCCTGATGAACACGC-3′ (AGG) (SEQ ID NO: 4)). For CHD8, all the mutant lines were generated using the same gRNA previously used for the generation of the HUES66 AC2 (CHD8 gRNA: 5′-TTCTTACTGTGTACCCGGGC-3′ (TGG) (SEQ ID NO: 1)).

### CRISPR-Mediated Gene Editing

Mutations in SUV420H1 were introduced in the Mito210, Mito294 and PGP1 iPSC lines. For the Mito210 and Mito294 SUV420H1 mutant lines, nanoblades generated as previously described⁷⁰ were mixed with 300 μl of mTeSR1 and 4 μg/ml of polybrene and added to 80% confluent cells. For selection of the edited clones, cells were enzymatically detached and plated at a ratio of ˜5,000 cells per 60 mm dish with 10 μM of ROCK inhibitor (Y-27632, Millipore-Sigma) to increase single-cell survival. When the colonies started to appear, each clone was manually collected and transferred into a single well of a 96-well plate. During colony picking, some cells were reserved for DNA extraction and clonal screening. The PGP1 SUV420H1 mutant line was generated in collaboration with the Harvard Stem Cell Institute (HSCI) iPS Core Facility. Briefly, parental cells were transfected with the Neon system (1,000v, 1,100v or 1,200v, 30 ms, 1 pulse). For 100,000 cells 6.25 pmol TrueCut™ Cas9 Protein v2 (Thermo Fisher Cat: A36496) and 12.5 pmol of sgRNA (Synthego) were used. Post transfection, the pools of cells were harvested to test knock-out efficiency. The best pool was then selected for low density plating (600 to 2,000 cells per 10 cm dish). A week later, colonies were picked into one 96 well plate. Clones were screened by PCR and Sanger sequencing. Heterozygous clones were expanded and the genotypes were re-confirmed post expansion.

Mito210 and Mito294 ARID1B edited lines were generated by the Broad Institute Stem Cell Facility. The guide RNA and Cas9 (EnGen Cas9 NLS from New England Biolabs) were transfected by using the NEON transfection system (Thermo Fisher Scientific, 1050 V, 30 ms, 2 pulses and 2.5×10⁵ cells).

Mutations in CHD8 were introduced in the Mito210 and Mito294 lines using the Amaxa 4D-Nucleofector® (Lonza), using the protocol optimized for pluripotent stem cell lines. Parental cell lines were transfected with gRNA-CHD8-Cas92APuro and immediately plated in mTeSR1 for 24 hours. Selection of transfected cells was done by adding 0.25-0.5 μg/ml of puromycin after 48 hours of transfection, for two days. Selection of the edited clones was performed according to the protocol described for the Mito210 and Mito294 SUV420H1 clones. The H1 and GM08330 CHD8 mutant lines were generated in collaboration with the HSCI iPS Core Facility following the protocol used to generate the PGP1 SUV420H1 mutant line.

### Sequence Confirmation of Edits

Insertions/deletions in individual clones were screened via PCR amplification using primers flanking the guide. For more details about Insertions/deletions see FIG. 29.

### Organoid Differentiation

Cortical organoids were generated as previously described^{14,66}. Embryoid bodies were formed in the same pluripotent media in which they were maintained for 1-2 days in order to better enable the formation of embryoid bodies from each line.

### Immunohistochemistry

Samples were prepared as previously described¹⁴. Cryosection thickness varied from 14-18 μm.

### Whole-Organoid Imaging

Organoids were processed using the SHIELD protocol⁷¹. Briefly, organoids were fixed for 30 minutes in 4% paraformaldehyde (PFA) at room temperature (RT) and were then treated with 3% (wt/v) polyglycerol-3-polyglycidyl ether (P3PE) for 48 hours in ice cold 0.1 M phosphate buffer (pH 7.2) at 4° C. then transferred to 0.3% P3PE in 0.1 M sodium carbonate (pH 10) for 24 hours at 37° C. Samples were rinsed and cleared in 0.2 M SDS in 50 μmM phosphate buffered saline (PBS, pH 7.3) for 48 hours at 55° C. Organoids were stained with Syto16 (Thermo Fisher Scientific #S7578) and anti-SOX2 using the SmartLabel system (Lifecanvas). Tissues were washed extensively for 24 hours in PBS+0.1% Triton-X-100 and antibodies were fixed to the tissue using a 4% PFA solution overnight at RT. Tissues were refractive index-matched in PROTOS solution (RI=1.519) and imaged using a SmartSPIM axially-swept light-sheet microscope (LifeCanvas Technologies). 3D image datasets were acquired using a 15×0.4 NA objective (ASI-Special Optics, #54-10-12). Optical sections from whole-organoid datasets are shown in FIG. 13A.

Microscopy and organoid size analysis

Images of organoids in culture were taken with an EVOS FL microscope (Invitrogen), Lionheart™ FX Automated Microscope (BioTek Instruments), or with an Axio Imager.Z2 (Zeiss). Immunofluorescence images were acquired with the latter two and analyzed with the Gen5 (BioTek Instruments) or Zen Blue (Zeiss) image processing software. ImageJ⁷² was used to quantify organoid size. Area values were obtained by tracing individual organoids on ImageJ software which measured area pixels. Measurements were plotted as a ratio to the average value for control organoids of each experimental batch.

### Western Blotting

Proteins were extracted from iPSC using N-PER™ Neuronal Protein Extraction Reagent (Thermo Fisher Scientific) supplemented with protease (cOmplete™ Mini Protease Inhibitor Cocktail, Roche) and phosphatase inhibitor (PhosSTOP, Sigma). Lysates were centrifuged for 10 minutes at 13,500 rpm at 4° C. Protein concentration was quantified using the Pierce™ BCA Protein Assay Kit (Thermo Fisher Scientific). 15-20 μg of protein lysates were separated on a NuPAGE™ 4-12%, Bis-Tris Gel (Invitrogen) or Mini-PROTEAN 4-15% Gels (Bio-Rad) and transferred onto PVDF membrane. Blots were blocked with 5% nonfat dry milk (Bio-Rad) and incubated with primary antibodies overnight. Blots were then washed and incubated at room temperature (RT) with secondary horseradish peroxidase-conjugated antibodies (Abcam) for 1 hour. Blots were developed using SuperSignal™ West Femto Maximum Sensitivity Substrate (Thermo Fisher Scientific) or ECL™ Prime Western Blotting System (Millipore), and a ChemiDoc System (Bio-Rad). Densitometry band quantification was done using Fiji software⁷³ v2.0 and normalized on housekeeping genes (GAPDH or P-actin). The bands used for quantification are marked with an asterisk in FIGS. 20D-20F.

### Calcium Imaging

Organoids were transduced with pAAV-CAG-SomaGCaMP6f2 (Addgene, #158757) by pipetting 0.2 μl of stock virus to 500 μl of Cortical Differentiation Medium IV (CDMIV, without matrigel) in a 24 well containing a single organoid. On the next day, each organoid was transferred to a 6-well plate filled with 2 ml of fresh medium. On the third day after transduction, organoids were transferred to low attachment 10-cm plates and on the seventh day, medium was switched to BrainPhys (5790 STEMCELL Technologies) supplemented with 1% N2 (17502-048 Thermo Fisher), 1% B27 (17504044 Thermo Fisher), GDNF (20 ng/ml, Cat. No. 78139 STEMCELL Technologies), BDNF (20 ng/ml, 450-02 Peprotech), cAMP (1 mM, 100-0244 Stemcell Technologies), Ascorbic acid (200 nM, Cat. No. 72132 STEMCELL Technologies), and laminin (1 μg/ml, 23017015 Life Technologies). Organoids were cultured in BrainPhys for at least 2 weeks before imaging.

Brain organoids were randomly selected and transferred to a recording chamber containing BrainPhys. Imaging was performed using a confocal scanner (CSU-W1, Andor confocal unit attached on an inverted microscope [Ti-Eclipse, Nikon]), while the organoids were kept at 37° C. using a heating platform and a controller (TC-324C, Warner Instruments). The use of a 10× objective (Plan Apo λ, 10×/0.45) resulted in a field of view of 1.3×1.3 mm² and a pixel size of 0.6 μm. Imaging took place in fast-time-lapse mode, with an exposure time of 100 ms, resulting in an acquisition rate of 8 frames/sec. Spontaneous activity was recorded in three different z-planes, for at least 22 minutes of baseline activity in total (with no pharmacology treatment).

Stock solutions of 2,3-dioxo-6-nitro-1,2,3,4-tetrahydrobenzo[f]quinoxaline-7-sulfonamide disodium salt (NBQX disodium salt, Abcam; 100 mM) and Tetrodotoxin citrate (TTX, Abcam; 10 mM) were prepared in ddH₂O. Bath application of NBQX (antagonist of AMPA/kainate glutamate receptors) and TTX (voltage-gated sodium-channel antagonist) was applied to achieve a final bath concentration of 20 μM and 2 μM, respectively.

Data were converted from nd2 format to tiff, and automated motion correction and cell segmentation were performed using Suite2p⁷⁴, followed by manual curation of segmented cells (the spatial footprint and temporal characteristics of each candidate cell was examined, as well as manually adding neurons with clear cell body morphology (see FIG. 14D)). Then, mean raw fluorescence for each cell was measured as a function of time.

### Analysis of Calcium Imaging Data

Analysis was done using in-house MATLAB scripts. Raw calcium signals for each cell, F(t), were converted to represent changes from baseline level, ΔF/F(t) defined as (F(t) −Fo(t))/Fo(t). The time varying baseline fluorescence, Fo(t), for each cell was a smoothed fluorescence trace obtained after applying a 10-second-order median filter centered at t. Calcium events elicited by action potentials were detected based on a threshold value given by their peak amplitude (5 times the standard deviation of the noise value) and their first time derivative (2.5 times the standard deviation of the noise value).

The analysis of network bursting was performed based on the population-averaged calcium signal along all segmented cells. A peak in the population signal was considered a network burst if it met the following criteria: (1) the peak amplitude was greater than 10 times the standard deviation of the noise value, (2) a set of bursting cells composed of at least 20% of total cells were active during that population calcium transient, and (3) a cell was considered part of the set of bursting cells only if it participated in at least 50% of the network bursts. Under these criteria, 89.3%±14% (range from 60.5% to 100%) and 95.5%±6.8% (range from 77.6% to 100%) of segmented cells participated in network bursting in control and mutant organoids, respectively.

The peaks of the network bursts were used to measure the inter-spike interval (ISI), and the burst frequency was obtained from the average ISI. The burst half-width was also measured from the population-averaged calcium signal by calculating the width of the transient at 50% of the burst peak amplitude.

For analyses of the synchronicity, the ΔF/F(t) signal was used to calculate the cross-correlation between all pairs of cells at zero lag (FIG. 24C) as well as the cross-correlogram between a reference cell and the rest of the cells (FIG. 24D). Along with the original signal, 10 active cells were randomly selected, their ΔF/F(t) signal was circularly shifted by random phases (keeping their internal temporal structure but altering their temporal relationship with the network) and they were used as control.

### Multi-Electrode Array

Extracellular neurophysiological signals were recorded using a Maxwell Biosystems CMOS-HD-MEA system⁷⁵ (MaxOne System, MaxWell Biosystems AG, Switzerland). MaxOne chip contains 26,400 platinum electrodes in a sensing area of 3.85×2.10 mm² with 17.5 m center-to-center pitch, 3265 electrodes/mm² density, and 1024 configurable low noise readout channels (2.4 μVr·m·s. in the 300 Hz-10 kHz band) with a sampling rate of 20 kHz/s at 10-bit resolution. Acute recordings were performed at room temperature, with the intact organoid in fresh BrainPhys.

For the recordings, MaxLab Live Software (v.20.1.6. MaxWell Biosystems AG, Switzerland) was used. Briefly, spontaneous activity of neurons was measured using the Activity Scan Assay where the whole chip area was scanned with a sparse recording (30 s/configuration, seven configurations). Active neurons were automatically identified, based on the firing rate and spike amplitude obtained from the Activity Scan. Based on the activity of the neurons, the most active electrodes were routed for the creation of the network configuration based on units of 4×3 electrodes each, with 1024 recording electrodes in total (FIG. 24E top). Selected electrodes were then simultaneously recorded using the Network Assay to investigate the spontaneous neuronal network activity.

For spike detection, the software used a finite impulse response bandpass filter between 300-3000 Hz to pre-process the raw data (FIG. 24E middle). The root mean square (RMS) noise per electrode was calculated and every negative peak larger than 6 RMS was considered a spike.

When extracting the waveform of the electrodes in a single unit (set of neighboring 4×3 electrodes; FIG. 24E bottom), the spike time of one selected electrode was used as a reference to extract the simultaneous signal across the different electrodes (instead of using their individual spike times).

All descriptive statistics and statistical tests were performed in Matlab (Version 9.5, R2018b, The MathWorks, Inc.), using the Statistics Toolbox (version 11.4, R2018b, The MathWorks, Inc.). The Lilliefors test was used to test for normality of data distributions. All datasets met the assumptions of the applied statistical tests. When comparing groups, the equality of the variance was tested at the 5% significance level by a two-tailed squared-ranks test. All statistical tests applied to the electrophysiological data were two-tailed, with a 5% significance level.

### Cell Lysis and Filter-Aided Sample Preparation (FASP) Digestion for Mass Spectrometry

For SUV420H1, 4 mutant and 4 control organoids, for CHD8, 3 mutant and 3 control organoids and for ARID1B, 5 mutant and 4 control organoids were used. Cells were placed into microTUBE-15 (Covaris) microtubes with TPP buffer (truXTRAC Protein Extraction Buffer TP, Covaris SKU: 520103) and lysed using a S220 Focused-ultrasonicator instrument (Covaris) with 125 W power over 180 seconds at 10% max peak power. Upon precipitation with chloroform/methanol, extracted proteins were weighed and digested according to the FASP protocol^{76,77} (100 μg for ARID1B and CHD8; 150 μg for SUV420H1). Briefly, the 10K filter was washed with 100 μl of 50 μmM triethylammonium bicarbonate (TEAB). Each sample was added, centrifuged, and the supernatant discarded. Then, 100 μl of 20 mM Tris (2-carboxyethyl) phosphine (TCEP) at 37° C. was added for one hour, centrifuged, and the supernatant discarded. While shielding from light, 100 μl of 10 mM IAcNH₂ was added for 1 hour followed by spinning and discarding the supernatant. Next, 150 μl of 50 μmM TEAB+3 μg of Sequencing Grade Trypsin (Promega) was added to each sample and left overnight at 38° C. The samples were then centrifuged and the supernatants collected. Finally, 50 μl of 50 μmM TEAB was added to the samples, followed by spinning and supernatant collection. The samples were then transferred to HPLC.

### TMT Mass Tagging Protocol Peptide Labeling

TMT (Tandem Mass Tag) Label Reagents (TMTPro, ThermoFisher Scientific, 16plex Label Reagent Set Catalog number: A44521) were equilibrated to RT and resuspended in anhydrous acetonitrile or ethanol (for the 0.8 and 5 mg vials, 41 μl and 256 μl were added, respectively). The reagent was dissolved for 5 minutes with occasional vortexing. TMT Label Reagent (41 μl, equivalent to 0.8 mg) was added to each 100-150 μg sample. The reaction was incubated for one hour at RT. The reaction was quenched after adding 8 μl of 5% hydroxylamine to the sample and incubating for 15 minutes. Samples were combined, dried in a speedvac (Eppendorf) and stored at −80° C.

### Hi-pH Separation and Mass Spectrometry Analysis

Before submission to Liquid Chromatography with tandem mass spectrometry (LC-MS/MS), each experiment sample was separated on a Hi-pH column (Thermo Fisher Scientific) according to the vendor's instructions. After separation into 40 (20 for the ARID1B experiment) fractions, each fraction was submitted for a single LC-MS/MS experiment performed on a Lumos Tribrid (Thermo Fisher Scientific) equipped with 3000 Ultima Dual nanoHPLC pump (Thermo Fisher Scientific). Peptides were separated onto a 150 μm inner diameter microcapillary trapping column packed first with approximately 3 cm of C18 Reprosil resin (5 μm, 100 Å, Dr. Maisch GmbH) followed by PharmaFluidics micropack analytical 50 cm column. Separation was achieved by applying a gradient from 5-27% acetonitrile (ACN) in 0.1% formic acid over 90 minutes at 200 nl per minute. Electrospray ionization was enabled by applying a voltage of 1.8 kV using a home-made electrode junction at the end of the microcapillary column and sprayed from stainless-steel tips (PepSep). The Lumos Orbitrap was operated in data-dependent mode for the MS methods. The MS survey scan was performed in the Orbitrap in the range of 400-1,800 m/z at a resolution of 6×104, followed by the selection of the 20 most intense ions (TOP20) for CID-MS2 fragmentation in the Ion trap using a precursor isolation width window of 2 m/z, AGC setting of 10,000, and a maximum ion accumulation of 50 μms. Singly-charged ion species were not subjected to CID fragmentation. Normalized collision energy was set to 35 V and an activation time of 10 ms. Ions in a 10 ppm m/z window around ions selected for MS2 were excluded from further selection for fragmentation for 90 seconds. The same TOP20 ions were subjected to HCD MS2 events in the Orbitrap part of the instrument. The fragment ion isolation width was set to 0.8 m/z, AGC was set to 50,000, the maximum ion time was 150 μms, normalized collision energy was set to 34 V and an activation time of 1 ms for each HCD MS2 scan.

### Mass Spectrometry Data Generation

Raw data were submitted for analysis in Proteome Discoverer 2.4 software (Thermo Fisher Scientific). Assignment of MS/MS spectra was performed using the Sequest HT algorithm by searching the data against a protein sequence database including all entries from the Human Uniprot database^{78,79} and other known contaminants such as human keratins and common lab contaminants. Sequest HT searches were performed using a 10 ppm precursor ion tolerance and requiring each peptides N-/C termini to adhere with Trypsin protease specificity, while allowing up to two missed cleavages. 16-plex TMTpro tags on peptide N termini and lysine residues (+304.207 Da) was set as static modifications while methionine oxidation (+15.99492 Da) was set as variable modification. A MS2 spectra assignment false discovery rate (FDR) of 1% on protein level was achieved by applying the target-decoy database search. Filtering was performed using a Percolator (64 bit version)⁸⁰. For quantification, a 0.02 m/z window centered on the theoretical m/z value of each of the 6 reporter ions and the intensity of the signal closest to the theoretical m/z value was recorded. Reporter ion intensities were exported in the result file of Proteome Discoverer 2.4 search engine as Excel tables. The total signal intensity across all peptides quantified was summed for each TMT channel, and all intensity values were normalized to account for potentially uneven TMT labeling and/or sample handling variance for each labeled channel.

### Mass Spectrometry Data Analysis

Potential contaminants were filtered out and proteins supported by at least two unique peptides for the SUV420H1 and CHD8 experiment and at least one for the ARID1B experiment were used for further analysis. Proteins that were missing were kept in at most one sample per condition. Data were transformed and normalized using variance stabilizing normalization using the DEP package of Bioconductor⁸¹. To perform statistical analysis, data were imputed for missing values using random draws from a Gaussian distribution with 0.3 width and a mean that was down-shifted from the sample mean by 1.8. To detect statistically significant differential protein abundance between conditions, a moderated t-test was performed using the LIMMA package of Bioconductor⁸², employing an FDR threshold of 0.1. GSEA was performed using the GSEA software⁸³. GO and KEGG pathway annotation were utilized to perform functional annotation of the significantly regulated proteins. GO terms and KEGG pathways with FDR q-values <0.05 were considered statistically significant.

To build protein interaction networks, the prize-collecting Steiner forest algorithm was used^{50,84} using the top 50 DEPs (ranked by adjusted p value) from each mutation as terminals, with the absolute value of their log fold change as prizes. This algorithm optimizes the network to include high-confidence protein interactions between protein nodes with large prizes. The PCSF R package v0.99.1⁸⁵ was used to calculate networks, with the STRING database as a background protein-protein interactome⁵, using parameters n=10, r=0.1, w=2, b=40, and mu=0.01. As by default in that package, the network was subclustered using the edge-betweenness clustering algorithm from the igraph package, and functional enrichment was performed on each cluster using the ENRICHR API. Cytoscape software version 3.8.2 was used for network visualization⁸⁶. To assess relationships between the three sets of differential proteins, a PPI-weighted gene distance⁵² was calculated between each pair of protein sets. A background distribution was calculated by drawing size-matched random lists of proteins from all detected proteins in each dataset and calculating the pMM between these sets. This was repeated 1000 times, and an empiracal p-value was calculated by evaluating the number of times randomized pMMs were lower than the value calculated using DEPs.

Dissociation of Brain Organoids and scRNA-Seq

Organoids were dissociated as previously described^{66,87}. Volumes of reagents were scaled down 25× for one month old organoids. Cells were loaded onto either a Chromium™ Single Cell B or G Chip (10× Genomics, PN-1000153, PN-1000120), and processed through the Chromium Controller to generate single cell GEMs (Gel Beads in Emulsion). scRNA-seq libraries were generated with the Chromium™ Single Cell 3′ Library & Gel Bead Kit v3 or v3.1 (10× Genomics, PN-1000075, PN-1000121), with the exception of a few libraries in the earlier experiments that were prepared with a v2 kit (10× Genomics, PN-120237). Libraries were pooled from different samples based on molar concentrations and sequenced them on a NextSeq 500 or NovaSeq instrument (Illumina) with 28 bases for read 1 (26 bases for v2 libraries), 55 bases for read 2 (57 bases for v2 libraries) and 8 bases for Index 1. If necessary, after the first round of sequencing, libraries were re-pooled based on the actual number of cells in each and re-sequenced with the goal of producing approximately equal number of reads per cell for each sample.

scRNA-Seq Data Analysis

Reads from scRNA-seq were aligned to the GRCh38 human reference genome and the cell-by-gene count matrices were produced with the Cell Ranger pipeline (10× Genomics)⁸⁸. Cell Ranger version 2.0.1 was used for experiments using the GM08330 control “single cell map” and for HUES66 CHD8 mutant and control organoids at 3.5 months, batch I, while version 3.0.2 was used for all other experiments. Default parameters were used, except for the ‘-cells’ argument. Data was analyzed using the Seurat R package v3.1.5⁸⁹ using R v3.6. Cells expressing a minimum of 500 genes were kept, and UMI counts were normalized for each cell by the total expression, multiplied by 10⁶, and log-transformed. Variable genes were found using the “mean.var.plot” method, and the ScaleData function was used to regress out variation due to differences in total UMIs per cell. Principal component analysis (PCA) was performed on the scaled data for the variable genes, and top principal components were chosen based on Seurat's ElbowPlots (at least 15 PCs were used in all cases). Cells were clustered in PCA space using Seurat's FindNeighbors on top principal components, followed by FindClusters with resolution=1.0 (briefly, a 20-nearest neighbor graph was constructed and modularity optimization using the Louvain algorithm was performed to identify clusters). Variation in the cells was visualized by t-distributed stochastic neighbor embedding (t-SNE) on the top principal components.

In the case of the GM08330 one month organoids (single-cell map), cells were demultiplexed using genotype clustering from cells from a different experiment that were sequenced in the same lane. To demultiplex, SNPs were called from CellRanger BAM files with the cellSNP tool v0.1.5, and then the vireo function was used with default parameters and n_donor=2, from the cardelino R library v0.4.0^{90.91} to assign cells to each genotype.

In two cases, one organoid was excluded from the analysis as outliers. See the Statistics & Reproducibility section for details.

For each dataset, upregulated genes in each cluster were identified using the VeniceMarker tool from the Signac package v0.0.7 from BioTuring (available at github.com/bioturing/signac). Cell types were assigned to each cluster by looking at the top most significant upregulated genes. In a few cases, clusters were further subclustered to assign identities at higher resolution. At one month, the excitatory projection neurons included a gradient of immature neurons, which were split into two clusters: the cluster representing the earlier developmental stage was labeled “newborn deep layer projection neurons” and the cluster representing the later stage was labeled “immature deep layer projection neurons”. At three months and beyond, excitatory projection neuron clusters could be identified as deep layer corticofugal neurons and upper-layer callosal projection neurons. For the GABAergic populations, one month organoids included neurons expressing broad markers of GABAergic identity (labelled as “GABAergic neurons”), progenitor cells expressing markers of GABAergic lineage identity (“GABAergic Neuron Progenitors”), and progenitor cells with high expression of cell cycle markers in addition to the progenitor identity markers (“Cycling GABAergic Neuron Progenitors”). At three months and beyond, GABAergic neurons expressed more specific markers of cortical interneurons (hence labelled “GABAergic Interneurons”), and GABAergic lineage progenitors at these ages were divided into “GABAergic Interneuron Progenitors” and “Cycling GABAergic Interneuron Progenitors”, based on level of expression of cell cycle markers.

To assess gene expression of ASD risk genes in GM08330 and Mito210 control organoids across timepoints, datasets from one, three, and six months were merged using Seurat v3.1.5, then batch corrected using Harmony v1.0 with default parameters⁹². Since the one month data are dominated by cell cycle signal, the ScaleData function was used to regress out variation due to both total UMI count per cell and to cell cycle stage differences, calculated using Seurat's CellCycleScore. Variation was visualized using t-SNE on the first 30 Harmony dimensions. Broad cell types were assigned as above, and mutual information was calculated between cell type assignments and individual organoids with the mpmi R package⁹³. Expression of the 102 ASD risk genes identified in the Satterstrom et. al. study⁶ was evaluated using Seurat's AddModuleScore function using default parameters. This function calculates the average expression level per cell of the set of genes (based on log-normalized, unscaled data), and then subtracts the average expression of a randomly-selected expression-matched control set of genes. A resulting score above zero indicates that the ASD risk gene set is expressed more highly in that cell than would be expected, given the average expression of the gene set across the dataset.

To compare cell type proportions between control and mutant organoids, for each cell type present in a dataset, the glmer function from the R package lme4 v1.1-2394 was used to estimate a mixed-effect logistic regression model⁹⁵. The output was a binary indicator of whether cells belong to this cell type, the control or mutant state of the cell was a fixed predictor, and the organoid that the cell belonged to was a random intercept. Another model was fit without the control-versus-mutant predictor, and the anova function was used to compare the two model fits. P-values for each cell type were then adjusted for multiple hypothesis testing using the Benjamini-Hochberg correction.

### Single Nucleus Isolation and Single-Cell ATAC-Seq

Nuclei from one month and three month organoids were extracted with two types of procedures according to their size differences. For the one month organoids, the nuclei were extracted following a protocol provided by 10× Genomics⁹⁶ to minimize material loss, while a sucrose-based nucleus isolation protocol⁹⁷ was used for the three month organoids to better remove debris. Single-nucleus ATAC-Seq libraries were prepared with the Chromium™ Single Cell ATAC Library & Gel Bead v1 Kit (10× Genomics, PN-1000110) and around 15,300 nuclei per channel were loaded to give an estimated recovery of 10,000 nuclei per channel. Libraries from different samples were pooled based on molar concentrations and sequenced with 1% PhiX spike-in on a NextSeq 500 instrument (Illumina) with 33 bases each for read 1 and read 2, 8 bases for Index 1 and 16 bases for Index 2.

### Single-Cell ATAC-Seq Data Analysis

Reads from scATAC-seq were aligned to the GRCh38 human reference genome and the cell-by-peak count matrices were produced with the Cell Ranger ATAC pipeline v2.0.0 (10× Genomics) with default parameters. Data were analyzed using the Signac R package v1.2.1⁹⁸ using R v4.0. Annotations from the EnsDb.Hsapiens.v86 package⁹⁹ were added to the object. After consideration of QC metrics recommended in that package, cells with 1500-20,000 fragments in peak regions, at least 35% of reads in peaks, a nucleosome signal of less than 4, and a TSS Enrichment score of greater than 2 were retained for further analysis. Latent semantic indexing (LSI) was performed to reduce data dimensionality (counts were normalized using term frequency inverse document frequency, all features were set as top features, and singular value decomposition (SVD) was performed). The top LSI component was discarded as it correlated strongly with sequencing depth, and components 2-30 were used for downstream analysis. Cells were clustered using Seurat's FindNeighbors, followed by FindClusters with the SLM algorithm (a 20-nearest neighbor graph was constructed and modularity optimization using the smart local moving algorithm was performed to identify clusters). Variation in the cells was visualized by UMAP (Uniform Manifold Approximation and Projection) on the top LSI components.

ScATAC-seq data were integrated with scRNA-seq data from the corresponding Mito210 dataset for each timepoint, using Seurat's TransferData to predict cell type labels for the ATAC profiles. Concurrently, differentially accessible (DA) peaks per cluster were called using FindMarkers with the logistic regression framework with the number of fragments in peak regions as a latent variable. These DA peaks were mapped to the closest genes. Top genes per cluster were used to confirm and refine cluster cell type assignments from those based on transferring RNA labels.

DA peaks between control and SUV420H1 mutant organoids were calculated per cell type, using the same method as above. It was noticed that most cell types had very few significantly differential regions, and that these were almost entirely overlapping regions in all cell types. Therefore, differentially accessible regions were calculated using all cells together to improve power. Differentially accessible regions were visualized using Signac's CoveragePlot function with default parameters.

To find transcription factor motifs enriched in differentially accessible regions, the top 400 up- and down-regulated peaks for each time point differentially accessible peaks were supplied to the HOMER software v4.11.1¹⁰⁰, using a 300 bp fragment size and masking repeats. In the case of upregulated regions in three month mutant organoids, only 341 regions were supplied, since that was the total number of regions with log FC>0.1 and p>0.1. The top 5 de novo motifs per cell type found by HOMER with a p value <=10⁻¹⁰ are reported, along with all TFs who's known binding sites match that motif with a score >=0.59.

### Pseudotime, Gene Module, and Differential Expression Analysis

Pseudotime analysis was performed using the Monocle3 v. 0.2.0 software package³⁶ with default parameters. The cells were first subset to contain an equal amount from control and mutant. A starting point for the trajectory was chosen manually by finding an endpoint of the tree located in the earliest developmental cell type (generally, cycling progenitors). Where the cells were split into more than one partition, the starting point was chosen within the partition of interest, and a new UMAP was calculated using just these cells. To test whether mutant trajectories were accelerated compared to control, a one-sided Kolmogorov-Smirnov test was applied comparing the distribution of psuedotime values of control vs. mutant cells, using the stats R package.

In order to learn patterns of coordinated gene regulation across the cells, WGCNA³⁷ was applied to each dataset. Where cells were split into partitions in the above pseudotime analysis, only cells belonging to the partition of interest were used. Normalized gene expression data was further filtered to remove outlying genes, mitochondrial and ribosomal genes. Outliers were identified by setting the upper (>9) and lower (<0.15) thresholds to the average normalized expression per gene. After processing, blockwiseModules function from the WGCNA v1.69 library was performed in R with the parameters networkType=“signed”, minModuleSize=4, corType=“Bicor”, maxPOutliers=0.1, deepSplit=3,trapErrors=T, and randomSeed=59069. Other than power, remaining parameters were left as the default setting. To pick an adequate power for each dataset, the pickSoftThreshold function from WGCNA was used to test values from 1 to 30. Final resolution was determined by choosing the resolution that captured most variation in the fewest total number of modules—this resulted in a power of 3 for SUV420H1 35 d.i.v., and 9 for ARID1B 35 d.i.v. and 12 for CHD8 109 d.i.v.

To calculate differential expression of modules, Seurat objects were downsampled to have an equal number of cells per organoid, and then the AddModuleScore function was used, using gene lists from WGCNA results. For each module, linear mixed-effect models were fit to the data, with the modules scores as the output, the organoid the cell belongs to as a random intercept, and with or without the control-versus-mutant state as a predictor. The anova function was used to compare the models, and p-values were then adjusted across modules using the Benjamini-Hochberg correction.

Differentially expressed genes between control and mutant organoids were assessed after datasets were subset to the cells from the partition of interest in the above pseudotime analysis, to the cells from each individual cell type, or not subset at all for pseudobulk analysis. Reads were then summed across cells in each organoid. Genes with less than 10 total reads were excluded, and DESeq2⁴⁵ was used to calculate DEGs, with each organoid as a sample⁴⁶. The clusterProfiler¹⁰¹ R package was used to find enriched biological processes in these gene sets, with the enrichGO function and the compareCluster function to highlight processes the gene sets might have in common.

### Statistics and Reproducibility

For organoid size analysis, see FIG. 30 for the number of organoids used.

For the proteomic analysis, four mutant and four control organoids were used for SUV420H1. Three mutant and three control and five mutant and four control organoids were used for CHD8 and ARID1B, respectively

For scATAC-seq, three SUV420H1 mutant and three control organoids were used for each of the one month and three month time points.

Finally, in each scRNA-seq dataset, three individual organoids per genotype were profiled. In two cases, one organoid was excluded from the analysis as an outlier: in PGP1 SUV420H1 organoids at one month, a mutant organoid was excluded due to very low average nUMI and nGene in that sequencing lane, and in the HUES66 CHD8 organoids at 3.5 months batch II, a mutant organoid was excluded because it mostly contained interneuron lineage cells, with very few projection neuron cells. Although an increase in interneuron-lineage cells was seen in all mutant organoids, this organoid was excluded to be conservative. This left a total of 124 single organoids that passed quality control and were considered in downstream analysis, with a total of 710,085 cells across both scATAC-seq and scRNA-seq.

### REFERENCES

1. Lord, C. et al. Autism spectrum disorder. Nat Rev Dis Primers 6, 5 (2020).
2. Rosenberg, R. E. et al. Characteristics and concordance of autism spectrum disorders among 277 twin pairs. Arch Pediatr Adolesc Med 163, 907-914 (2009).
3. Sanders, S. J. et al. De novo mutations revealed by whole-exome sequencing are strongly associated with autism. Nature 485, 237-241 (2012).
4. Ruzzo, E. K. et al. Inherited and De Novo Genetic Risk for Autism Impacts Shared Networks. Cell 178, 850-866.e26 (2019).
5. Grove, J. et al. Identification of common genetic risk variants for autism spectrum disorder. Nat Genet 51, 431-444 (2019).
6. Satterstrom, F. K. et al. Large-Scale Exome Sequencing Study Implicates Both Developmental and Functional Changes in the Neurobiology of Autism. Cell 180, 568-584.e23 (2020).
7. Cooper, D., Krawczak, M., Polychronakos, C., Tyler-Smith, C. & Kehrer-Sawatzki, H. Where genotype is not predictive of phenotype: towards an understanding of the molecular basis of reduced penetrance in human inherited disease. Human genetics 132, 1077-1130 (2013).
8. Zlotogora, J. Penetrance and expressivity in the molecular age. Genetics in medicine: official journal of the American College of Medical Genetics 5, 347-352 (2003).
9. Pasca, S. P. The rise of three-dimensional human brain cultures. Nature 553, 437-445 (2018).
10. Lancaster, M. A. & Knoblich, J. A. Organogenesis in a dish: Modeling development and disease using organoid technologies. Science 345, 1247125 (2014).
11. Velasco, S., Paulsen, B. & Arlotta, P. 3D Brain Organoids: Studying Brain Development and Disease Outside the Embryo. Annu Rev Neurosci 43, 375-389 (2020).
12. Quadrato, G., Brown, J. & Arlotta, P. The promises and challenges of human brain organoids as models of neuropsychiatric disease. Nat Med 22, 1220-1228 (2016).
13. Bourgeron, T. From the genetic architecture to synaptic plasticity in autism spectrum disorder. Nature reviews. Neuroscience 16, 551-563 (2015).
14. Velasco, S. et al. Individual brain organoids reproducibly form cell diversity of the human cerebral cortex. Nature 570, (2019).
15. de Rubeis, S. et al. Synaptic, transcriptional and chromatin genes disrupted in autism. Nature 515, 209-215 (2014).
16. Stessman, H. A. F. et al. Targeted sequencing identifies 91 neurodevelopmental-disorder risk genes with autism and developmental-disability biases. Nature Genetics 49, 515-526 (2017).
17. Yuen, R. K. C. et al. Whole genome sequencing resource identifies 18 new candidate genes for autism spectrum disorder. Nature Neuroscience 20, 602-611 (2017).
18. Sanders, S. J. et al. Insights into Autism Spectrum Disorder Genomic Architecture and Biology from 71 Risk Loci. Neuron 87, 1215-1233 (2015).
19. Bernier, R. et al. Disruptive CHD8 mutations define a subtype of autism early in development. Cell 158, 263-276 (2014).
20. Faundes, V. et al. Histone Lysine Methylases and Demethylases in the Landscape of Human Developmental Disorders. Am J Hum Genet 102, 175-187 (2018).
21. Vals, M. et al. Coffin-Siris Syndrome with obesity, macrocephaly, hepatomegaly and hyperinsulinism caused by a mutation in the ARID1B gene. European journal of human genetics: EJHG 22, 1327-1329 (2014).
22. Iossifov, I. et al. De novo gene disruptions in children on the autistic spectrum. Neuron 74, 285-299 (2012).
23. Jørgensen, S., Schotta, G. & Sprensen, C. S. Histone H4 Lysine 20 methylation: key player in epigenetic regulation of genomic integrity. Nucleic Acids Research 41, 2797-2806 (2013).
24. Wickramasekara, R. N. & Stessman, H. A. F. Histone 4 Lysine 20 Methylation: A Case for Neurodevelopmental Disease. Biology 8, 11 (2019).
25. Nicetto, D. et al. Suv4-20h Histone Methyltransferases Promote Neuroectodermal Differentiation by Silencing the Pluripotency-Associated Oct-25 Gene. PLOS Genetics 9, e1003188 (2013).
26. Rhodes, C. T. et al. Cross-species analyses unravel the complexity of H3K27me3 and H4K20me3 in the context of neural stem progenitor cells. Neuroepigenetics 6, 10-25 (2016).
27. Mashtalir, N. et al. Modular Organization and Assembly of SWI/SNF Family Chromatin Remodeling Complexes. Cell 175, 1272-1288.e20 (2018).
28. Staahl, B. T. & Crabtree, G. R. Creating a neural specific chromatin landscape by npBAF and nBAF complexes. Current opinion in neurobiology 23, 903-913 (2013).
29. Barnard, R. A., Pomaville, M. B. & O'Roak, B. J. Mutations and Modeling of the Chromatin Remodeler CHD8 Define an Emerging Autism Etiology. Frontiers in neuroscience 9, 477 (2015).
30. Breuss, M. W. & Gleeson, J. G. When size matters: CHD8 in autism. Nat Neurosci 19, 1430-1432 (2016).
31. Wade, A. A., Lim, K., Catta-Preta, R. & Nord, A. S. Common CHD8 Genomic Targets Contrast With Model-Specific Transcriptional Impacts of CHD8 Haploinsufficiency. Frontiers in Molecular Neuroscience 11, (2019).
32. Min, Z., Qian, C. & Ying, D. Novel ARID1B variant inherited from somatogonadal mosaic mother in siblings with Coffin-Siris syndrome 1. Exp Ther Med 21, 614 (2021).
33. Nagamani, S. C. S. et al. Interstitial deletion of 6q25.2-q25.3: a novel microdeletion syndrome associated with microcephaly, developmental delay, dysmorphic features and hearing loss. European Journal of Human Genetics 17, 573 (2009).
34. Lodato, S. & Arlotta, P. Generating neuronal diversity in the mammalian cerebral cortex. Annu Rev Cell Dev Biol 31, 699-720 (2015).
35. Greig, L. C., Woodworth, M. B., Galazo, M. J., Padmanabhan, H. & Macklis, J. D. Molecular logic of neocortical projection neuron specification, development and diversity. Nat Rev Neurosci 14, 755-769 (2013).
36. Cao, J. et al. The single-cell transcriptional landscape of mammalian organogenesis. Nature 566, 496-502 (2019).
37. Langfelder, P. & Horvath, S. WGCNA: an R package for weighted correlation network analysis. BMC Bioinformatics 9, 559 (2008).
38. Shemesh, O. A. et al. Precision Calcium Imaging of Dense Neural Populations via a Cell-Body-Targeted Calcium Indicator. Neuron 107, 470-486.e11 (2020).
39. Garaschuk, O., Linn, J., Eilers, J. & Konnerth, A. Large-scale oscillatory calcium waves in the immature cortex. Nature Neuroscience 2000 3:5 3, 452-459 (2000).
40. Adelsberger, H., Garaschuk, O. & Konnerth, A. Cortical calcium waves in resting newborn mice. Nature Neuroscience 2005 8:8 8, 988-990 (2005).
41. Yang, S. M., Alvarez, D. D. & Schinder, A. F. Reliable Genetic Labeling of Adult-Born Dentate Granule Cells Using AscllCreERT2 and GlastCreERT2 Murine Lines. Journal of Neuroscience 35, 15379-15390 (2015).
42. Wang, Z.-J. et al. Autism risk gene KMT5B deficiency in prefrontal cortex induces synaptic dysfunction and social deficits via alterations of DNA repair and gene transcription. Neuropsychopharmacology 2021 46:9 46, 1617-1626 (2021).
43. Villa, C. E. et al. CHD8 haploinsufficiency alters the developmental trajectories of human excitatory and inhibitory neurons linking autism phenotypes with transient cellular defects. bioRxiv 2020.11.26.399469 (2020) doi:10.1101/2020.11.26.399469.
44. Wang, P. et al. CRISPR/Cas9-mediated heterozygous knockout of the autism gene CHD8 and characterization of its transcriptional networks in cerebral organoids derived from iPS cells. Mol Autism 8, 11 (2017).
45. Love, M. I., Huber, W. & Anders, S. Moderated estimation of fold change and dispersion for RNA-seq data with DESeq2. Genome Biology 15, 550 (2014).
46. Lun, A. T. L. & Marioni, J. C. Overcoming confounding plate effects in differential expression analyses of single-cell RNA-seq data. Biostatistics 18, 451-464 (2017).
47. Lim, L., Mi, D., Llorca, A. & Marin, O. Development and Functional Diversification of Cortical Interneurons. Neuron 100, 294-313 (2018).
48. Xiao, A. et al. WSTF regulates the H2A.X DNA damage response via a novel tyrosine kinase activity. Nature 2008 457:7225 457, 57-62 (2008).
49. Ji, J.-H. et al. De novo phosphorylation of H2AX by WSTF regulates transcription-coupled homologous recombination repair. Nucleic Acids Research 47, 6299-6314 (2019).
50. Tuncbag, N. et al. Network-Based Interpretation of Diverse High-Throughput Datasets through the Omics Integrator Software Package. PLoS Computational Biology 12, (2016).
51. Szklarczyk, D. et al. STRING v11: protein-protein association networks with increased coverage, supporting functional discovery in genome-wide experimental datasets. Nucleic Acids Res 47, D607-d613 (2019).
52. Yoon, S. et al. GScluster: network-weighted gene-set clustering analysis. BMC Genomics 20, 352 (2019).
53. Velmeshev, D. et al. Single-cell genomics identifies cell type-specific molecular changes in autism. Science 364, 685-689 (2019).
54. Mariani, J. et al. FOXG1-Dependent Dysregulation of GABA/Glutamate Neuron Differentiation in Autism Spectrum Disorders. Cell 162, 375-390 (2015).
55. Marchetto, M. C. et al. Altered proliferation and networks in neural cells derived from idiopathic autistic individuals. Mol Psychiatry 22, 820-835 (2017).
56. Adhya, D. et al. Atypical Neurogenesis in Induced Pluripotent Stem Cells From Autistic Individuals. Biol Psychiatry (2020) doi:10.1016/j.biopsych.2020.06.014.
57. Moffat, J. J., Smith, A. L., Jung, E.-M., Ka, M. & Kim, W.-Y. Neurobiology of ARID1B haploinsufficiency related to neurodevelopmental and psychiatric disorders. Molecular Psychiatry 20211-14 (2021) doi:10.1038/s41380-021-01060-x.
58. Rubenstein, J. L. R. & Merzenich, M. M. Model of autism: increased ratio of excitation/inhibition in key neural systems. Genes, Brain and Behavior 2, 255-267 (2003).
59. Gogolla, N. et al. Common circuit defect of excitatory-inhibitory balance in mouse models of autism. Journal of neurodevelopmental disorders 1, 172-181 (2009).
60. Dani, V. S. et al. Reduced cortical activity due to a shift in the balance between excitation and inhibition in a mouse model of Rett syndrome. Proc Natl Acad Sci USA 102, 12560-12565 (2005).
61. Bocchi, R. et al. Perturbed Wnt signaling leads to neuronal migration delay, altered interhemispheric connections and impaired social behavior. Nature Communications 8, 1158 (2017).
62. Lodato, S. et al. Excitatory projection neuron subtypes control the distribution of local inhibitory interneurons in the cerebral cortex. Neuron 69, 763-779 (2011).
63. Chan, W. K., Griffiths, R., Price, D. J. & Mason, J. O. Cerebral organoids as tools to identify the developmental roots of autism. Molecular Autism 2020 11:1 11, 1-14 (2020).
64. Chen, A. E. et al. Optimal timing of inner cell mass isolation increases the efficiency of human embryonic stem cell derivation and allows generation of sibling cell lines. Cell stem cell 4, 103-106 (2009).
65. Church, G. M. The personal genome project. Molecular systems biology 1, 2005.0030-2005.0030 (2005).
66. Velasco, S., Paulsen, B. & Arlotta, P. Highly reproducible human brain organoids recapitulate cerebral cortex cellular diversity. Protocol Exchange (2019) doi:10.21203/rs.2.9542/v1.
67. Lovell-Badge, R. et al. ISSCR Guidelines for Stem Cell Research and Clinical Translation: The 2021 update. Stem Cell Reports 16, 1398-1408 (2021).
68. Doench, J. G. et al. Rational design of highly active sgRNAs for CRISPR-Cas9-mediated gene inactivation. Nature Biotechnology 32, 1262-1267 (2014).
69. Hsu, P. D. et al. DNA targeting specificity of RNA-guided Cas9 nucleases. Nature Biotechnology 31, 827-832 (2013).
70. Mangeot, P. E. et al. Genome editing in primary cells and in vivo using viral-derived Nanoblades loaded with Cas9-sgRNA ribonucleoproteins. Nature Communications 10, 45 (2019).
71. Park, Y.-G. et al. Protection of tissue physicochemical properties using polyfunctional crosslinkers. Nature Biotechnology 37, 73-83 (2019).
72. Schneider, C. A., Rasband, W. S. & Eliceiri, K. W. NIH Image to ImageJ: 25 years of image analysis. Nat Methods 9, 671-675 (2012).
73. Ohgane, K. Quantification of Gel Bands by an Image J Macro, Band/Peak Quantification Tool. (2019) doi:10.17504/PROTOCOLS.I0.7VGHN3W.
74. Pachitariu, M. et al. Suite2p: beyond 10,000 neurons with standard two-photon microscopy. bioRxiv 061507 (2017) doi:10.1101/061507.
75. Müller, J. et al. High-resolution CMOS MEA platform to study neurons at subcellular, cellular, and network levels. Lab on a Chip 15, 2767-2780 (2015).
76. UWPR. https://proteomicsresource.washington.edu/protocols03/FASPprotocols.php.
77. Wisniewski, J. R. Quantitative Evaluation of Filter Aided Sample Preparation (FASP) and Multienzyme Digestion FASP Protocols. Analytical Chemistry 88, 5438-5443 (2016).
78. Bairoch, A. & Apweiler, R. The SWISS-PROT protein sequence data bank and its supplement TrEMBL in 1999. Nucleic Acids Res 27, 49-54 (1999).
79. Consortium, T. U. UniProt: a worldwide hub of protein knowledge. Nucleic Acids Research 47, D506-D515 (2018).
80. Kall, L., Storey, J. D., MacCoss, M. J. & Noble, W. S. Posterior error probabilities and false discovery rates: two sides of the same coin. J Proteome Res 7, 40-44 (2008).
81. Zhang, X. et al. Proteome-wide identification of ubiquitin interactions using UbIA-MS. Nature Protocols 13, 530-550 (2018).
82. Ritchie, M. E. et al. limma powers differential expression analyses for RNA-sequencing and microarray studies. Nucleic acids research 43, e47 (2015).
83. Subramanian, A. et al. Gene set enrichment analysis: A knowledge-based approach for interpreting genome-wide expression profiles. Proceedings of the National Academy of Sciences 102, 15545-15550 (2005).
84. Tuncbag, N. et al. Simultaneous reconstruction of multiple signaling pathways via the prize-collecting steiner forest problem. Journal of computational biology 20, 124-36(2013).
85. Akhmedov, M. et al. PCSF: An R-package for network-based interpretation of high-throughput data. PLoS Computational Biology 13, (2017).
86. Shannon, P. et al. Cytoscape: A software Environment for integrated models of biomolecular interaction networks. Genome Research 13, 2498-2504 (2003).
87. Quadrato, G., Sherwood, J. L. & Arlotta, P. Long term culture and electrophysiological characterization of human brain organoids. Protocol Exchange (2017) doi:10.1038/protex.2017.049.
88. Zheng, G. X. Y. et al. Massively parallel digital transcriptional profiling of single cells. Nature Communications 8, 1-12 (2017).
89. Stuart, T. et al. Comprehensive Integration of Single-Cell Data. Cell 177, 1888-1902.e21 (2019).
90. McCarthy, D. J. et al. Cardelino: computational integration of somatic clonal substructure and single-cell transcriptomes. Nat Methods 17, 414-421 (2020).
91. Huang, Y., McCarthy, D. J. & Stegle, O. Vireo: Bayesian demultiplexing of pooled single-cell RNA-seq data without genotype reference. Genome Biology 20, 273 (2019).
92. Korsunsky, I. et al. Fast, sensitive and accurate integration of single-cell data with Harmony. Nature Methods 16, 1289-1296 (2019).
93. Pardy, C. mpmi: Mixed-Pair Mutual Information Estimators. (2020).
94. Bates, D., Mächler, M., Bolker, B. & Walker, S. Fitting Linear Mixed-Effects Models Using lme4. 2015 67, 48.
95. Fonseka, C. Y. et al. Mixed-effects association of single cells identifies an expanded effector CD4+ T cell subset in rheumatoid arthritis. Science Translational Medicine 10, (2018).
96. Genomics, 10×. Nuclei Isolation from Mouse Brain Tissue for Single Cell ATAC Sequencing Rev B. Demonstrated Protocol available at assets.ctfassets.net/an68im79xiti/zZh2iRV5TgWP8A96Easg6/2286fafe2eae406b0315 67a16272b8ab/CG000212_SingleCellATAC_Nuclei_Isolation_MouseBrain_Demons tratedProtocol_RevB.pdf (2019).
97. Corces, M. R. et al. An improved ATAC-seq protocol reduces background and enables interrogation of frozen tissues. Nature Methods 14, 959-962 (2017).
98. Stuart, T., Srivastava, A., Lareau, C. & Satija, R. Multimodal single-cell chromatin analysis with Signac. bioRxiv 2020.11.09.373613 (2020) doi:10.1101/2020.11.09.373613.
99. Rainer, J. EnsDb.Hsapiens.v86: Ensembl based annotation package. (2017).
100. Heinz, S. et al. Simple combinations of lineage-determining transcription factors prime cis-regulatory elements required for macrophage and B cell identities. Mol Cell 38, 576-589 (2010).
101. Yu, G., Wang, L.-G., Han, Y. & He, Q.-Y. clusterProfiler: an R package for comparing biological themes among gene clusters. Omics: a journal of integrative biology 16, 284-287 (2012).

What is claimed is:

1. A method of screening for pathological mechanisms in neurodevelopmental disorders comprising
a. introducing one or more genetic variations to an organoid; and
b. profiling the mutant organoids using single-cell RNA sequencing, thereby identifying phenotypic alterations as a result of the genetic variation.

2. The method of claim 1, wherein the genetic variation causes haploinsufficiency in a gene.

3. The method of claim 2, wherein the gene is selected from the group consisting of SUV420H1, PTEN, ARID1B and CHD8.

4. The method of claim 2, wherein the gene is selected from the group consisting of SUV420H1, ARID1B and CHD8.

5. The method of any one of claims 1-4, wherein the neurodevelopmental disorder is autism spectrum disorder (ASD).

6. The method of any one of claims 1-5, wherein the mutant organoids are screened one month after introduction of the genetic variation.

7. The method of any one of claims 1-5, wherein the mutant organoids are screened three months after introduction of the genetic variation.

8. The method of any one of claims 1-5, wherein the mutant organoids are screened six months after introduction of the genetic variation.

9. The method of any one of claims 1-8, wherein the mutant organoids are further profiled using immunohistochemistry.

10. The method of any one of claims 1-9, wherein the mutant organoids are further profiled using whole-proteome analysis.

11. The method of any one of claims 1-10, wherein the mutant organoids are further profiled using single cell Assay for Transposase-Accessible Chromatin sequencing (ATAC-seq).

12. The method of any one of claims 1-11, wherein the mutant organoids are further profiled using electrophysiological analysis.

13. The method of claim 1, wherein the phenotypic alternation comprises asynchronous development of a cortical neuronal lineage.

14. The method of claim 1, wherein the phenotypic alteration comprises asynchronous development of deep layer projection neuron lineage.

15. The method of claim 1, wherein the phenotypic alteration comprises premature expansion of GABAergic neuron lineage.

16. A method of screening a therapeutic agent, the method comprising:
a. contacting an organoid having one or more genetic variations with one or more candidate agents; and
b. testing effects of the one or more candidate agents on asynchronous development of a cortical neuronal lineage.

17. The method of claim 16, wherein the asynchronous development of a cortical neuronal lineage comprises asynchronous development of deep layer projection neuron lineage.

18. The method of claim 16, wherein the asynchronous development of a cortical neuronal lineage comprises premature expansion of GABAergic neuron lineage.

19. The method of claim 16, wherein the genetic variation causes haploinsufficiency in a gene.

20. The method of claim 19, wherein the gene is selected from the group consisting of SUV420H1, ARID1B and CHD8.
